(19) United States
(12) Patent Application Publication
(10) Pub. No.: US 2012/0245137 A1
(43) Pub. Date: Sep. 27, 2012

(54) ARYL SULPHONE DERIVATIVES AS CALCIUM CHANNEL BLOCKERS

(75) Inventors: Hassan A. Pajouhesh, West Vancouver (CA); Robert Galemmo, JR., Vancouver (CA); Richard Holland, Vancouver (CA); Yuanxi Zhou, Richmond (CA); Yongbao Zhu, Langley (CA); Eric Simonson, Vancouver (CA); Navjot Chahal, Vancouver (CA); Mike Grimwood, North Vancouver (CA)

(73) Assignee: Zalicus Pharmaceuticals Ltd., Vancover, BC (CA)

(21) Appl. No.: 13/496,729

(22) PCT Filed: Sep. 17, 2010
(86) PCT No.: PCT/US10/49339

§ 371 Date: May 17, 2012

Related U.S. Application Data
(60) Provisional application No. 61/243,973, filed on Sep. 18, 2009.

(51) Int. Cl. A61K 31/44; C07D 239/26; A61K 31/505; C07C 317/48; A61K 31/166; C07D 295/13; A61K 31/5375; C07D 205/04; A61K 31/397; C07D 265/36; A61K 31/538; A61P 25/00; A61P 25/08; A61P 25/16; A61P 25/24; A61P 25/18; A61P 25/06; C07D 213/82

(52) U.S. Cl. 514/210.17; 546/316; 514/355; 544/335; 514/256; 564/184; 514/617; 544/159; 514/237.5; 548/953; 544/105; 514/230.5

(57) ABSTRACT

Methods and compounds effective in ameliorating conditions characterized by unwanted calcium channel activity, particularly unwanted N-type and/or T-type calcium channel activity, are disclosed. Specifically, a series of compounds containing aryl sulphone derivatives, as exemplified by Formula (I).

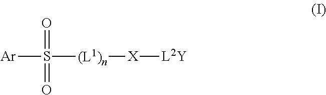

### CROSS-REFERENCE TO RELATED APPLICATIONS

This application claims benefit of U.S. Provisional Application No. 61/243,973, filed Sep. 18, 2009, which is hereby incorporated by reference.

### TECHNICAL FIELD

The invention relates to compounds useful in treating conditions associated with calcium channel function, and particularly conditions associated with N and T-type calcium channel activity. More specifically, the invention concerns compounds containing cycloalkyl aryl sulphone derivatives that are useful in treatment of conditions such as cardiovascular disease, epilepsy, cancer and pain.

### BACKGROUND OF THE INVENTION

Calcium channels mediate a variety of normal physiological functions and are also implicated in a number of human disorders. Examples of calcium-mediated human disorders include but are not limited to congenital migraine, cerebellar ataxia, angina, epilepsy, hypertension, ischemia, and some arrhythmias (see, e.g., Janis et al., Ion Calcium Channels: Their Properties, Functions, Regulation and Clinical Relevance (1991) CRC Press, London). T-type, or low voltage-activated, channels describe a broad class of molecules that transiently activate at negative potentials and are highly sensitive to changes in resting potential and are involved in various medical conditions. For example, in mice lacking the gene expressing the 3.1 subunit (Caᵥ 3.1), resistance to absence seizures was observed (Kim et al., Mol Cell Neurosci 18(2): 235-245, 2001). Other studies have also implicated the 3.2 subunit (Caᵥ 3.2) in the development of epilepsy (Su et al., J Neurosci 22: 3645-3655, 2002).

Novel allosteric modulators of calcium channels, e.g., N or T-type calcium channels, are thus desired. Modulators may affect the kinetics and/or the voltage potentials of, e.g., the Caᵥ3.2 channel.

The invention provides compounds that act at these N and T-type calcium channels and are useful to treat various conditions associated with these calcium channels, such as pain and epilepsy. It also provides pharmaceutical compositions containing these compounds and methods to use them either alone or in combination with other pharmaceutical agents.

### SUMMARY OF THE INVENTION

The invention relates to compounds useful in treating conditions modulated by calcium channel activity and in particular conditions mediated by T-type channel activity. The compounds of the invention are cycloalkyl aryl sulphone derivatives with structural features that enhance the calcium channel blocking activity of the compounds.

In a first aspect, the invention features a compound having a structure according to the following formula,

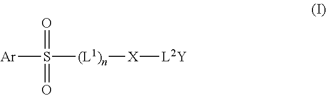

or a pharmaceutically acceptable salt, solvate, or prodrug thereof, or a stereoisomer thereof, or a conjugate thereof, where

Ar is an optionally substituted phenyl;

L¹ is methylenyl, ethylenyl, or propylenyl;

X is an optionally substituted cyclohexyl, an optionally substituted cyclobutyl, optionally substituted piperidinyl, or dimethylmethylenyl;

n is 0 or 1;

L² is (CH₂)₀₋₃CONR′(CH₂)₀₋₂, (CH₂)₀₋₃NR′CO, CH₂NR′CH₂CONR′, (CH₂)₀₋₃NR′CONR′, NR′COCH₂NR′, NR′CH₂CONR′, CH₂NHCH₂CONR′, NR′COO, NR′(CH₂)₁₋₃NR′CO, (CH₂)₀₋₃NR′SO₂, (CH₂)₀₋₃SO₂NR′(CH₂)₀₋₂, (CH₂)₁₋₂NR′ (CH₂)₀₋₁, (CH₂)₁₋₂SO₂, or imidazolyl;

Y is H or an optionally substituted C1-C10 alkyl, C2-C10 alkenyl, C2-C10 alkynyl, C2-C10 heteroalkyl, C2-C10 heteroalkenyl, C2-C10 heteroalkynyl, C4-C10 heterocycloalkyl, C6-C10 aryl, heteroaryl (5-12 ring members), C3-C10 cycloalkyl, heterocyclyl (5-12 ring members), aryl(5-12 ring members)-C1-C10 alkyl; or R′ from L² and Y may together form an optionally substituted heterocyclic ring (4-8 ring members); and

each R′ is, independently, H, methyl, ethyl or propyl.

In some embodiments, Ar includes a substituent selected from halo, CN, CF₃, OCF₃, COOR″, CONR″₂, OR″, SR″, SOR″, SO₂R″, C1-C6 alkyl, C2-C6 alkenyl, C2-C6 alkynyl, C2-C6 heteroalkyl, C2-C6 heteroalkenyl, C2-C6 heteroalkynyl; C6-C10 aryl, heteroaryl (5-12 ring members), O—(C6-C10)aryl, O-heteroaryl (5-12 ring members), C6-C10 aryl-C1-C6 alkyl, or heteroaryl (5-12 ring members)-alkyl (1-6C), and where each R″ is independently H or an optionally substituted group selected from C1-C6 alkyl, C2-C6 alkenyl, C2-C6 alkynyl, C2-C6 heteroalkyl, C2-C6 heteroalkenyl, or C2-C6 heteroalkynyl.

In some embodiments, Y includes a substituent selected from halo, CN, CF₃, OCF₃, COOR″, CONR″₂, OR″, SR″, SOR″, SO₂R″, C1-C6 alkyl, C2-C6 alkenyl, C2-C6 alkynyl, C2-C6 heteroalkyl, C2-C6 heteroalkenyl, C2-C6 heteroalkynyl; C6-C10 aryl, heteroaryl (5-12 ring members), O—(C6-C10)aryl, O-heteroaryl (5-12 ring members), C6-C10 aryl-C1-C6 alkyl, heteroaryl (5-12 ring members)-alkyl (1-6C), ═O, ═NOR″, NO₂, NR″₂, NR″(CO)R″, or NR″SO₂R″, and wherein each R″ is independently H or an optionally substituted group selected from C1-C6 alkyl, C2-C6 alkenyl, C2-C6 alkynyl, C2-C6 heteroalkyl, C2-C6 heteroalkenyl, C2-C6 heteroalkynyl.

In certain embodiments, the optional substituents on X are selected, independently, from halo, methyl, ethyl, propyl, and OR′, and each R′ is, independently, H, methyl, ethyl or propyl.

In some embodiments, Ar is phenyl substituted by F, CF₃, or OCF₃.

In other embodiments, when X is an optionally substituted cyclohexyl or optionally substituted piperidinyl, one of ArSO₂(L¹)ₙX-and-L² is located at C1 and the other is located at C4 or N4. In still other embodiments, when X is an optionally substituted cyclobutyl, one of ArSO₂(L¹)ₙX-and-L² is located at C1 and the other is located at C3.

In certain embodiments, when X is cyclohexyl, said cyclohexyl is unsubstituted or substituted by a methyl group.

In some embodiments, Y is phenyl, heteroaryl, or C1-C6 alkyl comprising a substituent selected from CF₃, F, Cl, OCF₃, SO₂Me, and SO₂(ⁱPr).

In still other embodiments, L² is —NHCO—, —NCH₃CO—, or —NHSO₂—.

In some embodiments, the compound has a structure according to the following formula,

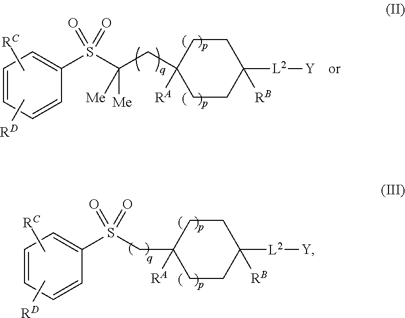

where each of R^{A} and R^{B} is selected, independently, from H, OH, optionally substituted C1-C3 alkyl, and halogen; R^{C} is CF₃ or OCF₃; R^{D} is H, halogen, or CF₃; both p are 0, or both p are 1; q is 0 or 1; L² is selected from —NR′CO—, —CONR′—, —NR′CH₂CONH—, —CH₂NR′CO—, —CH₂NR′CH₂CONR′—, —NR′COCH₂NR′—, —NR′CONR′—, —NR′COO—, —NR′SO₂—; each R′ is selected, independently, from H or CH₃; and Y is H, optionally substituted phenyl, optionally substituted heteroaryl, unsubstituted C1-C6 alkyl, C1-C6 haloalkyl, C3-C6 cycloalkyl, or heterocyclyl.

In some embodiments, both p are 0.

In certain embodiments, both p are 1.

In other embodiments, q is 0.

In still other embodiments, q is 1.

In some embodiments, each R′ is, independently, H or CH₃.

In still other embodiments, the compound has a structure according to:

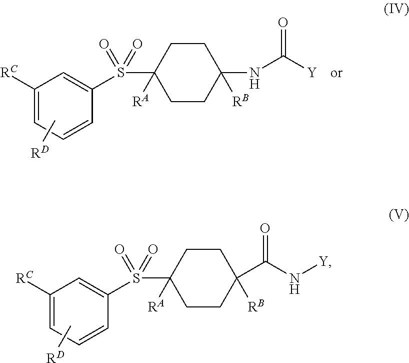

where each of R^{A} and R^{B} is selected, independently, from H, OH, optionally substituted C1-C3 alkyl, and halogen; R^{C} is CF₃ or OCF₃; and R^{D} is H, halogen, or CF₃.

In certain embodiments, the compound has a structure according to:

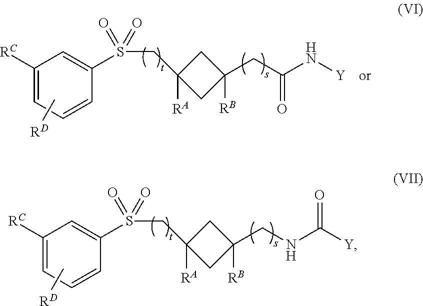

where s is 0 or 1; t is 0 or 1; each of R^{A} and R^{B} is selected, independently, from H, OH, optionally substituted C1-C3 alkyl, and halogen; R^{C} is CF₃ or OCF₃; and R^{D} is H, halogen, or CF₃. In further embodiments, t is 0 and s is 0, or t is 0 and s is 1. In other embodiments, t is 1 and s is 0, or t is 1 and s is 1.

In other embodiments, the compound has a structure according to the following formula,

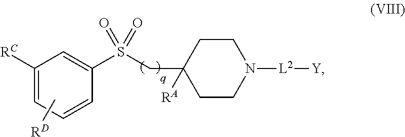

where R^{A} is H, OH, optionally substituted C1-C3 alkyl, and halogen; q is 0, 1, or 2; R^{C} is CF₃ or OCF₃; and R^{D} is H, halogen, or CF₃.

In still other embodiments, the compound has a structure according to the following formula,

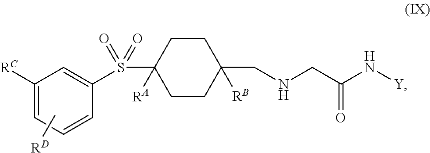

where each of R^{A} and R^{B} is selected, independently, from H, OH, optionally substituted C1-C3 alkyl, and halogen; R^{C} is CF₃ or OCF₃; and R^{D} is H, halogen, or CF₃.

In some embodiments, the compound has a structure according to the following formula,

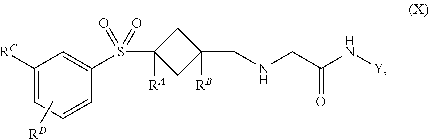

where each of R^{A} and R^{B} is selected, independently, from H, OH, optionally substituted C1-C3 alkyl, and halogen; R^{C} is CF₃ or OCF₃; and R^{D} is H, halogen, or CF₃.

In certain embodiments, the compound has a structure according to the following formula,

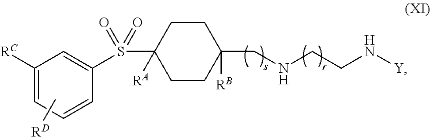

where r is 1 or 2; s is 0 or 1; each of R^{A} and R^{B} is selected, independently, from H, OH, optionally substituted C1-C3 alkyl, and halogen; R^{C} is CF₃ or OCF₃; and R^{D} is H, halogen, or CF₃.

In still other embodiments, the compound has a structure according to the following formula,

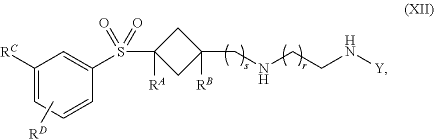

where r is 1 or 2; s is 0 or 1; each of R^{A} and R^{B} is selected, independently, from H, OH, optionally substituted C1-C3 alkyl, and halogen; R^{C} is CF₃ or OCF₃; and R^{D} is H, halogen, or CF₃.

In some embodiments, the compound has a structure according to

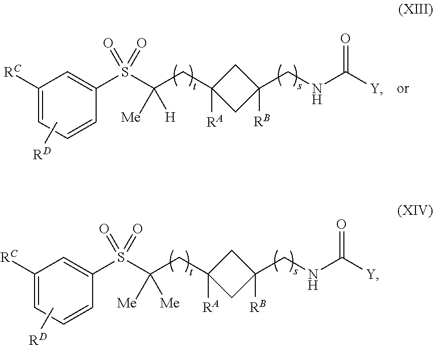

where s is 0 or 1; t is 0 or 1; each of R^{A} and R^{B} is selected, independently, from H, OH, optionally substituted C1-C3 alkyl, and halogen; R^{C} is CF₃ or OCF₃; and R^{D} is H, halogen, or CF₃. In further embodiments, t is 0 and s is 0, or t is 0 and s is 1. In other embodiments, t is 1 and s is 0, or t is 1 and s is 1.

In other embodiments, the compound has a structure according to

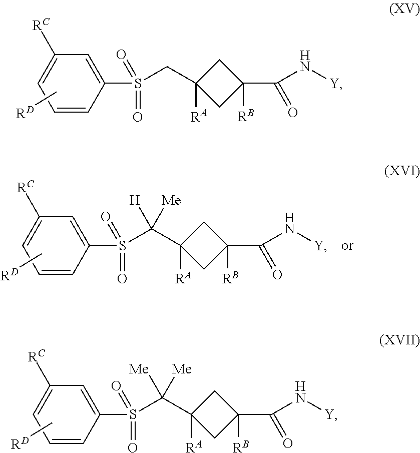

where each of R^{A} and R^{B} is selected, independently, from H, OH, optionally substituted C1-C3 alkyl, and halogen; R^{C} is CF₃ or OCF₃; and R^{D} is H, halogen, or CF₃.

In still other embodiments, the compound has a structure according to the following formula,

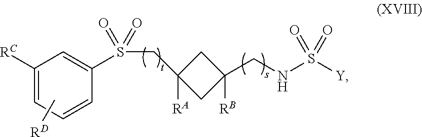

where s is 0 or 1; t is 0 or 1; each of R^{A} and R^{B} is selected, independently, from H, OH, optionally substituted C1-C3 alkyl, and halogen; R^{C} is CF₃ or OCF₃; and R^{D} is H, halogen, or CF₃.

In certain embodiments, the compound has a structure according to the following formula,

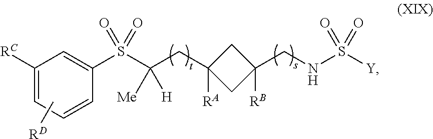

where s is 0 or 1; t is 0 or 1; each of R^{A} and R^{B} is selected, independently, from H, OH, optionally substituted C1-C3 alkyl, and halogen; R^{C} is CF₃ or OCF₃; and R^{D} is H, halogen, or CF₃.

In some embodiments, the compound has a structure according to the following formula,

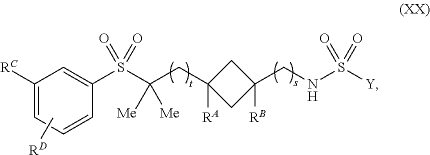

where s is 0 or 1; t is 0 or 1; each of R^{A} and R^{B} is selected, independently, from H, OH, optionally substituted C1-C3 alkyl, and halogen; R^{C} is CF₃ or OCF₃; and R^{D} is H, halogen, or CF₃. In certain embodiments, t is 0 and s is 0, or t is 0 and s is 1. In other embodiments, t is 1 and s is 0, or t is 1 and s is 1.

In still other embodiments, the compound has a structure according to

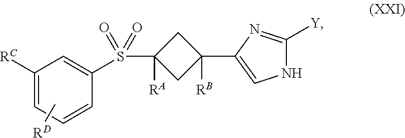

where each of R^{A} and R^{B} is selected, independently, from H, OH, optionally substituted C1-C3 alkyl, and halogen; R^{C} is CF₃ or OCF₃; and R^{D} is H, halogen, or CF₃.

In some embodiments, R^{A} is H, F, or CH₃. In certain embodiments, R^{A} is F or CH₃. In other embodiments, R^{A} is H.

In certain embodiments, R^{B} is H, OH, or CH₃.

In other embodiments, R^{A} and R^{B} are both H.

In still other embodiments, the compound has a structure according to

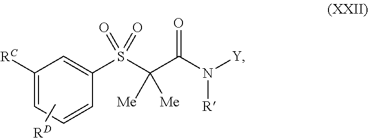

where R′ is H or CH₃; R^{C} is CF₃ or OCF₃; and R^{D} is H, halogen, or CF₃.

In some embodiments, the compound has a structure according to

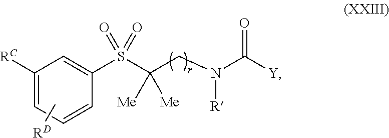

where r is 1 or 2; R′ is H or CH₃; R^{C} is CF₃ or OCF₃; and R^{D} is H, halogen, or CF₃.

In other embodiments, the compound has a structure according to

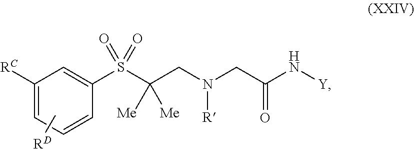

where R′ is H or CH₃; R^{C} is CF₃ or OCF₃; and R^{D} is H, halogen, or CF₃.

In still other embodiments, compound has a structure according to

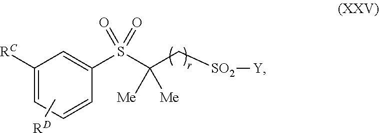

where r is 1 or 2; R′ is H or CH₃; R^{C} is CF₃ or OCF₃; and R^{D} is H, halogen, or CF₃.

In certain embodiments, the compound has a structure according to

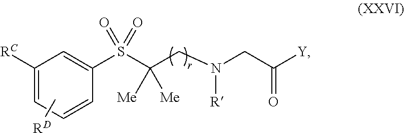

where r is 1 or 2; R′ is H or CH₃; R^{C} is CF₃ or OCF₃; and R^{D} is H, halogen, or CF₃.

In other embodiments, the compound has a structure according to

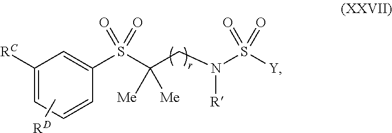

where r is 1 or 2; R′ is H or CH₃; R^{C} is CF₃ or OCF₃; and R^{D} is H, halogen, or CF₃.

In some embodiments, Y is optionally substituted C1-C10 alkyl or optionally substituted C2-C10 heteroalkyl. In other embodiments, Y is optionally substituted C1-C5 alkyl or optionally substituted C2-C6 heteroalkyl.

In other embodiments, Y is optionally substituted C6-C10 aryl, optionally substituted heteroaryl, optionally substituted C3-C10 cycloalkyl, or optionally substituted heterocyclyl (5-12 ring members). In certain embodiments, Y is optionally substituted tetrahydropyranyl, optionally substituted 1,4-morpholino, optionally substituted cyclobutyl, optionally substituted cyclopentyl, optionally substituted cyclohexyl, optionally substituted phenyl, optionally substituted pyrimidinyl, optionally substituted pyridyl, optionally substituted pyrazolyl, optionally substituted oxazolyl, optionally substituted isoxazolyl, optionally substituted benzimidazolyl, optionally substituted triazolyl, optionally substituted thiazolyl, optionally substituted isothiazolyl, optionally substituted furyl, optionally substituted thienyl, optionally substituted imidazolyl, optionally substituted imidazo[1,2-a]pyridine, optionally substituted 1,6-naphthyridine, optionally substituted 2,3-dihydroindolyl, optionally substituted phthalimido, or optionally substituted oxo-isoindolyl. In further embodiments, Y is optionally substituted phenyl, optionally substituted pyrimidinyl, or optionally substituted pyridyl. In some embodiments, Y is substituted by F, Cl, CF₃, —SO₂Me, or —SO₂¹Pr, and optionally substituted by halogen, C1-C3 alkoxy, C1-C3 alkyl, C1-C3 haloalkyl, C3-C6 cycloalkyl, halophenyl, or —SO₂(C1-C4 alkyl). In still other embodiments, Y is unsubstituted or substituted by NH₂, halo, optionally substituted phenyl, optionally substituted benzyl, or optionally substituted pyridyl.

In some embodiments, R^{A} and R^{B} are cis to each other.

In other embodiments, R^{A} and R^{B} are trans to each other.

In certain embodiments, the carbon substituted by R^{A} has the S configuration.

In still other embodiments, the carbon substituted by R^{A} has the R configuration.

In some embodiments, the carbon substituted by R^{B} has the S configuration.

In other embodiments, the carbon substituted by R^{B} has the R configuration.

In some embodiments, R^{C} is CF₃.

In still other embodiments, R^{C} is OCF₃.

In certain embodiments, the compound has the structure of any of compounds 1-780 in Table 1, or a pharmaceutically acceptable salt, solvate, or prodrug thereof, or a stereoisomer thereof, or a conjugate thereof.

In some embodiments, the compound is

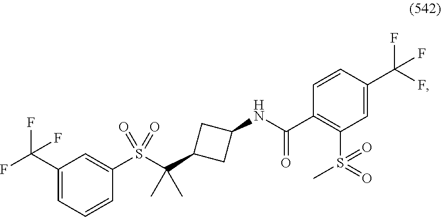

or a pharmaceutically acceptable salt, solvate, or prodrug thereof, or a stereoisomer thereof, or a conjugate thereof.

In another aspect, the invention features a pharmaceutical composition that includes any of the compounds described herein (e.g., a compound of any of Formulas (I)-(XXVII) or any of compounds 1-780 in Table 1), or a pharmaceutically acceptable salt, solvate, or prodrug thereof, or a stereoisomer thereof, or a conjugate thereof, and a pharmaceutically acceptable carrier or excipient. In some embodiments, the pharmaceutical composition is formulated in unit dosage form. In further embodiments, the unit dosage form is a tablet, caplet, capsule, lozenge, film, strip, gelcap, or syrup.

The invention is also directed to the use of the compounds described herein (e.g., a compound of any of Formulas (I)-(XXVII) or any of compounds 1-780 in Table 1) for the preparation of medicaments for the treatment of conditions requiring modulation of calcium channel activity, and in particular N or T-type calcium channel activity.

In another aspect, the invention features a method to treat a condition modulated by calcium channel activity, where the method includes administering to a subject in need of such treatment an effective amount of any of the compounds described herein (e.g., a compound of any of Formulas (I)-(XXVII) or any of compounds 1-780 in Table 1), or a pharmaceutically acceptable salt, solvate, or prodrug thereof, or a stereoisomer thereof, or a conjugate thereof, or any pharmaceutical composition thereof. In some embodiments, the calcium channel is a T-type calcium channel (e.g., the CaV 3.1, CaV 3.2, or CaV 3.3 channel).

In other embodiments, the calcium channel is an N-type calcium channel (e.g., the CaV 2.2 channel).

In some embodiments, the condition is pain, epilepsy, Parkinson's disease, depression, psychosis (e.g, schizophrenia), or tinnitus.

In some embodiments, the condition is pain or epilepsy. In certain embodiments, the pain is inflammatory pain or neuropathic pain.

In still other embodiments, the pain is chronic pain (e.g., peripheral neuropathic pain; central neuropathic pain, musculoskeletal pain, headache, visceral pain, or mixed pain). In some embodiments, the peripheral neuropathic pain is post-herpetic neuralgia, diabetic neuropathic pain, neuropathic cancer pain, failed back-surgery syndrome, trigeminal neuralgia, or phantom limb pain. In other embodiments, the central neuropathic pain is multiple sclerosis related pain, Parkinson disease related pain, post-stroke pain, post-traumatic spinal cord injury pain, or pain in dementia. In still other embodiments, the musculoskeletal pain is osteoarthritic pain and fibromyalgia syndrome; inflammatory pain such as rheumatoid arthritis, or endometriosis. In some embodiments, the headache is migraine, cluster headache, tension headache syndrome, facial pain, or headache caused by other diseases. In certain embodiments, the visceral pain is interstitial cystitis, irritable bowel syndrome, or chronic pelvic pain syndrome. In other embodiments, the mixed pain is lower back pain, neck and shoulder pain, burning mouth syndrome, or complex regional pain syndrome.

In some embodiments, the headache is migraine.

In other embodiments, the pain is acute pain (e.g., nociceptive pain or post-operative pain). In some embodiments, the acute pain is post-operative pain.

In some embodiments, the condition is epilepsy.

As used herein, the term “alkyl,” “alkenyl” and “alkynyl” include straight-chain, branched-chain and cyclic monovalent substituents, as well as combinations of these, containing only C and H when unsubstituted. Examples include methyl, ethyl, isobutyl, cyclohexyl, cyclopentylethyl, 2-propenyl, 3-butynyl, and the like. Typically, the alkyl, alkenyl and alkynyl groups contain 1-10C (alkyl) or 2-10C (alkenyl or alkynyl). In some embodiments, they contain 1-8C, 1-6C, 1-4C, 1-3C or 1-2C (alkyl); or 2-8C, 2-6C, 2-4C or 2-3C (alkenyl or alkynyl). Further, any hydrogen atom on one of these groups can be replaced with a halogen atom, and in particular a fluoro or chloro, and still be within the scope of the definition of alkyl, alkenyl and alkynyl. For example, CF₃ is a 1C alkyl. These groups may be also be substituted by other substituents.

Heteroalkyl, heteroalkenyl and heteroalkynyl are similarly defined and contain at least one carbon atom but also contain one or more O, S or N heteroatoms or combinations thereof within the backbone residue whereby each heteroatom in the heteroalkyl, heteroalkenyl or heteroalkynyl group replaces one carbon atom of the alkyl, alkenyl or alkynyl group to which the heteroform corresponds. In some embodiments, the heteroalkyl, heteroalkenyl and heteroalkynyl groups have C at each terminus to which the group is attached to other groups, and the heteroatom(s) present are not located at a terminal position. As is understood in the art, these heteroforms do not contain more than three contiguous heteroatoms. In some embodiments, the heteroatom is O or N.

The designated number of carbons in heteroforms of alkyl, alkenyl and alkynyl includes the heteroatom count. For example, if heteroalkyl is defined as 1-6C, it will contain 1-6 C, N, O, or S atoms such that the heteroalkyl contains at least one C atom and at least one heteroatom, for example 1-5C and 1N or 1-4C and 2N. Similarly, when heteroalkyl is defined as 1-6C or 1-4C, it would contain 1-5C or 1-3C respectively, i.e., at least one C is replaced by O, N or S. Accordingly, when heteroalkenyl or heteroalkynyl is defined as 2-6C (or 2-4C), it would contain 2-6 or 2-4 C, N, O, or S atoms, since the heteroalkenyl or heteroalkynyl contains at least one carbon atom and at least one heteroatom, e.g. 2-5C and 1N or 2-4C and 20. Further, heteroalkyl, heteroalkenyl or heteroalkynyl substituents may also contain one or more carbonyl groups. Examples of heteroalkyl, heteroalkenyl and heteroalkynyl groups include CH₂OCH3, CH2N(CH3)2, CH2OH, (CH2)nNR2, OR, COOR, CONR2, (CH2)n OR, (CH2)nCOR, (CH2)nCOOR, (CH2)nSR, (CH2)nSOR, (CH2)nSO2R, (CH2)nCONR2, NRCOR, NRCOOR, OCONR2, OCOR and the like wherein the R group contains at least one C and the size of the substituent is consistent with the definition of alkyl, alkenyl and alkynyl as described herein.

As used herein, the terms “alkylene,” “alkenylene” and “alkynylene” refers to divalent or trivalent groups having a specified size, typically 1-2C, 1-3C, 1-4C, 1-6C or 1-8C for the saturated groups and 2-3C, 2-4C, 2-6C or 2-8C for the unsaturated groups. They include straight-chain, branched-chain and cyclic forms as well as combinations of these, containing only C and H when unsubstituted. Because they are divalent, they can link together two parts of a molecule, as exemplified by X in the compounds described herein. Examples are methylene, ethylene, propylene, cyclopropan-1,1-diyl, ethylidene, 2-butene-1,4-diyl, and the like. These groups can be substituted by the groups typically suitable as substituents for alkyl, alkenyl and alkynyl groups as set forth herein. Thus C═O is a C1 alkylene that is substituted by ═O, for example.

Heteroalkylene, heteroalkenylene and heteroalkynylene are similarly defined as divalent groups having a specified size, typically 1-3C, 1-4C, 1-6C or 1-8C for the saturated groups and 2-3C, 2-4C, 2-6C or 2-8C for the unsaturated groups. They include straight chain, branched chain and cyclic groups as well as combinations of these, and they further contain at least one carbon atom but also contain one or more O, S or N heteroatoms or combinations thereof within the backbone residue, whereby each heteroatom in the heteroalkylene, heteroalkenylene or heteroalkynylene group replaces one carbon atom of the alkylene, alkenylene or alkynylene group to which the heteroform corresponds. As is understood in the art, these heteroforms do not contain more than three contiguous heteroatoms.

“Aromatic” moiety or “aryl” moiety refers to any monocyclic or fused ring bicyclic system which has the characteristics of aromaticity in terms of electron distribution throughout the ring system and includes a monocyclic or fused bicyclic moiety such as phenyl or naphthyl; “heteroaromatic” or “heteroaryl” also refers to such monocyclic or fused bicyclic ring systems containing one or more heteroatoms selected from O, S and N. The inclusion of a heteroatom permits inclusion of 5-membered rings to be considered aromatic as well as 6-membered rings. Thus, typical aromatic/heteroaromatic systems include pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, isoxazolyl, benzoxazolyl, benzoisoxazolyl, imidazolyl and the like. Because tautomers are theoretically possible, phthalimido is also considered aromatic. Typically, the ring systems contain 5-12 ring member atoms or 6-10 ring member atoms. In some embodiments, the aromatic or heteroaromatic moiety is a 6-membered aromatic rings system optionally containing 1-2 nitrogen atoms. More particularly, the moiety is an optionally substituted phenyl, pyridyl, indolyl, pyrimidyl, pyridazinyl, benzothiazolyl or benzimidazolyl, pyrazolyl, imidazolyl, isoxazolyl, thiazolyl, benzothiazolyl, indolyl. Even more particularly, such moiety is phenyl, pyridyl, or pyrimidyl and even more particularly, it is phenyl.

“O-aryl” or “O-heteroaryl” refers to aromatic or heteroaromatic systems which are coupled to another residue through an oxygen atom. A typical example of an O-aryl is phenoxy. Similarly, “arylalkyl” refers to aromatic and heteroaromatic systems which are coupled to another residue through a carbon chain, saturated or unsaturated, typically of 1-8C, 1-6C or more particularly 1-4C or 1-3C when saturated or 2-8C, 2-6C, 2-4C or 2-3C when unsaturated, including the heteroforms thereof. For greater certainty, arylalkyl thus includes an aryl or heteroaryl group as defined above connected to an alkyl, heteroalkyl, alkenyl, heteroalkenyl, alkynyl or heteroalkynyl moiety also as defined above. Typical arylalkyls would be an aryl(6-12C)alkyl(1-8C), aryl(6-12C)alkenyl(2-8C), or aryl(6-12C)alkynyl(2-8C), plus the heteroforms. A typical example is phenylmethyl, commonly referred to as benzyl.

Typical optional substituents on aromatic or heteroaromatic groups include independently halo, CN, NO₂, CF₃, OCF₃, COOR′, CONR′₂, OR′, SR′, SOR′, SO₂R′, NR′₂, NR′(CO)R′,NR′C(O)OR′, NR′C(O)NR′₂, NR′SO₂NR′₂, or NR′SO₂R′, wherein each R′ is independently H or an optionally substituted group selected from alkyl, alkenyl, alkynyl, heteroalkyl, heteroalkenyl, heteroalkynyl, heteroaryl, and aryl (all as defined above); or the substituent may be an optionally substituted group selected from alkyl, alkenyl, alkynyl, heteroalkyl, heteroalkenyl, heteroalkynyl, aryl, heteroaryl, O-aryl, O-heteroaryl and arylalkyl.

Optional substituents on a non-aromatic group, are typically selected from the same list of substituents suitable for aromatic or heteroaromatic groups and may further be selected from ═O and ═NOR′ where R′ is H or an optionally substituted group selected from alkyl, alkenyl, alkynyl, heteroalkyl, heteroalkenyl, heteralkynyl, heteroaryl, and aryl (all as defined above).

Halo may be any halogen atom, especially F, Cl, Br, or I, and more particularly it is fluoro, chloro or bromo and even more particularly it is fluoro or chloro.

In general, any alkyl, alkenyl, alkynyl, or aryl (including all heteroforms defined above) group contained in a substituent may itself optionally be substituted by additional substituents. The nature of these substituents is similar to those recited with regard to the substituents on the basic structures above. Thus, where an embodiment of a substituent is alkyl, this alkyl may optionally be substituted by the remaining substituents listed as substituents where this makes chemical sense, and where this does not undermine the size limit of alkyl per se; e.g., alkyl substituted by alkyl or by alkenyl would simply extend the upper limit of carbon atoms for these embodiments, and is not included. However, alkyl substituted by aryl, amino, halo and the like would be included.

In general, a substituent group (e.g., alkyl, alkenyl, alkynyl, or aryl (including all heteroforms defined above) may itself optionally be substituted by additional substituents. The nature of these substituents is similar to those recited with regard to the substituents on the basic structures above. Thus, where an embodiment of a substituent is alkyl, this alkyl may optionally be substituted by the remaining substituents listed as substituents where this makes chemical sense, and where this does not undermine the size limit of alkyl per se; e.g., alkyl substituted by alkyl or by alkenyl would simply extend the upper limit of carbon atoms for these embodiments, and is not included. However, alkyl substituted by aryl, amino, halo and the like would be included. For example, where a group is substituted, the group may be substituted with 1, 2, 3, 4, 5, or 6 substituents. Optional substituents include, but are not limited to: 1C-6C alkyl or heteroaryl, 2C-6C alkenyl or heteroalkenyl, 2C-6C alkynyl or heteroalkynyl, halogen; aryl, heteroaryl, azido(—N3), nitro (—NO₂), cyano (—CN), acyloxy(—OC(═O)R′), acyl (—C(═O)R′), alkoxy (—OR′), amido (—NR′C(═O)R″ or —C(═O)NRR′), amino (—NRR′), carboxylic acid (—CO2H), carboxylic ester (—CO2R′), carbamoyl (—OC(═O)NR′R″ or —NRC(═O)OR′), hydroxy (—OH), isocyano (—NC), sulfonate (—S(═O)₂OR), sulfonamide (—S(═O)₂NRR′ or —NRS(═O)₂R′), or sulfonyl (—S(═O)₂R), where each R or R′ is selected, independently, from H, 1C-6C alkyl or heteroaryl, 2C-6C alkenyl or heteroalkenyl, 2C-6C alkynyl or heteroalkynyl, aryl, or heteroaryl. A substituted group may have, for example, 1, 2, 3, 4, 5, 6, 7, 8, or 9 substituents.

The term an “effective amount” of an agent (e.g., a compound of any of Formulas (I)-(XXVII) or any of compounds 1-780 in Table 1), as used herein, is that amount sufficient to effect beneficial or desired results, such as clinical results, and, as such, an “effective amount” depends upon the context in which it is being applied. For example, in the context of administering an agent that is a modulator of a calcium channel (e.g., Caᵥ 3.1, Caᵥ 3.2, or Caᵥ 3.3, or Caᵥ 2.2), an effective amount of an agent is, for example, an amount sufficient to achieve a change in calcium channel activity as compared to the response obtained without administration of the agent.

The term “pharmaceutical composition,” as used herein, represents a composition containing a compound described herein (e.g., a compound of any of Formulas (I)-(XXVII) or any of compounds 1-780 in Table 1) formulated with a pharmaceutically acceptable excipient. In some embodiments, the pharmaceutical composition is manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, refers any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable prodrugs” as used herein, represents those prodrugs of the compounds of the present invention that are, within the scope of sound medical judgment, suitable for use in contact with the tissues of humans and animals with undue toxicity, irritation, allergic response, and the like, commensurate with a reasonable benefit/risk ratio, and effective for their intended use, as well as the zwitterionic forms, where possible, of the compounds of the invention.

The term “pharmaceutically acceptable salt,” as use herein, represents those salts of the compounds described here (e.g., a compound of any of Formulas (I)-(XXVII) or any of compounds 1-780 in Table 1) that are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting the free base group with a suitable organic acid.

The compounds of the invention (e.g., a compound of any of Formulas (I)-(XXVII) or any of compounds 1-780 in Table 1) may have ionizable groups so as to be capable of preparation as pharmaceutically acceptable salts. These salts may be acid addition salts involving inorganic or organic acids or the salts may, in the case of acidic forms of the compounds of the invention be prepared from inorganic or organic bases. Frequently, the compounds are prepared or used as pharmaceutically acceptable salts prepared as addition products of pharmaceutically acceptable acids or bases. Suitable pharmaceutically acceptable acids and bases are well-known in the art, such as hydrochloric, sulphuric, hydrobromic, acetic, lactic, citric, or tartaric acids for forming acid addition salts, and potassium hydroxide, sodium hydroxide, ammonium hydroxide, caffeine, various amines, and the like for forming basic salts. Methods for preparation of the appropriate salts are well-established in the art.

Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts and the like. Representative alkali or alkaline earth metal salts include sodium, lithium, potassium, calcium, magnesium and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine and the like.

The term “pharmaceutically acceptable solvate” as used herein means a compound as described herein (e.g., a compound of any of Formulas (I)-(XXVII) or any of compounds 1-780 in Table 1) where molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the molecule is referred to as a “hydrate.”

The term “prevent,” as used herein, refers to prophylactic treatment or treatment that prevents one or more symptoms or conditions of a disease, disorder, or conditions described herein (for example, pain (e.g., chronic or acute pain), epilepsy, Alzheimer's disease, Parkinson's disease, cardiovascular disease, diabetes, cancer, sleep disorders, obesity, psychosis such as schizophrenia, overactive bladder, renal disease, neuroprotection, addiction, and male birth control). Preventative treatment can be initiated, for example, prior to (“pre-exposure prophylaxis”) or following (“post-exposure prophylaxis”) an event that precedes the onset of the disease, disorder, or conditions. Preventive treatment that includes administration of a compound described herein (e.g., a compound of any of Formulas (I)-(XXVII) or any of compounds 1-780 in Table 1), or a pharmaceutically acceptable salt or solvate thereof, or a pharmaceutical composition thereof, can be acute, short-term, or chronic. The doses administered may be varied during the course of preventative treatment.

The term “prodrug,” as used herein, represents compounds that are rapidly transformed in vivo to the parent compound of the above formula, for example, by hydrolysis in blood. Prodrugs of the compounds described herein (e.g., a compound of any of Formulas (I)-(XXVII) or any of compounds 1-780 in Table 1) may be conventional esters. Some common esters that have been utilized as prodrugs are phenyl esters, aliphatic (C1-C8 or C8-C24) esters, cholesterol esters, acyloxymethyl esters, carbamates, and amino acid esters. For example, a compound that contains an OH group may be acylated at this position in its prodrug form. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and Judkins et al., Synthetic Communications 26(23):4351-4367, 1996, each of which is incorporated herein by reference. Preferably, prodrugs of the compounds of the present invention are suitable for use in contact with the tissues of humans and animals with undue toxicity, irritation, allergic response, and the like, commensurate with a reasonable benefit/risk ratio, and effective for their intended use.

In addition, the compounds of the invention may be coupled through conjugation to substances designed to alter the pharmacokinetics, for targeting, or for other reasons. Thus, the invention further includes conjugates of these compounds. For example, polyethylene glycol is often coupled to substances to enhance half-life; the compounds may be coupled to liposomes covalently or noncovalently or to other particulate carriers. They may also be coupled to targeting agents such as antibodies or peptidomimetics, often through linker moieties. Thus, the invention is also directed to compounds (e.g., a compound of any of Formulas (I)-(XXVII) or any of compounds 1-780 in Table 1) when modified so as to be included in a conjugate of this type.

As used herein, and as well understood in the art, “to treat” a condition or “treatment” of the condition (e.g., the conditions described herein such as pain (e.g., chronic or acute pain), epilepsy, Alzheimer's disease, Parkinson's disease, cardiovascular disease, diabetes, cancer, sleep disorders, obesity, psychosis such as schizophrenia, overactive bladder, renal disease, neuroprotection, addiction, and male birth control) is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

The term “unit dosage form” refers to a physically discrete unit suitable as a unitary dosage for human subjects and other mammals, each unit containing a predetermined quantity of active material (e.g., a compound of any of Formulas (I)-(XXVII) or any of compounds 1-780 in Table 1) calculated to produce the desired therapeutic effect, in association with any suitable pharmaceutical excipient or excipients. Exemplary, non-limiting unit dosage forms include a tablet (e.g., a chewable tablet), caplet, capsule (e.g., a hard capsule or a soft capsule), lozenge, film, strip, gelcap, and syrup.

In some cases, the compounds of the invention contain one or more chiral centers. The invention includes each of the isolated stereoisomeric forms as well as mixtures of stereoisomers in varying degrees of chiral purity, including racemic mixtures. It also encompasses the various diastereomers and tautomers that can be formed.

Other features and advantages of the invention will be apparent from the following detailed description, the drawing, and the claims.

### DETAILED DESCRIPTION OF THE INVENTION

### Compounds

The invention features compounds that have a structure according to the following formula,

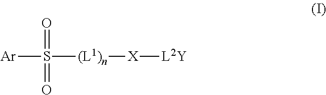

or a pharmaceutically acceptable salt, solvate, or prodrug thereof, or a stereoisomer thereof, or a conjugate thereof, where

Ar is an optionally substituted phenyl;

L¹ is methylenyl, ethylenyl, or propylenyl;

X is an optionally substituted cyclohexyl, an optionally substituted cyclobutyl, optionally substituted piperidinyl, or dimethylmethylenyl;

n is 0 or 1;

L² is (CH₂)₀₋₃CONR′(CH₂)₀₋₂, (CH₂)₀₋₃NR′CO, CH₂NR′CH₂CONR′, (CH₂)₀₋₃NR′CONR′, NR′COCH₂NR′, NR′CH₂CONR′, CH₂NHCH₂CONR′, NR′COO, NR′(CH₂)₁₋₃NR′CO, (CH₂)₀₋₃NR′SO₂, (CH₂)₀₋₃SO₂NR′(CH₂)₀₋₂, (CH₂)₁₋₂NR′ (CH₂)₀₋₁, (CH₂)₁₋₂SO₂, or imidazolyl;

Y is H or an optionally substituted C1-C10 alkyl, C2-C10 alkenyl, C2-C10 alkynyl, C2-C10 heteroalkyl, C2-C10 heteroalkenyl, C2-C10 heteroalkynyl, C4-C10 heterocycloalkyl, C6-C10 aryl, heteroaryl (5-12 ring members), C3-C10 cycloalkyl, heterocyclyl (5-12 ring members), aryl(5-12 ring members)-C1-C10 alkyl; or R′ from L² and Y may together form an optionally substituted heterocyclic ring (4-8 ring members); and

each R′ is, independently, H, methyl, ethyl or propyl.

Other compounds of the invention can be according to any of the following formulas described herein:

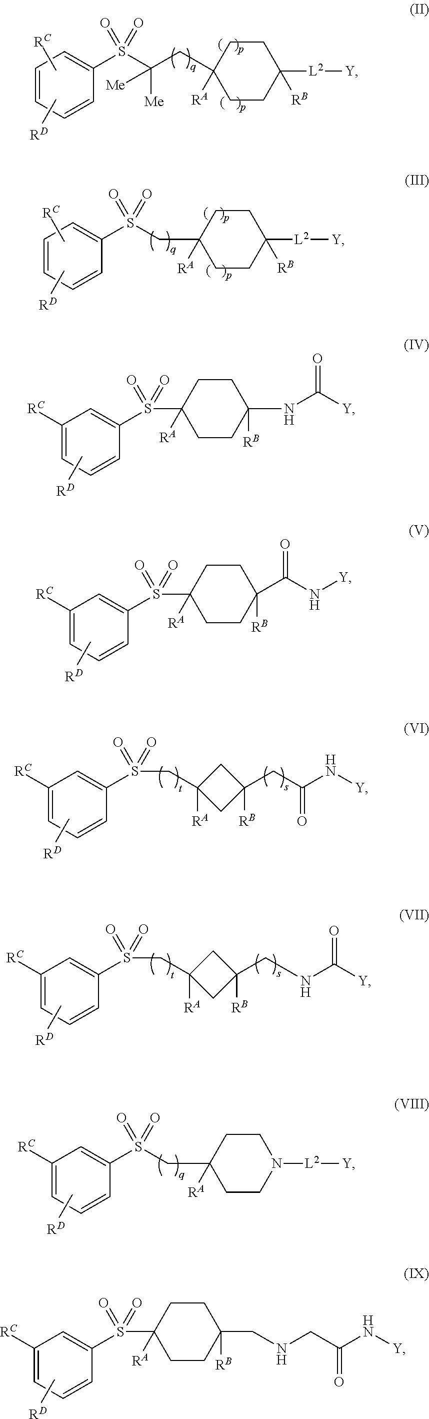

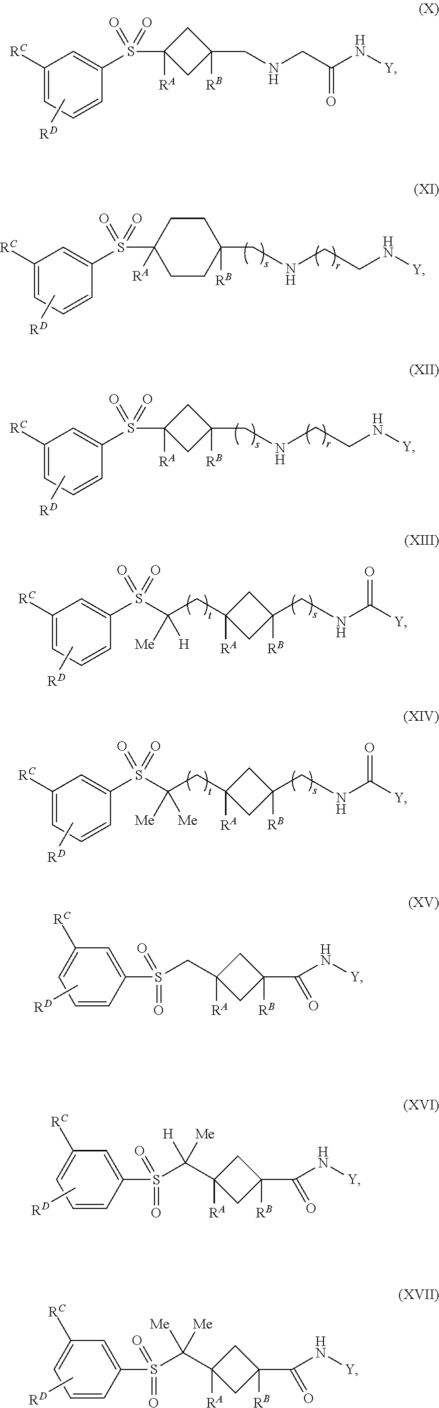

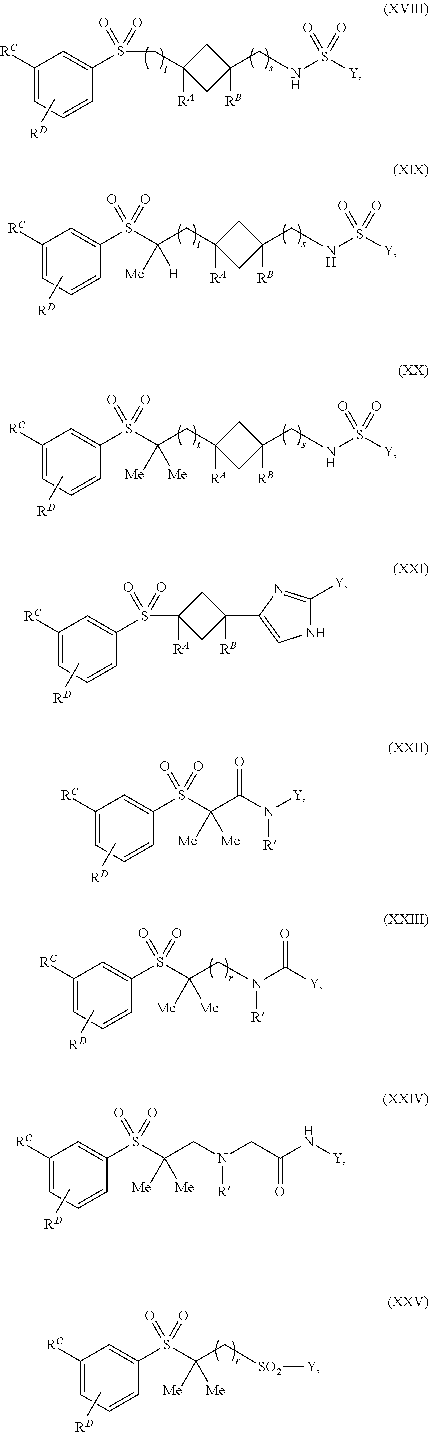

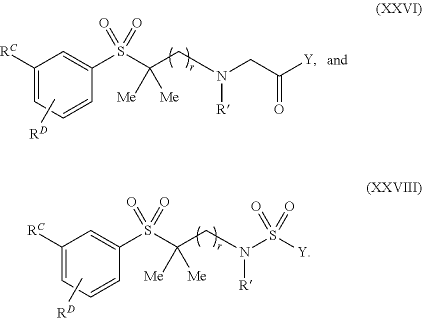

### Utility and Administration

The compounds described herein are useful in the methods of the invention and, while not bound by theory, are believed to exert their desirable effects through their ability to modulate the activity of calcium channels, particularly the activity of N and/or T-type calcium channels. This makes them useful for treatment of certain conditions where modulation of N- or T-type calcium channels is desired, including pain, epilepsy, migraine, Parkinson's disease, depression, schizophrenia, psychosis, and tinnitus.

### Modulation of Calcium Channels

The entry of calcium into cells through voltage-gated calcium channels mediates a wide variety of cellular and physiological responses, including excitation-contraction coupling, hormone secretion and gene expression (e.g., Miller et al., Science 235:46-52 (1987); Augustine et al., Annu Rev Neurosci 10: 633-693 (1987)). In neurons, calcium channels directly affect membrane potential and contribute to electrical properties such as excitability, repetitive firing patterns and pacemaker activity. Calcium entry further affects neuronal functions by directly regulating calcium-dependent ion channels and modulating the activity of calcium-dependent enzymes such as protein kinase C and calmodulin-dependent protein kinase II. An increase in calcium concentration at the presynaptic nerve terminal triggers the release of neurotransmitter, which also affects neurite outgrowth and growth cone migration in developing neurons.

Calcium channels mediate a variety of normal physiological functions, and are also implicated in a number of human disorders as described herein. For example, calcium channels also have been shown to mediate the development and maintenance of the neuronal sensitization and hyperexcitability processes associated with neuropathic pain, and provide attractive targets for the development of analgesic drugs (reviewed in Vanegas et al., Pain 85: 9-18 (2000)). Native calcium channels have been classified by their electrophysiological and pharmacological properties into T-, L-, N-, P/Q- and R-types (reviewed in Catterall, Annu Rev Cell Dev Biol 16: 521-555, 2000; Huguenard, Annu Rev Physiol 58: 329-348, 1996). The L-, N- and P/Q-type channels activate at more positive potentials (high voltage-activated) and display diverse kinetics and voltage-dependent properties (Id.).

The modulation of ion channels by the compounds described herein (e.g., a compound of any of Formulas (I)-(XXVII) or any of compounds 1-780 in Table 1) can be measured according to methods known in the art (e.g., in the references provided herein). Modulators of ion channels, e.g., voltage gated calcium ion channels, and the medicinal chemistry or methods by which such compounds can be identified, are also described in, for example: Birch et al., Drug Discovery Today, 9(9):410-418 (2004); Audesirk, “Chapter 6-Electrophysiological Analysis of Ion Channel Function,” Neurotoxicology: Approaches and Methods, 137-156 (1995); Camerino et al., “Chapter 4: Therapeutic Approaches to Ion Channel Diseases,” Advances in Genetics, 64:81-145 (2008); Petkov, “Chapter 16-Ion Channels,” Pharmacology: Principles and Practice, 387-427 (2009); Standen et al., “Chapter 15-Patch Clamping Methods and Analysis of Ion Channels,” Principles of Medical Biology, Vol. 7, Part 2, 355-375 (1997); Xu et al., Drug Discovery Today, 6(24):1278-1287 (2001); and Sullivan et al., Methods Mol. Biol. 114:125-133 (1999). Exemplary experimental methods are also provided in the Examples.

### T-Type Calcium Channels

T-type channels can be distinguished by having a more negative range of activation and inactivation, rapid inactivation, slow deactivation, and smaller single-channel conductances. There are three subtypes of T-type calcium channels that have been molecularly, pharmacologically, and elecrophysiologically identified: these subtypes have been termed α1G, α1H, and α1I (alternately called CaV 3.1, CaV 3.2 and CaV 3.3 respectively).

T-type calcium channels are involved in various medical conditions. In mice lacking the gene expressing the 3.1 subunit, resistance to absence seizures was observed (Kim et al., Mol. Cell Neurosci. 18(2): 235-245 (2001)). Other studies have also implicated the 3.2 subunit in the development of epilepsy (Su et al., J. Neurosci. 22: 3645-3655 (2002)). There is also evidence that some existing anticonvulsant drugs, such as ethosuximide, function through the blockade of T-type channels (Gomora et al., Mol. Pharmacol. 60: 1121-1132 (2001)).

Low voltage-activated calcium channels are highly expressed in tissues of the cardiovascular system. There is also a growing body of evidence that suggests that T-type calcium channels are abnormally expressed in cancerous cells and that blockade of these channels may reduce cell proliferation in addition to inducing apoptosis. Recent studies also show that the expression of T-type calcium channels in breast cancer cells is proliferation state dependent, i.e. the channels are expressed at higher levels during the fast-replication period, and once the cells are in a non-proliferation state, expression of this channel is minimal. Therefore, selectively blocking calcium channel entry into cancerous cells may be a valuable approach for preventing tumor growth (e.g., PCT Patent Publication Nos. WO 05/086971 and WO 05/77082; Taylor et al., World J. Gastroenterol. 14(32): 4984-4991 (2008); Heo et al., Biorganic & Medicinal Chemistry Letters 18:3899-3901 (2008)).

T-type calcium channels may also be involved in still other conditions. A recent study also has shown that T-type calcium channel antagonists inhibit high-fat diet-induced weight gain in mice. In addition, administration of a selective T-type channel antagonist reduced body weight and fat mass while concurrently increasing lean muscle mass (e.g., Uebele et al., The Journal of Clinical Investigation, 119(6):1659-1667 (2009)). T-type calcium channels may also be involved in pain (see for example: US Patent Publication No. 2003/0086980; PCT Publication Nos. WO 03/007953 and WO 04/000311). In addition to cardiovascular disease, epilepsy (see also US Patent Publication No. 2006/0025397), cancer, and chronic or acute pain, T-type calcium channels have been implicated in diabetes (US Patent Publication No. 2003/0125269), sleep disorders (US Patent Publication No. 2006/0003985), Parkinson's disease and psychosis such as schizophrenia (US Patent Publication No. 2003/0087799); overactive bladder (Sui et al., British Journal of Urology International 99(2): 436-441 (2007); US Patent Publication No. 2004/0197825), renal disease (Hayashi et al., Journal of Pharmacological Sciences 99: 221-227 (2005)), anxiety and alcoholism (US Patent Publication No. 2009/0126031), neuroprotection, and male birth control.

### N-Type Calcium Channels

Mutations in calcium channel α1 subunit genes in animals can provide important clues to potential therapeutic targets for pain intervention. Genetically altered mice null for the. α1B N-type calcium channel gene have been reported by several independent groups (Ino et al., Proc. Natl. Acad. Sci. USA 98:5323-5328 (2001); Kim et al., Mol Cell Neurosci 18:235-245 (2001); Kim et al., Neuron 31:35-45 (2001); Saegusa et al., Proc. Natl. Acad. Sci. USA 97:6132-6137 (2000); and Hatakeyama et al., NeuroReport 12:2423-2427 (2001)). These studies indicate that the N-type channel may be a potential target for mood disorders as well as pain.

In a variety of animal models, the selective block of N-type channels via intrathecal administration of ziconotide significantly depresses the formalin phase 2 response, thermal hyperalgesia, mechanical allodynia and post-surgical pain (e.g., Malmberg et al., J Neurosci 14: 4882-4890 (1994); Bowersox et al., J Pharmacol Exp Ther 279: 1243-1249 (1996); Sluka, J Pharmacol Exp Ther 287:232-237 (1998); and Wang et al., Soc Neurosci Abstr 24: 1626 (1998)).

Gabapentin (1-(aminomethyl)cyclohexaneacetic acid (Neurontin®)), is an anticonvulsant that also acts on N-type channels. Though not specific for N-type calcium channels, subsequent work has demonstrated that gabapentin is also successful at preventing hyperalgesia in a number of different animal pain models, including chronic constriction injury (CCI), heat hyperalgesia, inflammation, diabetic neuropathy, static and dynamic mechanical allodynia associated with postoperative pain (e.g., Cesena et al., Neurosci Lett 262: 101-104 (1999); Field et al., Pain 80: 391-398 (1999); Cheng et al., Anesthesiology 92: 1126-1131 (2000); and Nicholson, Acta Neurol Scand 101: 359-371 (2000)).

### Diseases and Conditions

Exemplary conditions that can be treated using the compounds described herein include pain (e.g., chronic or acute pain), epilepsy, Alzheimer's disease, Parkinson's disease, diabetes; cancer; sleep disorders; obesity; psychosis such as schizophrenia; overactive bladder; renal disease, neuroprotection, and addiction. For example, the condition can be pain (e.g., neuropathic pain or post-surgery pain), epilepsy, migraine, Parkinson's disease, depression, schizophrenia, psychosis, or tinnitus.

Epilepsy as used herein includes but is not limited to partial seizures such as temporal lobe epilepsy, absence seizures, generalized seizures, and tonic/clonic seizures.

Cancer as used herein includes but is not limited to breast carcinoma, neuroblastoma, retinoblastoma, glioma, prostate carcinoma, esophageal carcinoma, fibrosarcoma, colorectal carcinoma, pheochromocytoma, adrenocarcinoma, insulinoma, lung carcinoma, melanoma, and ovarian cancer.

Acute pain as used herein includes but is not limited to nociceptive pain and post-operative pain. Chronic pain includes but is not limited by: peripheral neuropathic pain such as post-herpetic neuralgia, diabetic neuropathic pain, neuropathic cancer pain, failed back-surgery syndrome, trigeminal neuralgia, and phantom limb pain; central neuropathic pain such as multiple sclerosis related pain, Parkinson disease related pain, post-stroke pain, post-traumatic spinal cord injury pain, and pain in dementia; musculoskeletal pain such as osteoarthritic pain and fibromyalgia syndrome; inflammatory pain such as rheumatoid arthritis and endometriosis; headache such as migraine, cluster headache, tension headache syndrome, facial pain, headache caused by other diseases; visceral pain such as interstitial cystitis, irritable bowel syndrome and chronic pelvic pain syndrome; and mixed pain such as lower back pain, neck and shoulder pain, burning mouth syndrome and complex regional pain syndrome.

In treating osteoarthritic pain, joint mobility can also improve as the underlying chronic pain is reduced. Thus, use of compounds of the present invention to treat osteoarthritic pain inherently includes use of such compounds to improve joint mobility in patients suffering from osteoarthritis.

The compounds described herein can be tested for efficacy in any standard animal model of pain. Various models test the sensitivity of normal animals to intense or noxious stimuli (physiological or nociceptive pain). These tests include responses to thermal, mechanical, or chemical stimuli. Thermal stimuli usually involve the application of hot stimuli (typically varying between 42-55° C.) including, for example: radiant heat to the tail (the tail flick test), radiant heat to the plantar surface of the hindpaw (the Hargreaves test), the hotplate test, and immersion of the hindpaw or tail into hot water. Immersion in cold water, acetone evaporation, or cold plate tests may also be used to test cold pain responsiveness. Tests involving mechanical stimuli typically measure the threshold for eliciting a withdrawal reflex of the hindpaw to graded strength monofilament von Frey hairs or to a sustained pressure stimulus to a paw (e.g., the Ugo Basile analgesiometer). The duration of a response to a standard pinprick may also be measured. When using a chemical stimulus, the response to the application or injection of a chemical irritant (e.g., capsaicin, mustard oil, bradykinin, ATP, formalin, acetic acid) to the skin, muscle joints or internal organs (e.g., bladder or peritoneum) is measured.

In addition, various tests assess pain sensitization by measuring changes in the excitability of the peripheral or central components of the pain neural pathway. In this regard, peripheral sensitization (i.e., changes in the threshold and responsiveness of high threshold nociceptors) can be induced by repeated heat stimuli as well as the application or injection of sensitizing chemicals (e.g., prostaglandins, bradykinin, histamine, serotonin, capsaicin, or mustard oil). Central sensitization (i.e., changes in the excitability of neurons in the central nervous system induced by activity in peripheral pain fibers) can be induced by noxious stimuli (e.g., heat), chemical stimuli (e.g., injection or application of chemical irritants), or electrical activation of sensory fibers.

Various pain tests developed to measure the effect of peripheral inflammation on pain sensitivity can also be used to study the efficacy of the compounds (Stein et al., Pharmacol. Biochem. Behav. (1988) 31: 445-451; Woolf et al., Neurosci. (1994) 62: 327-331). Additionally, various tests assess peripheral neuropathic pain using lesions of the peripheral nervous system. One such example is the “axotomy pain model” (Watson, J. Physiol. (1973) 231:41). Other similar tests include the SNL test which involves the ligation of a spinal segmental nerve (Kim and Chung, Pain (1992) 50: 355), the Seltzer model involving partial nerve injury (Seltzer, Pain (1990) 43: 205-18), the spared nerve injury (SNI) model (Decosterd and Woolf, Pain (2000) 87:149), chronic constriction injury (CCI) model (Bennett (1993) Muscle Nerve 16: 1040), tests involving toxic neuropathies such as diabetes (streptozocin model), pyridoxine neuropathy, taxol, vincristine, and other antineoplastic agent-induced neuropathies, tests involving ischaemia to a nerve, peripheral neuritis models (e.g., CFA applied peri-neurally), models of post-herpetic neuralgia using HSV infection, and compression models.

In all of the above tests, outcome measures may be assessed, for example, according to behavior, electrophysiology, neurochemistry, or imaging techniques to detect changes in neural activity.

Exemplary models of pain are also described in the Examples provided herein.

In addition to being able to modulate a particular calcium channel (e.g., Caᵥ 3.1, Caᵥ 3.2, or Caᵥ 3.3), it may be desirable that the compound has very low activity with respect to the hERG K⁺ channel, which is expressed in the heart: compounds that block this channel with high potency may cause reactions which are fatal. See, e.g., Bowlby et al., “hERG (KCNH2 or Kᵥ11.1 K⁺ Channels: Screening for Cardiac Arrhythmia Risk,” Curr. Drug Metab. 9(9):965-70 (2008)). Thus, for a compound that modulates calcium channel activity, it may also be shown that the hERG K⁺ channel is not inhibited or only minimally inhibited as compared to the inhibition of the primary channel targeted. Similarly, it may be desirable that the compound does not inhibit cytochrome p450, an enzyme that is required for drug detoxification. Such compounds may be particularly useful in the methods described herein.

The compounds of the invention modulate the activity of calcium channels; in general, said modulation is the inhibition of the ability of the channel to transport calcium. As described below, the effect of a particular compound on calcium channel activity can readily be ascertained in a routine assay whereby the conditions are arranged so that the channel is activated, and the effect of the compound on this activation (either positive or negative) is assessed. Exemplary assays are also described in the Examples.

### Libraries and Screening

The compounds of the invention can be synthesized individually using methods known in the art per se, or as members of a combinatorial library.

Synthesis of combinatorial libraries is known in the art. Suitable descriptions of such syntheses are found, for example, in Wentworth et al., Current Opinion in Biol. (1993) 9:109-115, and Salemme et al., Structure (1997) 5:319-324. The libraries contain compounds with various substituents and various degrees of unsaturation, as well as different chain lengths. The libraries, which may contain as few as 10 but typically several hundred members to several thousand members, may then be screened for compounds which are particularly effective against a specific subtype of calcium channel, e.g., the N- or T-type channel. In addition, using standard screening protocols, the libraries may be screened for compounds that block additional channels or receptors such as sodium channels, potassium channels and the like.

Methods of performing these screening functions are well known in the art. These methods can also be used for individually ascertaining the ability of a compound to agonize or antagonize the channel. Typically, the channel to be targeted is expressed at the surface of a recombinant host cell such as human embryonic kidney cells. The ability of the members of the library to bind the channel to be tested is measured, for example, by the ability of the compound in the library to displace a labeled binding ligand such as the ligand normally associated with the channel or an antibody to the channel. More typically, ability to antagonize the channel is measured in the presence of calcium, barium or other permeant divalent cation and the ability of the compound to interfere with the signal generated is measured using standard techniques. In more detail, one method involves the binding of radiolabeled agents that interact with the calcium channel and subsequent analysis of equilibrium binding measurements including, but not limited to, on rates, off rates, Kd values and competitive binding by other molecules.

Another method involves the screening for the effects of compounds by electrophysiological assay whereby individual cells are impaled with a microelectrode and currents through the calcium channel are recorded before and after application of the compound of interest.

Another method, high-throughput spectrophotometric assay, utilizes loading of the cell lines with a fluorescent dye sensitive to intracellular calcium concentration and subsequent examination of the effects of compounds on the ability of depolarization by potassium chloride or other means to alter intracellular calcium levels.

As described above, a more definitive assay can be used to distinguish inhibitors of calcium flow which operate as open channel blockers, as opposed to those that operate by promoting inactivation of the channel or as resting channel blockers. The methods to distinguish these types of inhibition are more particularly described in the examples below. In general, open-channel blockers are assessed by measuring the level of peak current when depolarization is imposed on a background resting potential of about −100 mV in the presence and absence of the candidate compound. Successful open-channel blockers will reduce the peak current observed and may accelerate the decay of this current. Compounds that are inactivated channel blockers are generally determined by their ability to shift the voltage dependence of inactivation towards more negative potentials. This is also reflected in their ability to reduce peak currents at more depolarized holding potentials (e.g., −70 mV) and at higher frequencies of stimulation, e.g., 0.2 Hz vs. 0.03 Hz. Finally, resting channel blockers would diminish the peak current amplitude during the very first depolarization after drug application without additional inhibition during the depolarization.

Accordingly, a library of compounds of, e.g., formula (I) can be used to identify a compound having a desired combination of activities that includes activity against at least one type of calcium channel. For example, the library can be used to identify a compound having a suitable level of activity on N and/or T-type calcium channels while having minimal activity on HERG K+ channels.

### Utility and Administration

### Pharmaceutical Compositions

For use as treatment of human and animal subjects, the compounds of the invention can be formulated as pharmaceutical or veterinary compositions. Depending on the subject to be treated, the mode of administration, and the type of treatment desired—e.g., prevention, prophylaxis, or therapy—the compounds are formulated in ways consonant with these parameters. A summary of such techniques is found in Remington: The Science and Practice of Pharmacy, 21st Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

The compounds described herein (e.g., a compound of any of Formulas (I)-(XXVII) or any of compounds 1-780 in Table 1) may be present in amounts totaling 1-95% by weight of the total weight of the composition. The composition may be provided in a dosage form that is suitable for intraarticular, oral, parenteral (e.g., intravenous, intramuscular), rectal, cutaneous, subcutaneous, topical, transdermal, sublingual, nasal, vaginal, intravesicular, intraurethral, intrathecal, epidural, aural, or ocular administration, or by injection, inhalation, or direct contact with the nasal, genitourinary, gastrointestinal, reproductive or oral mucosa. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

In general, for use in treatment, the compounds described herein (e.g., a compound of any of Formulas (I)-(XXVII) or any of compounds 1-780 in Table 1) may be used alone, as mixtures of two or more compounds or in combination with other pharmaceuticals. An example of other pharmaceuticals to combine with the compounds described herein (e.g., a compound of any of Formulas (I)-(XXVII) or any of compounds 1-780 in Table 1) would include pharmaceuticals for the treatment of the same indication. For example, in the treatment of pain, a compound may be combined with another pain relief treatment such as an NSAID, or a compound which selectively inhibits COX-2, or an opioid, or an adjuvant analgesic such as an antidepressant. Another example of a potential pharmaceutical to combine with the compounds described herein (e.g., a compound of any of Formulas (I)-(XXVII) or any of compounds 1-780 in Table 1) would include pharmaceuticals for the treatment of different yet associated or related symptoms or indications. Depending on the mode of administration, the compounds will be formulated into suitable compositions to permit facile delivery. Each compound of a combination therapy may be formulated in a variety of ways that are known in the art. For example, the first and second agents of the combination therapy may be formulated together or separately. Desirably, the first and second agents are formulated together for the simultaneous or near simultaneous administration of the agents.

The compounds of the invention may be prepared and used as pharmaceutical compositions comprising an effective amount of a compound described herein (e.g., a compound of any of Formulas (I)-(XXVII) or any of compounds 1-780 in Table 1) and a pharmaceutically acceptable carrier or excipient, as is well known in the art. In some embodiments, the composition includes at least two different pharmaceutically acceptable excipients or carriers.

Formulations may be prepared in a manner suitable for systemic administration or topical or local administration. Systemic formulations include those designed for injection (e.g., intramuscular, intravenous or subcutaneous injection) or may be prepared for transdermal, transmucosal, or oral administration. The formulation will generally include a diluent as well as, in some cases, adjuvants, buffers, preservatives and the like. The compounds can be administered also in liposomal compositions or as microemulsions.

For injection, formulations can be prepared in conventional forms as liquid solutions or suspensions or as solid forms suitable for solution or suspension in liquid prior to injection or as emulsions. Suitable excipients include, for example, water, saline, dextrose, glycerol and the like. Such compositions may also contain amounts of nontoxic auxiliary substances such as wetting or emulsifying agents, pH buffering agents and the like, such as, for example, sodium acetate, sorbitan monolaurate, and so forth.

Various sustained release systems for drugs have also been devised. See, for example, U.S. Pat. No. 5,624,677, which is herein incorporated by reference.

Systemic administration may also include relatively noninvasive methods such as the use of suppositories, transdermal patches, transmucosal delivery and intranasal administration. Oral administration is also suitable for compounds of the invention. Suitable forms include syrups, capsules, and tablets, as is understood in the art.

For administration to animal or human subjects, the dosage of the compounds of the invention may be, for example, 0.01-50 mg/kg (e.g., 0.01-15 mg/kg or 0.1-10 mg/kg). For example, the dosage can be 10-30 mg/kg.

Each compound of a combination therapy, as described herein, may be formulated in a variety of ways that are known in the art. For example, the first and second agents of the combination therapy may be formulated together or separately.

The individually or separately formulated agents can be packaged together as a kit. Non-limiting examples include, but are not limited to, kits that contain, e.g., two pills, a pill and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Two or more compounds may be mixed together in a tablet, capsule, or other vehicle, or may be partitioned. In one example, the first compound is contained on the inside of the tablet, and the second compound is on the outside, such that a substantial portion of the second compound is released prior to the release of the first compound.

Formulations for oral use may also be provided as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles. Generally, when administered to a human, the oral dosage of any of the compounds of the combination of the invention will depend on the nature of the compound, and can readily be determined by one skilled in the art. Typically, such dosage is normally about 0.001 mg to 2000 mg per day, desirably about 1 mg to 1000 mg per day, and more desirably about 5 mg to 500 mg per day. Dosages up to 200 mg per day may be necessary. Administration of each drug in a combination therapy, as described herein, can, independently, be one to four times daily for one day to one year, and may even be for the life of the patient. Chronic, long-term administration may be indicated.

### EXAMPLES

### Synthesis of the Invention Compounds

The following reaction schemes and examples are intended to illustrate the synthesis of a representative number of compounds. Accordingly, the following examples are intended to illustrate but not to limit the invention. Additional compounds not specifically exemplified may be synthesized using conventional methods in combination with the methods described hereinbelow.

Purification of crude organic mixtures was conducted by a High Throughput Organic Purification (HiTOP) Laboratory using reversed phase preparative HPLC. Two approaches were utilized depending on the nature of the target; a high pH approach or a low pH approach. Analytical scale chromatography was used to determine the type of preparative method required for each sample as well as to conduct final purity checks and product confirmation on collected final material. The analytical methodology is not discussed here, but is known in the art.

### Example 1

### General Procedure for the Synthesis of 2-chloroacetamides (2a-f) as Exemplified by the Preparation of N-tert-butyl-2-chloroacetamide (2a)

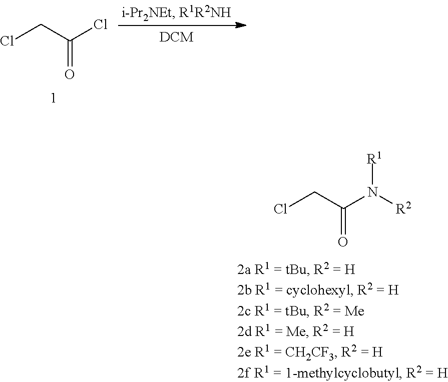

tert-Butylamine (5 g, 68.3 mmol) and DIPEA (23.8 mL, 136.7 mmol) were stirred in DCM (200 mL) at 0° C. 2-Chloroacetyl chloride (6.53 mL, 82.03 mmol) was added dropwise, and the reaction stirred for 16 hours allowing to warm to room temperature. The mixture was concentrated in vacuo, and the residue was taken up in EtOAc. The organic layer was washed with 2N HCl, dried (Na₂SO₄), and concentrated in vacuo to give N-tert-butyl-2-chloroacetamide (3) quantitatively. This material was used without further purification. The 2-chloroacetamides (2) were purified as required using automated flash chromatography and an appropriate organic eluent.

Commercially available 2-chloroacetamides were also utilized in addition to compounds (2a-f).

### Example 2

### Procedure for the Synthesis of N-(2-chloroethyl)pivalamide (5)

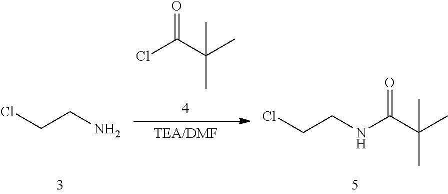

2-Chloroethanamine hydrochloride (3) (8.50 g, 73.3 mmol), TEA (25.5 mL, 183 mmol), and pivaloyl chloride (4) (9.02 mL, 73.3 mmol) were stirred in DMF (250 mL) at room temperature for 16 hours. The reaction was concentrated in vacuo, the residue was taken up in EtOAc, washed with saturated aqueous NaHCO₃, dried over Na₂SO₄, and the solvent was removed under reduced pressure. The crude material was purified by automated flash chromatography (50% EtOAc/PE) to give N-(2-chloroethyl)pivalamide (5) (3.71 g, 31%); ¹H NMR (300 mHz, CDCl₃) δ 1.21 (s, 9H), 3.62 (m, 4H), 6.08 (br s, 1H).

### Example 3

### Procedure for the Synthesis of 3-(methylsulfonyl)-5-(trifluoromethyl)picolinic acid (7)

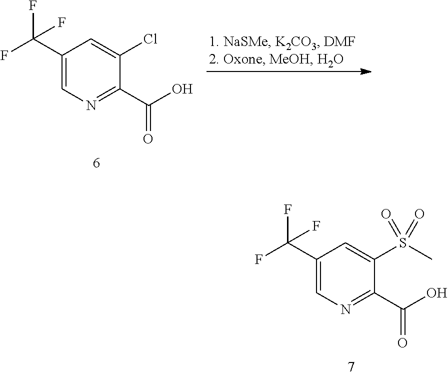

3-Chloro-5-(trifluoromethyl)picolinic acid (6) (2.11 g, 10.0 mmol), K₂CO₃ (1.38 g, 10.0 mmol), and NaSMe (1.20 g, 25.0 mmol) were stirred in DMF (15 mL) at 110° C. for 16 hours. The reaction was concentrated in vacuo, and the residue was dissolved in MeOH (80 mL) and H₂O (80 mL). Oxone monopersulfate (30 g, 49 mmol) was added, and the reaction was stirred at room temperature for 16 hours. The solid was removed by filtration, and the filtrate basified with 10% NaOH for 30 min. The MeOH was removed in vacuo, and the aqueous portion acidified to pH 1 with 6 N HCl and extracted with EtOAc (3×80 mL). The organics were dried (Na₂SO₄), concentrated in vacuo, and the residue recrystallized (with 1 eq. DMF) from EtOAc/hexanes to give 3-(methylsulfonyl)-5-(trifluoromethyl)picolinic acid containing one DMF molecule (1.70 g, 51%); ¹H NMR (300 MHz, CD₃OD) δ 2.88 (s, 3H, DMF), 3.01 (s, 3H, DMF), 3.45 (s, 3H), 8.00 (s, 1H, DMF), 8.73 (s, 1H), 9.22 (s, 1H).

### Example 4

### Procedure for the Synthesis of 2-(methylsulfonyl)-6-(trifluoromethyl)nicotinic acid (9)

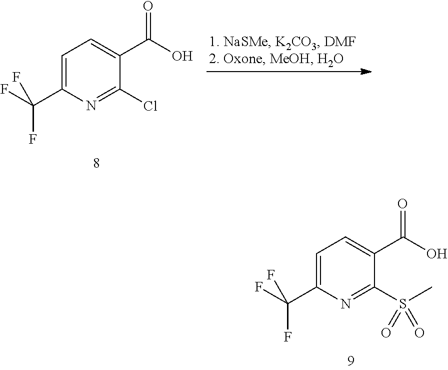

3-(Methylsulfonyl)-5-(trifluoromethyl)picolinic acid (9) was prepared in an analogous fashion using 2-chloro-6-(trifluoromethyl)nicotinic acid (8) (5.35 g, 25.3 mmol) to provide the required product (5.97 g, 69%) (containing 1 eq. of DMF); ¹H NMR (300 MHz, CD₃OD) δ 2.88 (s, 3H, DMF), 3.01 (s, 3H, DMF), 3.40 (s, 3H), 8.00 (s, 1H, DMF), 8.22 (d, 1H, J=7.5 Hz), 8.49 (d, 1H, J=7.5 Hz).

### Example 5

### Procedure for the Synthesis of 2-(isopropylsulfonyl)-6-(trifluoro methyl)nicotinic acid (10)

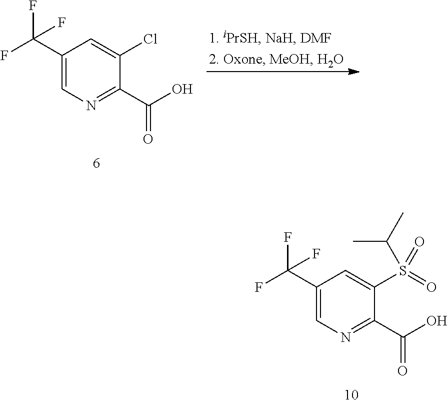

2-(isopropylsulfonyl)-6-(trifluoromethyl)nicotinic acid (10) was prepared in an analogous fashion using 3-chloro-5-(trifluoromethyl)picolinic acid (6) (1.50 g, 7.09 mmol) to give the product (1.4 g, 62%); ¹H NMR (300 MHz, CDCl₃) δ 9.06 (s, 1H), 8.56 (s, 1H), 4.09 (m, 1H), 1.31 (d, 6H, J=6.8 Hz).

### Example 6

### Procedure for the Synthesis of 2-(methylsulfonyl)-6-(trifluoromethyl) isonicotinic acid (13)

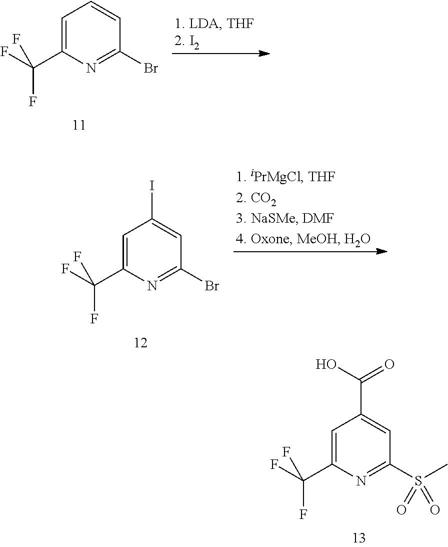

### A. Preparation of 2-bromo-4-iodo-6-(trifluoromethyl)pyridine (12)

Diisopropylamine (2.83 g, 28.0 mmol) was stirred under argon in dry THF (60 mL) at −85° C. nBuLi (1.6 M in hexanes, 17.5 mL, 28 mmol) was added dropwise, and the reaction stirred for 1 hour. 2-Bromo-6-(trifluoromethyl)pyridine (11) (3.00 g, 13.3 mmol) in dry THF (6 mL) was added dropwise, and the reaction stirred for 2 hours. I₂ (3.37 g, 13.3 mmol) was added in portions. The reaction stirred for 30 minutes, quenched with H₂O and extracted with EtOAc (3×30 mL). The organics were dried (Na₂SO₄), concentrated in vacuo and purified by automated column chromatography (EtOAc/PE, 1:8) to give 2-bromo-4-iodo-6-(trifluoromethyl)pyridine (2.3 g, 49%); ¹H NMR (300 MHz, CDCl₃) δ 7.98 (s, 1H), 8.03 (s, 1H).

### B. Preparation of 2-(methylsulfonyl)-6-(trifluoromethyl)isonicotinic acid (13)

2-Bromo-4-iodo-6-(trifluoromethyl)pyridine (12) (2.70 g, 7.67 mmol) was stirred under argon in dry THF (30 mL) at −10° C. iPrMgCl (2.0 M, THF, 4.5 mL, 9.0 mmol) was added, and the mixture was stirred at 0° C. for 30 min. CO₂ was bubbled through the reaction, and stirring continued for 1.5 hours. The reaction was then allowed to warm to room temperature. The reaction was concentrated in vacuo, taken up in DMF (20 mL), and stirred with NaSMe (0.90 g, 19 mmol) at 100° C. for 2 hours. The reaction was concentrated in vacuo, taken up in MeOH (50 mL) and H₂O (50 mL) with oxone monopersulfate (30 g, 49 mmol), and stirred at room temperature for 3 hours. The reaction was filtered, the filtrate basified with 10% NaOH for 30 min, and the MeOH removed in vacuo. The aqueous residue was acidified with 6 N HCl and extracted with EtOAc (3×50 mL). The organics were dried (Na₂SO₄), concentrated in vacuo, and the residue recrystallized from EtOAc/hexanes with the presence of 1 eq. DMF to give 2-(methylsulfonyl)-6-(trifluoromethyl)isonicotinic acid (13) (1.70 g, 51%). ¹H NMR (300 MHz, CD₃OD) δ 2.88 (s, 3H, DMF), 3.01 (s, 3H, DMF), 3.34 (s, 3H), 8.00 (s, 1H, DMF), 8.52 (s, 1H), 8.73 (s, 1H).

### Example 7

### Procedure for the Synthesis of 2-methyl-2-(3-(trifluoromethyl)phenylsulfonyl) propanoic acid (18)

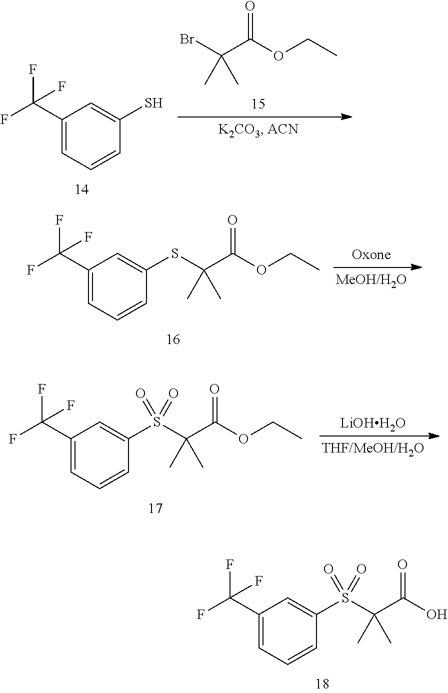

### A. Preparation of ethyl 2-methyl-2-(3-(trifluoromethyl)phenylthio)propanoate (16)

3-(Trifluoromethyl)benzenethiol (14) (25 g, 140.3 mmol), ethyl 2-bromo-2-methylpropanoate (15) (27.4 g, 140.3 mmol), and K₂CO₃ (24.2 g, 175.4 mmol) were heated at reflux in MeCN (400 mL) for 16 hours. The reaction was cooled, filtered, concentrated in vacuo, and the residue was purified by column chromatography (PE/DCM (80/20)) to give ethyl 2-methyl-2-(3-(trifluoromethyl)phenylthio)propanoate (16) as a clear oil (34.9 g, 85%). ¹H NMR (300 mHz —CH₃Cl) δ 1.49 (s, 6H), 3.65 (s, 3H), 7.45 (t, 1H, J=7.74 Hz), 7.63 (m, 2 H), 7.07 (s, 1H).

### B. Preparation of ethyl 2-methyl-2-(3-(trifluoromethyl)phenylsulfonyl) propanoate (17)

Ethyl 2-methyl-2-(3-(trifluoromethyl)phenylthio)propanoate (16) (34.9 g, 119.4 mmol) and Oxone (220.2 g, 358.2 mmol) were stirred in H₂O/MeOH (330 mL/550 mL) at room temperature for 72 hours. The reaction was filtered, the MeOH removed in vacuo, and the aqueous layer extracted with EtOAc. The organics were dried (Na₂SO₄) and concentrated in vacuo to give ethyl 2-methyl-2-(3-(trifluoromethyl)phenylsulfonyl)propanoate (17) as a clear colorless oil which was used without further purification (38.4 g, 100%). ¹H NMR (300 mHz —CH₃Cl) δ 1.63 (s, 6H), 3.70 (s, 3H), 7.73 (t, 1H, J=7.86 Hz), 7.95 (d, 1H, J=7.83 Hz), 8.06 (d, 1H, J=7.98 Hz), 8.11 (s, 1H).

### C. Preparation of 2-methyl-2-(3-(trifluoromethyl)phenylsulfonyl)propanoic acid (18)

Ethyl 2-methyl-2-(3-(trifluoromethyl)phenylsulfonyl)propanoate (17) (20 g, 61.7 mmol) and LiOH.H₂O (3.9 g, 92.5 mmol) were stirred in THF/MeOH/H₂O (175 mL, 3/1/1) for 16 hours at room temperature. The reaction was concentrated in vacuo, the residue was dissolved in H₂O, acidified to pH 2 with 6M HCl, and the product extracted with EtOAc. The organics were dried (Na₂SO₄) and concentrated in vacuo to give 2-methyl-2-(3-(trifluoromethyl)phenylsulfonyl)propanoic acid (18) (17.1 g, 93%), which was used without further purification. ¹H NMR (300 mHz —CH3Cl) δ 1.65 (s, 6H), 7.74 (t, 1H, J=7.71 Hz), 7.95 (d, 1H, J=7.83 Hz), 8.12 (d, 1H, J=8.04 Hz), 8.16 (s, 1H).

### Example 8

### General Procedure for the Preparation of 6-phenoxypyridin-3-amines (22)

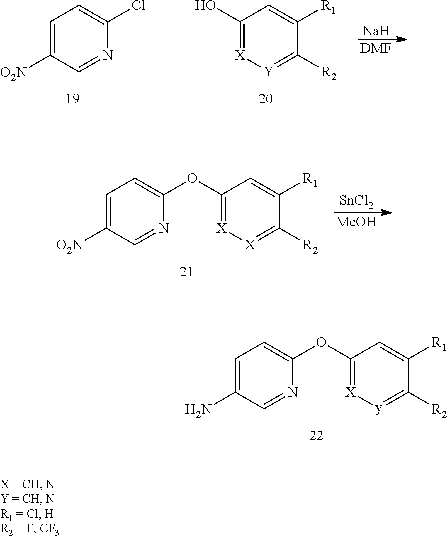

Exemplified by the procedure for 6-(3-chloro-4-fluorophenoxy)pyridin-3-amine (25)

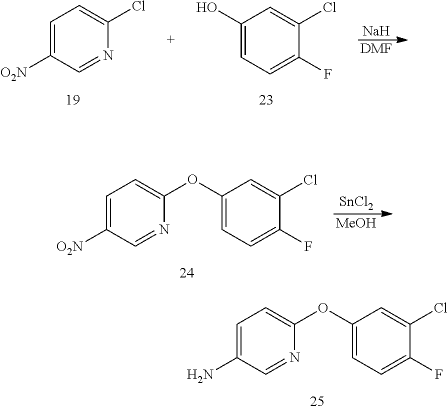

### A. Preparation of 2-(3-chloro-4-fluorophenoxy)-5-nitropyridine (24)

2-Chloro-5-nitropyridine (19) (1.0 g, 6.31 mmol), 3-chloro-4-fluorophenol (23) (0.92 g, 6.31 mmol), and NaH (60% dispersion in mineral oil) (250 mg, 6.9 mmol) were stirred under argon in DMF (20 mL) at reflux for 3 hours. The reaction was quenched with H₂O, extracted with EtOAc (3×10 mL). The organics were dried (Na₂SO₄), concentrated in vacuo, and the residue purified by automated flash chromatography (5% EtOAc/PE) to give 2-(3-chloro-4-fluorophenoxy)-5-nitropyridine (24) (0.92 g. 54%). ¹H NMR (300 mHz, CDCl₃) δ 7.04-7.10 (m, 2H), 7.19-7.25 (m, 2H), 8.52 (dd, 1H, J=2.79, 9.00 Hz), 9.03 (d, 1H, J=2.55 Hz).

### B. Preparation of 6-(3-chloro-4-fluorophenoxy)pyridin-3-amine (25)

2-(3-Chloro-4-fluorophenoxy)-5-nitropyridine (24) (0.92 g, 3.4 mmol) and SnCl₂ (3.1 g, 13.73 mmol) were stirred in MeOH (15 mL) at reflux for 16 hours. The reaction was concentrated in vacuo and stirred in NaHCO₃(sat)/CH₂Cl₂ (1:1) at room temperature for 45 min. The resulting suspension was filtered through Celite, and the filtrate partitioned between DCM and H₂O. The organics were dried (Na₂SO₄), concentrated in vacuo, and the residue purified by automated flash chromatography (5% EtOAc/PE) to give 6-(3-chloro-4-fluorophenoxy)pyridin-3-amine (25) (0.43 g, 82%). ¹H NMR (300 mHz, CDCl₃) δ6.79 (d, 1H, J=8.58 Hz), 6.97 (m, 1H), 7.08 (m, 3H), 7.70 (d, 1H, J=2.88 Hz). LCMS m/z 238.8 (calcd. for C₁₁H₈ClFN₂O 238.0).

### Example 9

### Procedure for the Synthesis of (4-(3-(trifluoromethyl)phenylsulfonyl)cyclohexyl)methanamine (30)

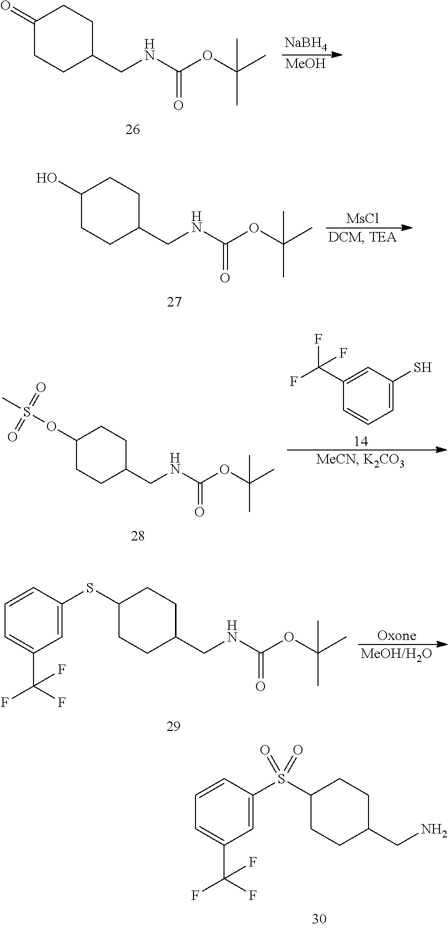

### A. Preparation of tert-butyl (4-hydroxycyclohexyl)methylcarbamate (27)

Sodium borohydride (60 mg, 1.5 mmol) was added to a solution of tert-butyl (4-oxocyclohexyl)methylcarbamate (26) (310 mg, 1.4 mmol) in MeOH (10 mL) at room temperature. The reaction was stirred for 30 minutes under argon, concentrated in vacuo, and the residue partitioned between DCM and H₂O. The organics were dried (MgSO₄) and concentrated in vacuo to give tert-butyl (4-hydroxycyclohexyl)methylcarbamate (27) (270 mg, 84%). The product was used without further purification or spectrographic confirmation.

### B. Preparation of 4-((tert-butoxycarbonylamino)methyl)cyclohexyl methanesulfonate (28)

tert-Butyl (4-hydroxycyclohexyl)methylcarbamate (27) (270 mg, 1.17 mmol) and TEA (391 μL, 2.8 mmol) were stirred in DCM at room temperature. Methanesulfonyl chloride (110 μL, 1.4 mmol) was added, and the reaction stirred at room temperature for 16 hours. The reaction was concentrated in vacuo, and the crude material purified by automated flash chromatography (35% EtOAc/PE) to give 4-((tert-butoxycarbonylamino)methyl)cyclohexyl methanesulfonate (28) (200 mg, 58%); ¹H NMR (300 mHz, CDCl₃) δ 1.50 (m, 14H), 1.86 (d, 2H, J=13.1 Hz), 2.20 (m, 3H), 2.98 (m, 5H), 4.59 (m, 2H)).

### C. Preparation of tert-butyl (4-(3-(trifluoromethyl)phenylthio)cyclohexyl)methylcarbamate (29)

4-((tert-Butoxycarbonylamino)methyl)cyclohexyl methanesulfonate (28) (200 mg, 0.67 mmol), 3-(trifluoromethyl)benzenethiol (9) (142 mg, 0.67 mmol), and K₂CO₃ (110 mg, 0.8 mmol) were heated in MeCN at reflux for 16 hours. The reaction was cooled, concentrated in vacuo, and partitioned between DCM and H₂O. The organics were separated, dried (MgSO₄), concentrated in vacuo, and the residue purified by automated flash chromatography (10% EtOAC/PE) to give tert-butyl (4-(3-(trifluoromethyl)phenylthio)cyclohexyl)methylcarbamate (29) (200 mg, 77%); ¹H NMR (300 mHz, CDCl₃) δ 1.51 (m, 19H), 1.79 (m, 5H), 3.03 (m, 3H), 3.60 (m, 1 H), 4.68 (m, 2H), 7.39 (m, 2H), 7.51 (d, 2H, J=7.17 Hz), 7.58 (s, 1H).

### D. Preparation of (4-(3-(trifluoromethyl)phenylsulfonyl)cyclohexyl)methanamine (30)

tert-Butyl (4-(3-(trifluoromethyl)phenylthio)cyclohexyl)methylcarbamate (29) (200 mg, 0.52 mmol) and Oxone® (1 g, 1.6 mmol) were stirred in MeOH/H₂O (15 mL, 3/2 vv) at room temperature for 16 hours. The reaction was filtered, the filtrate concentrated in vacuo, and the residue partitioned between DCM and H₂O. The organics were washed with saturated NaHCO₃, dried (MgSO₄) and concentrated in vacuo to give of (4-(3-(trifluoromethyl)phenylsulfonyl)cyclohexyl)methanamine (30) (40 mg, 24%), which was used without further purification.

### Example 10

### Procedure for the Synthesis of cis (4-(3-(trifluoromethyl)phenylsulfonyl)cyclohexyl)methanamine hydrochloride (35)

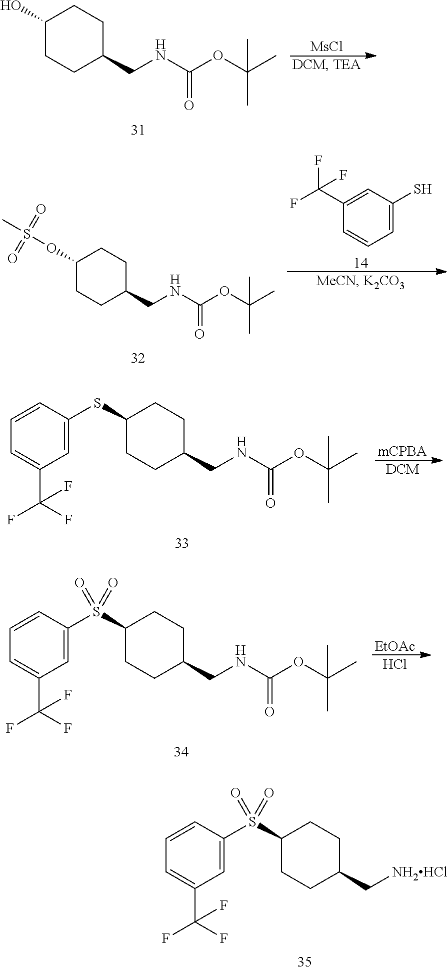

### A. Preparation of trans 4-((tert-butoxycarbonylamino)methyl)cyclohexyl methanesulfonate (32)

Trans tert-butyl (4-hydroxycyclohexyl)methylcarbamate (31) (1.5 g, 6.6 mmol) and TEA (2.8 mL, 20 mmol) were stirred in DCM (25 mL) at room temperature. Methanesulfonyl chloride (621 μL, 8.0 mmol) was added, and the reaction stirred at room temperature for 16 hours. The reaction was concentrated in vacuo, and the crude material purified by automated flash chromatography (20% EtOAc/PE) to give trans 4-((tert-butoxycarbonylamino)methyl)cyclohexyl methanesulfonate (32) (1.77 g, 91%).

### B. Preparation of cis tert-butyl (4-(3-(trifluoromethyl)phenylthio)cyclohexyl)methylcarbamate (33)

Trans 4-((tert-butoxycarbonylamino)methyl)cyclohexyl methanesulfonate (32) (1.77 g, 6 mmol), 3-(trifluoromethyl)benzenethiol (14) (1.24 g, 6.0 mmol), and K₂CO₃ (1 g, 7.2 mmol) were heated in MeCN at reflux for 16 hours. The reaction was cooled and filtered. The filtrate was concentrated in vacuo, and the crude material purified by automated flash chromatography (5% EtOAC/PE) to give cis tert-butyl (4-(3-(trifluoromethyl)phenylthio)cyclohexyl)methylcarbamate (33) (1.56 g, 67%). ¹H NMR (300 mHz, CDCl₃) δ 1.65 (m, 20H), 1.81 (m, 5H), 2.07 (m, 2H), 3.05 (m, 3H), 3.61 (m, 1H), 4.62 (bs, 1H), 5.66 (s, 1H), 7.45 (m, 2H), 7.54 (d, 2H), 7.60 (s, 1H).

### C. Preparation of cis tert-butyl (4-(3-(trifluoromethyl)phenylsulfonyl)cyclohexyl)methylcarbamate (34)

Cis tert-butyl (4-(3-(trifluoromethyl)phenylthio)cyclohexyl)methylcarbamate (33) (1.56 g, 4.0 mmol) and mCPBA (77%, 2.1 g, 12.0 mmol) were stirred in DCM (50 mL) at room temperature for 16 hours. The reaction was filtered, additional DCM (50 mL) added, and the organics washed sequentially with 2M NaOH, H₂O and saturated NaCl solution. The organics were dried (MgSO₄) and concentrated in vacuo to give cis tert-butyl (4-(3-(trifluoromethyl)phenylsulfonyl)-cyclohexyl)methylcarbamate (34) (1.27 g, 82%); ¹H NMR (300 mHz, CDCl₃) δ 1.53 (m, 21H), 1.77 (m, 5H), 3.06 (m, 5H), 4.58 (bs, 1H), 7.74 (t, 1H, J=7.77 Hz), 7.93 (d, 1H, J=7.65 Hz), 8.08 (d, 1H, J=8.04 Hz), 8.15 (s, 1H). The product was used without further purification.

### D. Preparation of cis-(4-(3-(trifluoromethyl)phenylsulfonyl)cyclohexyl)-methanamine hydrochloride (35)

Cis tert-butyl (4-(3-(trifluoromethyl)phenylsulfonyl)cyclohexyl)methylcarbamate (34) (1.27 g, 3.3 mmol) was stirred in EtOAc (30 mL) at room temperature. Gaseous HCl was bubbled through the solution for 1 minute, and then the reaction stirred at room temperature for 20 minutes. The solvent was removed in-vacuo to give cis (4-(3-(trifluoromethyl)phenylsulfonyl)cyclohexyl)-methanamine hydrochloride (35) (1.18 g, 100%). The product was confirmed by subsequent derivitization and was used without further purification.

### Example 11

### Procedure for the Synthesis of (1-methyl-4-(3-(trifluoromethyl)phenylsulfonyl)cyclohexyl)methanamine (44)

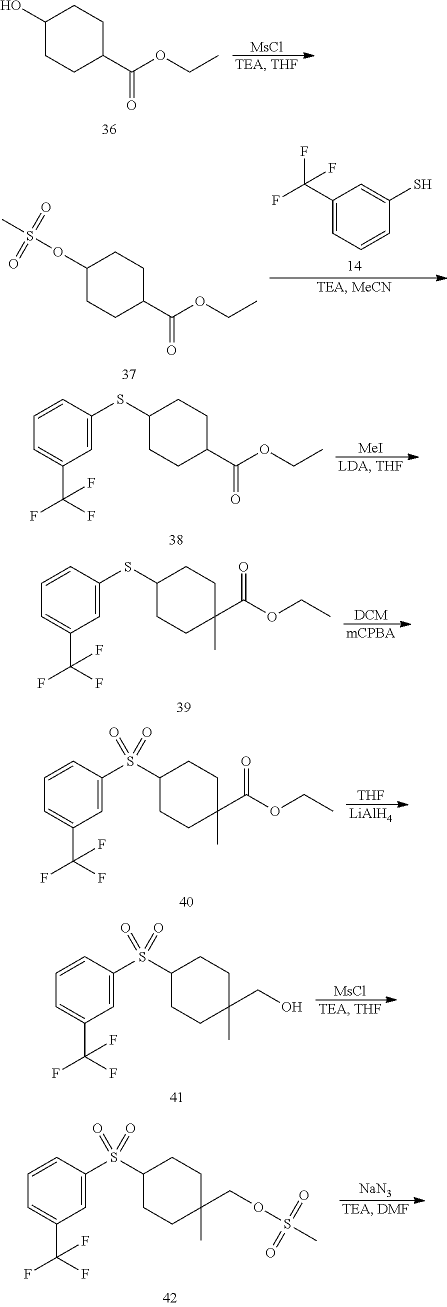

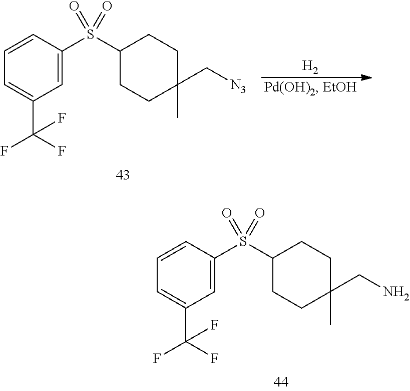

### A. Preparation of ethyl 4-(methylsulfonyloxy)cyclohexanecarboxylate (37)

Ethyl 4-hydroxycyclohexanecarboxylate (36) (mixture of cis and trans) (10 g, 58 mmol) and TEA (16.1 mL, 116 mmol) were stirred in THF (150 mL) at room temperature. Methanesulfonyl chloride (4.97 mL, 64 mmol) was added, and the reaction stirred at room temperature for 30 minutes. The precipitate was removed by filtration, washed with additional THF (80 mL), and the filtrate was concentrated in vacuo. The residue was taken up in DCM (150 mL) and washed sequentially with saturated NH₄Cl solution and saturated NaHCO₃ solution. The organics were separated, dried (MgSO₄), and concentrated in vacuo to give ethyl 4-(methylsulfonyloxy)cyclohexanecarboxylate (37) (14.5 g, 100%). ¹H NMR (300 mHz, CDCl₃) δ 1.23 (t, 3H, J=14.3 Hz), 1.98 (m, 10H), 3.0 (s, 3H), 4.10 (q, 2H, J=7.56 Hz), 4.60 (m, 0.5H), 4.90 (m, 0.5H). The product was used without further purification.

### B. Preparation of ethyl 4-(3-(trifluoromethyl)phenylthio)cyclohexanecarboxylate (38)

Ethyl 4-(methylsulfonyloxy)cyclohexanecarboxylate (37) (14.5 g, 58 mmol), 3-(trifluoromethyl)benzenethiol (14) (10.3 g, 58.0 mmol), and TEA (16 mL, 116 mmol) were heated in MeCN at reflux for 16 hours. The reaction was cooled and concentrated in vacuo. The residue was taken up in DCM (150 mL) and washed sequentially with saturated NH₄Cl solution and saturated NaHCO₃ solution. The organics were separated, dried (MgSO₄), concentrated in vacuo, and the crude material purified by automated flash chromatography (5% EtOAc/PE) to give ethyl 4-(3-(trifluoromethyl)phenylthio)cyclohexanecarboxylate (38) (8.24 g, 43%). ¹H NMR (300 mHz, CDCl₃) δ 1.24 (m, 3H), 1.44 (m, 2H), 1.73 (m, 3H), 2.05 (m, 3H), 2.26 (m, 0.5H), 2.44 (m, 0.5H), 3.08 (m, 0.5H), 3.44 (m, 0.5H), 4.12 (m, 2H).

### C. Preparation of ethyl 1-methyl-4-(3-(trifluoromethyl)phenylthio)cyclohexanecarboxylate (39)

4-Ethyl 4-(3-(trifluoromethyl)phenylthio)cyclohexanecarboxylate (38) (8.24 g, 24.8 mmol) was stirred under argon in dry THF (100 mL) at −78° C. LDA (2.0 M solution in heptane/THF/ethylbenzene; 37 mL, 74 mmol) was added dropwise, and the solution stirred for 30 minutes. Iodomethane (3.1 mL, 50 mmol) in dry THF (20 mL) was added dropwise, and the reaction stirred at −78° C. for 1 hour then allowed to warm to room temperature. The reaction was quenched with H₂O, extracted with Et₂O, the organics separated, dried (MgSO₄), and concentrated in vacuo. The crude product was purified by automated flash chromatography (3% EtOAc/PE) to give ethyl 1-methyl-4-(3-(trifluoromethyl)phenylthio)cyclohexanecarboxylate (39) (6.14 g, 72%). ¹H NMR (300 mHz, CDCl₃) δ 1.13 (s, 3H), 1.15 (t, 3H, J=4.32 Hz), 1.35 (q, 2H, J=10.53 Hz), 1.85 (d, 2H, J=5.61 Hz), 2.19 (d, 2H, J=14.1 Hz), 2.99 (m, 1 H), 4.07 (q, 2H, J=7.14 Hz), 7.34 (m, 2H), 7.47 (d, 1H, J=7.53 Hz), 7.54 (s, 1H).

### D. Preparation of ethyl 1-methyl-4-(3-(trifluoromethyl)phenylsulfonyl)cyclohexanecarboxylate (40)

Ethyl 1-methyl-4-(3-(trifluoromethyl)phenylthio)cyclohexanecarboxylate (39) (6.14 g, 17.7 mmol) and mCPBA (77%, 9.4 g, 53.2 mmol) were stirred in DCM (50 mL) at room temperature for 16 hours. The resultant precipitate was removed by filtration, the filtrate washed sequentially with 10% NaOH solution and H₂O, dried (MgSO₄), and concentrated in vacuo to give ethyl 1-methyl-4-(3-(trifluoromethyl)phenylsulfonyl)cyclohexanecarboxylate (40) (5.9 g, 88%). ¹H NMR (300 mHz, CDCl₃) δ 1.10 (m, 9H), 1.42 (q, 2H, J=13.23 Hz), 1.91 (d, 2H, J=11.46 Hz), 2.27 (d, 2H, J=13.32 Hz), 2.84 (m, 1H), 4.03 (q, 2H, J=7.08 Hz), 7.66 (t, 1H, J=7.83 Hz), 7.85 (d, 1H, J=7.77 Hz), 7.98 (d, 1H, J=7.83 Hz), 8.05 (s, 1 H). The product was used without further purification.

### E. Preparation of (1-methyl-4-(3-(trifluoromethyl)phenylsulfonyl)cyclohexyl)methanol (41)

Ethyl 1-methyl-4-(3-(trifluoromethyl)phenylsulfonyl)cyclohexane carboxylate (40) (2.0 g, 5.3 mmol) was stirred under argon in dry THF (30 mL) at room temperature. LiAlH₄ (239 mg, 6.3 mmol) was added, and the reaction stirred for 30 minutes. The reaction was quenched with the dropwise addition of 10% NaOH, and filtered, washing with additional THF. The filtrate was concentrated in vacuo. The residue was taken up in EtOAc, washed sequentially with NH₄Cl saturated solution, NaHCO₃ saturated solution and H₂O, and dried (MgSO₄). The organics were concentrated in-vacuo to give (1-methyl-4-(3-(trifluoromethyl)phenylsulfonyl)cyclohexyl)methanol (41). (1.69 g, 95%). ¹H NMR (300 mHz, CDCl₃) δ 0.92 (s, 3H), 1.14 (m, 3H), 1.28 (t, 1H, J=5.52 Hz), 1.74 (m, 10H), 2.92 (m, 1H), 3.47 (d, 2H, J=5.40 Hz), 3.75 (m, 1H), 7.74 (t, 1H, J=7.83 Hz), 7.93 (d, 1H, J=7.77 Hz), 8.08 (d, 1H, J=7.83), 8.15 (s, 1H). The product was used without further purification.

### F. Preparation of (1-methyl-4-(3-(trifluoromethyl)phenylsulfonyl)cyclohexyl)methyl methane-sulfonate (42)

1-Methyl-4-(3-(trifluoromethyl)phenylsulfonyl)cyclohexyl)methanol (41) (1.69 g, 5.0 mmol) and TEA (2.1 mL, 15 mmol) were stirred in THF (30 mL) at room temperature. Methanesulfonyl chloride (466 μL, 6 mmol) was added and stirred for 30 minutes. The resultant precipitate was removed by filtration, the filtrate concentrated in vacuo, taken up in EtOAc and washed sequentially with NH₄Cl saturated solution, NaHCO₃ saturated solution and H₂O. The organics were dried (MgSO₄) and concentrated in vacuo to give (1-methyl-4-(3-(trifluoromethyl)phenylsulfonyl)cyclohexyl)methyl methanesulfonate (42) (2.07 g, 100%). ¹H NMR (300 mHz, CDCl₃) δ 0.92 (s, 3H), 1.17 (m, 3H), 1.32 (m, 6H), 1.70 (m, 11H), 2.72 (s, 1H), 2.97 (m, 5H), 3.81 (t, 2H, J=6.03 Hz), 3.96 (s, 2H), 7.68 (t, 1H, J=7.83 Hz), 7.87 (d, 1H, J=7.77), 8.01 (d, 1H, J=7.83 Hz), 8.07 (s, 1 H). The product was used without further purification.

### G. Preparation of 1-(4-(azidomethyl)-4-methylcyclohexylsulfonyl)-3-(trifluoromethyl)benzene (43)

(1-Methyl-4-(3-(trifluoromethyl)phenylsulfonyl)cyclohexyl)methyl methanesulfonate (42) (2.07 g, 5.0 mmol), NaN₃ (1.63 g, 25 mmol), and TEA (2.1 mL, 15 mmol) were heated at reflux in DMF (15 mL) for 16 hours. The reaction was cooled, concentrated in vacuo, taken up in EtOAc and washed sequentially with H₂O, NH₄Cl saturated solution, and NaHCO₃ saturated solution. The organics were dried (MgSO₄), concentrated in vacuo, and the crude material purified by automated flash chromatography (10% EtOAc/PE) to give 1-(4-(azidomethyl)-4-methylcyclohexylsulfonyl)-3-(trifluoromethyl)benzene (43) (1.11 g, 61%). ¹H NMR (300 mHz, CDCl₃) δ 0.94 (s, 3H), 1.20 (m, 3H), 1.75 (m, 7H), 2.05 (s, 1H), 2.90 (m, 1H), 3.26 (s, 2H), 7.75 (t, 1H, J=7.80 Hz), 7.94 (d, 1H, J=7.77 Hz), 8.08 (d, 1 H, J=7.83 Hz), 8.15 (s, 1H).

### H. Preparation of (1-methyl-4-(3-(trifluoromethyl)phenylsulfonyl)cyclohexyl)methanamine (44)

1-(4-(Azidomethyl)-4-methylcyclohexylsulfonyl)-3-(trifluoromethyl)benzene (43) (1.11 g, 3.1 mmol) and Pd(OH)₂ (100 mg, cat) were taken up in EtOH (20 mL) and placed in a parr hydrogenator. The reaction was agitated under H₂ (50 PSI) for 1 h at room temperature. The reaction was filtered through celite, and the filtrate concentrated in vacuo to give (1-methyl-4-(3-(trifluoromethyl)phenylsulfonyl)cyclohexyl)methanamine (44) (1.01 g, 97%) which was used without further purification.

### Example 12

### Procedure for the Synthesis of 1-methyl-4-(3-(trifluoromethyl)phenylsulfonyl)cyclohexanamine hydrochloride (46)

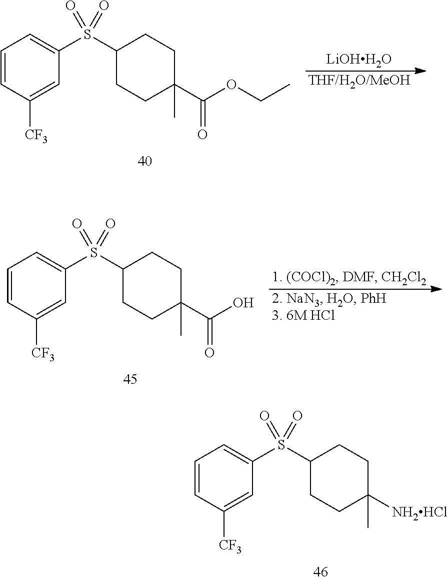

### A. Preparation of 1-methyl-4-(3-(trifluoromethyl)phenylsulfonyl)cyclohexane carboxylic acid (45)

Ethyl 1-methyl-4-(3-(trifluoromethyl)phenylsulfonyl)cyclohexane carboxylate (40) (3.1 g, 8.2 mmol) and LiOH.H₂O (447 mg, 10.7 mmol) were stirred in THF/H₂O/MeOH (3/3/1, 20 ml) at room temperature for 16 hours. Additional LiOH.H₂O (1.3 g, 31 mmol) was added, and the reaction heated at reflux for 16 hours. The organics were removed in vacuo, the reaction diluted with H₂O, acidified to pH 1 with conc HCl, and extracted with EtOAc. The organics were dried (Na₂SO₄) and concentrated in vacuo to give 1-methyl-4-(3-(trifluoromethyl)phenylsulfonyl)cyclohexanecarboxylic acid (45); ¹H NMR (300 mHz, CDCl₃) δ 1.02 (m, 6H), 1.63 (m, 6H), 2.18 (m, 2H), 2.74 (t, 1H, J=11.9 Hz), 7.56 (t, 1H, J=7.8 Hz), 7.76 (d, 1H, J=8.04 Hz), 7.90 (d, 1H, J=7.68 Hz), 7.97 (s, 1H).

### B. Preparation of 1-methyl-4-(3-(trifluoromethyl)phenylsulfonyl)cyclohexanamine hydrochloride (46)

1-Methyl-4-(3-(trifluoromethyl)phenylsulfonyl)cyclohexanecarboxylic acid (45) (1.60 g, 4.57 mmol) and DMF (1 drop, cat.) were stirred under argon in DCM (25 mL) at room temperature. (COCl)₂ (2.5 mL, excess) was added, and the reaction was heated to reflux for one hour. The reaction was concentrated in vacuo and dried under high vacuum for two hours. The residue was stirred in benzene (10 mL) under argon at 0° C. NaN₃ (0.59 g, 9.1 mmol) in water (7 mL) was added slowly, and the reaction was allowed to warm to room temperature and stirred for two hours. The organics were separated, washed with saturated NaHCO₃ solution, dried (Na₂SO₄), and then heated to 65° C. for two hours. The reaction was treated with 6 M HCl (30 mL) and heated to 90° C. for 16 hours. The reaction was then concentrated in vacuo to give 1-methyl-4-(3-(trifluoromethyl)phenylsulfonyl)cyclohexanamine hydrochloride (46) (1.37 g, 84%). ¹H NMR (300 mHz, CDCl₃) δ 1.35 (s, 1H), 1.69 (m, 2H), 1.85 (m, 2H), 2.02 (m, 4H), 3.37 (m, 1H), 7.92 (t, 1H, J=7.92 Hz), 8.11 (d, 1H, J=7.70 Hz), 8.21 (m, 2H). The product was used without additional purification.

### Example 13

### Procedure for the Synthesis of (4-fluoro-1-methyl-4-(3-(trifluoromethyl)phenylsulfonyl)cyclohexyl)methanamine (51)

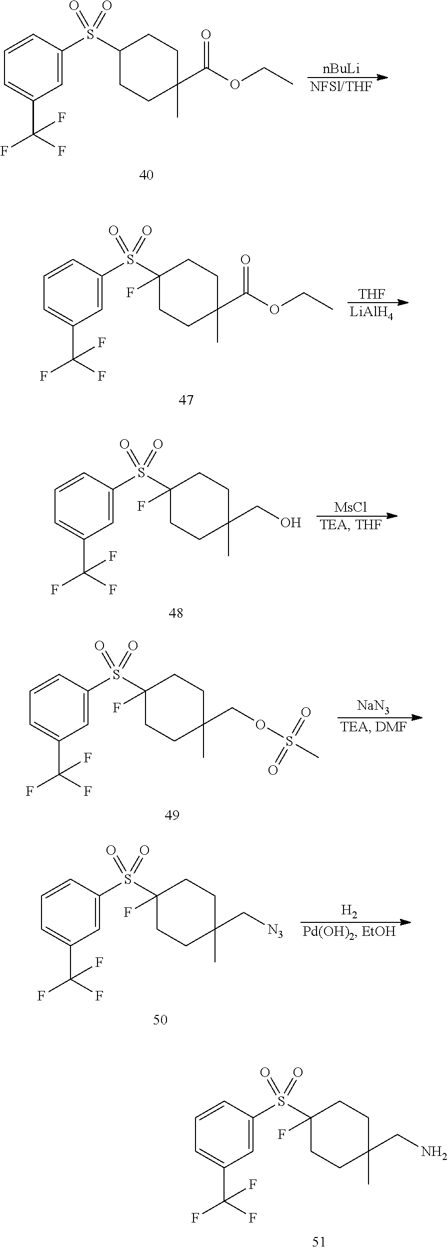

### A. Preparation of ethyl 4-fluoro-1-methyl-4-(3-(trifluoromethyl)phenylsulfonyl)cyclohexane-carboxylate (47)

Ethyl 1-methyl-4-(3-(trifluoromethyl)phenylthio)cyclohexanecarboxylate (40) (2.5 g, 6.6 mmol) was stirred under argon in dry THF (30 mL) at −78° C. nBuLi (1.6 M solution in hexane) (5 mL, 7.9 mmol) was added dropwise, and the reaction stirred for 30 minutes. N-Fluorobenzenesulfonimide (NFSI) (2.48 g, 7.9 mmol) in dry THF (20 mL) was added dropwise. The reaction was then stirred at −78° C. for 30 minutes, followed by 1 hour allowing to warm to room temperature. The reaction was cooled to 0° C., quenched with NH₄Cl saturated solution, and extracted with EtOAc. The organics were dried (MgSO₄), concentrated in vacuo, and the residue purified by automated flash chromatography (10% EtOac/PE) to give ethyl 4-fluoro-1-methyl-4-(3-(trifluoromethyl)phenylsulfonyl)cyclohexanecarboxylate (47) (1.17 g, 45%). ¹H NMR (300 mHz, CDCl₃) δ 1.27 (m, 7H), 2.08 (m, 10H), 4.14 (q, 2H, J=4.71 Hz), 7.77 (t, 1H, J=7.86 Hz), 7.98 (d, 1H, J=7.83 Hz), 8.13 (d, 1H, J=7.83 Hz), 8.19 (s, 1H).

### B. Preparation of (4-fluoro-1-methyl-4-(3-(trifluoromethyl)phenylsulfonyl)cyclohexyl)methanol (48)

Ethyl 4-fluoro-1-methyl-4-(3-(trifluoromethyl)phenylsulfonyl)cyclohexanecarboxylate (47) (1.17 g, 3.0 mmol) was stirred under argon in dry THF (20 mL) at room temperature. LiAlH₄ (133 mg, 3.5 mmol) was added, and the reaction stirred for 30 minutes. The reaction was quenched with the dropwise addition of 10% NaOH and then filtered, washing with additional THF. The filtrate was concentrated in vacuo. The residue was taken up in EtOAc, washed sequentially with NH₄Cl saturated solution, NaHCO₃ saturated solution, and H₂O, and dried (MgSO₄). The organics were concentrated in vacuo to give (4-fluoro-1-methyl-4-(3-(trifluoromethyl)phenylsulfonyl)cyclohexyl)methanol (48) (0.93 g, 88%). ¹H NMR (300 mHz, CDCl₃) δ 1.00 (s, 3H), 1.26 (m, 1H), 1.55 (m, 7H), 1.90 (m, 2H), 2.05 (s, 1H), 2.23 (m, 3H), 3.34 (s, 2H), 4.13 (m, 1H), 7.76 (t, 1H, J=7.86 Hz), 7.98 (d, 1H, J=7.86 Hz), 8.14 (d, 1H, J=7.89 Hz), 8.20 (s, 1H). The product was used without further purification.

### C. Preparation of (4-fluoro-1-methyl-4-(3-(trifluoromethyl)phenylsulfonyl)cyclohexyl)methyl methanesulfonate (49)

(4-fluoro-1-methyl-4-(3-(trifluoromethyl)phenylsulfonyl)cyclohexyl)methanol (48) (0.93 g, 2.63 mmol) and TEA (920 μL, 6.6 mmol) were stirred in THF (20 mL) at room temperature. Methanesulfonyl chloride (218 μL, 2.8 mmol) was added, and the reaction was stirred for 30 minutes. The resultant precipitate was removed by filtration. The filtrate was concentrated in vacuo, taken up in DCM, and washed sequentially with NH₄Cl saturated solution, NaHCO₃ saturated solution, and H₂O. The organics were dried (MgSO₄) and concentrated in vacuo to give (4-fluoro-1-methyl-4-(3-(trifluoromethyl)phenylsulfonyl)cyclohexyl)methyl methanesulfonate (49) (0.97 g, 85%). ¹H NMR (300 mHz, CDCl₃) δ 0.87 (s, 3H), 1.04 (t, 2H, J=7.14 Hz), 1.36 (m, 5H), 1.68 (m, 2H), 1.82 (s, 2H), 2.34 (m, 2H), 2.77 (s, 3H), 3.67 (s, 2H), 3.92 (q, 1H, J=4.35 Hz), 7.55 (t, 1H, J=7.86 Hz), 7.76 (d, 1H, J=7.86 Hz), 7.91 (d, 1H, J=7.92 Hz), 8.07 (s, 1H). The product was used without further purification.

### D. Preparation of 1-(4-(azidomethyl)-1-fluoro-4-methylcyclohexylsulfonyl)-3-(trifluoromethyl)-benzene (50)

(4-Fluoro-1-methyl-4-(3-(trifluoromethyl)phenylsulfonyl)cyclohexyl)methyl methanesulfonate (49) (0.97 g, 2.2 mmol), NaN₃ (600 mg, 9.2 mmol), and TEA (1.3 mL, 9.2 mmol) were heated at reflux in DMF (15 mL) for 16 hours. The reaction was cooled, diluted with EtOAc (50 mL), and washed with H₂O (2×30 mL). The organics were dried (MgSO₄), concentrated in vacuo and the crude material purified by automated flash chromatography (5% EtOAc/PE) to give 1-(4-(azidomethyl)-1-fluoro-4-methylcyclohexylsulfonyl)-3-(trifluoromethyl)benzene (50) (630 mg, 72%). ¹H NMR (300 mHz, CDCl₃) δ 0.96 (s, 3H), 1.19 (t, 1H, J=7.17 Hz), 1.45 (m, 5H), 1.83 (m, 2H), 1.97 (s, 1H), 2.12 (m, 2H), 3.06 (s, 2H), 4.05 (q, 1H, J=7.14 Hz), 7.69 (t, 1H, J=7.86 Hz), 7.91 (d, 1H, J=7.83 Hz), 8.06 (d, 1H, J=7.89 Hz), 8.12 (s, 1H).

### E. Preparation of (4-fluoro-1-methyl-4-(3-(trifluoromethyl)phenylsulfonyl)cyclohexyl)methan-amine (51)

1-(4-(Azidomethyl)-1-fluoro-4-methylcyclohexylsulfonyl)-3-(trifluoromethyl)benzene (50) (630 mg, 1.66 mmol) and Pd(OH)₂ (60 mg, cat) were taken up in EtOH (20 mL) and placed in a Parr hydrogenator. The reaction was agitated under H₂ (50 PSI) for 1 hour at room temperature. The reaction was filtered through Celite, and the filtrate concentrated in vacuo to give (4-fluoro-1-methyl-4-(3-(trifluoromethyl)phenylsulfonyl)cyclohexyl)methanamine (51) (520 mg, 89%) which was used without further purification.

### Example 14

### Procedure for the Synthesis of 2-chloro-1-(4-methyl-4-(3-(trifluoromethyl)phenylsulfonyl)piperidin-1-yl)ethanone (58)

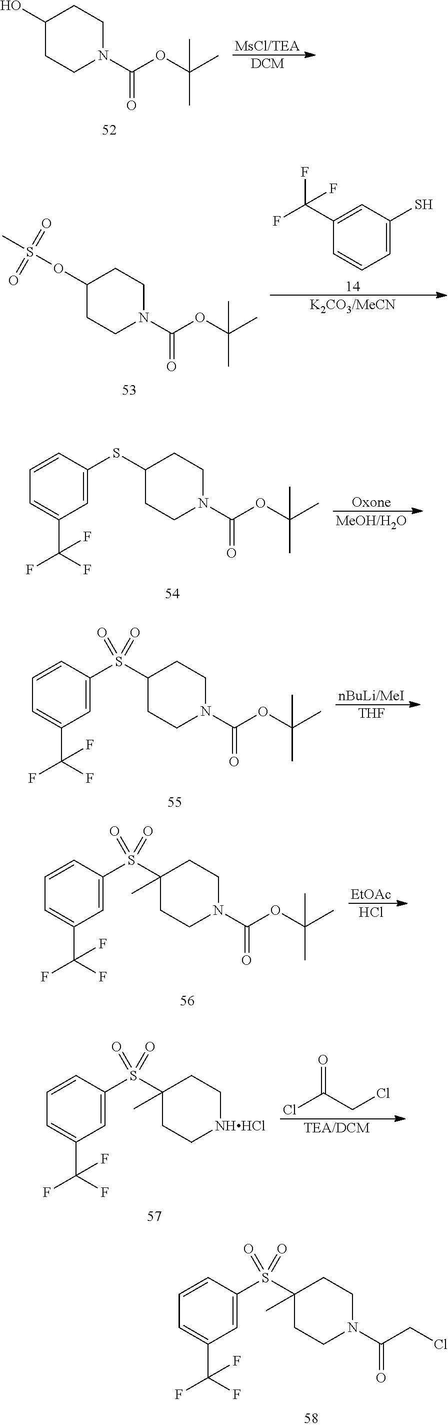

### A. Preparation of tert-butyl 4-(methylsulfonyloxy)piperidine-1-carboxylate (53)

tert-Butyl 4-hydroxypiperidine-1-carboxylate (52) (12.03 g, 59.8 mmol) and TEA (12.5 mL, 89.7 mmol) were stirred under argon in DCM (100 mL) at 0° C. Methanesulfonyl chloride was added, and the reaction stirred for 55 minutes. The reaction was washed with saturated NaHCO₃ solution, dried (Na₂SO₄), and concentrated in vacuo to give tert-butyl 4-(methylsulfonyloxy)piperidine-1-carboxylate (53) (16.0 g, 96%). ¹H NMR (300 mHz, CDCl₃) 1.45 (s, 9H), 1.82 (m, 2 H), 1.95 (m, 2H), 3.03 (s, 3H), 3.29 (m, 2H), 3.69 (m, 2H), 4.89 (m, 1H). The product was used without further purification.

### B. Preparation of tert-butyl 4-(3-(trifluoromethyl)phenylthio)piperidine-1-carboxylate (54)

tert-Butyl 4-(methylsulfonyloxy)piperidine-1-carboxylate (53) (2.0 g, 7.2 mmol), 3-(trifluoromethyl)benzenethiol (9) (1.28 g, 7.2 mmol), and K₂CO₃ (2.1 g, 156 mmol) were heated in MeCN at reflux for 2 hours. The reaction was concentrated in vacuo, and the residue partitioned between DCM and H₂O. The organics were separated, dried (MgSO₄), and concentrated in vacuo. The crude material was purified by automated flash chromatography (5% EtOAc/PE) to give tert-butyl 4-(3-(trifluoromethyl)phenylthio)piperidine-1-carboxylate (54) (2.0 g, 77%). ¹H NMR (300 mHz, CDCl₃) 1.51 (m, 14H), 1.93 (m, 2H), 2.95 (m, 2H), 3.24 (m, 1H), 3.96 (m, 2H), 7.49 (m, 2H), 7.57 (d, 1H, J=7.62 Hz), 7.64 (s, 1H).

### C. Preparation of tert-butyl 4-(3-(trifluoromethyl)phenylsulfonyl)piperidine-1-carboxylate (55)

tert-Butyl 4-(3-(trifluoromethyl)phenylthio)piperidine-1-carboxylate (54) (2.0 g, 5.5 mmol) and Oxone® 10.2 g, 16.6 mmol) were stirred in MeOH/H₂O (50 mL, 3/2 vv) at room temperature for 16 hours. The reaction was filtered, the MeOH removed in vacuo, and the aqueous layer extracted with EtOAc. The organics were separated, dried (MgSO₄), and concentrated in vacuo to give tert-butyl 4-(3-(trifluoromethyl)phenylsulfonyl)piperidine-1-carboxylate (55) (1.24 g, 57%). ¹H NMR (300 mHz, CDCl₃) 1.37 (s, 9H), 1.57 (m, 4H), 1.91 (d, 2H, J=12.78 Hz), 2.60 (m, 2H), 3.00 (m, 1H), 4.18 (d, 2H, J=11.31 Hz), 7.69 (t, 1H, J=7.83 Hz), 7.88 (d, 1H, J=7.86 Hz), 8.00 (d, 1H, J=7.86 Hz), 8.08 (s, 1H). The product was used without further purification.

### D. Preparation of tert-butyl 4-methyl-4-(3-(trifluoromethyl)phenylsulfonyl)piperidine-1-carboxylate (56)

tert-Butyl 4-(3-(trifluoromethyl)phenylsulfonyl)piperidine-1-carboxylate (55) (676 mg, 1.72 mmol) was stirred under argon in dry THF (10 mL) at −78° C. nBuLi (1.6 M solution in hexane; 1.3 mL, 2.08 mmol) was added, and the reaction stirred for 30 minutes. Iodomethane (0.5 mL, 8.1 mmol) was added, and the reaction stirred for 16 hours allowing to warm to room temperature. The reaction was quenched with NH₄Cl saturated solution, the organics separated, diluted (EtOAc), washed with brine, dried (Na₂SO₄) and concentrated in vacuo to give tert-butyl 4-methyl-4-(3-(trifluoromethyl)phenylsulfonyl)piperidine-1-carboxylate (56) The product was used without further purification.

### E. Preparation of 4-methyl-4-(3-(trifluoromethyl)phenylsulfonyl)piperidine hydrochloride (57)

tert-Butyl 4-methyl-4-(3-(trifluoromethyl)phenylsulfonyl)piperidine-1-carboxylate (56) (730 mg, 1.8 mmol) was stirred in EtOAc (10 mL) at room temperature. Gaseous HCl was bubbled through the solution for 1 minute then stirred at room temperature for 20 minutes. The reaction was concentrated in vacuo to give 4-methyl-4-(3-(trifluoromethyl)phenylsulfonyl)piperidine hydrochloride (57) (566 mg, 91%).

### F. Preparation of 2-chloro-1-(4-methyl-4-(3-(trifluoromethyl)phenylsulfonyl)piperidin-1-yl)ethanone (58)

4-methyl-4-(3-(trifluoromethyl)phenylsulfonyl)piperidine hydrochloride (57) (518 mg, 1.5 mmol) and DIPEA (0.6 mL, 3.3 mmol) were stirred in DCM (15 mL) at 0° C. Chloro acetylchloride (130 μL, 1.63 mmol) was added and the reaction was stirred for 15 hours and then allowed to warm to room temperature. The reaction was concentrated in vacuo and purified by automated flash chromatography (20% EtOAc/DCM) to give 2-chloro-1-(4-methyl-4-(3-(trifluoromethyl)phenylsulfonyl)piperidin-1-yl)ethanone (58) (400 mg, 69%). ¹H NMR (300 mHz, CDCl₃) 1.36 (s, 3H), 1.59 (m, 2H), 2.10 (m, 2H), 2.81 (t, 1H, J=11.28 Hz), 3.21 (t, 1H, J=11.31 Hz), 3.84 (d, 1H, J=13.98 Hz), 3.99 (s, 2H), 4.42 (d, 1H, J=13.71 Hz), 7.70 (t, 1H, J=7.83 Hz), 7.90 (d, 1H, J=7.77 Hz), 8.00 (d, 1 H, J=7.83 Hz), 8.04 (s 1H).

### Example 15

### Procedure for the Synthesis of 2-chloro-1-(4-(3-(trifluoromethyl)phenylsulfonyl)piperidin-1-yl)ethanone (60)

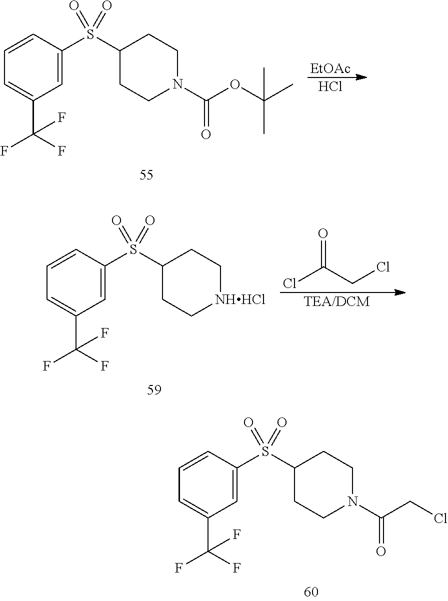

### A. Preparation of 4-(3-(trifluoromethyl)phenylsulfonyl)piperidine hydrochloride (59)

tert-Butyl 4-(3-(trifluoromethyl)phenylsulfonyl)piperidine-1-carboxylate (55) (1.5 g, 3.93 mmol) was stirred in EtOAc (10 mL) at room temperature. Gaseous HCl was bubbled through the solution for 0.5 minutes then the reaction stirred at room temperature for 20 minutes. The reaction was concentrated in vacuo to give 4-(3-(trifluoromethyl)phenylsulfonyl)piperidine hydrochloride (59) (1.29 g, 100%).

### B. Preparation of 2-chloro-1-(4-(3-(trifluoromethyl)phenylsulfonyl)piperidin-1-yl)ethanone (60)

4-(3-(Trifluoromethyl)phenylsulfonyl)piperidine hydrochloride (59) (1.29 g, 3.93 mmol) and TEA (1.67 mL, 12 mmol) were stirred in DCM (20 mL) at room temperature. Chloroacetyl chloride (313 μL, 3.93 mmol) was added, and the reaction stirred for 16 hours. The reaction was concentrated and the crude material purified by automated flash chromatography (50% EtOAc/PE) to give 2-chloro-1-(4-(3-(trifluoromethyl)phenylsulfonyl)piperidin-1-yl)ethanone (60) (700 mg, 48%). ¹H NMR (300 mHz, CDCl₃) 1.74 (m, 5H), 2.35 (m, 2H), 3.17 (m, 2H), 4.05 (m, 3H), 4.69 (d, 2H), 7.78 (t, 1H, J=7.86 Hz), 7.98 (d, 1H, J=8.04 Hz), 8.09 (d, 1H, J=7.86 Hz), 8.16 (s, 1H).

### Example 16

### Procedure for the Synthesis of 4-(3-(trifluoromethyl)phenyl sulfonyl)cyclohexanamine (68)

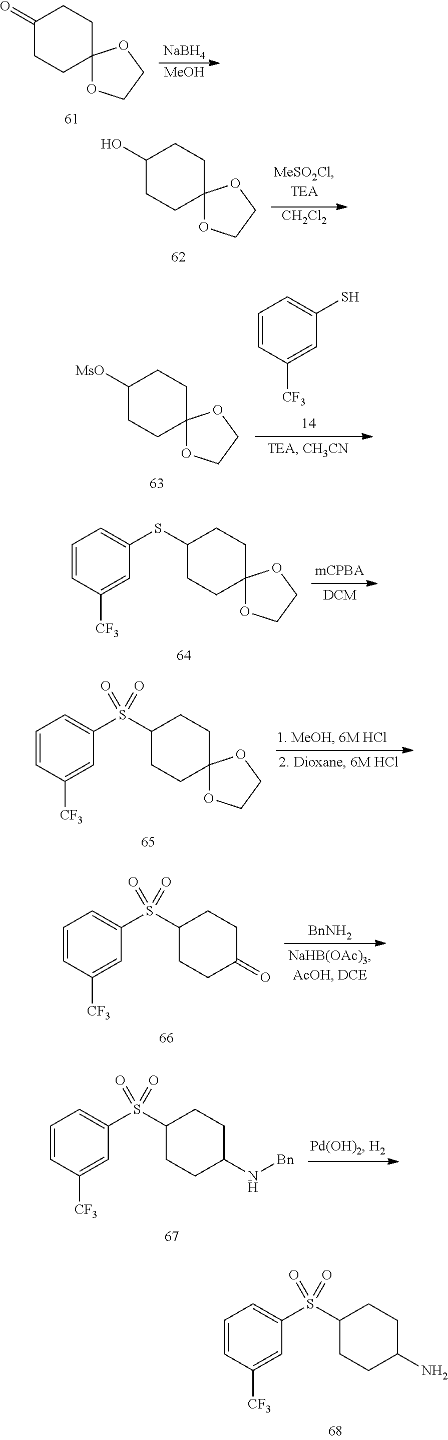

### A. Preparation of 1,4-dioxaspiro[4.5]decan-8-ol (62)

1,4-Dioxaspiro[4.5]decan-8-one (61) (12.44 g, 79.65 mmol) was stirred in MeOH (80 mL) at room temperature. NaBH₄ (1.51 g, 39.83 mmol) was added portion wise, and the reaction stirred for 25 minutes. The solvent was removed in vacuo, and the residue was treated with 5% aqueous NaOH and extracted with EtOAc (twice). The organics were combined, dried (Na₂SO₄), filtered, and concentrated in vacuo to give 1,4-dioxaspiro[4.5]decan-8-ol (62) (11.10 g, 88%). ¹H NMR (300 mHz, CDCl₃) δ 1.59 (m, 4H), 1.81 (m, 4H), 3.78 (m, 1H), 3.93 (br s, 4H).

### B. Preparation of 1,4-dioxaspiro[4.5]decan-8-yl methanesulfonate (63)

1,4-Dioxaspiro[4.5]decan-8-ol (62) (11.10 g, 70.17 mmol) and TEA (14.67 mL, 105.3 mmol) were stirred under argon in dry DCM (150 mL) at 0° C. Methanesulfonyl chloride (5.70 mL, 73.7 mmol) was added slowly, and the reaction was stirred for 1 hour. The reaction was washed sequentially with saturated aqueous NH₄Cl, saturated aqueous NaHCO₃, dried (Na₂SO₄), filtered, and concentrated in vacuo to give 1,4-dioxaspiro[4.5]decan-8-yl methanesulfonate (63) (15.12 g, 91%). ¹H NMR (300 mHz, CDCl₃) δ 1.64 (m, 2H), 1.85 (m, 2H), 1.99 (m, 4H), 3.01 (s, 3H), 3.94 (br t, 4H, J=3.74 Hz), 4.84 (m, 1H).

### C. Preparation of 8-(3-(trifluoromethyl)phenylthio)-1,4-dioxaspiro[4.5]decane (64)

1,4-Dioxaspiro[4.5]decan-8-yl methanesulfonate (63) (15.12 g, 63.99 mmol), 3-(trifluoromethyl)benzenethiol (14) (11.40 g, 63.99 mmol), TEA (14.10 mL, 83.19 mmol), and KI (cat.) were heated at reflux under argon in MeCN (200 mL) for 4 hours. Additional 3-(trifluoromethyl)benzenethiol (14) (2.00 g, 11.2 mmol), TEA (5.0 mL, 29 mmol), and K₂CO₃ (2.00 g, 14.5 mmol) were added, and the reaction refluxed for a further 16 hours. The solvent was removed in vacuo. The residue was dissolved in EtOAc, washed sequentially with H₂O, 2M aqueous NaOH (twice), and brine. The organics were dried (Na₂SO₄), filtered, concentrated in vacuo and the crude material purified by automated flash chromatography (R_{f}=0.65 in 5:1 PE:EtOAc) to give 8-(3-(trifluoromethyl)phenylthio)-1,4-dioxaspiro[4.5]decane (64) (14.85 g, 73%). ¹H NMR (300 mHz, CDCl₃) δ 1.72 (m, 6H), 2.00 (m, 2H), 3.25 (m, 1H), 3.94 (s, 4H), 7.43 (m, 2H), 7.56 (d, 1H, J=7.70 Hz), 7.63 (br s, 1H).

### D. Preparation of 8-(3-(trifluoromethyl)phenylsulfonyl)-1,4-dioxaspiro[4.5]decane (65)

8-(3-(Trifluoromethyl)phenylthio)-1,4-dioxaspiro[4.5]decane (64) (3.32 g, 10.43 mmol), NaHCO₃ (4.38 g, 52.2 mmol) and m-CPBA (5.84 g, 26.08 mmol) were stirred in DCM (130 mL) at room temperature for 16 hours. H₂O (50 mL) and MeOH (20 mL) were added, and the mixture stirred for 30 minutes. The organic layer was washed sequentially with saturated aqueous NaHCO₃, saturated aqueous Na₂S₂O₅, saturated aqueous NaHCO₃, dried (Na₂SO₄), filtered, and concentrated in vacuo to give 8-(3-(trifluoromethyl)phenylsulfonyl)-1,4-dioxaspiro[4.5]decane (65) (3.54 g, 97%). ¹H NMR (300 mHz, CDCl₃) δ 1.53 (m, 2H), 1.83 (m, 4H), 2.07 (m, 2H), 2.97 (m, 1H), 3.92 (br t, 4H, J=3.52 Hz), 7.74 (t, 1H, J=7.70 Hz), 7.93 (d, 1H, J=7.92 Hz), 8.08 (d, 1H, J=8.36 Hz), 8.15 (s, 1H).

### E. Preparation of 4-(3-(trifluoromethyl)phenylsulfonyl)cyclohexanone (66)

8-(3-(trifluoromethyl)phenylsulfonyl)-1,4-dioxaspiro[4.5]decane (65) (18.64 g, 53.20 mmol) was stirred in MeOH (120 mL) and 6 M aqueous HCl (80 mL) at reflux for 20 hours. The reaction was diluted with water and extracted twice with DCM. The organics were combined, dried (Na₂SO₄), filtered, and the solvent removed in vacuo. The crude material was heated in 1,4-dioxane (150 mL) and 6 M aqueous HCl (100 mL) at reflux for 16 hours. The reaction was concentrated to half volume and extracted twice with DCM. The organics were dried (Na₂SO₄), filtered, and concentrated in-vacuo to give 4-(3-(trifluoromethyl)phenylsulfonyl)cyclohexanone (66) (15.25 g, 94%). ¹H NMR (300 mHz, CDCl₃) δ 2.02 (m, 2H), 2.36 (m, 4H), 2.59 (m, 2H), 3.39 (m, 1H), 7.78 (t, 1H, J=8.14 Hz), 7.97 (m, 1H), 8.14 (m, 2H).

### F. Preparation of N-benzyl-4-(3-(trifluoromethyl)phenylsulfonyl)cyclohexanamine (67)

4-(3-(Trifluoromethyl)phenylsulfonyl)cyclohexanone (66) (2.77 g, 9.04 mmol), benzyl amine (0.99 mL, 9.04 mmol), AcOH (0.52 mL, 9.04 mmol), and NaHB(OAc)₃ (2.68 g, 12.7 mmol) were stirred in DCE (100 mL) at room temperature for 3 hours. The reaction was quenched with 1 M NaOH (100 mL), and extracted twice with Et₂O. The organics were washed with brine, dried (Na₂SO₄), filtered, and concentrated in-vacuo to give N-benzyl-4-(3-(trifluoromethyl)phenylsulfonyl)cyclohexanamine (67) (3.45 g, 96%). ¹H NMR (300 mHz, CDCl₃) δ 1.78 (m, 2H), 1.49 (m, 2H), 1.93 (m, 6H), 2.94 (m, 2H), 3.70 (s, 2 H), 7.27 (m, 5H), 7.74 (m, 1H), 7.94 (m, 1H), 8.09 (m, 1H), 8.16 (m, 1H).

### G. Preparation of 4-(3-(trifluoromethyl)phenylsulfonyl)cyclohexanamine (68)

N-benzyl-4-(3-(trifluoromethyl)phenylsulfonyl)cyclohexanamine (67) (2.77 g, 6.97 mmol) and Pd(OH)₂ (cat.) were stirred in MeOH (100 mL) at room temperature. H₂ was bubbled through the mixture for 10 minutes, followed by stirring under H₂ (1 atm) for 16 hours. The reaction was filtered through Celite and concentrated in-vacuo to give 4-(3-(trifluoromethyl)phenylsulfonyl)cyclohexanamine (68) (2.15 g, quantitative); ¹H NMR (300 mHz, MeOD) δ 1.57 (m, 2H), 1.86 (m, 2 H), 3.05 (m, 1H), 3.36 (s, 1H), 7.89 (m, 1H), 8.08 (m, 1H), 8.18 (m, 2H).

### Example 17

### Procedure for the Synthesis of 4-methyl-4-(3-(trifluoromethyl)phenylsulfonyl)cyclohexanamine (72)

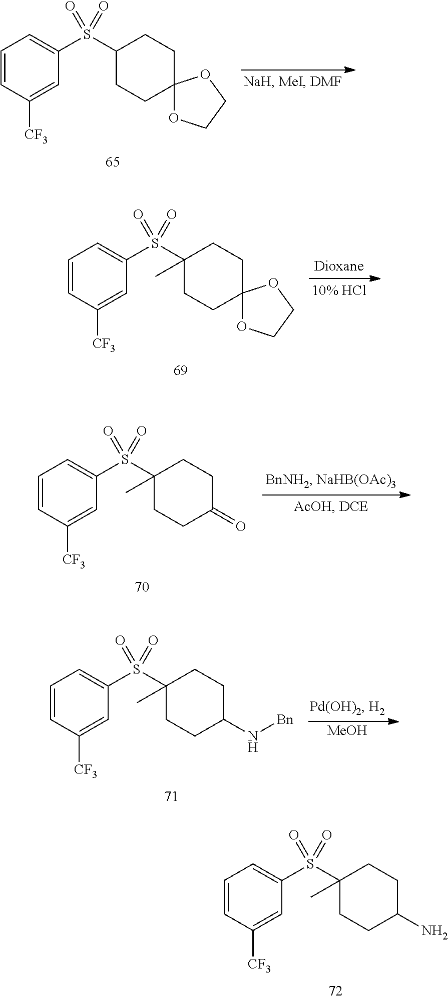

### A. Preparation of 8-methyl-8-(3-(trifluoromethyl)phenylsulfonyl)-1,4-dioxaspiro[4.5]decane (69)

8-(3-(Trifluoromethyl)phenylsulfonyl)-1,4-dioxaspiro[4.5]decane (65) (6.50 g, 18.6 mmol) and MeI (6.76 mL, 108 mmol) were stirred under argon in dry DMF (80 mL) at room temperature. NaH (60% dispersion in mineral oil; 4.70 g, 117 mmol) was added, and the suspension stirred for 120 hours. The reaction was diluted with H₂O, extracted with EtOAc, and the organics washed with H₂O, brine (twice), dried (Na₂SO₄), filtered, and concentrated in vacuo. The crude product was purified by automated flash chromatography (R_{f}=0.7 in 1:1 PE:EtOAc) to give 8-methyl-8-(3-(trifluoromethyl)phenylsulfonyl)-1,4-dioxaspiro[4.5]decane (69) (4.63 g, 68%). ¹H NMR (300 mHz, CDCl₃) δ 1.36 (s, 3H), 1.64 (m, 4H), 1.81 (m, 2H), 2.24 (m, 2H), 3.92 (s, 4H), 7.72 (t, 1H, J=7.92), 7.92 (d, 1H, J=7.92), 8.00 (d, 1H, J=7.92), 8.14 (br s, 1H).

### B. Preparation of 4-methyl-4-(3-(trifluoromethyl)phenylsulfonyl)cyclohexanone (70)

8-Methyl-8-(3-(trifluoromethyl)phenylsulfonyl)-1,4-dioxaspiro[4.5]decane (69) (4.63 g, 12.7 mmol) was stirred in dioxane (150 mL) and 10% aqueous HCl (50 mL) at room temperature for 120 hours. The reaction was concentrated to half volume, and extracted with EtOAc. The organics were washed with saturated aqueous NaHCO₃, dried (Na₂SO₄), filtered, and the solvent removed in vacuo to give 4-methyl-4-(3-(trifluoromethyl)phenylsulfonyl)cyclohexanone (70) (4.25 g, quantitative); ¹H NMR (300 mHz, CDCl₃) δ 1.50 (s, 3H), 1.98 (m, 2H), 2.38 (m, 4 H), 2.57 (m, 2H), 7.76 (m, 1H), 7.95 (m, 1H), 8.11 (m, 2H).

### C. Preparation of N-benzyl-4-methyl-4-(3-(trifluoromethyl)phenylsulfonyl)cyclohexanamine (71)

4-Methyl-4-(3-(trifluoromethyl)phenylsulfonyl)cyclohexanone (70) (3.03 g, 9.46 mmol), benzyl amine (1.03 mL, 9.46 mmol), AcOH (0.54 mL, 9.46 mmol), and NaHB(OAc)₃ (2.81 g, 13.2 mmol) were stirred in DCE (80 mL) at room temperature for 2 hours. The reaction was quenched with 1 M aqueous NaOH (100 mL) and extracted with Et₂O (twice). The organics were washed with brine, dried (Na₂SO₄), filtered, and concentrated in vacuo. The crude product was purified by automated flash chromatography (R_{f}=0.2 in 95:5 CH₂Cl₂:MeOH) to give N-benzyl-4-methyl-4-(3-(trifluoromethyl)phenylsulfonyl)cyclohexanamine (71) (2.89 g, 74%). ¹H NMR (300 mHz, CDCl₃) δ 1.36 (m, 7H), 1.61 (m, 2H), 1.81 (m, 2H), 2.31 (m, 1H), 3.70 (m, 2H), 7.26 (m, 5H), 7.72 (m, 1H), 7.93 (m, 1H), 8.12 (m, 2H).

### D. Preparation of 4-methyl-4-(3-(trifluoromethyl)phenylsulfonyl)cyclohexanamine (72)

N-Benzyl-4-methyl-4-(3-(trifluoromethyl)phenylsulfonyl)cyclohexanamine (71) (2.31 g, 5.61 mmol)) and Pd(OH)₂ (cat.) were stirred in MeOH (100 mL). H₂ was bubbled through the suspension for 10 minutes, and then the reaction was stirred under H₂ (1 atm) for 16 hours. The reaction was filtered through Celite and concentrated in vacuo to give methyl-4-(3-(trifluoromethyl)phenylsulfonyl)cyclohexanamine (72) (1.76 g, 98%). ¹H NMR (300 mHz, MeOD) δ 1.23 (s, 3H), 1.50 (m, 2H), 1.80 (m, 4H), 2.27 (m, 2H), 3.37 (s, 2 H), 7.89 (m, 1H), 8.13 (m, 3H).

### Example 18

### Procedure for the Synthesis of 4-fluoro-1-methyl-4-(3-(trifluoromethyl)phenylsulfonyl)cyclohexanamine (77)

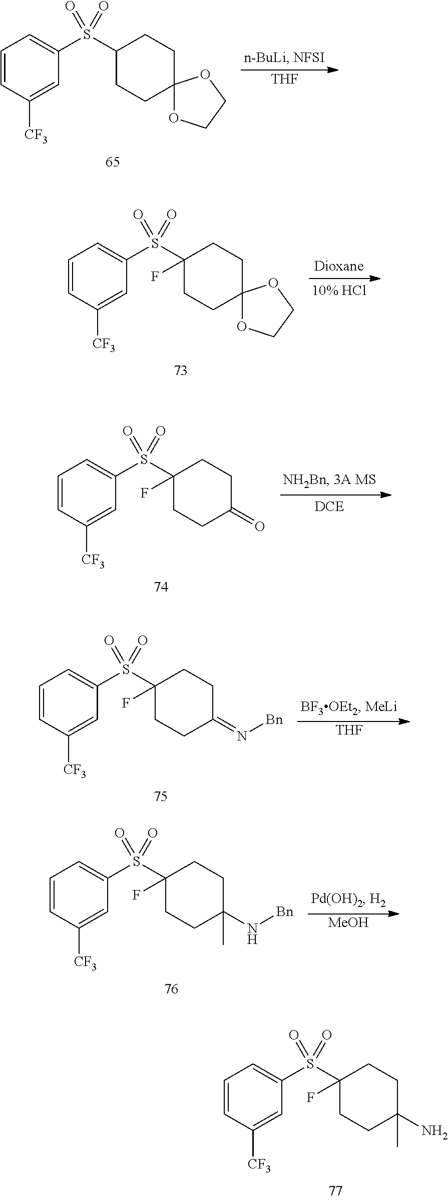

### A. Preparation of 8-fluoro-8-(3-(trifluoromethyl)phenylsulfonyl)-1,4-dioxaspiro[4.5]decane (73)

8-(3-(Trifluoromethyl)phenylsulfonyl)-1,4-dioxaspiro[4.5]decane (65) (7.5 g, 21.4 mmol) was stirred under argon in THF (150 mL) at −78° C. Butyl lithium (1.6 M solution in hexane) (16 mL, 25.7 mmol) was added drop-wise, and the reaction stirred at −78° C. for 30 minutes. N-Fluorobenzenesulfonimide (6.75 g, 21.4 mmol) in THF (10 mL) was added dropwise, and the residue was allowed to warm to room temperature. Stirring was continued for 1 hour. The reaction was quenched with NH₄Cl saturated solution (50 mL) and then extracted with EtOAc (3×150 mL). The organics were washed with brine, dried (Na₂SO₄), and concentrated to one third volume. The resultant precipitate was removed by filtration, and the filtrate concentrated in vacuo. The crude material was re-crystallized in 20% EtOAc-hexane to give 8-fluoro-8-(3-(trifluoromethyl)phenyl-sulfonyl)-1,4-dioxaspiro[4.5]decane (73) (6.31 g, 82%); ¹H NMR (300 mHz CDCl₃) δ 1.82 (dd, 4 H, J=2.7, 7.8 Hz), 2.12 (m, 2H), 2.31 (m, 1H), 2.45 (m, 1H), 7.76 (t, 1H, J=7.83 Hz), 7.97 (d, 1H, J=7.80 Hz), 8.13 (d, 1H, J=7.80 Hz), 8.20 (s, 1H).

### B. Preparation of 4-fluoro-4-(3-(trifluoromethyl)phenylsulfonyl)cyclohexanone (74)

8-Fluoro-8-(3-(trifluoromethyl)phenylsulfonyl)-1,4-dioxaspiro[4.5]decane (73) (6.3 g, 17.12 mmol) in MeOH (120 mL) and 6 M aqueous HCl (80 mL) was heated at reflux for 20 hours. The reaction was diluted with H₂O and extracted twice with DCM. The organics were dried (Na₂SO₄), filtered, and concentrated in vacuo. The crude material was dissolved in 1,4-dioxane (150 mL) and 6 M aqueous HCl (100 mL) and refluxed for 16 hours. The reaction was concentrated to half volume and extracted with DCM (twice). The organic fractions combined, dried (Na₂SO₄), filtered, and concentrated in vacuo to give 4-fluoro-4-(3-(trifluoromethyl)phenylsulfonyl)-cyclohexanone (74) (4.0 g, 73%). ¹H NMR (300 mHz CDCl₃) δ 2.37 (m 2H), 2.58 (m, 6H), 7.81 (t, 1H, J=7.80 Hz), 8.02 (d, 1H, J=7.83 Hz), 8.18 (d, 1H, J=7.89 Hz), 8.24 (s, 1H).

### C. Preparation of N-(4-fluoro-4-(3-(trifluoromethyl)phenylsulfonyl)cyclohexylidene)-1-phenyl-methanamine (75)

4-Fluoro-4-(3-(trifluoromethyl)phenylsulfonyl)cyclohexanone (74) (2.62 g, 8.08 mmol), benzyl amine 0.88 mL, 8.08 mmol), and 3 Å molecular sieves (3.5 g) were stirred in DCM (50 mL) at room temperature for 16 hours. The reaction was filtered through celite and concentrated in vacuo to give N-(4-fluoro-4-(3-(trifluoromethyl)phenylsulfonyl)cyclohexylidene)-1-phenyl-methanamine (75) (3.34 g, quantitative). ¹H NMR (300 mHz, CDCl₃) δ 2.29 (m, 4H), 2.59 (m, 2H), 3.00 (m, 2H), 4.56 (s, 2H), 7.29 (m, 5H), 7.79 (m, 1H), 7.99 (m, 1H), 8.20 (m, 2H).

### D. Preparation of N-benzyl-4-fluoro-1-methyl-4-(3-(trifluoromethyl)phenylsulfonyl)cyclohexan-amine (76)

N-(4-Fluoro-4-(3-(trifluoromethyl)phenylsulfonyl)cyclohexylidene)-1-phenylmethanamine (75) (2.52 g, 6.10 mmol) was stirred under argon in dry THF (100 mL) at −78° C. BF₃.OEt₂ (1.51 mL, 12.2 mmol) was added, and the reaction stirred for 1 hour. MeLi (1.6 M solution in Et₂O) (11.4 mL, 18.3 mmol) was added, and the reaction stirred at −78° C. for 1 h, then allowed to warm to room temperature for 35 minutes. The reaction was quenched with 10% aqueous NaOH and extracted with Et₂O (twice). The organics were washed with brine, dried (Na₂SO₄), filtered, and concentrated in vacuo. The crude material was purified by automated flash chromatography (CH₂Cl₂:MeOH) to give N-benzyl-4-fluoro-1-methyl-4-(3-(trifluoromethyl)phenylsulfonyl)cyclohexan-amine (76) (1.12 g, 43%).

### E. Preparation of 4-fluoro-1-methyl-4-(3-(trifluoromethyl)phenylsulfonyl)cyclohexanamine (77)

N-Benzyl-4-fluoro-1-methyl-4-(3-(trifluoromethyl)phenylsulfonyl)cyclohexanamine (76) (0.37 g, 0.86 mmol) (55) and Pd(OH)₂ were stirred in MeOH (50 mL) under H₂ (1 atm) for 16 hours. The reaction was filtered through celite and concentrated in vacuo to give -fluoro-1-methyl-4-(3-(trifluoromethyl)phenylsulfonyl)cyclohexanamine (77) (260 mg, 89%). The product was confirmed by LCMS and used without further purification.

### Example 19

### Procedure for the Synthesis of (cis)-4-(3-(trifluoromethyl)phenylsulfonyl)cyclohexanamine (83)

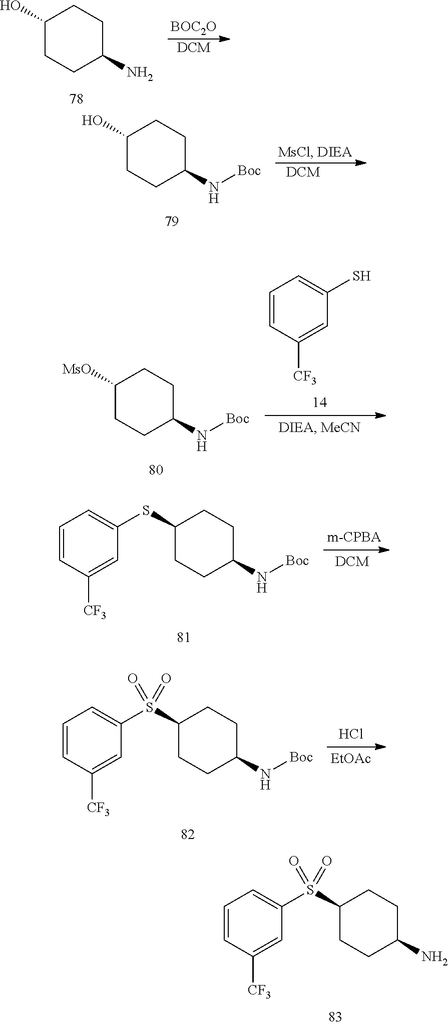

### A. Preparation of tert-butyl (trans)-4-hydroxycyclohexylcarbamate (79)

(Trans)-4-aminocyclohexanol (78) (24.44 g, 212.2 mmol) and (BOC)₂O (50.95 g, 233.4 mmol) were stirred in DCM (600 mL), MeOH, (200 mL), and dioxane (200 mL) at room temperature for 16 hours. The reaction was concentrated in vacuo, and the crude material triturated with EtOH to give tert-butyl (trans)-4-hydroxycyclohexylcarbamate (79) (21.04 g, 46%). ¹H NMR (300 mHz, CDCl₃) δ 1.17 (m, 2H), 1.42 (m, 11H), 2.00 (m, 4H), 3.41 (br s, 1H), 3.60 (m, 1H), 4.35 (br s, 1H).

### B. Preparation of (trans)-4-(tert-butoxycarbonylamino)cyclohexyl methanesulfonate (80)

tert-Butyl (trans)-4-hydroxycyclohexylcarbamate (79) (7.53 g, 35.0 mmol) and DIEA (3.25 mL, 42.0 mmol) were stirred under argon in dry THF (170 mL) at 0° C. MsCl (2.98 mL, 38.5 mmol). was added, and the reaction stirred at room temperature for 6 h. At this time, the reaction was then diluted with EtOAc, washed with H₂O and saturated aqueous NaHCO₃, dried (Na₂SO₄), filtered, and the solvent removed in vacuo to give (trans)-4-(tert-butoxycarbonylamino)cyclohexyl methanesulfonate (80) (10.26 g, quantitative). ¹H NMR (300 mHz, CDCl₃) δ 1.04 (m, 2H), 1.21 (s, 9H), 1.46 (m, 2H), 1.89 (m, 4H), 2.79 (s, 3H), 3.25 (br s, 1H), 4.18 (br s, 1H), 4.40 (m, 1H).

### C. Preparation of tert-butyl (cis)-4-(3-(trifluoromethyl)phenylthio)cyclohexylcarbamate (81)

(Trans)-4-(tert-butoxycarbonylamino)cyclohexyl methanesulfonate (80) (10.26 g, 35.0 mmol), DIEA (18.3 mL, 105 mmol), and 3-(trifluoromethyl)benzenethiol (14) (7.48 g, 42.0 mmol) were stirred under argon in MeCN (250 mL) at reflux for 16 hours. The reaction was concentrated, taken up in EtOAc, washed with 5% aqueous NaOH (twice), brine, dried (Na₂SO₄), filtered, and concentrated in vacuo. The crude material was purified by automated flash chromatography (R_{f}=0.65 in 5:1 PE:EtOAc) to give tert-butyl (cis)-4-(3-(trifluoromethyl)phenylthio)cyclohexylcarbamate (81) (3.27 g, 25%). ¹H NMR (300 mHz, CDCl₃) δ 1.45 (s, 9H), 1.77 (m, 8H), 3.47 (m, 1H), 3.60 (br s, 1H), 4.57 (br s, 1H), 7.42 (m, 2H), 7.53 (m, 1H), 7.61 (m, 1H).

### D. Preparation of tert-butyl (cis)-4-(3-(trifluoromethyl)phenylsulfonyl)cyclohexylcarbamate (82)

tert-Butyl (cis)-4-(3-(trifluoromethyl)phenylthio)cyclohexylcarbamate (81) (3.27 g, 8.71 mmol), NaHCO₃ (2.20 g, 26.1 mmol) and m-CPBA (77%, 4.88 g, 21.8 mmol) were stirred in DCM (150 mL) at room temperature for 2 hours. H₂O (50 mL) and MeOH (20 mL) were added, and the reaction stirred for an additional 30 minutes. The organics were washed sequentially with saturated aqueous Na₂S₂O₅ (twice) and saturated aqueous NaHCO₃ (twice). The organics were then dried (Na₂SO₄), filtered, and concentrated in vacuo to give tert-butyl (cis)-4-(3-(trifluoromethyl)phenylsulfonyl)cyclohexylcarbamate (82) (3.35 g, 94%). ¹H NMR (300 mHz, CDCl₃) δ 1.44 (s, 9H), 1.55 (m, 2H), 1.86 (m, 6H), 2.96 (m, 1H), 3.81 (br s, 1H), 4.69 (br s, 1H), 7.75 (m, 1H), 7.93 (m, 1H), 8.08 (m, 1H), 8.15 (m, 1H).

### E. Preparation of (cis)-4-(3-(trifluoromethyl)phenylsulfonyl)cyclohexanamine (83)

tert-Butyl (cis)-4-(3-(trifluoromethyl)phenylsulfonyl)cyclohexylcarbamate (82) (3.35 g, 8.22 mmol) was stirred in EtOAc (100 mL). Gaseous HCl was bubbled through the solution for 50 seconds then stirred for 25 minutes. The reaction was concentrated to in vacuo to give (cis)-4-(3-(trifluoromethyl)phenylsulfonyl)cyclohexanamine (83) as the HCl salt. (2.83 g, quantitative); ¹H NMR (300 mHz, MeOD) δ 1.93 (m, 4H), 2.08 (m, 4H), 3.36 (m, 1 H), 3.44 (m, 1H), 7.92 (m, 1H), 8.11 (m, 1H), 8.22 (m, 2H).

### Example 20

### Procedure for the Synthesis of (trans)-4-(3-(trifluoromethyl)phenylsulfonyl)cyclohexanamine (89)

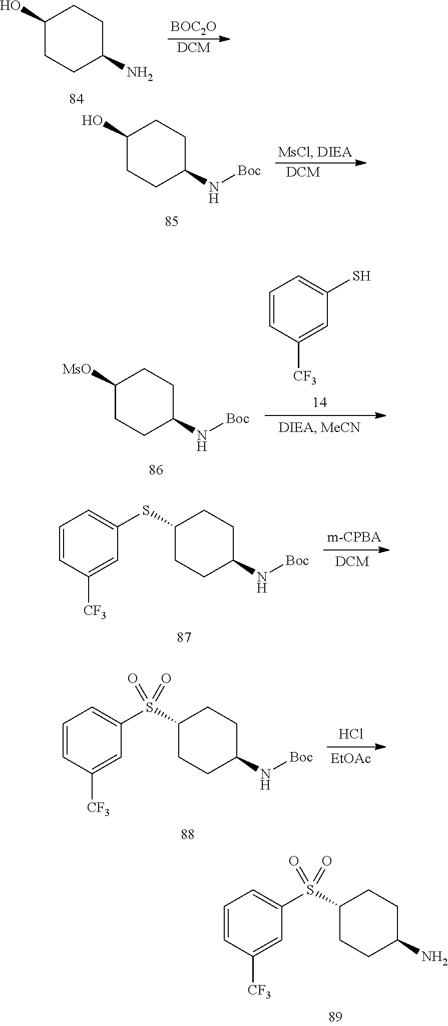

### A. Preparation of tert-butyl (cis)-4-hydroxycyclohexylcarbamate (85)

(Cis)-4-aminocyclohexanol hydrochloride (84) (7.34 g, 48.4 mmol) and TEA (13.5 mL, 96.8 mmol) were stirred in dioxane (100 mL) and MeOH (40 mL) at room temperature. (BOC)₂O (11.6 g, 53.3 mmol) in DCM (40 mL) was added, and the reaction stirred for 3 hours. The reaction was concentrated in vacuo, H₂O was added, and the mixture extracted with EtOAc (twice). The organics were dried (Na₂SO₄), filtered, and concentrated in vacuo to give tert-butyl (cis)-4-hydroxycyclohexylcarbamate (85) (10.42 g, quantitative); ¹H NMR (300 mHz, CDCl₃) δ 1.46 (s, 9H), 1.67 (m, 8H), 3.54 (br s, 1H), 3.71 (s, 1H), 3.90 (m, 1H), 4.54 (br s, 1H).

### B. Preparation of (cis)-4-(tert-butoxycarbonylamino)cyclohexyl methanesulfonate (86)

tert-Butyl (cis)-4-hydroxycyclohexylcarbamate (85) (4.36 g, 20.3 mmol) and DIEA (7.05 mL, 40.5 mmol) were stirred under argon in dry DCM (120 mL) at 0° C. MsCl (1.72 mL, 22.3 mmol) was added and the reaction stirred for 2 hours at 0° C., and 1 h at room temperature. The reaction was washed sequentially with 1 M aqueous HCl, saturated aqueous NaHCO₃, dried (Na₂SO₄), filtered, and concentrated in vacuo to give (cis)-4-(tert-butoxycarbonylamino)cyclohexyl methanesulfonate (86) (5.55 g, 93%). ¹H NMR (300 mHz, CDCl₃) δ 1.45 (s, 9H), 1.60 (m, 2H), 1.78 (m, 4 H), 2.05 (m, 2H), 3.02 (s, 3H), 3.53 (br s, 1H), 4.47 (br s, 1H), 4.89 (m, 1H).

### C. Preparation of tert-butyl (trans)-4-(3-(trifluoromethyl)phenylthio)cyclohexylcarbamate (87)

(Cis)-4-(tert-butoxycarbonylamino)cyclohexyl methanesulfonate (86) (5.55 g, 18.9 mmol), DIEA (4.94 mL, 28.4 mmol), and 3-(trifluoromethyl)benzenethiol (14) (4.04 g, 22.7 mmol) were stirred under argon MeCN (120 mL) at reflux for 18 hours. The reaction was concentrated in vacuo and taken up in EtOAc. The organic layer was washed sequentially with 1 M aqueous HCl, 5% aqueous NaOH, saturated NaHCO₃, and brine. The organic layer was dried (Na₂SO₄), filtered, and concentrated in vacuo. The crude material was purified by automated flash chromatography (R_{f}=0.65 in 5:1 PE:EtOAc) to give tert-butyl (trans)-4-(3-(trifluoromethyl)phenylthio)cyclohexylcarbamate (87) (2.67 g, 38%). ¹H NMR (300 mHz, CDCl₃) δ 1.20 (m, 2H), 1.43 (m, 11H), 2.06 (m, 4H), 3.04 (m, 1 H), 3.45 (br s, 1H), 4.39 (br s, 1H), 7.44 (m, 2H), 7.55 (m, 1H), 7.62 (m, 1H).

### D. Preparation of tert-butyl (trans)-4-(3-(trifluoromethyl)phenylsulfonyl)cyclohexylcarbamate (88)

tert-Butyl (trans)-4-(3-(trifluoromethyl)phenylthio)cyclohexylcarbamate (87) (2.67 g, 7.11 mmol), NaHCO₃ (1.79 g, 21.3 mmol) and m-CPBA (77%, 3.98 g, 17.8 mmol) were stirred in DCM (125 mL) at room temperature for 16 hours. H₂O (50 mL) and MeOH (20 mL) were added, and the mixture stirred for an additional 30 minutes. The organics were washed sequentially with saturated aqueous Na₂S₂O₅ (twice) and saturated aqueous NaHCO₃. The organics were dried (Na₂SO₄), filtered, and concentrated in vacuo to give tert-butyl (trans)-4-(3-(trifluoromethyl)phenylsulfonyl)cyclohexylcarbamate (88) (2.70 g, 93%). ¹H NMR (300 mHz, CDCl₃) δ 1.04 (m, 2H), 1.34 (s, 9H), 1.46 (m, 2H), 2.06 (m, 4H), 2.82 (m, 1H), 3.28 (br s, 1H), 4.34 (m, 1H), 7.67 (m, 1H), 7.87 (m, 1H), 8.00 (m, 1H), 8.06 (m, 1H).

### E. Preparation of (trans)-4-(3-(trifluoromethyl)phenylsulfonyl)cyclohexanamine (89)

tert-Butyl (cis)-4-(3-(trifluoromethyl)phenylsulfonyl)cyclohexylcarbamate (88) (2.70 g, 6.65 mmol) was stirred in EtOAc (80 mL) at room temperature. Gaseous HCl was bubbled through the solution for 50 seconds then stirred at room temperature for 45 minutes. The reaction was concentrated in-vacuo to give (trans)-4-(3-(trifluoromethyl)phenylsulfonyl)cyclohexanamine (89) as the HCl salt. (2.28 g, quantitative); ¹H NMR (300 mHz, MeOD) δ 1.46 (m, 2H), 1.63 (m, 2H), 2.16 (m, 4 H), 3.11 (m, 1H), 7.91 (m, 1H), 8.11 (m, 1H), 8.20 (m, 2H).

### Example 21

### Procedure for the Synthesis of (trans)-4-((3-(trifluoromethyl)phenylsulfonyl)methyl)cyclohexanamine (97)

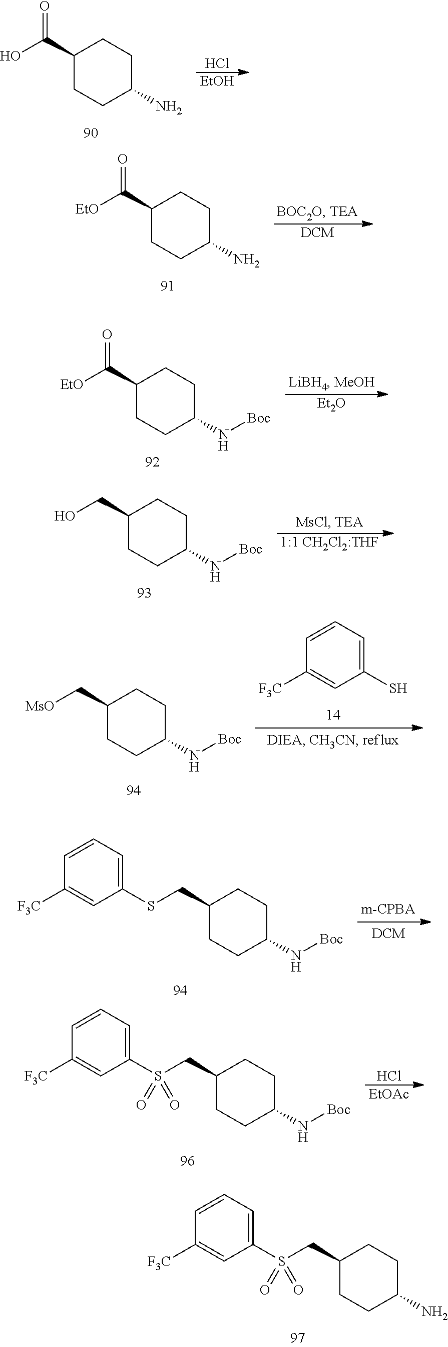

### A. Preparation of (trans)-ethyl 4-(tert-butoxycarbonylamino)cyclohexanecarboxylate (91)

(Trans)-4-aminocyclohexanecarboxylic acid hydrochloride (90) (7.45 g, 41.5 mmol) was stirred in absolute EtOH (80 mL) at room temperature. Gaseous HCl was bubbled through the solution for 1 minute, the reaction heated at reflux for 20 hours and then concentrated in vacuo. The crude residue was stirred in DCM (100 mL) with TEA (17.3 mL, 124 mmol) and (BOC)₂O (10.86 g, 49.76 mmol) at room temperature for 6 hours. The reaction was concentrated in vacuo, taken up in EtOAc, washed with 1 M aqueous HCl and saturated aqueous NaHCO₃. The organic layer was dried (Na₂SO₄), filtered. and concentrated in vacuo to provide (rans)-ethyl 4-(tert-butoxycarbonylamino)cyclohexanecarboxylate (91) (11.25 g, quantitative); ¹H NMR (300 mHz, CDCl₃) δ 1.11 (m, 2H), 1.25 (t, 3H, J=7.70 Hz), 1.44 (s, 9H), 2.05 (m, 5H), 2.20 (m, 1H), 3.41 (br s, 1H), 4.12 (q, 2H, J=7.70 Hz), 4.38 (bs, 1 H).

### B. Preparation of tert-butyl (trans)-4-(hydroxymethyl)cyclohexylcarbamate (92)

(Trans)-ethyl 4-(tert-butoxycarbonylamino)cyclohexanecarboxylate (91) (11.25 g, 41.46 mmol) and MeOH (1.00 mL, 25.3 mmol) were stirred in Et₂O (200 mL) at room temperature. LiBH₄ (3.00 g, 124 mmol) was added, followed by dropwise addition of MeOH (4.04 mL, 98.7 mmol). The reaction was stirred for 45 minutes, quenched with MeOH, and concentrated in vacuo. The residue was taken up in 10% aqueous NaOH and extracted with EtOAc (twice). The organics were washed with brine, dried (Na₂SO₄), filtered, and concentrated in vacuo to give provide tert-butyl (trans)-4-(hydroxymethyl)cyclohexylcarbamate (92) (9.15 g, quantitative); ¹H NMR (300 mHz, MeOD) δ 1.05 (m, 2H), 1.21 (m, 2H), 1.45 (s, 9H), 1.84 (m, 2H), 1.94 (m, 2H), 3.26 (m, 1H), 3.37 (d, 2H, J=6.38 Hz), 3.62 (m, 1H), 4.64 (br s, 1 H).

### C. Preparation of ((trans)-4-(tert-butoxycarbonylamino)cyclohexyl)methyl methanesulfonate (93)

tert-Butyl (trans)-4-(hydroxymethyl)cyclohexylcarbamate (92) (5.00 g, 21.8 mmol) and TEA (4.56 mL, 32.71 mmol) were stirred under argon in 1:1 CH₂Cl₂:THF (110 mL) at 0° C. MsCl (1.77 mL, 22.9 mmol) was added, and the reaction stirred for 30 minutes at 0° C. then allowed to warm to room temperature. The reaction was stirred for 16 hours. TEA (6.08 mL, 43.6 mmol) and MsCl (1.77 mL, 22.9 mmol) were added, and stirring continued for an additional 6 hours. The reaction was concentrated in vacuo, taken up in EtOAc, washed with 1 M aqueous HCl, saturated NaHCO₃, dried (Na₂SO₄), filtered, and concentrated in vacuo to give ((trans)-4-(tert-butoxycarbonylamino)cyclohexyl)methyl methanesulfonate (93) (7.45 g, quantitative). ¹H NMR (300 mHz, CDCl₃) δ 1.12 (m, 4H), 1.44 (s, 9H), 1.51 (m, 1 H), 1.71 (bs, 1H), 1.86 (m, 2H), 2.06 (m, 2H), 3.01 (s, 3H), 3.40 (bs, 1H), 4.03 (d, 2H, J=6.38 Hz), 4.39 (bs, 1H).

### D. Preparation of tert-butyl (trans)-4-((3-(trifluoromethyl)phenylthio)methyl)cyclohexylcarbamate (94)

((Trans)-4-(tert-butoxycarbonylamino)cyclohexyl)methyl methanesulfonate (93) (6.70 g, 21.8 mmol), DIEA (7.59 mL, 43.6 mmol) and 3-(trifluoromethyl)benzenethiol (14) (4.66 g, 26.15 mmol) were stirred under argon in MeCN (240 mL) at reflux for 16 hours. The solvent was removed in vacuo, the residue was taken up in EtOAc, washed with 1 M aqueous HCl, saturated NaHCO₃, dried (Na₂SO₄), filtered, and concentrated. The crude product was purified by automated flash chromatography (5:1 PE:EtOAc) to give tert-butyl (trans)-4-((3-(trifluoromethyl)phenylthio)methyl)cyclohexylcarbamate (94) (4.31 g, 51%). ¹H NMR (300 mHz, CDCl₃) δ 1.03 (m, 4H), 1.37 (s, 9H), 1.43 (m, 2H), 1.92 (m, 4H), 2.77 (d, 2H, J=6.82 Hz), 3.31 (br s, 1H), 4.30 (m, 1H), 7.33 (m, 4H).

### E. Preparation of tert-butyl (trans)-4-((3-(trifluoromethyl)phenylsulfonyl)methyl)cyclohexyl-carbamate (95)

tert-Butyl (trans)-4-((3-(trifluoromethyl)phenylthio)methyl)cyclohexyl carbamate (94) (4.31 g, 11.1 mmol), NaHCO₃ (2.79 g, 33.2 mmol) and m-CPBA (77%, 6.20 g, 27.7 mmol) were stirred in DCM (300 mL) at room temperature for 16 hours. H₂O (50 mL) and MeOH (20 mL) were added and the mixture stirred for 30 minutes. The organics were washed sequentially with saturated aqueous Na₂S₂O₅ (twice) and saturated aqueous NaHCO₃. The organics were then dried (Na₂SO₄), filtered, and concentrated in vacuo to give tert-butyl (trans)-4-((3-(trifluoromethyl)phenylsulfonyl)methyl)cyclohexyl-carbamate (95) (2.70 g, 93%). ¹H NMR (300 mHz, CDCl₃) δ 1.16 (m, 4H), 1.44 (s, 9H), 2.01 (m, 5H), 3.00 (d, 2H, J=6.60 Hz), 3.37 (br s, 1H), 4.40 (m, 1H), 7.74 (m, 1H), 7.92 (m, 1H), 8.11 (m, 1 H), 8.19 (m, 1H).

### F. Preparation of (trans)-4-((3-(trifluoromethyl)phenylsulfonyl)methyl)cyclohexanamine (96)

tert-Butyl (trans)-4-((3-(trifluoromethyl)phenylsulfonyl)methyl)cyclohexyl carbamate (95) (4.27 g, 10.13 mmol) was stirred in EtOAc (200 mL) at room temperature. Gaseous HCl was bubbled through the solution 40 seconds, and the reaction was then stirred for 25 minutes. The reaction was concentrated in vacuo to give (trans)-4-((3-(trifluoromethyl)phenylsulfonyl)methyl)cyclohexanamine (96) as the HCl salt (3.63 g, quantitative); ¹H NMR (300 mHz, MeOD δ 1.36 (m, 4H), 2.04 (m, 5H), 3.07 (m, 1H), 3.27 (d, 2H, J=6.60 Hz), 7.90 (m, 1H), 8.09 (m, 1H), 8.24 (m, 2H).

### Example 22

### Procedure for the Synthesis of (cis)-4-((3-(trifluoromethyl)phenyl sulfonyl)methyl)cyclohexanamine (104)

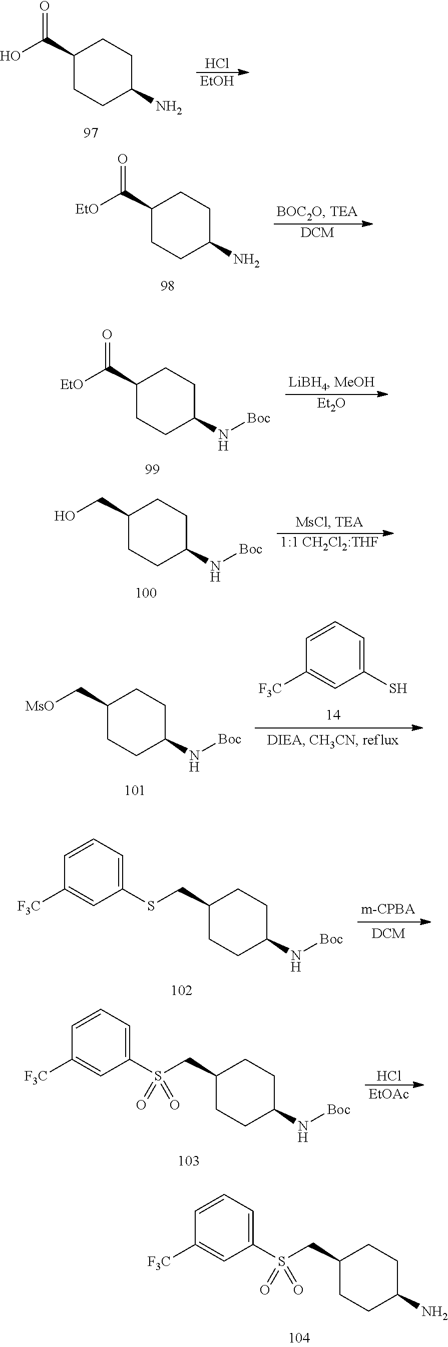

### A. Preparation of (cis)-ethyl 4-(tert-butoxycarbonylamino)cyclohexanecarboxylate (98)

(Cis)-4-aminocyclohexanecarboxylic acid (97) (5.00 g, 34.9 mmol) was stirred in absolute EtOH (60 mL) at room temperature. Gaseous HCl was bubbled through 60 seconds, then the reaction was heated at reflux for 3 hours. The reaction was concentrated in vacuo, and the residue was taken up in DCM (100 mL). TEA (14.6 mL, 105 mmol) and (BOC)₂O (9.15 g, 41.9 mmol) were added, and the reaction stirred at room temperature for 16 hours. The reaction was concentrated in vacuo, taken up in EtOAc, washed with 1 M aqueous HCl, saturated aqueous NaHCO₃, brine, dried (Na₂SO₄), filtered, and concentrated in vacuo to give (cis)-ethyl 4-(tert-butoxycarbonylamino)cyclohexanecarboxylate (98) (9.48 g, quantitative); ¹H NMR (300 mHz, CDCl₃) δ 1.25 (t, 3H, J=7.04 Hz), 1.43 (s, 9H), 1.55 (m, 2H), 1.69 (m, 4H), 1.84 (m, 2H), 2.43 (m, 1H), 3.63 (br s, 1H), 4.12 (q, 2H, J=7.04 Hz), 4.59 (br s, 1H).

### B. Preparation of tert-butyl (cis)-4-(hydroxymethyl)cyclohexylcarbamate (99)

(Cis)-ethyl 4-(tert-butoxycarbonylamino)cyclohexanecarboxylate (98) (10.02 g, 36.93 mmol) and MeOH (2.00 mL, 50.6 mmol) were stirred in Et₂O (100 mL) at room temperature. LiBH₄ (2.68 g, 111 mmol) was added followed by the dropwise addition of MeOH (2.49 mL, 60.4 mmol). The reaction was stirred for 1 h, quenched with MeOH and concentrated in vacuo. The residue was treated with 10% aqueous NaOH, extracted with EtOAc (twice), and the organics washed with brine, dried (Na₂SO₄), filtered, and concentrated in vacuo to give tert-butyl (cis)-4-(hydroxymethyl)cyclohexylcarbamate (99) (8.00 g, 94%). ¹H NMR (300 mHz, MeOD) δ 1.26 (m, 2H), 1.45 (s, 9H), 1.62 (m, 8H), 3.52 (d, 2H, J=6.16 Hz), 3.76 (bs, 1H), 4.65 (br s, 1H).

### C. Preparation of ((cis)-4-(tert-butoxycarbonylamino)cyclohexyl)methyl methanesulfonate (100)

tert-Butyl (cis)-4-(hydroxymethyl)cyclohexylcarbamate (99) (5.00 g, 21.8 mmol) and TEA (9.12 mL, 65.4 mmol) were stirred under argon in dry THF (120 mL) at 0° C. MsCl (2.53 mL, 32.71 mmol) was added, and the reaction stirred for 1 h, then allowed to warm to room temperature and stirred for an additional 16 hours. The reaction was concentrated in vacuo, taken up in EtOAc, washed with 1 M aqueous HCl, saturated NaHCO₃, dried (Na₂SO₄), filtered, and concentrated in-vacuo to give ((cis)-4-(tert-butoxycarbonylamino)cyclohexyl)methyl methanesulfonate (100) (6.77 g, quantitative). ¹H NMR (300 mHz, CDCl₃) δ 1.30 (m, 2H), 1.44 (s, 9H), 1.66 (m, 6 H), 1.85 (m, 1H), 3.02 (s, 3H), 3.76 (bs, 1H), 4.09 (d, 2H, J=6.38 Hz), 4.62 (bs, 1 H).

### D. Preparation of tert-butyl (cis)-4-((3-(trifluoromethyl)phenylthio)methyl)cyclohexylcarbamate (101)

((Cis)-4-(tert-butoxycarbonylamino)cyclohexyl)methyl methanesulfonate (100) (6.77 g, 22.0 mmol), DIEA (7.67 mL, 44.1 mmol) and 3-(trifluoromethyl)benzenethiol (14) (4.71 g, 26.4 mmol) were stirred under argon in MeCN (400 mL) at reflux for 16 hours. The reaction was concentrated in vacuo. The residue was taken up in EtOAc, washed with saturated aqueous NaHCO₃ (twice), dried (Na₂SO₄), filtered, and concentrated. The crude product was purified by automated flash chromatography (R_{f}=0.55 in 5:1 PE:EtOAc) to give tert-butyl (cis)-4-((3-(trifluoromethyl)phenylthio)methyl)cyclohexylcarbamate (101) (7.08 g, 83%). ¹H NMR (300 mHz, CDCl₃) δ 1.33 (m, 2H), 1.45 (s, 9H), 1.66 (m, 8H), 2.90 (d, 2H, J=6.82 Hz), 3.73 (bs, 1H), 4.62 (bs, 1H), 7.41 (m, 3H), 7.52 (m, 1H).

### E. Preparation of tert-butyl (cis)-4-((3-(trifluoromethyl)phenylsulfonyl)methyl)cyclohexyl-carbamate (102)

tert-Butyl (cis)-4-((3-(trifluoromethyl)phenylthio)methyl)cyclohexyl carbamate (101) (2.46 g, 6.32 mmol), NaHCO₃ (1.59 g, 19.0 mmol), and m-CPBA (77%, 3.54 g, 15.8 mmol) were stirred in DCM (200 mL) at room temperature for 16 hours. H₂O (50 mL) and MeOH (20 mL) were added and the mixture stirred for an additional 30 minutes. The organics were washed sequentially with saturated aqueous Na₂S₂O₅ (twice), saturated aqueous NaHCO₃, dried (Na₂SO₄), filtered and concentrated in vacuo to give tert-butyl (cis)-4-((3-(trifluoromethyl)phenylsulfonyl)methyl)cyclohexyl-carbamate (102) (2.36 g, 89%). ¹H NMR (300 mHz, CDCl₃) δ 1.30 (m, 11H), 1.55 (m, 7H), 2.04 (br s, 1H), 2.93 (d, 2H, J=6.60 Hz), 3.54 (br s, 1 H), 4.45 (br s, 1H), 7.61 (m, 1H), 7.80 (m, 1H), 7.98 (m, 1H), 8.05 (m, 1H).

### F. Preparation of (cis)-4-((3-(trifluoromethyl)phenylsulfonyl)methyl)cyclohexanamine (103)

tert-Butyl (cis)-4-((3-(trifluoromethyl)phenylsulfonyl)methyl)cyclohexyl carbamate (102) (7.50 g, 17.8 mmol) was stirred in EtOAc (200 mL) at room temperature. Gaseous HCl was bubbled through the solution for 45 seconds, and the reaction was then stirred for 25 minutes. The reaction was concentrated in vacuo to (cis)-4-((3-(trifluoromethyl)phenylsulfonyl)methyl)cyclohexanamine (103) as the HCl salt. (6.13 g, 96%). ¹H NMR (300 mHz, MeOD δ 1.75 (m, 8H), 2.29 (m, 1H), 3.27 (m, 1H), 3.37 (d, 2H, J=6.60 Hz), 7.91 (m, 1H), 8.09 (m, 1H), 8.26 (m, 2H).

### Example 23

### Procedure for the Synthesis of 4-(4-(3-(trifluoromethyl)phenyl sulfonyl)cyclohexyl)piperazin-2-one (107)

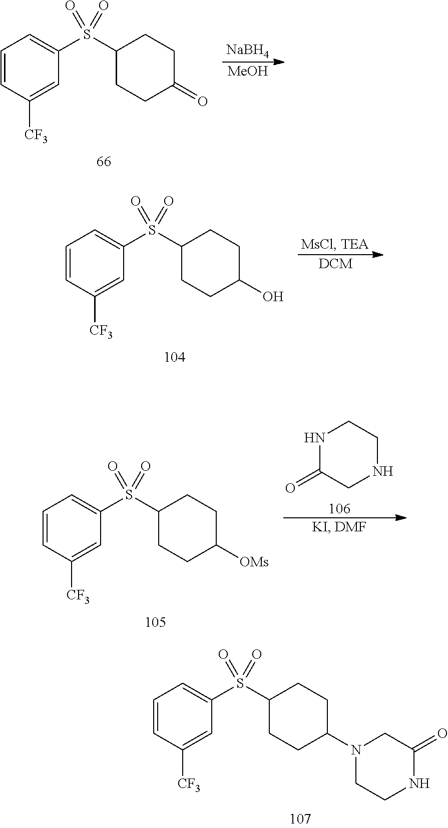

### A. Preparation of 4-(3-(trifluoromethyl)phenylsulfonyl)cyclohexanol (104)

4-(3-(Trifluoromethyl)phenylsulfonyl)cyclohexanone (66) (1.05 g, 3.43 mmol) was stirred in MeOH (30 mL) at room temperature. NaBH₄ (65 mg, 1.7 mmol) was added, and the reaction stirred for 25 minutes. The reaction was concentrated in vacuo, the residue was taken up in EtOAc, washed sequentially with 10% aqueous HCl and saturated aqueous NaHCO₃, dried (Na₂SO₄), and concentrated in vacuo to give 4-(3-(trifluoromethyl)phenylsulfonyl)cyclohexanol (104) (1.05 g, 99%). ¹H NMR (300 mHz, CDCl₃) δ 1.26 (m, 2H), 1.58 (m, 2H), 1.90 (m, 1H), 2.10 (m, 3H), 2.93 (m, 1H), 3.11 (s, 0.5H), 3.18 (s, 0.5H), 3.61 (m, 1H), 7.75 (m, 1 H), 7.95 (m, 1H), 8.09 (m, 2H), 8.16 (m, 1H).

### B. Preparation of 4-(3-(trifluoromethyl)phenylsulfonyl)cyclohexyl methanesulfonate (105)

4-(3-(trifluoromethyl)phenylsulfonyl)cyclohexanol (104) (1.05 g, 3.41 mmol) and TEA (0.71 mL, 5.12 mmol) were stirred under argon in dry CH₂Cl₂ (50 mL) at 0° C. MsCl (0.28 mL) was added, and the reaction stirred for 1 hour. The reaction was quenched with H₂O, washed sequentially with 1 M aqueous HCl and saturated aqueous NaHCO₃, dried (Na₂SO₄), and concentrated in vacuo to give to 4-(3-(trifluoromethyl)phenylsulfonyl)cyclohexyl methanesulfonate (105) (1.24 g, 94%). ¹H NMR (300 mHz, CDCl₃) δ 1.63 (m, 4H), 2.18 (m, 2H), 2.32 (m, 2H), 3.02 (s, 3H), 3.14 (m, 1H), 4.59 (m, 1H), 7.77 (m, 1H), 7.95 (m, 1H), 8.08 (m, 1H), 8.15 (m, 1H).

### C. Preparation of 4-(4-(3-(trifluoromethyl)phenylsulfonyl)cyclohexyl)piperazin-2-one (107)

4-(3-(Trifluoromethyl)phenylsulfonyl)cyclohexyl methanesulfonate (105) (0.72 g, 1.86 mmol), piperazin-2-one (106) (1.00 g, 9.99 mmol) and KI (cat.) were heated in DMF (4 mL) at 140° C. for 1 hour in a sealed container in a microwave reactor. The reaction was diluted with EtOAc, washed sequentially with H₂O and brine, dried (Na₂SO₄), and concentrated in vacuo. The crude material was purified by reverse phase HLPC to give 4-(4-(3-(trifluoromethyl)phenylsulfonyl)-cyclohexyl)piperazin-2-one (107) (6.40 mg, 0.9%).

### Example 24

### General procedure for the synthesis of cis- and trans-4-fluoro-4-(3-(trifluoromethyl)phenylsulfonyl)-cyclohexanamines (110 and 111)

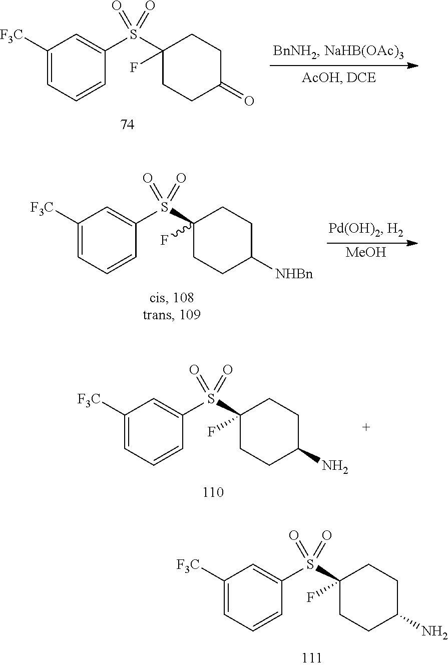

### A. Preparation of cis and trans N-benzyl-4-fluoro-4-(3-(trifluoromethyl)phenylsulfonyl)-cyclohexanamine (108 and 109)

4-Fluoro-4-(3-(trifluoromethyl)phenylsulfonyl)-cyclohexanone (74) (4.0 g, 12.24 mmol), benzyl amine (1.4 mL, 12.8 mmol), AcOH (0.7 mL, 12.04 mmol), and NaHB(OAc)₃ (3.85 g, 17.24 mmol) were stirred in DCE (100 mL) at room temperature for 3 hours. The reaction was quenched with NaHCO₃ saturated solution and extracted with Et₂O (twice). The organics were combined, washed with brine, dried (Na₂SO₄), filtered, and concentrated in vacuo. The crude material was purified by automated flash chromatography to give cis N-benzyl-4-fluoro-4-(3-(trifluoromethyl)-phenylsulfonyl)-cyclohexanamine 108 (2.6 g, 49%). ¹H NMR (300 mHz CDCl₃) δ 1.86 (m 6H), 2.44 (m, 1H), 2.59 (m, 1H), 2.97 (s, 1H), 3.78 (s, 2H), 7.33 (m, 5H), 7.75 (t, 1H, J=7.80 Hz), 7.97 (d, 1H, J=7.92 Hz), 8.13 (d, 1H, J=7.77 Hz), 8.21 (s, 1H). MS (M+H=416.0) (calcd for C₂₀H₂₁F₄NO₂S, 415.12) and trans N-benzyl-4-fluoro-4-(3-(trifluoromethyl)-phenylsulfonyl)-cyclohexanamine 109 (2.5 g, 48%). ¹H NMR (300 mHz CDCl₃) δ1.39 (m, 2H), 2.15 (m 6H), 2.62 (t, 1H, J=11.61 Hz), 3.85 (s, 2H), 7.35 (m, 5H), 7.75 (t, 1H, J=7.87 Hz), 7.97 (d, 1H, J=7.77 Hz), 8.13 (d, 1H, J=7.80 Hz), 8.18 (s, 1H). MS (M+H=416.0) (calcd for C₂₀H₂₁F₄NO₂S, 415.12).

### B. Preparation of cis- and trans-4-fluoro-4-(3-(trifluoromethyl)phenylsulfonyl)cyclohexanamines (110 and 111)

Cis N-benzyl-4-fluoro-4-(3-(trifluoromethyl)-phenylsulfonyl)-cyclohexanamine 108 (2.6 g, 6.26 mmol) and Pd(OH)₂ (cat.) were taken up in MeOH (100 mL) and placed in a parr hydrogenator. The reaction was agitated under H₂ (50 PSI) for 28 h at room temperature. The reaction was filtered through celite and the filtrate concentrated in vacuo to give cis-4-fluoro-4-(3-(trifluoromethyl)phenylsulfonyl)cyclohexanamines (110) (1.6 g, 80%); ¹H NMR (300 mHz CDCl₃) δ 1.78 (m 6H), 2.44 (m, 1H), 2.56 (m, 1H), 3.29 (s, 1H), 7.75 (t, 1H, J=7.84 Hz), 7.97 (d, 1H, J=7.71 Hz), 8.13 (d, 1H, J=7.77 Hz), 8.21 (s, 1H). MS (M+H=325.9) (calcd for C₁₃H₁₅F₄NO₂S, 325.08).

The same procedure was followed for (109) (2.5 g, 6.00 mmol) to give trans-4-fluoro-4-(3-(trifluoromethyl)phenylsulfonyl)cyclohexanamine (111); ¹H NMR (300 mHz CDCl₃) δ1.23 (m, 2H), 1.96 (m 6H), 2.72 (m, 1H), 7.66 (t, 1H, J=7.84 Hz), 7.90 (d, 1H, J=7.80 Hz), 8.04 (d, 1H, J=7.83 Hz), 8.11 (s, 1H). MS (M+H=325.9) (calcd for C₁₃H₁₅F₄NO₂S, 325.08).

### Example 25

### General procedure for the synthesis of cis- and trans-4-fluoro-4-(3-(trifluoromethoxy)phenylsulfonyl)-cyclohexanamines (112 and 113)

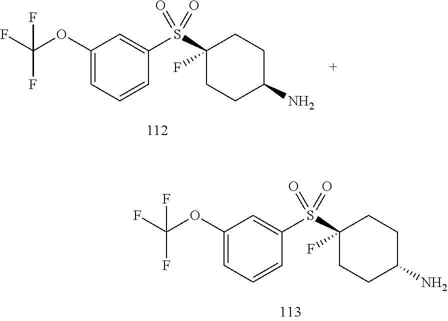

Cis- and trans-4-fluoro-4-(3-(trifluoromethoxy)phenylsulfonyl)-cyclohexanamines (112 and 113) were prepared in analogous manner to that described for cis- and trans-4-fluoro-4-(3-(trifluoromethyl)phenylsulfonyl)-cyclohexanamines (110 and 111) by using 3-(trifluoromethoxy)benzenethiol in place of 3-(trifluoromethyl)benzenethiol.

### Example 26

### Procedure for the Synthesis of cis-4-fluoro-4-(3-fluoro-5-(trifluoromethyl)phenylsulfonyl)cyclohexanamine HCl salt (124)

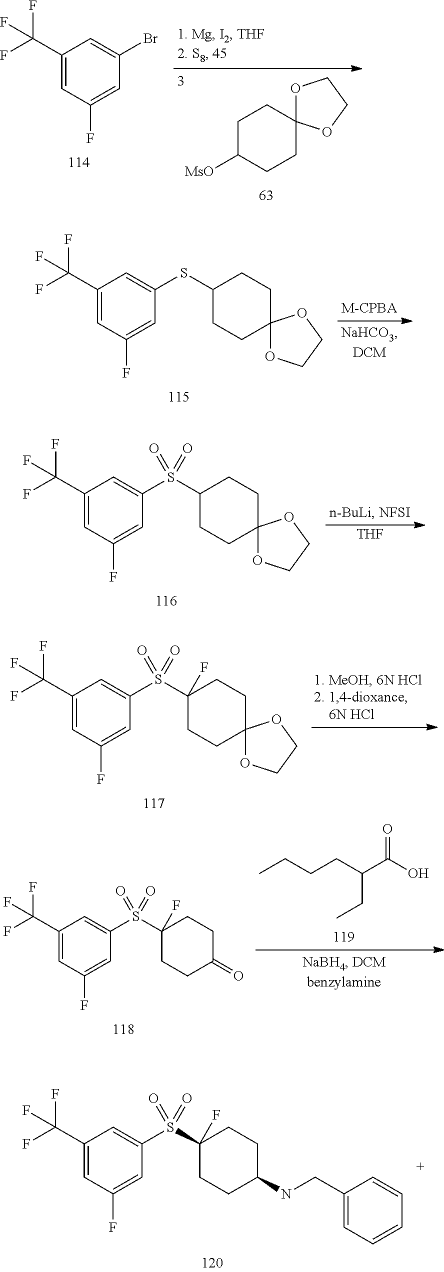

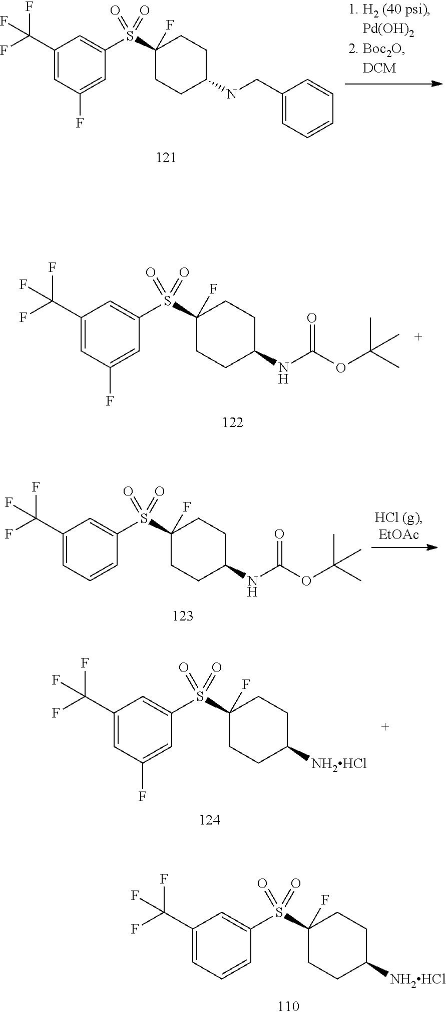

### A. Preparation of 8-(3-fluoro-5-(trifluoromethyl)phenylsulfonyl)-1,4-dioxaspiro[4.5]decane (116)

Mg ribbon (1.09 g, 44.9 mmol) (cleaned with hexane/Et₂O) and I₂(initiator) was stirred in dry THF (75 mL) at room temperature. 1-Bromo-3-fluoro-5-(trifluoromethyl)benzene (114) (10.0 g, 41.2 mmol) was added dropwise, and the reaction stirred for 2 h at room temperature (reaction was initiated with heat gun). Sulfur (1.32 g, 41.2 mmol) was added, and the reaction stirred at room temperature for 45 min. 1,4-Dioxaspiro[4.5]decan-8-yl methanesulfonate (63) (10.5 g, 44.5 mmol) was added and the reaction stirred at reflux for 16 hours. The reaction was then filtered through Celite, washing with EtOAc. The filtrate was washed with brine (200 mL), dried (Na₂SO₄), and concentrated in vacuo. The residue was dissolved in DCM (1 L) and stirred with NaHCO₃ (20 g) and m-CPBA (max 77%, 30 g, 130 mmol) at room temperature for 16 hours. 1.5 N NaOH (100 mL) and saturated Na₂S₂O₃ (50 mL) was added, stirred for 30 minutes and the organics separated, washed with brine (150 mL), dried (Na₂SO₄), concentrated in vacuo and the residue purified by automated column chromatography (EtOAc/PE, 1:3), followed by recrystallization from EtOAc/PE to give 8-(3-fluoro-5-(trifluoromethyl)phenyl sulfonyl)-1,4-dioxaspiro[4.5]decane (116) (10.6 g, 70%); ¹H NMR (300 MHz, CDCl₃) δ 1.56 (m, 2H), 1.84 (m, 4H), 2.08 (m, 2H), 2.99 (m, 1H), 3.93 (m, 4H), 7.64 (d, 1H, 6.9 Hz) 7.80 (d, 1H, J=6.9 Hz), 7.96 (s, 1H).

### B. Preparation of 8-fluoro-8-(3-fluoro-5-(trifluoromethyl)phenylsulfonyl)-1,4-dioxaspiro[4.5]decane (117)

8-(3-Fluoro-5-(trifluoromethyl)phenylsulfonyl)-1,4-dioxaspiro[4.5]decane (116) (20.0 g, 54.3 mmol) was stirred under argon in dry THF (160 mL) at −78° C. n-BuLi (10 M in hexanes, 5.7 mL, 57 mmol) was added dropwise and the reaction stirred for 30 min. N-fluorobenzenesulfonimide (16.7 g, 53.0 mmol) in dry THF (20 mL) was added, the reaction stirred at −78° C. for 30 min then warmed to room temperature an additional 6 hours. EtOAc (100 mL) was added and the mixture filtered through celite, washing EtOAc. The filtrate was concentrated in vacuo, and the residue purified by automated column chromatography (EtOAc/petroleum ether, 1:5) followed by recrystallization from EtOAc/petroleum ether to give 8-fluoro-8-(3-fluoro-5-(trifluoromethyl)phenylsulfonyl)-1,4-dioxaspiro[4.5]decane (117) (12.8 g, 61%); ¹H NMR (300 MHz, CDCl₃) δ 1.82 (m, 4H), 2.04 (m, 2H), 2.33 (m, 1H), 2.46 (m, 1H), 3.98 (s, 4H), 7.68 (d, 1H, 6.9 Hz), 7.85 (d, 1H, J=6.9 Hz), 8.01 (s, 1H).

### C. Preparation of 4-fluoro-4-(3-fluoro-5-(trifluoromethyl)phenylsulfonyl)cyclohexanone (118)

8-Fluoro-8-(3-fluoro-5-(trifluoromethyl)phenylsulfonyl)-1,4-dioxaspiro[4.5]decane (117) (12.8 g, 33.0 mmol) and 6 N HCl (50 mL) were stirred in MeOH (150 mL) at reflux for 2 hours. The reaction was diluted with H₂O (200 mL), extracted with DCM (3×100 mL) and the organics concentrated in vacuo. The residue was taken up in 1,4-dioxane (150 mL) with 6 N HCl (50 mL) and stirred at reflux for 3 hours. The reaction was extracted with DCM (3×100 mL), the organics washed sequentially with H₂O (150 mL) and saturated NaHCO₃ solution (150 mL), dried (Na₂SO₄) and concentrated in vacuo to give 4-fluoro-4-(3-fluoro-5-(trifluoromethyl)phenyl-sulfonyl)cyclohexanone (118) (11.1 g, 97%); ¹H NMR (300 MHz, CDCl₃) δ 2.4 (m, 2H), 2.6 (m, 6H), 7.73 (d, 1H, 6.9 Hz), 7.90 (d, 1H, J=6.9 Hz), 8.05 (s, 1H).

### D. Preparation of cis, trans-N-benzyl-4-fluoro-4-(3-fluoro-5-(trifluoromethyl)phenylsulfonyl)-cyclohexanamine (120+121)

NaBH₄ (3.78 g, 100 mmol) was stirred under argon in DCM (200 mL) at room temperature. 2-Ethylhexanoic acid (119) (50.5 g, 350 mL) was added over 30 min, and the resultant suspension stirred at room temperature for 16 hours. The reaction was filtered and added to 4-fluoro-4-(3-fluoro-5-(trifluoromethyl)phenylsulfonyl)-cyclohexanone (118) (11.1 g, 32.1 mmol) and benzylamine (4.8 g, 45 mmol) in DCM (50 mL). The reaction was stirred at room temperature for 2 h, washed sequentially with 2 N NaOH (100 mL) and brine (200 mL), dried (Na₂SO₄), and concentrated in vacuo. The residue was taken up in Et₂O (300 mL), and acidified with HCl (2 M in Et₂O, 40 mL). The resultant precipitate was collected by filtration, dissolved in H₂O (200 mL) with the minimum amount of MeOH, basified to pH=13 with 2 N NaOH and extracted with DCM (3×150 mL). The organics were dried (Na₂SO₄), concentrated in vacuo and the residue was purified by automated column chromatography (EtOAc/PE, 1:3-1:1, MeOH/DCM, 1:20), to give cis-N-benzyl-4-fluoro-4-(3-fluoro-5-(trifluoromethyl)phenylsulfonyl)cyclohexanamine (120) (10.0 g, 72%). ¹H NMR (300 MHz, CDCl₃) δ 1.8 (m, 6H), 2.5 (m, 1H), 2.6 (m, 1H), 7.3 (m, 5H), 3.76 (s, 2H), 7.67 (d, 1H, 6.9 Hz), 7.86 (d, 1H, J=6.9 Hz), 8.02 (s, 1H). and trans-N-benzyl-4-fluoro-4-(3-fluoro-5-(trifluoromethyl)phenylsulfonyl)cyclohexanamine (121) (3.1 g, 22%).

### E. Preparation of cis-4-fluoro-4-(3-fluoro-5-(trifluoromethyl)phenylsulfonyl)cyclohexanamine hydrochloride salt (124)

Cis-N-benzyl-4-fluoro-4-(3-fluoro-5-(trifluoromethyl)phenylsulfonyl)cyclohexanamine (120) (10.0 g, 23.0 mmol) in MeOH (50 mL) was hydrogenated in the presence of Pd(OH)₂/C (20 wt. %, 1.5 g, 3.8 mmol) under H₂ (40 psi) for 72 h at room temperature. The reaction was filtered through a Celite and the filtrate concentrated in vacuo. The residue was dissolved in DCM (100 mL), DIPEA (2 mL) and Boc₂O (7.0 g, 32 mmol) added, the reaction stirred for 30 min at room temperature, washed sequentially with saturated NH₄Cl (100 mL) and H₂O (100 mL), dried (Na₂SO₄), concentrated in vacuo and the residue purified by automated column chromatography (EtOAc/PE, 0:10-1:10) to give tert-butyl cis 4-fluoro-4-(3-fluoro-5-(trifluoromethyl)phenylsulfonyl)-cyclohexylcarbamate (122) (6.6 g, 65%) and tert-butyl cis-4-fluoro-4-(3-(trifluoromethyl)phenylsulfonyl)cyclo hexylcarbamate (123) (3.4 g, 35%). Cis-4-Fluoro-4-(3-fluoro-5-(trifluoromethyl)-phenylsulfonyl)cyclohexylcarbamate (122) was dissolved in EtOAc (30 mL), HCl (g) bubbled through the solution for 30 s, the reaction stirred for 30 min at room temperature then concentrated in vacuo to give cis-4-fluoro-4-(3-fluoro-5-(trifluoromethyl)phenyl sulfonyl)cyclohexanamine hydrochloride (124) (5.5 g, 65%). ¹H NMR (300 MHz, CD₃OD) δ 2.1 (m, 6H), 2.4 (m, 2H), 3.5 (m, 1H), 8.0 (m, 3H).

### Example 27

### Procedure for the Synthesis of cis-4-fluoro-4-(3-fluoro-5-(trifluoromethyl)phenylsulfonyl)-N-methylcyclohexanamine (125)

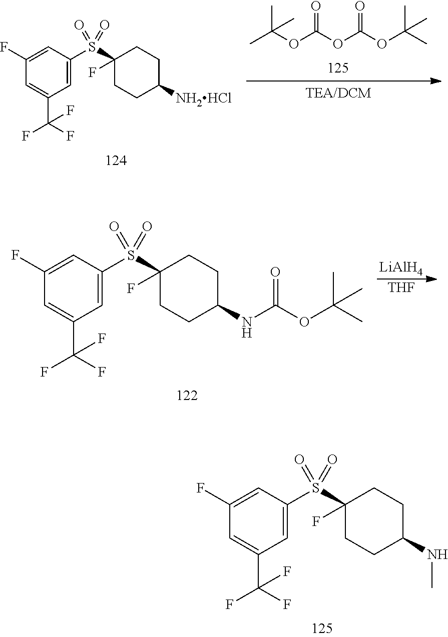

### A. Preparation of tert-butyl cis-4-fluoro-4-(3-fluoro-5-(trifluoromethyl)phenyl sulfonyl)cyclohexyl carbamate (122)

### Cis-4-Fluoro-4-(3-fluoro-5-(trifluoromethyl)phenylsulfonyl)cyclohexanamine hydrochloride (124) (1.5 g, 4.0 mmol), di-tert-butyl-dicarbonate (125) (960 mg, 4.4 mmol) and TEA (1.23 mL, 8.8 mmol) were stirred at room temperature for 5 days. The reaction was diluted with DCM, washed sequentially with 1 M HCl, NaHCO₃ saturated solution and NaCl, saturated solution, separated, dried, and concentrated in vacuo to give tert-butyl cis-4-fluoro-4-(3-fluoro-5-(trifluoromethyl)phenylsulfonyl)cyclo-hexyl carbamate (122) (1.46 g, 83.0%) which was used without further purification; ¹H NMR (300 mHz CDCl₃) δ 130 (s 9H), 1.87 (m, 7H), 2.21 (m, 2H), 3.85 (bs, 1H), 4.63 (bs, 1H) 7.69 (d, 1H, J=7.83 Hz), 7.84 (d, 1H, J=7.05 Hz), 8.00 (s, 1H).

### B. Preparation of cis-4 fluoro-4-(3 fluoro-5-(trifluoromethyl)phenylsulfonyl)-N-methylcyclo-hexanamine (125)

tert-Butyl cis-4-fluoro-4-(3-fluoro-5-(trifluoromethyl)phenylsulfonyl)cyclo-hexyl carbamate (122) (750 mg, 1.7 mmol) and LiAlH₄ (77 mg, 2.04 mmol) were heated under argon at reflux in dry THF (10 mL) for 30 minutes. The reaction was cooled, quenched with 1 M NaOH, filtered, and concentrated in vacuo. The residue was taken up in EtOAc, washed sequentially with NH₄Cl saturated solution and NaHCO₃ saturated solution, separated, dried (Na₂SO₄) and concentrated in vacuo to give cis-4-fluoro-4-(3-fluoro-5-(trifluoromethyl)phenylsulfonyl)-N-methylcyclo-hexanamine (125) (510 mg, 84%) which was used without further purification. The required product was confirmed by subsequent derivitisation.

### Example 28

### Procedure for the Synthesis of (3-(3-(trifluoromethyl)phenylsulfonyl)cyclobutyl)methanamine (135)

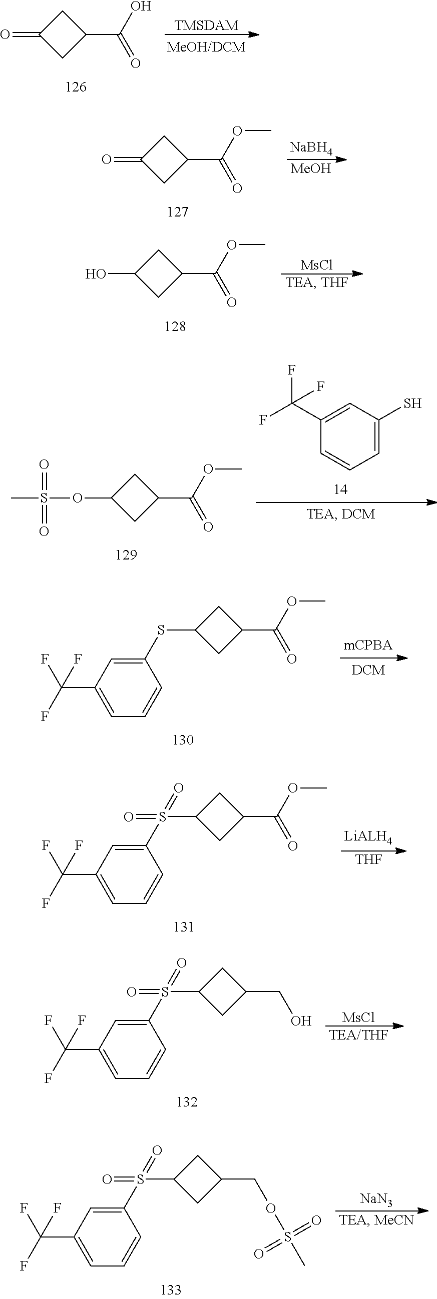

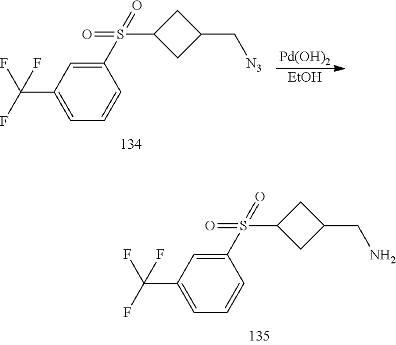

### A. Preparation of methyl 3-oxocyclobutanecarboxylate (127)

Methyl 3-oxocyclobutanecarboxylate (126) (5.0 g, 44 mmol) was stirred in MeOH/DCM (1:6 v/v, 30 mL) at room temperature. Trimethylsilyldiazomethane (2.0 M solution, 23 mL, 46 mmol) was added dropwise with stirring until a persistent yellow colour developed. AcOH was added until the solution decolorized, then the reaction was concentrated in vacuo to give methyl 3-oxocyclobutanecarboxylate (127) (4.65 g, 83%). ¹H NMR (300 mHz CDCl₃) δ 3.25 (m, 5H), 3.68 (s 3H).

### B. Preparation of methyl 3-hydroxycyclobutanecarboxylate (128)

Methyl 3-oxocyclobutanecarboxylate (127) (2.65 g, 21 mmol) was stirred in MeOH (15 mL) at 0° C. NaBH₄ (800 mg, 21 mmol) was added in portions, and the reaction stirred for 20 minutes. H₂O (2 mL) was added and the reaction concentrated in vacuo. The residue was taken up in EtOAc and washed sequentially with NH₄Cl saturated solution and NaHCOₛ saturated solution, the organics separated, dried (MgSO₄), and concentrated in vacuo to give methyl 3-hydroxycyclobutanecarboxylate (128) (1.23 g, 45%). ¹H NMR (300 mHz CDCl₃) δ 2.12 (m, 2H), 2.12 (m 3H), 2.80 (bs, 1H), 3.62 (s, 3H), 4.08 (m, 1H).

### C. Preparation of methyl 3-(methylsulfonyloxy)cyclobutanecarboxylate (129)

Methyl 3-hydroxycyclobutanecarboxylate (128) (4 g, 30.5 mmol), and TEA (8.5 mL, 61.1 mmol) were stirred in THF (50 mL) at room temperature. Methanesulfonyl chloride (2.5 mL, 32 mmol) was added and the reaction stirred at room temperature for 1 hour. The reaction was filtered and the filtrate concentrated in vacuo. The residue was taken up in DCM and washed sequentially with saturated NH₄CL solution and saturated NaHCO₃ solution. The organics were dried (MgSO₄) and concentrated in vacuo to give methyl 3-(methylsulfonyloxy)cyclobutanecarboxylate (129) (3.36 g, 53%). ¹H NMR (300 mHz CDCl₃) δ 2.73 (m, 6H), 2.98 (s 3H), 3.68 (s, 3H), 4.90 (m, 1H). The product was used without further purification.

### D. Preparation of methyl 3-(3-(trifluoromethyl)phenylthio)cyclobutanecarboxylate (130)

Methyl 3-(methylsulfonyloxy)cyclobutanecarboxylate (129) (3.36 g, 16.2 mmol), TEA (4.7 mL, 34 mmol) and 3-(trifluoromethyl)benzenethiol (14) (2.9 g, 16.2 mmol) were stirred in MeCN at reflux for 16 hours. The reaction was concentrated in vacuo, and the residue purified by automated flash chromatography (2% EtOAc/PE) to give methyl 3-(3-(trifluoromethyl)phenylthio)cyclo-butanecarboxylate (130) (1.05 g, 22%); ¹H NMR (300 mHz CDCl₃) δ 2.23 (m, 2H), 2.74 (m 2H), 3.26 (m, 1H), 3.64 (s, 3H), 4.00 (m, 1H), 7.30 (m, 4H).

### E. Preparation of methyl 3-(3-(trifluoromethyl)phenylsulfonyl)cyclobutane carboxylate (131)

Methyl 3-(3-(trifluoromethyl)phenylthio)cyclobutanecarboxylate (130) (1.05 g, 4.1 mmol) and m-CPBA (77%, 2.2 g, 12.3 mmol) were stirred in DCM (30 mL) at room temperature for 16 hours. The reaction was filtered, washed with DCM (30 mL), and the organics washed twice with 10% NaOH solution and dried (MgSO₄). The DCM was removed in vacuo to give methyl 3-(3-(trifluoromethyl)phenylsulfonyl)cyclobutanecarboxylate (131) (920 mg, 70%). ¹H NMR (300 mHz CDCl₃) δ 2.50 (m, 2H), 2.76 (m 2H), 3.25 (m, 1H), 3.64 (s, 3H), 3.85 (m, 1H), 7.68 (t, 1H, J=7.86 Hz), 7.87 (d, 1H, J=7.89 Hz), 8.02 (d, 1H, J=7.86 Hz), 8.09 (s, 1H). The product was used without further purification.

### F. Preparation of (3-(3-(trifluoromethyl)phenylsulfonyl)cyclobutyl)methanol (132)

Methyl 3-(3-(trifluoromethyl)phenylsulfonyl)cyclobutanecarboxylate (131) (920 mg, 2.9 mmol) was stirred under argon in dry THF. LiAlH₄ (122 mg, 3.2 mmol) was added. The reaction stirred for 1 hour then quenched with the dropwise addition of 10% NaOH solution. The resultant precipitate was removed by filtration, washing with additional THF and the organics concentrated in vacuo. The crude residue was taken up in EtOAc, washed sequentially with saturated NH₄Cl solution and saturated NaHCO₃ solution, dried (MgSO₄) and concentrated in vacuo to give (3-(3-(trifluoromethyl)phenylsulfonyl)cyclobutyl)methanol (132) (700 mg, 81%). ¹H NMR (300 mHz CDCl₃) δ 2.08 (m, 2H), 2.60 (m, 2H), 3.59 (d, 2H, J=5.31 Hz), 3.73 (m, 1H), 4.16 (d, 2H, J=5.61 Hz), 7.67 (t, 1H, J=7.83 Hz), 7.86 (d, 1H, J=7.86 Hz), 8.01 (d, 1H, J=7.86 Hz), 8.08 (s, 1H). The product was used without further purification.

### G. Preparation of (3-(3-(trifluoromethyl)phenylsulfonyl)cyclobutyl)methyl methanesulfonate (133)

(3-(3-(Trifluoromethyl)phenylsulfonyl)cyclobutyl)methanol (132) (700 mg, 2.4 mmol) and TEA (420 μL, 3.0 mmol) were stirred at room temperature in DCM (15 mL). Methanesulfonyl chloride (222 μL, 2.9 mmoL) was added and the reaction stirred for 30 minutes, filtered and concentrated in vacuo. The crude material was taken up in DCM, washed sequentially with saturated NH₄CL solution and saturated NaHCO₃ solution, dried (MgSO₄) and concentrated in vacuo to give (3-(3-(trifluoromethyl)phenylsulfonyl)cyclobutyl)methyl methanesulfonate (133) (870 mg, 98%). ¹H NMR (300 mHz CDCl₃) δ 2.17 (m, 2H), 2.67 (m, 2H), 2.96 (s, 3H), 3.75 (m, 1H), 7.66 (t, 1H, J=7.86 Hz), 7.84 (d, 1H, J=7.86 Hz), 8.01 (d, 1H, J=7.86 Hz), 8.08 (s, 1H). The product was used without further purification.

### H. Preparation of 1-(3-(azidomethyl)cyclobutylsulfonyl)-3-(trifluoromethyl)benzene (134)

(3-(3-(Trifluoromethyl)phenylsulfonyl)cyclobutyl)methyl methanesulfonate (133) (870 mg, 2.4 mmol), TEA (500 μL, 3.6 mmol) and NaN₃ (312 mg, 4.8 mmol) were stirred in MeCN (15 mL) at reflux for 16 hours. The reaction was concentrated in vacuo, the residue was taken up in EtOAc, washed with H₂O, dried (MgSO₄) and concentrated in vacuo to give 1-(3-(azidomethyl)cyclobutylsulfonyl)-3-(trifluoromethyl)benzene (134) (630 mg, 82%). ¹H NMR (300 mHz CDCl₃) δ 2.10 (m, 2H), 2.64 (m, 2H), 3.33 (d, 2H, J=5.67 Hz), 3.73 (m, 1H), 7.67 (t, 1H, J=7.83 Hz), 7.86 (d, 1H, J=7.80 Hz), 8.02 (d, 1H, J=7.68 Hz), 8.08 (s, 1H). The product was used without further purification.

### I. Preparation of (3-(3-(trifluoromethyl)phenylsulfonyl)cyclobutyl)methanamine (135)

1-(3-(Azidomethyl)cyclobutylsulfonyl)-3-(trifluoromethyl)benzene (134) (570 mg, 1.8 mmol) and Pd(OH)₂ (60 mg, cat) were taken up in EtOH (20 mL) and placed in a parr hydrogenator. The reaction was agitated under H₂ (50 psi) for 1 h at room temperature. The reaction was filtered through celite and the filtrate concentrated in vacuo to give (3-(3-(trifluoromethyl)phenylsulfonyl)-cyclobutyl)methanamine (135) (520 mg, 100%) which was used without further purification.

### Example 29

### Procedure for the Synthesis of trans-(3-(3-(trifluoromethyl)phenyl sulfonyl)cyclobutyl)methanamine (142)

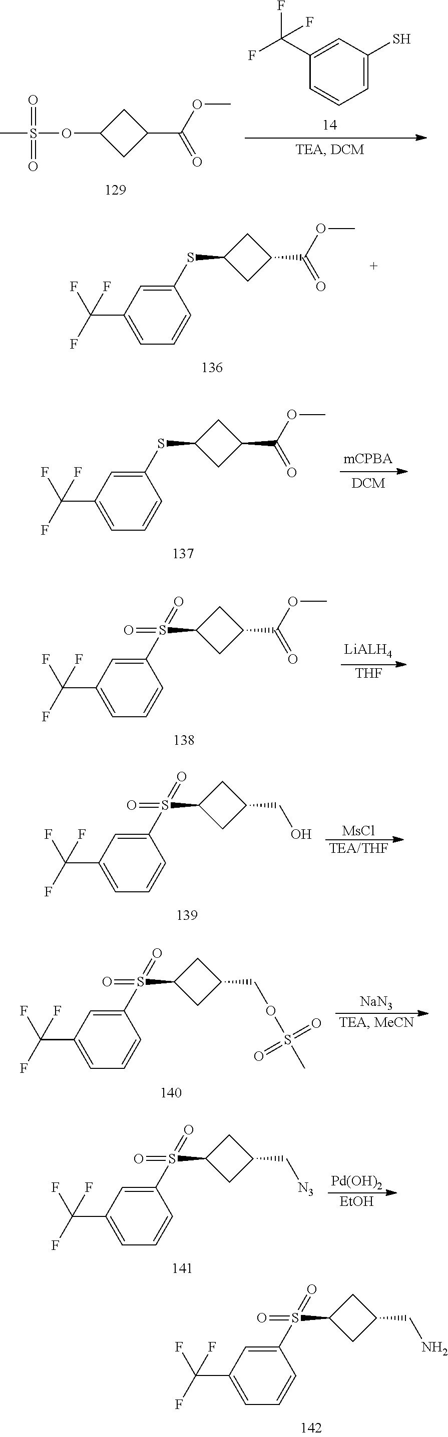

### A. Preparation of cis and trans-methyl 3-(3-(trifluoromethyl)phenylthio)cyclobutane carboxylate (136 and 137)

Methyl 3-(methylsulfonyloxy)cyclobutanecarboxylate (129) (1.53 g, 7.4 mmol), TEA (2.1 mL, 15 mmol) and 3-(trifluoromethyl)benzenethiol (14) (1.3 g, 7.4 mmol) were stirred in MeCN at reflux for 16 hours. The reaction was concentrated in vacuo, and the residue purified by automated flash chromatography (2% EtOAc/PE) to give trans-methyl 3-(3-(trifluoromethyl)phenylthio)-cyclobutanecarboxylate (136) (870 mg, 40.5%). ¹H NMR (300 mHz CDCl₃) δ 2.24 (m, 2H), 2.74 (m, 2H), 3.27 (m, 1H), 3.64 (s, 3H), 3.96 (m, 1H), 7.32 (m, 4H) and cis-methyl 3-(3-(trifluoromethyl)phenylthio)-cyclobutanecarboxylate (137) (70 mg, 3.3%). ¹H NMR (300 mHz CDCl₃) δ 2.33 (m, 2H), 2.66 (m, 2H), 3.03 (m, 1H), 3.61 (s, 3H), 3.69 (m, 1H), 7.34 (m, 4H). The two isomers were confirmed using selective NOE decoupling experiments.

### B. Preparation of trans-(3-(3-(trifluoromethyl)phenylsulfonyl)cyclobutyl)methanamine (142)

Trans-(3-(3-(trifluoromethyl)phenylsulfonyl)cyclobutyl)methanamine (142) was prepared using trans-methyl 3-(3-(trifluoromethyl)phenylthio)-cyclobutanecarboxylate (136) following an identical synthetic protocol to that described for (3-(3-(trifluoromethyl)phenyl sulfonyl)cyclobutyl)methanamine (135).

### Example 30

### Procedure for the Synthesis of trans-methyl 3-(3-fluoro-5-(trifluoromethyl)phenylthio)cyclobutanecarboxylate (152)

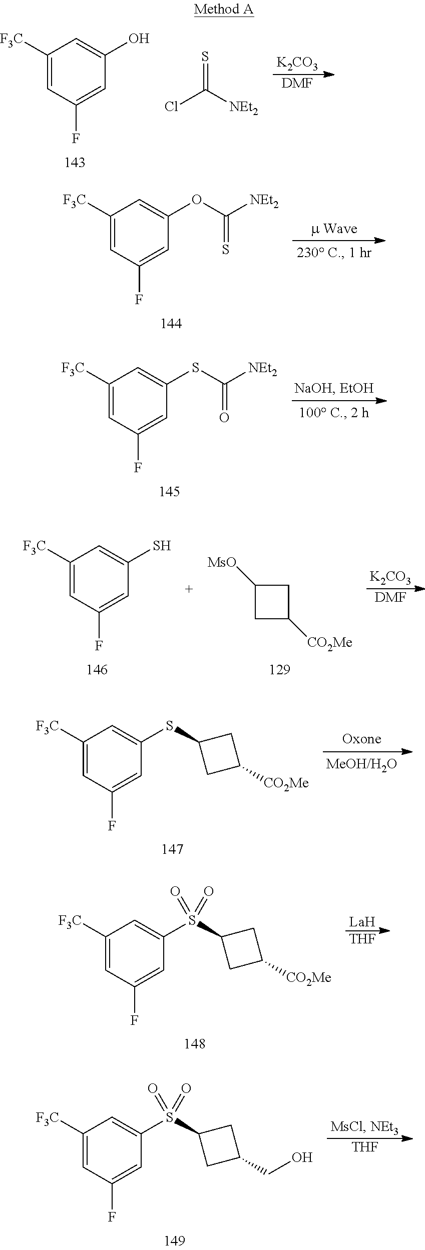

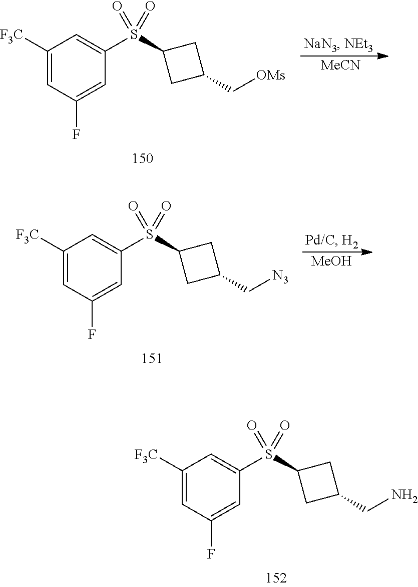

### A. Preparation of O-3-fluoro-5-(trifluoromethyl)phenyl diethylcarbamothioate (144)

3-Fluoro-5-(trifluoromethyl)phenol (143) (10.0 g, 55.55 mmol) and diethylcarbamothioic chloride (9.27 g, 61.04 mmol) were stirred in DMF (100 mL) at room temperature for 16 hours. The reaction was diluted with H₂O (100 mL), extracted with Et₂O (3×50 mL), the organics dried (Na₂SO₄), and concentrated in vacuo to give 3-fluoro-5-(trifluoromethyl)phenyl diethylcarbamothioate (144) (15.3 g, 93%). ¹H NMR (300 MHz, CDCl₃) δ 1.34 (t, 6H, J=7.08 Hz), 3.71 (q, 2H, J=7.05 Hz), 3.88 (q, 2H, J=7.14 Hz), 7.04 (d, 1H, J=8.88 Hz), 7.22 (s, 1H), 7.23 (d, 1H, J=8.01 Hz). The product was used without further purification.

### B. Preparation of S-3-fluoro-5-(trifluoromethyl)phenyl diethylcarbamothioate (145)

3-fluoro-5-(trifluoromethyl)phenyl diethylcarbamothioate (144) (4.7 g, 15.9 mmol) was heated in a sealed vessel at 230° C. for 1 h in a microwave reactor. The crude was then purified by automated flash chromatography (5% EtOAc/PE) to give S-3-fluoro-5-(trifluoromethyl)phenyl diethylcarbamothioate (145) (2.79 g, 60%). ¹H NMR (300 MHz, CDCl₃) δ 1.2-1.3 (m, 6H), 3.44 (q, 4H, J=7.14 Hz), 7.34 (d, 1H, J=8.19 Hz), 7.47 (d, 1H, J=8.31 Hz), 7.57 (s, 1H).

### C. Preparation of 3-fluoro-5-(trifluoromethyl)benzenethiol (146)

S-3-Fluoro-5-(trifluoromethyl)phenyl diethylcarbamothioate (145) (0.1 g, 0.3 mmol) and NaOH (70 mg, 1.69 mmol) was heated at relux in degassed EtOH (10 mL)/H₂O (2 mL) for 2 h. The reaction was diluted with H₂O, acidified with 1 M HCl and extracted with Et₂O (3×10 mL). The organics were dried (Na₂SO₄) and concentrated in vacuo to give 3-fluoro-5-(trifluoromethyl)benzenethiol (146); ¹H NMR (300 mHz, CDCl₃) δ 7.11 (d, 1H, J=8.34 Hz), 7.16 (d, 1H, J=8.73 Hz), 7.31 (s, 1H). The product was used without further purification.

### D. Preparation of trans-methyl 3-(3-fluoro-5-(trifluoromethyl)phenylthio)cyclobutanecarboxylate (147)

3-fluoro-5-(trifluoromethyl)benzenethiol (146) (0.82 g, 4.18 mmol), mesylate (129) (0.87 g, 4.18 mmol) and K₂CO₃ (0.84 g, 6.12 mmol) were heated at reflux in MeCN (50 mL) for 3 h. The reaction was filtered, the filtereate concentrated in vacuo and the residue partitioned between EtOAc and H₂O. The organics were dried (Na₂SO₄), concentrated in vacuo, and the residue purified by automated flash chromatography (5% EtOAc/PE) to give trans-methyl 3-(3-fluoro-5-(trifluoromethyl)phenylthio)cyclobutanecarboxylate (147) (0.26 g, 20%). ¹H NMR (300 mHz, CDCl₃) δ 2.31-2.38 (m, 2H), 2.84-2.87 (m, 2H), 3.32-3.34 (m, 1H), 3.73 (s, 3H), 4.06 (m, 1H), 7.03 (d, 1H, J=8.88 Hz), 7.10 (d, 1H, J=8.34 Hz), 7.19 (s, 1H).

Trans-3-(3-fluoro-5-(trifluoromethyl)phenylsulfonyl)cyclobutyl)methanamine (152) was prepared using 3-(3-fluoro-5-(trifluoromethyl)phenylthio)cyclobutanecarboxylate (147) following an identical synthetic protocol to that described for (3-(3-(trifluoromethyl)phenylsulfonyl)cyclobutyl)methanamine (135).

### Method B

### Procedure for the Synthesis of trans-methyl 3-(3-fluoro-5-(trifluoromethyl)phenylthio)-cyclobutanecarboxylate (147)

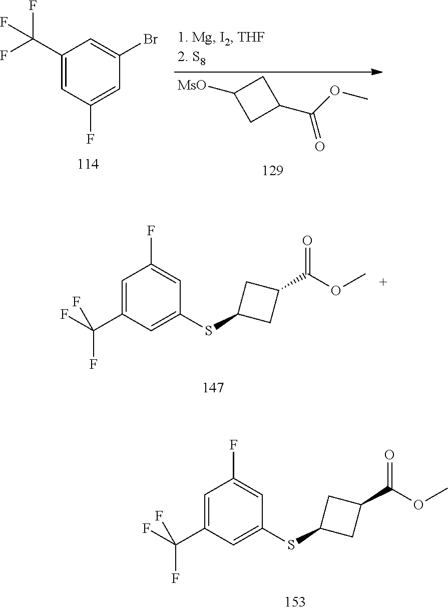

Mg ribbon (1.20 g, 50.0 mmol; cleaned with hexane/Et₂O) and I₂ (initiator) was stirred in dry THF (75 mL) at room temperature. 1-bromo-3-fluoro-5-(trifluoromethyl)benzene (114) (10.9 g, 45.0 mmol) was added dropwise, and the reaction stirred for 3 h at room temperature (Reaction initiated with heat gun). Sulfur (1.44 g, 45.0 mmol) was added and the reaction stirred at room temperature for 1 hour. added methyl 3-(methylsulfonyloxy)cyclobutanecarboxylate (129) (9.00 g, 43.3 mmol) was added and the reaction stirred at reflux for 72 h. 0.5 N HCl (200 mL) was added, the reaction extracted with EtOAc (3×100 mL) and the organics dried (Na₂SO₄), concentrated n-vacuo and the residue purified by automated column chromatography (EtOAc/PE, 0:20-1:20) to give trans-methyl 3-(3-fluoro-5-(trifluoromethyl)phenylthio)cyclobutanecarboxylate (147) (7.0 g, 52%). ¹H NMR (300 MHz, CDCl₃) δ 2.3 (m, 2H), 2.9 (m, 2H), 3.4 (m, 1H), 3.74 (s, 3H), 4.07 (m, 1H), 7.03 (d, 1H, J=8.7 Hz), 7.10 (d, 1H, J=8.7 Hz), 7.19 (s, 1H); and cis-methyl 3-(3-fluoro-5-(trifluoromethyl)phenylthio)cyclobutanecarboxylate (153) (0.7 g, 5%), ¹H NMR (300 MHz, CDCl₃) δ 2.4 (m, 2H), 2.8 (m, 2H), 3.2 (m, 1H), 3.71 (s, 3H), 3.86 (m, 1H), 7.1 (m, 2H), 7.23 (s, 1H).

### Example 31

### Procedure for the Synthesis of cis 3-(3-(trifluoromethyl)phenylsulfonyl)cyclobutyl)methanamine (161)

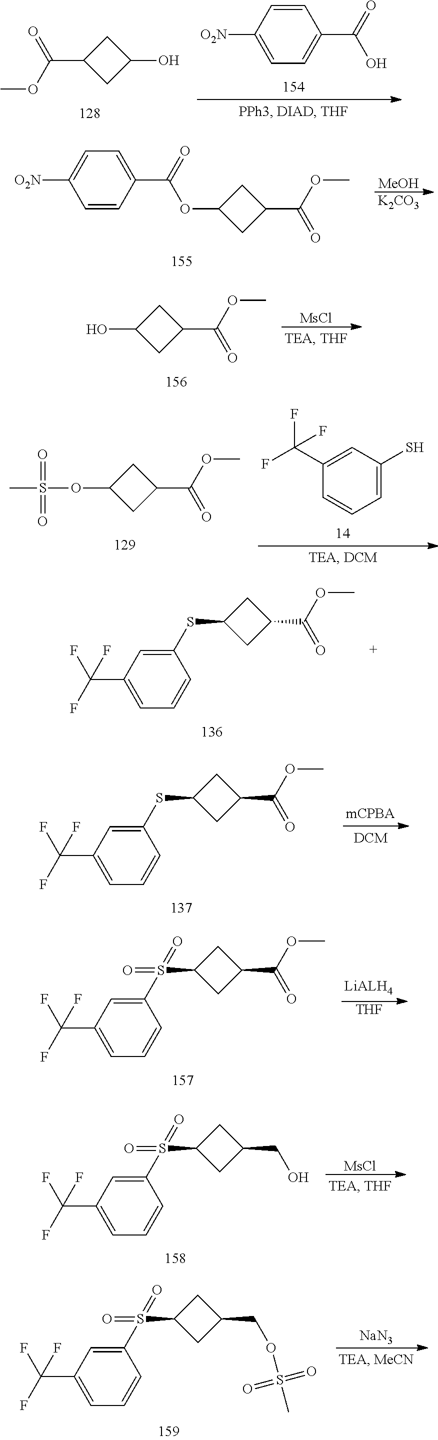

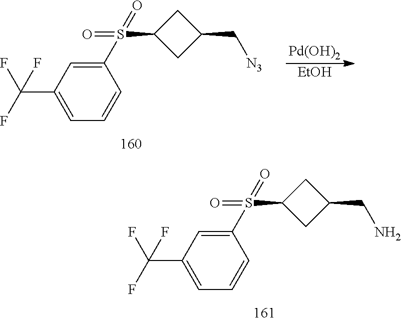

### A. Preparation of 3-(methoxycarbonyl)cyclobutyl 4-nitrobenzoate (155)

Methyl 3-oxocyclobutanecarboxylate (128) (5 g, 38.5 mmol), p-nitrobenzoic acid (154) (6.4 g, 38.5 mmol) and triphenylphosphine (11.09 g, 42.3 mmol) were stirred under argon in dry THF at 0° C. DIAD (8.3 mL, 42.3 mmol) in dry THF (10 mL) was added dropwise, the reaction stirred for 16 h, and then allowed to warm to room temperature. The reaction was concentrated in vacuo and the residue purified by automated column chromatography (10% EtOAc/PE) to give 3-(methoxycarbonyl)cyclobutyl 4-nitrobenzoate (155) (7.52 g, 70.0%). ¹H NMR (300 mHz CDCl₃) δ 2.54 (m, 2H), 2.81 (m, 2H), 3.23 (m, 1H), 3.75 (s, 3H), 5.48 (m, 1 H), 8.21 (d, 2H, J=8.37 Hz), 8.30 (d, 2H, J=7.92 Hz).

### B. Preparation of methyl 3-oxocyclobutanecarboxylate (156)

3-(Methoxycarbonyl)cyclobutyl 4-nitrobenzoate (155) (11.0 g, 39.6 mmol) and K₂CO₃ were stirred in MeOH at room temperature for 3 h. The reaction was concentrated in vacuo, taken up in DCM (approx 15 mL). The resultant precipitate was removed by filtration, washing with additional DCM. The filtrate was concentrated in vacuo, and the residue purified by automated column chromatography (20% EtOAc/PE) to give methyl 3-oxocyclobutanecarboxylate (156) (2.07 g, 41%). ¹H NMR (300 mHz CDCl₃) δ 2.21 (m, 3H), 2.56 (m, 2H), 3.03 (m, 1H), 3.69 (s, 3H), 4.55 (m, 1H).

### C. Preparation of methyl 3-(methylsulfonyloxy)cyclobutanecarboxylate (129)

Methyl 3-oxocyclobutanecarboxylate (156) (2.27 g, 17.6 mmol) and TEA (3.7 mL, 26.3 mmol) were stirred under argon in dry THF (20 mL) at room temperature. Methane sulfonyl chloride (1.64 mL, 21.1 mmol) was added, and the reaction stirred at room temperature for 1 hour. The reaction was filtered, the filtrate concentrated in vacuo, and the residue was taken up in DCM and washed sequentially with saturated NH₄CL solution and saturated NaHCO₃ solution. The organics were dried (MgSO₄) and concentrated in vacuo to give methyl 3-(methylsulfonyloxy)cyclobutanecarboxylate (129) (3.66 g, 100%). ¹H NMR (300 mHz CDCl₃) δ 2.65 (m, 4H), 3.0 (s 3H), 3.15 (m, 1H), 3.72 (s, 3H), 5.24 (m, 1H). The product was used without further purification.

### D. Preparation of cis-methyl 3-(3-(trifluoromethyl)phenylthio)cyclobutane carboxylate (137)

Methyl 3-(methylsulfonyloxy)cyclobutanecarboxylate (129) (3.66 g, 17.6 mmol), K₂CO₃ (4.8 g, 35 mmol) and 3-(trifluoromethyl)benzenethiol (14) (3.1 g, 17.6 mmol) were stirred in DMF at 90° C. for 16 hours. The reaction was diluted with Et₂O and washed sequentially with saturated NaHCO₃ and saturated NaCl solution. The organics were dried (Na₂SO₄), concentrated in vacuo and the residue purified by automated flash chromatography (5% EtOAc/PE) to give cis-methyl 3-(3-(trifluoromethyl)phenylthio)-cyclobutanecarboxylate (137) (1.56 g, 30%). ¹H NMR (300 mHz CDCl₃) δ 2.33 (m, 2H), 2.66 (m, 2H), 3.03 (m, 1H), 3.61 (s, 3H), 3.69 (m, 1H), 7.34 (m, 4H)

### E. Preparation of cis-(3-(3-(trifluoromethyl)phenylsulfonyl)cyclobutyl)methanamine (161)

Cis-(3-(3-(trifluoromethyl)phenylsulfonyl)cyclobutyl)methanamine (161) was prepared using cis-methyl 3-(3-(trifluoromethyl)phenylthio)-cyclobutanecarboxylate (137) following an identical synthetic protocol to that described for (3-(3-(trifluoromethyl)phenylsulfonyl)cyclobutyl)methanamine (135).

### Example 32

### Procedure for the Synthesis of trans-3-((3-(trifluoromethyl)phenyl sulfonyl)methyl)cyclobutanamine hydrochloride (165)

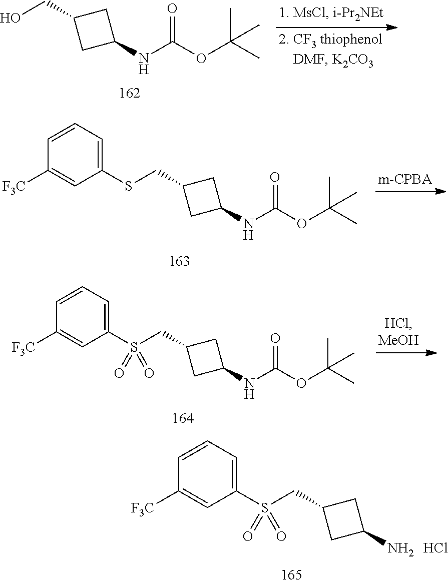

### A. Preparation of tert-butyl trans-3-((3-(trifluoromethyl)-phenylthio)methyl)-cyclobutylcarbamate (163)

tert-Butyl trans-3-(hydroxymethyl)cyclobutylcarbamate (162) (480 mg, 2.38 mmol) and i-Pr₂NEt (0.87 mL, 4.78 mmol) were stirred under argon in dry CH₂Cl₂ (20 mL) at 0° C. MeSO₂Cl (0.22 mL, 2.89 mmol) was added slowly, and the reaction stirred for 1 hour, then washed sequentially with saturated aqueous NH₄Cl and saturated aqueous NaHCO₃, dried (Na₂SO₄), and concentrated in-vacu. The resultant mesylate, 3-(trifluoromethyl)benzenethiol (0.85 g, 4.77 mmol), and K₂CO₃ (658 mg, 4.7 mmol) were stirred under argon in DMF (5 mL) at 90° C. for 18 hours. The solvent was removed in vacuo, and the residue was taken up in EtOAc, washed sequentially with water and brine. The organics were dried (Na₂SO₄), and the residue purified by automated flash chromatography (5:1 PE:EtOAc) to give tert-butyl trans-3-((3-(trifluoromethyl)-phenylthio)methyl)-cyclobutylcarbamate (163) (621 mg, 100%). ¹H NMR (300 mHz CDCl₃) δ 1.46 (s, 9H), 2.03 (m, 2H), 2.2 (m, 2H), 2.4 (m, 1H), 3.1 (d, 2H, J=7.92 Hz), 4.22 (m, 1H), 4.7 (bs, 1H), 7.40 (m, 3H), 7.52 (m, 1H).

### B. Preparation of tert-butyl trans-3-((3-(trifluoromethyl)phenylsulfonyl)methyl)cyclobutyl-carbamate (164)

tert-Butyl-trans-3-((3-(trifluoromethyl)phenylthio)methyl)cyclobutyl carbamate (163) (621 g, 2.38 mmol) and m-CPBA (1.2 g, 70%, 4.8 mmol) were stirred in DCM (100 mL) at room temperature for 16 hours. The reaction was diluted with DCM and the organics washed sequentially with 10% NaOH and brine, dried (Na₂SO₄) and concentrated in vacuo to give tert-butyl trans-3-((3-(trifluoromethyl)phenylsulfonyl)methyl)cyclobutyl-carbamate (164) (1.1 g, 100%). ¹H NMR (300 mHz CDCl₃) δ1.46 (s, 9H), 2.09 (m, 4H), 2.66 (m, 1H), 3.2 (d, 2H, J=7.47 Hz), 4.08 (m, 1H), 4.63 (bs, 1H), 7.70 (t, 1H, J=7.55 Hz), 7.86 (d, 1H, J=7.68 Hz), 8.03 (d, 1H, J=7.95 Hz), 8.08 (s, 1H).

### C. Preparation of trans-3-(3-(trifluoromethyl)phenylsulfonyl)methyl)cyclobutanamine hydrochloride (165)

tert-Butyl-trans-3-((3-(trifluoromethyl)-phenylsulfonyl)methyl)cyclobutyl-carbamate (164) (1.1 g, 2.38 mmol) was dissolved in MeOH at room temperature. HCl gas was bubbled through the solution for 5 min and the reaction stirred at r.t for 1 hour. The solvent was removed in vacuo to give trans-3-(3-(trifluoromethyl)phenylsulfonyl)methyl)cyclobutanamine hydrochloride (164) (600 mg, 100%). MS (M+H=293.9.0) (calcd for C₁₂H₁₄F₃NO₂S, 293.07). The product was used without further purification.

### Example 33

### Procedure for the Synthesis of trans-3-(-1-(3-(trifluoromethyl)phenylsulfonyl)ethyl)cyclobutanamine (168)

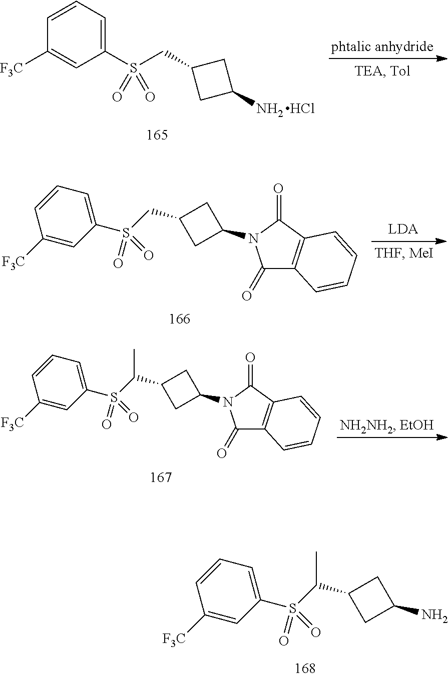

### A. Preparation of 2-(trans-3-((3-(trifluoromethyl)phenylsulfonyl)-methyl)cyclobutyl)-isoindoline-1,3-dione (166)

trans-3-(3-(Trifluoromethyl)phenylsulfonyl)methyl)cyclobutanamine hydrochloride (165) (730 mg, 2.2 mmol), phthalic anhydride (328 mg, 2.2 mmol), and Et₃N (0.35 mL, 2.2 mmol) were stirred under argon in toluene (20 mL at reflux for 2 hours. The reaction was concentrated in vacuo, taken up in EtOAc, washed sequentially with H₂O and brine, dried (Na₂SO₄), concentrated in vacuo, and the crude purified by automated flash chromatography (4:1 PE:EtOAc) to give 2-(trans-3-((3-(trifluoromethyl)phenylsulfonyl)-methyl)cyclobutyl)-isoindoline-1,3-dione (166) (701 mg, 83%); ¹H NMR (300 mHz CDCl₃) δ 2.15 (m, 2H), 3.01 (m, 3H), 3.32 (d, 2 H, J=7.17 Hz), 4.82 (m, 1H), 7.64 (m, 2H), 7.74 (m, 3H), 7.86 (d, 1H, J=7.35 Hz), 8.07 (d, 1H, J=7.98 Hz), 8.12 (s, 1H).

### B. Preparation of 2-(trans-3(-1-(3-(trifluoromethyl)-phenyl-sulfonyl)-ethyl)cyclobutyl)isoindoline-1,3-dione (167)

2-(Trans-3-((3-(trifluoromethyl)phenylsulfonyl)-methyl)cyclobutyl)-isoindoline-1,3-dione (166) (701 g, 1.66 mmol) was stirred under argon in THF (7 mL) at −78° C. LDA (0.66 M, 8 mL, 5.28 mmol) was added drop-wise, and the reaction stirred at −78° C. for 30 min. MeI (0.3 mL, 4.8 mmol) was added dropwise, the reaction stirred for 2 hours at 0° C. then subsequently quenched with saturated NH₄Cl (30 mL). The aqueous was extracted with EtOAc, the organics washed with brine, dried (Na₂SO₄), concentrated in vacuo and the crude material purified by automated flash chromatography (5:1 PE:EtOAc) to give 2-(trans-3(-1-(3-(trifluoromethyl)-phenyl-sulfonyl)-ethyl)cyclobutyl)isoindoline-1,3-dione (167) (220 mg, 30%).

### C. Preparation of trans-3-(-1-(3-(trifluoromethyl)phenylsulfonyl)ethyl)cyclobutanamine (168)

2-(trans-3(-1-(3-(trifluoromethyl)-phenyl-sulfonyl)-ethyl)cyclobutyl)isoindoline-1,3-dione (167) (220 mg, 0.5 mmol) and NH₂NH₂ (0.1 mL, 1.8 mmol) were stirred in EtOH (5 mL) at room temperature for 18 hours. The reaction was filtered, the filtrate concentrated in vacuo, and the residue purified by automated flash chromatography (5:1 DCM:MeOH) to give trans-3-(-1-(3-(trifluoromethyl)phenylsulfonyl)ethyl)cyclobutanamine (168) (130 mg, 89%). MS (M+H=307.9) (calcd for C₁₃H₁₆F₃NO₂S, 307.09).

### Example 34

### Procedure for the Synthesis of trans-3-(2-(3-(trifluoromethyl)phenylsulfonyl)propan-2-yl)cyclobutanamine (178)

### Method A

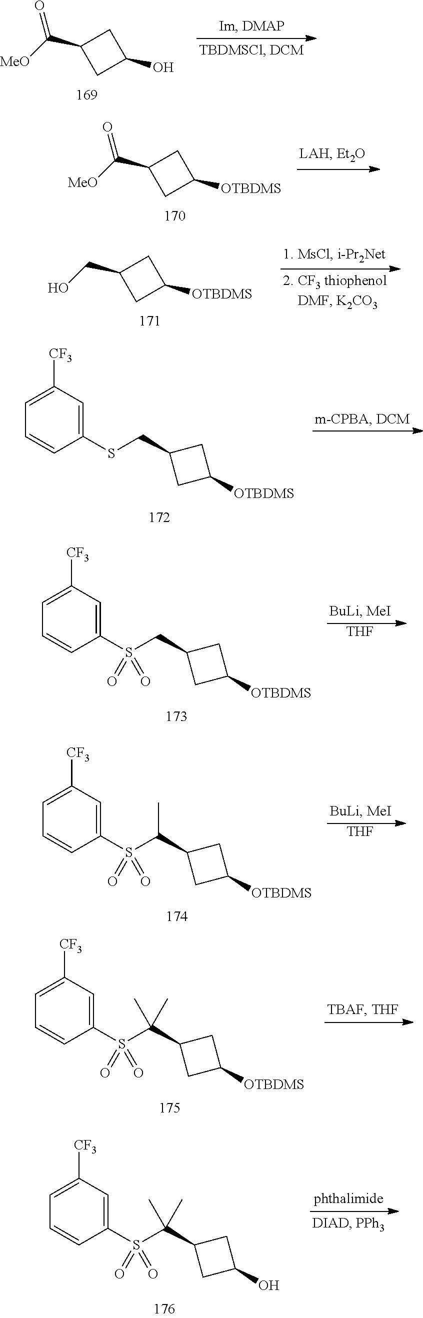

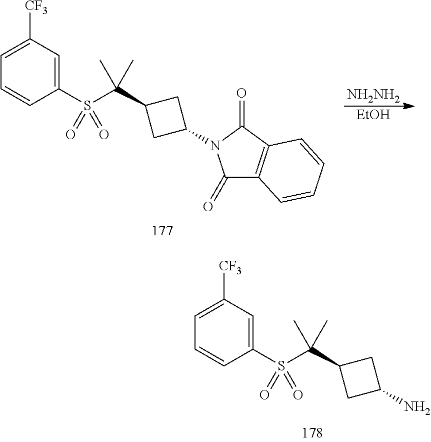

### A. Preparation of cis-methyl 3-(tert-butyldimethylsilyloxy)cyclobutanecarboxylate (170)

Cis-methyl 3-hydroxycyclobutanecarboxylate (169) (3.3 g, 25.19 mmol), imidazole (1.72 g, 25.19 mmol) and DMAP (cat.) were stirred under argon in DCM (100 mL) at room temperature. tert-Butyl dimethylsilyl chloride (3.8 g, 25.19 mmol) was added, the resultant suspension stirred for 3 hours. The reaction was quenched with saturated NaHCO₃ (30 mL) and extracted with EtOAc. The organics were washed with brine, dried (Na₂SO₄), concentrated in vacuo and the crude residue purified by automated flash chromatography (5/1 PE:EtOAc) to give cis-methyl 3-(tert-butyldimethylsilyloxy)cyclobutanecarboxylate (170) (3.5 g, 57%). ¹H NMR (300 mHz CDCl₃) δ 0.00 (s, 6H), 1.20 (s, 9H), 2.17 (m, 2H), 2.46 (m, 3H), 3.64 (s, 3H), 4.10 (m, 1H).

### B. Preparation of cis-3-(tert-butyldimethylsilyloxy)cyclobutyl)methanol (171)

Cis-methyl-3-(tert-butyldimethylsilyloxy)cyclobutanecarboxylate (170) (2.63 g, 10.78 mmol) was stirred under argon in Et₂O (60 mL) at 0° C. LAH (517 mg, 12.94 mmol) was added in portions and the reaction stirred for 2 hours allowing to warm to room temperature. The reaction was cooled to 0° C., quenched sequentially with H₂O (0.52 mL) and 1M NaOH (15%, 1.56 mL) and H₂O (0.52 mL), stirred for 3 hours then filtered through celite, washing with EtOAc (300 mL). The organics were separated, dried (Na₂SO₄) and concentrated in vacuo to give cis-3-(tert-butyldimethylsilyloxy)cyclobutyl)methanol (171) (1.92 g, 82%). ¹H NMR (300 mHz CDCl₃) δ 0.00 (s, 6H), 0.89 (s, 9H), 1.53 (m, 3H), 1.91 (m, 1H), 2.29 (m, 2H), 3.56 (s, 2H), 4.10 (m, 1H).

### C. Preparation of tert-butyldimethyl(cis-3-((3-(trifluoromethyl)phenylthio)methyl)-cyclobutoxy)silane (172)

Cis-3-(tert-butyldimethylsilyloxy)cyclobutyl)methanol (171) (1.92 g, 8.88 mmol) and i-Pr₂NEt (2.5 mL, 13.67 mmol) were stirred under argon in dry DCM (30 mL) at 0° C. MeSO₂Cl (0.85 mL, 11.10 mmol) was added, the reaction stirred for 1 hour, washed sequentially with saturated NH₄Cl solution and saturated NaHCO₃ solution, dried (Na₂SO₄), and concentrated in-vacuo. The meslyate, 3-(trifluoromethyl)benzenethiol (3.2 g, 17.95 mmol) and K₂CO₃ (2.5 g, 17.95 mmol) were stirred under argon in DMF (20 mL) at 90° C. under argon for 18 hours. The solvent was removed in vacuo, the residue was dissolved in EtOAc, the organics washed sequentially with water and brine, dried (Na₂SO₄), concentrated in vacuo and purified by automated flash chromatography (5:1 PE:EtOAc) to give tert-butyldimethyl(cis-3-((3-(trifluoromethyl)phenyl-thio)methyl)-cyclobutoxy)silane (172) (3.2 g, 96%). ¹H NMR (300 mHz CDCl₃) δ 0.00 (s, 6H), 0.90 (s, 9H), 1.64 (m, 2H), 1.98 (m, 1H), 2.42 (m, 2H), 3.05 (d, 2H, J=7.26 Hz), 4.05 (m, 1H), 7.37 (m, 2H), 7.45 (m, 1H), 7.53 (s, 1H).

### D. Preparation of tert-butyldimethyl-cis-3-((3-(trifluoromethyl)phenyl sulfonyl)methyl)cyclobutoxy silane (173)

tert-butyl-dimethyl(cis-3-((3-(trifluoromethyl)phenylthio)methyl)-cyclobutoxy)silane (172) (1.9 g, 5.05 mmol) and m-CPBA (3.1 g, 70% pure, 12.6 mmol) were stirred in DCM (70 mL) at room temperature for 16 hours. Additional DCM (100 mL) was added, and the organics washed sequentially with 10% NaOH and brine, dried (Na₂SO₄), and concentrated in vacuo to give tert-butyldimethyl-cis-3-((3-(trifluoromethyl)phenylsulfonyl)methyl)cyclobutoxy)silane (173) (2.04 g, 100%). ¹H NMR (300 mHz CDCl₃) δ 0.00 (s, 6H), 0.90 (s, 9H), 1.64 (m, 2H), 2.16 (m, 1H), 2.42 (m, 2H), 3.28 (d, 2H, J=7.2 Hz), 4.10 (m, 1H), 7.74 (t, 1H, J=7.98 Hz)), 7.92 (d, 1H, J=7.74 Hz), 8.11 (d, 1H, J=7.77 Hz), 8.17 (s, 1H).

### E. Preparation of tert-butyl-dimethyl-cis-3-1-(3-(trifluoromethyl)phenyl sulfonyl)ethyl)cyclobutoxy) silane (174)

tert-butyldimethyl-cis-3-((3-(trifluoromethyl)phenylsulfonyl)-methyl)-cyclobutoxy)silane (173) (1.3 g, 3.014 mmol) was stirred under argon in THF (25 mL) at −78° C. n-Butyl lithium (2.3 M solution in hexane) (1.9 mL, 4.37 mmol) was added drop-wise, and the reaction stirred for 30 minutes. MeI (0.4 mL, 6.4 mmol) was added dropwise, and the reaction stirred for 2 hour at 0° C. then quenched with saturated NH₄Cl (30 mL) solution. The aqueous was extracted with EtOAc and the organics washed with brine, dried (Na₂SO₄), concentrated and the crude purified by automated flash chromatography (5:1 PE:EtOAc) to give tert-butyldimethyl-cis-3-1-(3-(trifluoromethyl)phenylsulfonyl)ethyl)cyclobutoxy)silane (174) (1.05 mg, 86%); ¹H NMR (300 mHz CDCl₃) δ 0.00 (s, 6H), 0.90 (s, 9H), 1.22 (d, 2H, J=6.93), 1.73 (m, 2H), 2.03 (m, 1H), 2.34 (m, 2H), 3.12 (m, 1H), 4.06 (m, 1H), 7.74 (t, 1H, J=7.95 Hz)), 7.92 (d, 1H, J=7.92 Hz), 8.06 (d, 1H, J=7.98 Hz), 8.14 (s, 1H).

### F. Preparation of tert-butyldimethyl-cis-3-(2-(3-(trifluoromethyl)phenyl sulfonyl)propan-2-yl)cyclo butoxy)silane (175)

tert-Butyl-dimethyl-cis-3-1-(3-(trifluoromethyl)-phenyl-sulfonyl)ethyl)cyclobutoxy) silane (174) (1.1 g, 2.6 mmol) was stirred under argon in THF (25 mL) at −78° C. n-Butyl lithium (2.3 M solution in hexane) (1.8 mL, 3.9 mmol) was added drop-wise and the reaction stirred at −78° C. for 30 min. MeI (0.5 mL, 8.01 mmol) was added dropwise, and the reaction stirred for 2 hours at 0° C. then quenched with saturated NH₄Cl solution (30 mL). The aqueous was extracted with EtOAc, the organics washed with brine, dried (Na₂SO₄), concentrated in vacuo and the crude purified by automated flash chromatography (5:1 PE:EtOAc) to give tert-butyldimethyl-cis-3-(2-(3-(trifluoromethyl)phenylsulfonyl)propan-2-yl)cyclobutoxy)silane (175) (999 mg, 87%); ¹H NMR (300 mHz CDCl₃) δ 0.00 (s, 6 H), 0.90 (s, 9H), 1.22 (s, 6H), 1.73 (m, 2H), 2.23 (m, 3H), 4.06 (m, 1H), 7.74 (t, 1 H, J=7.86 Hz)), 7.92 (d, 1H, J=7.71 Hz), 8.06 (d, 1H, J=7.74 Hz), 8.14 (s, 1H).

### G. Preparation of cis-3-(2-(3-(trifluoromethyl)phenylsulfonyl)propan-2-yl)cyclobutanol (176)

tert-Butyldimethyl-cis-3-(2-(3-(trifluoromethyl)phenylsulfonyl)-propan-2-yl)cyclobutoxy)silane (175) (990 mg, 2.27 mmol) was stirred under argon in THF (20 mL) at 0° C. TBAF (1 M solution in THF, 2.4 mL, 2.5 mmol) was added, and the reaction stirred for 18 hours allowing to warm to room temperature. The solvent was removed in vacuo, and the residue was dissolved in EtOAc, washed sequentially with H₂O and brine, dried (Na₂SO₄), concentrated in vacuo and the crude material purified by automated flash chromatography (3:1 PE:EtOAc) to give cis-3-(2-(3-(trifluoromethyl)phenylsulfonyl)propan-2-yl)cyclobutanol (176) (490 mg, 81%): ¹H NMR (300 mHz CDCl₃) δ 1.30 (s, 6H), 1.76 (m, 3H), 2.33 (m, 3H), 4.16 (m, 1H), 7.70 (t, 1H, J=7.85 Hz)), 7.92 (d, 1H, J=7.68 Hz), 8.08 (d, 1H, J=7.83 Hz), 8.14 (s, 1H).

### H. Preparation of product 2-trans-3-(2-(3-(trifluoromethyl)-phenyl-sulfonyl)propan-2-yl)cyclobutyl)isoindoline-1,3-dione (177)

Cis-3-(2-(3-(trifluoromethyl)phenylsulfonyl)propan-2-yl)-cyclobutanol (176) (606 mg, 1.88 mmol), Ph₃P (1.5 g, 5.64 mmol) and phthalimide (837 mg, 5.64 mmol) were stirred under argon in THF (25 mL) at 0° C. DIAD (1.14 mL, 5.64 mmol) was added, and the reaction stirred for 18 hours allowing to warm to room temperature. The solvent was removed in vacuo, the residue was dissolved in EtOAc, washed sequentially with H₂O and brine, dried (Na₂SO₄), concentrated in vacuo and the crude purified by automated flash chromatography (4:1 PE:EtOAc) to give 2-trans-3-(2-(3-(trifluoromethyl)-phenyl-sulfonyl)propan-2-yl)cyclobutyl)isoindoline-1,3-dione (177) (696 mg, 82%); ¹H NMR (300 mHz CDCl₃) δ1.40 (s, 6H), 2.6 (m, 2H), 2.87 (m, 2H), 3.18 (m, 1H), 4.73 (m, 1H), 7.73 (m, 3H), 7.83 (m, 2H), 7.93 (d, 1H, J=7.68 Hz), 8.08 (d, 1H, J=7.86 Hz), 8.15 (s, 1H).

### I. Preparation of trans-3-(2-(3-(trifluoromethyl)phenylsulfonyl)propan-2-yl)-cyclobutanamine (178)

Trans-3-(2-(3-(trifluoromethyl)-phenyl-sulfonyl)-propan-2-yl)cyclobutyl)isoindoline-1,3-dione (177) (696 mg, 1.54 mmol), and NH₂NH₂ (0.5 mL, 9.90 mmol) were stirred in in EtOH (15 mL) at room temperature for 18 hours. The reaction was filtered, the filtrate concentrated in vacuo and the crude purified by automated flash chromatography (5:1 DCM:MeOH) to give trans-3-(2-(3-(trifluoromethyl)phenylsulfonyl)propan-2-yl)-cyclobutanamine (178) (395 mg, 80%); ¹H NMR (300 mHz CDCl₃) δ 1.40 (s, 6H), 1.80 (m, 2H), 2.24 (m, 2H), 2.92 (m, 1H), 3.46 (m, 1H), 7.72 (t, 1H, J=7.78 Hz)), 7.92 (d, 1H, J=7.77 Hz), 8.05 (d, 1H, J=7.68 Hz), 8.12 (s, 1H).

### Method B: Procedure for the synthesis of cis3-(2-(3-trifluoromethyl)phenyl sulfonyl)propan-2-yl)cyclobutanol (176)

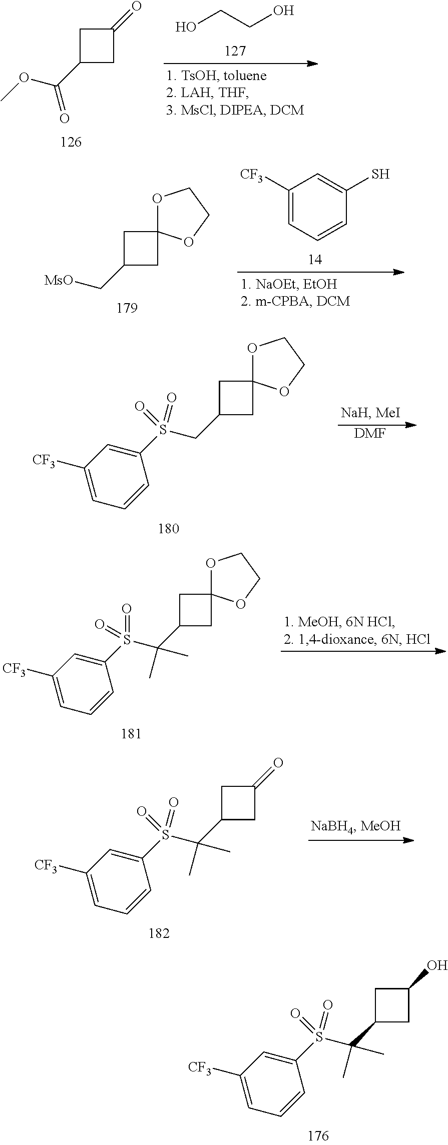

### J. Preparation of 5,8-dioxaspiro[3.4]octan-2-ylmethyl methanesulfonate (179)

Methyl 3-oxocyclobutanecarboxylate (126) (21 g, 160 mmol), TsOH (3.00 g, 15.6 mmol) and ethylene glycol (29.0 g, 468 mmol) were stirred in toluene (200 mL) at reflux in a Dean-Stark apparatus for 16 hours. Saturated NaHCO₃ (100 mL) was added, and the mixture extracted with Et₂O (3×100 mL). The organics were washed with brine (150 mL), dried (Na₂SO₄), and concentrated in vacuo to give a mixture of methyl and ethylene glycol esters, which were used without isolation or purification. The residue was stirred under argon in dry Et₂O (400 mL) at 0° C. LAH (6.83 g, 180 mmol) was added in portions, and the reaction stirred at room temperature for 16 hours. The reaction was quenched with H₂O (7 mL), 4 N NaOH (21 mL) and H₂O (7 mL), and the reaction stirred for 16 hours. The mixture was filtered through Celite washing with DCM/MeOH (1:1V), the filtrate concentrated in vacuo and the residue purified by automated column chromatography (EtOAc/PE, 2:1) to give the alcohol (17.4 g, 76%). The alcohol was stirred in dry DCM (200 mL) at 0° C. DIPEA (19.4 g, 150 mmol) and MsCl (15.6 g, 136 mmol) were added, the reaction stirred at room temperature for 1 hour then washed sequentially with 1 M HCl (100 mL) and brine (2×100 mL) and concentrated in vacuo to give 5,8-dioxaspiro[3.4]octan-2-ylmethyl methanesulfonate (179) (29.5 g, 98% (from alcohol)); ¹H NMR of (300 MHz, CDCl₃) δ 2.2 (m, 2H), 2.5 (m, 3H), 3.02 (s, 3H), 3.90 (s, 4H), 4.27 (d, 2H, J=6.9 Hz).

### K. Preparation of 2-((3-trifluoromethyl)phenylsulfonyl)methyl)-5,8-dioxaspiro[3.4]octane (180)

Na (3.41 g 148 mmol) was stirred under argon in EtOH (400 mL) at room temperature. 3-(trifluoromethyl)benzenethiol (14) (24.6 g, 138 mmol) was added. The reaction stirred at room temperature for 30 min then 5,8-dioxaspiro[3.4]octan-2-ylmethyl methanesulfonate (179) (28.7 g, 129 mmol) was added, and the mixture heated at 90° C. for 1 hour. EtOAc (300 mL) was added, the reaction filtered through Celite, the filtrate concentrated in vacuo, and the residue was dissolved in DCM (1 L). NaHCO₃ (100 g) and m-CPBA (max 77%, 80 g, 345 mmol) was added at 0° C. and the reaction stirred at room temperature for 16 hours. 1 N NaOH (600 mL) and saturated Na₂S₂O₃ (50 mL) were added, the mixture stirred for 30 minutes, the organic layer separated and the aqueous layer was extracted with DCM (2×100 mL). The combined organics were with brine (300 mL), dried (Na₂SO₄), concentrated in vacuo and the residue purified by automated column chromatography (EtOAc/PE, 1:2-1:1), to give 2-((3-trifluoromethyl)phenylsulfonyl)methyl)-5,8-dioxaspiro[3.4]octane (180) (38.6 g, 89%). ¹H NMR of (300 MHz, CDCl₃) δ 2.1 (m, 2H), 2.5 (m, 3H), 3.34 (m, 2H), 3.85 (s, 4H), 7.74 (t, 1H, J=7.5 Hz), 7.93 (d, 1H, J=7.5 Hz), 8.10 (d, 1H, J=7.5 Hz), 8.17 (s, 1H).

### L. Preparation of 2-(2-(3-trifluoromethyl)phenylsulfonyl)propan-2-yl)-5,8-dioxaspiro[3.4]octane (181)

2-((3-trifluoromethyl)phenylsulfonyl)methyl)-5,8-dioxaspiro[3.4]octane (180) (37.0 g, 110 mmol) was stirred under argon in dry DMF (180 mL) at room temperature. NaH (60% in mineral oil, 5.20 g, 130 mmol) was added in portions, and the reaction stirred at room temperature for 30 min. MeI (17.0 g, 120 mmol) was added, and the reaction stirred at room temperature for 16 hours and 60° C. for 1 hour. The reaction was quenched with H₂O and the solvent removed. H₂O (250 mL) was added and the reaction extracted with EtOAc (3×250 mL). The organics were washed with brine (200 mL), dried (Na₂SO₄) and concentrated in vacuo. The residue (a mixture of starting material, mono-methylated and di-methylated material), was dissolved in DMF, and treated with NaH and MeI as above for an additional 3 times. The crude product was purified on a silica gel plug, eluting sequentially with EtOAc/PE, 1:10 and EtOAc/PE, 1:1 to give 2-(2-(3-trifluoromethyl)phenylsulfonyl)propan-2-yl)-5,8-dioxaspiro[3.4]octane (181) (38.7 g, 97%); ¹H NMR of (300 MHz, CDCl₃) δ 1.30 (s, 6H), 2.3 (m, 4H), 2.6 (m, 1H), 3.9 (m, 4H), 7.73 (t, 1H, J=7.5 Hz), 7.92 (d, 1H, J=7.5 Hz), 8.07 (d, 1H, J=7.5 Hz), 8.13 (s, 1H).

### M. Preparation of 3-(2-(3-trifluoromethyl)phenylsulfonyl)propan-2-yl)cyclobutanone (182)

The product was prepared in an analogous fashion to 4-fluoro-4-(3-fluoro-5-(trifluoromethyl)phenylsulfonyl)cyclohexanone (118) using 2-(2-(3-trifluoromethyl)phenylsulfonyl)propan-2-yl)-5,8-dioxaspiro[3.4]octane (181) (38.5 g, 106 mmol). Crystallization from EtOAc/petroleum ether gave 3-(2-(3-trifluoromethyl)phenylsulfonyl)propan-2-yl)cyclobutanone (182) (33.1 g, 97%); ¹H NMR of (300 MHz, CDCl₃) δ 1.30 (s, 6H), 3.0 (m, 1H), 3.10 (m, 4H), 7.77 (t, 1H, J=7.5 Hz), 7.97 (d, 1H, J=7.5 Hz), 8.10 (d, 1H, J=7.5 Hz), 8.16 (s, 1H).

### N. Preparation of cis-3-(2-(3-trifluoromethyl)phenylsulfonyl)propan-2-yl)cyclobutanol (176)

3-(2-(3-trifluoromethyl)phenylsulfonyl)propan-2-yl)cyclobutanone (182) (6.40 g, 20.0 mmol) was stirred in MeOH (80 mL) at 0° C. NaBH₄ (1.10 g, 30.0 mmol) was added and the reaction stirred at 0° C. for 30 min. H₂O (100 mL) was added, The MeOH removed in vacuo and the aqueous layer extracted with EtOAc (3×50 mL). The organics were dried (Na₂SO₄), and concentrated in vacuo to give cis-3-(2-(3-trifluoromethyl)phenylsulfonyl)propan-2-yl)cyclobutanol (176) (6.60 g, 100%); ¹H NMR of (300 MHz, CDCl₃) δ 1.27 (s, 6H), 1.8 (m, 2H), 2.3 (m, 3H), 4.1 (m, 1H), 7.73 (t, 1H, J=7.5 Hz), 7.93 (d, 1H, J=7.5 Hz), 8.07 (d, 1H, J=7.5 Hz), 8.14 (s, 1H).

### Example 35

### Procedure for the Synthesis of cis-3-(2-(3-(trifluoromethyl)phenyl sulfonyl)propan-2-yl)cyclo-butanamine hydrochloride (184)

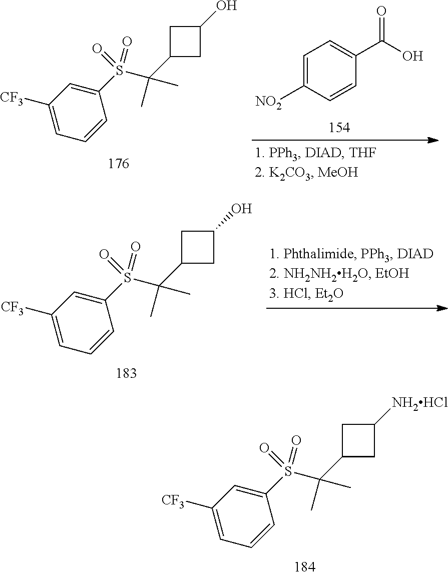

### A. Preparation of trans-3-(2-(3-trifluoromethyl)phenylsulfonyl)propan-2-yl)cyclobutanol (183)

Cis-3-(2-(3-trifluoromethyl)phenylsulfonyl)propan-2-yl)cyclobutanol (176) (5.60 g, 17.5 mmol), Ph₃P (6.88 g, 26.3 mmol), and 4-nitrobenzoic acid (154) (3.84 g, 23.0 mmol) were stirred under argon in THF (60 mL) at 0° C. DIAD (5.31 g, 26.3 mmol) was added dropwise, and the reaction stirred at room temperature for 16 hours. The solvent was removed in vacuo. The residue was taken up in MeOH (150 mL), K₂CO₃ (1.6 g, 12 mmol) added, and the reaction stirred at room temperature for 1 hour. The reaction was concentrated in vacuo, and the residue purified by automated column chromatography (EtOAc/PE, 1:2-2:1), to give trans-3-(2-(3-trifluoromethyl)phenylsulfonyl)propan-2-yl)cyclobutanol (183) (5.0 g, 89%). ¹H NMR of (300 MHz, CDCl₃) δ 1.30 (s, 6H), 2.0 (m, 2H), 2.3 (m, 2H), 2.9 (m, 1H), 4.3 (m, 1H), 7.73 (t, 1H, J=7.5 Hz), 7.93 (d, 1H, J=7.5 Hz), 8.06 (d, 1H, J=7.5 Hz), 8.12 (s, 1H).

### B. Preparation of cis-3-(2-(3-(trifluoromethyl)phenylsulfonyl)propan-2-yl)cyclobutanamine hydrochloride (184)

The product was prepared in an analogous fashion to trans-3-(2-(3-(trifluoromethyl)phenyl sulfonyl)propan-2-yl)cyclobutanamine hydrochloride (178) using trans-3-(2-(3-trifluoromethyl)phenylsulfonyl)propan-2-yl)cyclobutanol (183) (4.80 g, 14.9 mmol) to give cis-3-(2-(3-(Trifluoromethyl)phenylsulfonyl)propan-2-yl)cyclobutanamine hydrochloride (184) (5.0 g, 94%); ¹H NMR (300 MHz CD₃OD) δ 1.29 (s, 6H), 2.1 (m, 2H), 2.4 (m, 2H), 2.7 (m, 1H), 3.65 (m, 1H), 7.92 (t, 1H, J=7.5 Hz), 8.1 (m, 3H), 8.19 (d, 1H, J=7.5 Hz).

### Example 36

### Procedure for the Synthesis of trans-3-(3-(trifluoromethyl)phenylsulfonyl)cyclobutanamine hydrochloride (189)

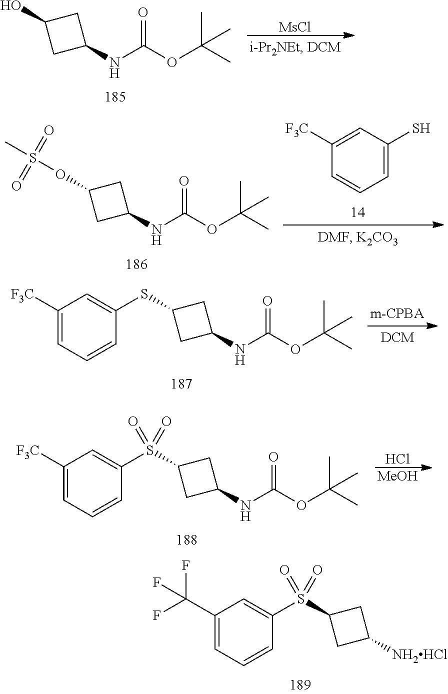

### A. Preparation of trans-3-(tert-butoxycarbonylamino)cyclobutyl methanesulfonate (186)

tert-Butyl cis-3-hydroxycyclobutylcarbamate (185) was synthesized according to the procedure detailed in WO2005/116009A1.

tert-Butyl cis-3-hydroxycyclobutylcarbamate (185) (720 mg, 3.89 mmol) and i-Pr₂NEt (1 mL, 5.40 mmol) were stirred under argon in dry CH₂Cl₂ (15 mL) at 0° C. Methanesulfonyl chloride (0.37 mL, 4.85 mmol) was added and the reaction stirred for 1 hour. The organics were washed sequentially with saturated aqueous NH₄Cl and saturated aqueous NaHCO₃, dried (Na₂SO₄), filtered, and concentrated in vacuo to give trans-3-(tert-butoxycarbonylamino)cyclobutyl methanesulfonate (186) which was used without confirmation and further purification.

### B. Preparation of tert-butyl trans-3-(3-(trifluoromethyl)phenylthio)cyclobutylcarbamate (187)

Crude trans-3-(tert-butoxycarbonylamino)cyclobutyl methanesulfonate (186), prepared above, 3-(trifluoromethyl)benzenethiol (14) (1.4 g, 7.85 mmol), and K₂CO₃ (1.1 g, 7.78 mmol) were stirred under argon in DMF (5 mL) at 90° C. for 18 hours. The reaction was concentrated in vacuo, the residue was dissolved in EtOAc, and washed sequentially with H₂O and brine. The organics were dried (Na₂SO₄), filtered, concentrated in vacuo and the crude material purified by automated flash chromatography (5:1 PE/EtOAc) to give tert-butyl trans-3-(3-(trifluoromethyl)phenylthio)-cyclobutylcarbamate (187) (1.1 g, 82%). ¹H NMR (300 mHz CDCl₃) δ 1.46 (s, 9H), 2.36 (m, 4H), 3.77 (m, 1H), 3.82 (bs, 1H), 4.68 (bs, 1H), 7.30 (m, 4 H).

### C. Preparation of tert-butyl trans-3-(3-(trifluoromethyl)phenylsulfonyl)cyclobutylcarbamate (188)

tert-Butyl trans-3-(3-(trifluoromethyl)phenylthio)cyclobutylcarbamate (187) (1.1 g, 3.17 mmol) and m-CPBA (1.9 g, 70%, 7.7 mmol) were stirred in DCM (100 mL) at room temperature for 16 hours. Additional DCM (100 mL) was added, and the organics washed sequentially with 10% NaOH and brine, dried (Na₂SO₄), filtered, and concentrated in vacuo to give tert-butyl trans-3-(3-(trifluoromethyl)phenylsulfonyl)cyclobutylcarbamate (188) (1.3 g, 100%). ¹H NMR (300 mHz CDCl₃) δ 1.40 (s, 9H), 2.36 (m, 2H), 2.79 (m, 2H), 3.71 (m, 1H), 4.24 (m, 1H), 4.68 (bs, 1H), 7.66 (t, 1H, J=7.78 Hz), 7.86 (d, 1H, J=7.98 Hz), 8.01 (d, 1H, J=7.89), 8.09 (s, 1H).

### D. Preparation of trans-3-(3-(trifluoromethyl)phenylsulfonyl)cyclobutanamine hydrochloride (189)

tert-Butyl trans-3-(3-(trifluoromethyl)phenylsulfonyl)cyclobutylcarbamate (188) (1.3 g, 3.17 mmol) was stirred in MeOH (20 mL) at room temperature. Gaseous HCl was bubbled in through the solution for 5 minutes, the reaction stirred for 1 hour then concentrated in vacuo to give trans-3-(3-(trifluoromethyl)-phenylsulfonyl)-cyclobutanamine hydrochloride (189) (998 mg, 100%). ¹H NMR (300 mHz —CD₃OD) δ 2.60 (m, 2H), 2.83 (m, 2H), 4.06 (m, 2H), 7.88 (t, 1H, J=8.20 Hz), 8.13 (d, 1H, J=7.92 Hz), 8.23 (s, 2H).

### Example 37

### Procedure for the Synthesis of cis-3-(3-(trifluoromethyl)phenylsulfonyl)cyclobutanamine hydrochloride (193)

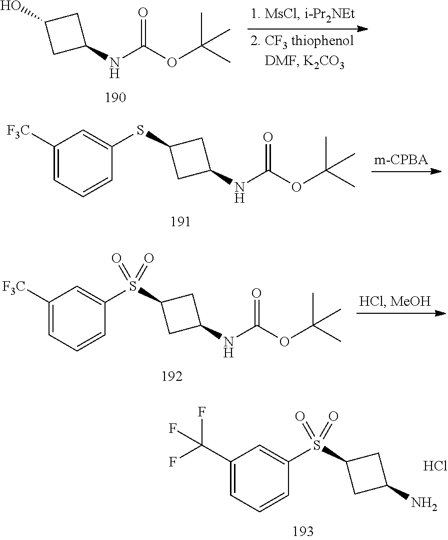

### A. Preparation of tert-butyl-cis-3-(3-(trifluoromethyl)phenylthio)cyclobutylcarbamate (191)

tert-butyl-trans-3-hydroxycyclobutylcarbamate (190) was synthesized according to WO2005/116009A1.

tert-Butyl-trans-3-hydroxycyclobutylcarbamate (190) (587 mg, 3.14 mmol) and i-Pr₂NEt (1.1 mL, 6.28 mmol) were stirred under argon in dry DCM (30 mL) at 0° C. MeSO₂Cl (0.37 mL, 4.85 mmol) was added dropwise, and the reaction stirred for 1 hour. The reaction was then washed sequentially with saturated NH₄Cl solution and saturated aqueous NaHCO₃ solution. The organics were dried (Na₂SO₄), and concentrated in vacuo. The mesylate, 3-(trifluoromethyl)benzenethiol (0.84 g, 4.7 mmol) and K₂CO₃ (866 mg, 6.36 mmol) were stirred under argon in DMF (10 mL) at 90° C. for 18 hours. The solvent was removed in vacuo, the residue was dissolved in EtOAc, washed sequentially with water and brine, dried (Na₂SO₄), concentrated in vacuo and the residue purified by automated flash chromatography (5:1 PE:EtOAc) to give provide the tert-butyl-cis-3-(3-(trifluoromethyl)phenylthio)cyclobutylcarbamate (191) (700 g, 71%). ¹H NMR (300 mHz CDCl₃) δ 1.43 (s, 9 H), 1.93 (m, 2H), 2.93 (m, 2H), 3.52 (m, 1H), 4.14 (bs, 1H), 4.72 (bs, 1H), 7.46 (m, 4H).

### B. Preparation of tert-butyl-cis-3-(3-(trifluoromethyl)phenylsulfonyl)cyclobutylcarbamate (192)

tert-Butyl-cis-3-(3-(trifluoromethyl)phenylthio)cyclobutylcarbamate (191) (700 mg, 2.02 mmol) and m-CPBA (1.2 g, 70% pure, 5.04 mmol) were stirred in DCM (100 mL) at room temperature for 16 hours. Additional DCM (100 mL) was added, the organics washed sequentially with 10% NaOH and brine, dried (Na₂SO₄), and concentrated in vacuo to give tert-butyl-cis-3-(3-(trifluoromethyl)phenylsulfonyl)cyclobutylcarbamate (192) (828 mg, 100%). ¹H NMR (300 mHz CDCl₃) δ 1.40 (s, 9H), 2.36 (m, 2H), 2.57 (m, 2H), 3.41 (m, 1H), 4.10 (m, 1H), 4.85 (bs, 1H), 7.66 (t, 1H, J=7.67 Hz), 7.86 (d, 1H, J=7.17 Hz), 7.97 (d, 1H, J=7.44), 8.06 (s, 1H).

### C. Preparation of cis-3-(3-(trifluoromethyl)phenylsulfonyl)cyclobutanamine hydrochloride (193)

tert-Butyl-cis-3-(3-(trifluoromethyl)phenylsulfonyl)cyclobutylcarbamate (192) (828 mg, 2.02 mmol) was dissolved in MeOH. HCl gas was bubbled through the solution for 5 minutes then the reaction was stirred at room temperature for 1 hour. The solvent was removed in vacuo to give cis-3-(3-(trifluoromethyl)phenylsulfonyl)cyclobutanamine hydrochloride (193) (470 mg, 75%). MS (M+H=279.8) (calcd for C₁₁H₁₂F₃NO₂S, 279.05); Purity: 100% based on LC/MS.

### Example 38

### Procedure for the Synthesis of trans-1-(aminomethyl)-4-(3-(trifluoromethyl)phenylsulfonyl)cyclohexanol (195)

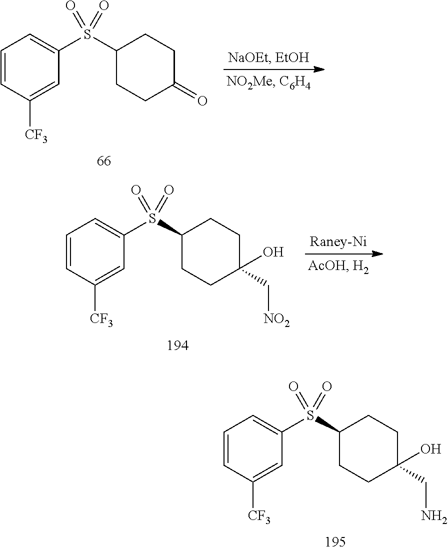

### A. Preparation of trans-1-(nitromethyl)-4-(3-(trifluoromethyl)phenylsulfonyl)cyclohexanol (194)

4-(3-(Trifluoromethyl)phenylsulfonyl)cyclohexanone (66) (1.81 g, 6.0 mmol) and nitromethane (524 μL, 9.75 mmoL) were stirred under argon in benzene at room temperature. NaOEt solution (prepared from Na (184 mg, 8 mmol) and EtOH (10 mL)) was added dropwise and the reaction stirred for 16 hours. Et₂O (80 mL) was added, the reaction stirred for 1 hour, and the resultant precipitate collected by filtration. The collected solid was taken up in H₂O (50 mL), AcOH (0.5 mL) added and stirred for 30 minutes. The resultant precipitate was collected by filtration and dried under high vacuum to give trans-1-(nitromethyl)-4-(3-(trifluoromethyl)phenylsulfonyl)cyclohexanol (194) (1.19 g, 54%). ¹H NMR (300 mHz —CD₃OD) δ 1.49 (m, 8H), 4.42 (d, 2H), 5.17 (bs, 1H), 8.18 (m, 4H). The product was used without further purification.

### B. Preparation of trans-1-(aminomethyl)-4-(3-(trifluoromethyl)phenyl sulfonyl)cyclohexanol (195)

Trans-1-(nitromethyl)-4-(3-(trifluoromethyl)phenylsulfonyl)cyclohexanol (194) (1.19 g, 3.2 mmol) and Raney-Ni (cat) were taken up in AcOH (40 mL) and placed in a Parr hydrogenator. The reaction was agitated under H₂ (50 PSI) for 2 hours at room temperature. The reaction was filtered through celite and the filtrate concentrated in vacuo to give trans-1-(aminomethyl)-4-(3-(trifluoromethyl)phenylsulfonyl)cyclohexanol (195) (520 mg, 100%) which was used without further purification.

### Example 39

### Procedure for the Synthesis of 2-bromo-1-(3-(3-(trifluoromethyl)phenyl sulfonyl)cyclobutyl)ethanone (197)

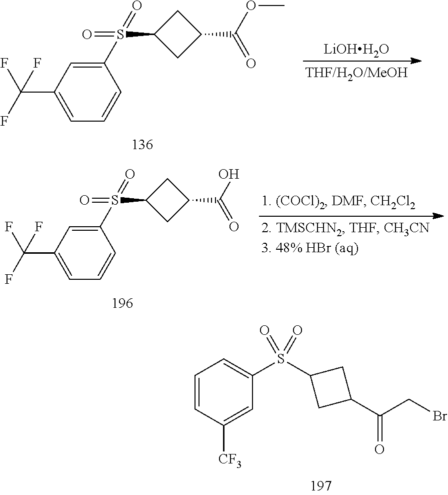

### A. Preparation of trans-3-(3-(trifluoromethyl)phenylsulfonyl)cyclobutanecarboxylic acid (196)

Trans-methyl 3-(3-(trifluoromethyl)phenylsulfonyl)cyclobutane carboxylate (136) (6.8 g, 21.1 mmol) and LiOH.H₂O (1.26 g, 36 mmol) were stirred in THF/H₂O/MeOH (3/3/1, 50 mL) at room temperature for 16 hours. The organics were removed in vacuo, the aqueous made pH 1 with 1M HCl and extracted with EtOAce (2×100 mL). The organics were dried (Na₂SO₄) and concentrated in vacuo to give trans-3-(3-(trifluoromethyl)phenylsulfonyl)cyclobutanecarboxylic acid (196) (6.14 g, 94.5%). ¹H NMR (300 mHz CDCl₃) δ 2.57 (m, 2H), 2.86 (m, 2H), 3.35 (m, 1H), 3.92 (m, 1H), 7.75 (t, 1H, J=7.8 Hz), 7.94 (d, 1H, J=7.71 Hz), 8.09 (d, 1H, J=7.89 Hz), 8.16 (s, 1H). The product was used without further purification.

### B. Preparation of 2-bromo-1-(3-(3-(trifluoromethyl)phenylsulfonyl)cyclobutyl)ethanone (197)

Trans-3-(3-(trifluoromethyl)phenylsulfonyl)cyclobutanecarboxylic acid (196) (2.00 g, 6.49 mmol) and DMF (1 drop, cat.) were stirred under argon in DCM (20 mL). (COCl)₂ (3 mL, excess) was added, and the reaction heated to reflux for 1.5 hours. The reaction was concentrated in vacuo and the residue was dissolved in THF/dry CH₃CN (1/1, 25 mL) under argon at 0° C. TMSCHN₂ (2.0 M solution in Et₂O) (7.14 mL, 14.3 mmol) was added, and the reaction stirred at 0° C. for 6 hours. 48% HBr (10 mL) was added slowly, the residue was allowed to warm to room temperature and stirred for 16 hours. The reaction was concentrated under in vacuo, the residue was dissolved in CH₂Cl₂, washed with sequentially with H₂O, saturated NaHCO₃ solution and brine, dried (Na₂SO₄), concentrated in vacuo and the residue purified by automated flash chromatography to 2-bromo-1-(3-(3-(trifluoromethyl)phenyl sulfonyl)cyclobutyl)ethanone (197) (1.21 g, 48%). ¹H NMR (300 mHz, CDCl₃) δ 2.53 (m, 2H), 2.81 (m, 2H), 3.52 (m, 0.5H), 3.81 (m, 1.5H), 3.90 (s, 2H), 7.75 (t, 1H, J=8.14 Hz), 7.94 (d, 1H, J=7.70 Hz), 8.08 (d, 1H, J=7.48 Hz), 8.15 (s, 1H).

### Example 40

### Procedure for the Synthesis of 5-substituted-2-(trifluoromethyl)-6,7-dihydropyrazolo[1,5-a]pyrazin-4(5H)-one (198)

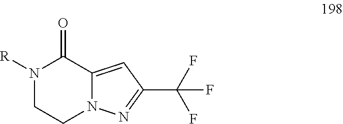

### Method A: Exemplified by the Synthesis of 5-(cis-4-fluoro-4-(3-fluoro-5-(trifluoromethyl)phenylsulfonyl)cyclohexyl)-2-(trifluoromethyl)-6,7-dihydropyrazolo[1,5-a]pyrazin-4(5H)-one (202)

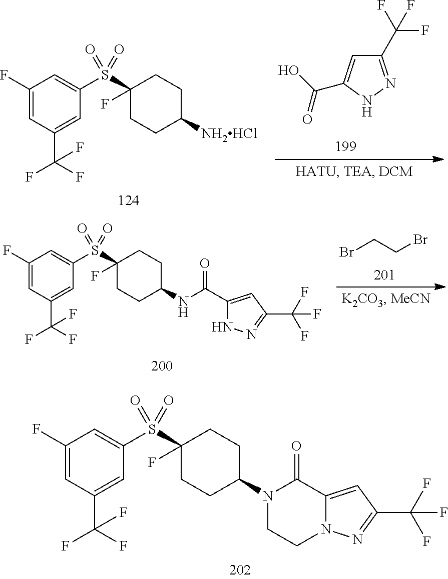

### A. Preparation of N-(cis-4-fluoro-4-(3-fluoro-5-(trifluoromethyl)phenylsulfonyl)cyclohexyl)-3-(trifluoromethyl)-1H-pyrazole-5-carboxamide (200)

Cis-4-fluoro-4-(3-fluoro-5-(trifluoromethyl)phenylsulfonyl)cyclohexanamine hydrochloride (124) (500 mg, 1.32 mmol), 3-trifluoromethyl-1H-pyrazole-5-carboxylic acid (199) (238 mg, 1.32 mmol), HATU (668 mg, 1.8 mmol), and TEA (740 μL, 5.3 mmol) were stirred in DCM (15 mL) at room temperature for 16 hours. The reaction was diluted with DCM, washed sequentially with NH₄Cl saturated solution and Na₂HCO₃ saturated solution, dried (Na₂SO₄), and concentrated in vacuo. The residue purified by automated flash chromatography (20% EtOAc/PE) to give N-(cis-4-fluoro-4-(3-fluoro-5-(trifluoromethyl)phenylsulfonyl)cyclohexyl)-3-(trifluoromethyl)-1H-pyrazole-5-carboxamide (200) (530 mg, 82%).

### B. Preparation of 5-(cis-4-fluoro-4-(3-fluoro-5-(trifluoromethyl)phenylsulfonyl)cyclohexyl)-2-(trifluoromethyl)-6,7-dihydropyrazolo[1,5-a]pyrazin-4(5H)-one (202)

N-(cis-4-fluoro-4-(3-fluoro-5-(trifluoromethyl)phenylsulfonyl)cyclohexyl)-3-(trifluoro-methyl)-1H-pyrazole-5-carboxamide (200) (100 mg, 0.2 mmol), K₂CO₃ (55 mg, 0.4 mmol), and dibromoethane (201) (18 μL, 0.2 mmol) were heated in a sealed vessel at 175° C. for 30 minutes in a microwave. The reaction was portioned between EtOAc and H₂O, the organics separated, dried (Na₂SO₄) and concentrated in vacuo. The crude material was purified by reverse phase HPLC to give 5-(cis-4-fluoro-4-(3-fluoro-5-(trifluoromethyl)phenyl sulfonyl)cyclohexyl)-2-(trifluoro-methyl)-6,7-dihydropyrazolo[1,5-a]pyrazin-4(5H)-one (202).

### Method B

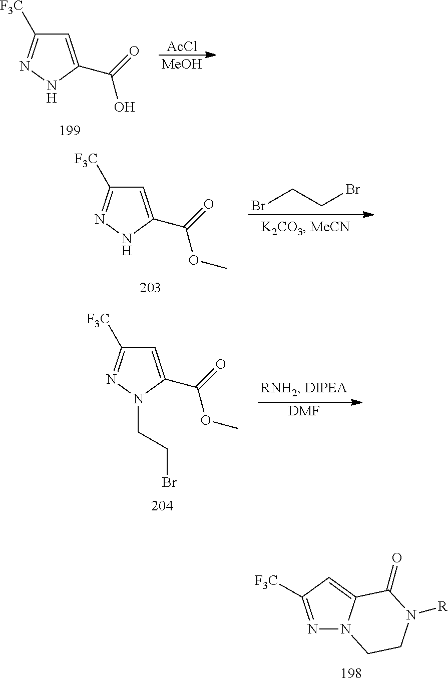

### C. Preparation of methyl 3-(trifluoromethyl)-1H-pyrazole-5-carboxylate (203)

3-(Trifluoromethyl)-1H-pyrazole-5-carboxylic acid (199) (1.0 g, 8.33 mmol) was stirred in MeOH (50 mL) at room temperature. AcCl (1.18 mL, 16.67 mmol) was added dropwise and the reaction stirred at reflux for 2 hours. The reaction was concentrated in vacuo, partitioned between EtOAc and saturated NaHCO₃ solution, and the organics dried (Na₂SO₄) and concentrated in vacuo to give methyl 3-(trifluoromethyl)-1H-pyrazole-5-carboxylate (203) (1.0 g, 93%). ¹H NMR (300 MHz, CDCl₃) δ 3.98 (s, 3H), 7.10 (s, 1H). The product was used without purification.

### D. Preparation of methyl 1-(2-bromoethyl)-3-(trifluoromethyl)-1H-pyrazole-5-carboxylate (204)

Methyl-3-(trifluoromethyl)-1H-pyrazole-5-carboxylate (203) (1.0 g, 5.15 mmol), 1,2-dibromoethane (2.22 mL, 25.77 mmol) and K₂CO₃ (1.42 g, 10.31 mmol) were stirred in MeCN (50 mL) at reflux for 3 hours. The reaction was concentrated in vacuo, the residue partitioned between EtOAc and H₂O, the organics dried (Na₂SO₄) and concentrated in vacuo to give methyl 1-(2-bromoethyl)-3-(trifluoromethyl)-1H-pyrazole-5-carboxylate (204) (1.21 g, 78%). ¹H NMR (300 MHz, CDCl₃) δ 3.74 (t, 2H, J=6.78 Hz), 3.94 (s, 3H), 5.02 (t, 2H, J=6.75 Hz), 7.10 (s, 1H). The product was used without further purification.

### E. Preparation of dihydropyrazolo[1,5-a]pyrazines (198)

Methyl 1-(2-bromoethyl)-3-(trifluoromethyl)-1H-pyrazole-5-carboxylate (203) (100 mg, 0.33 mmol), DIPEA (0.29 mL, 1.67 mmol) and 1 equivalent of amine were stirred in DMF (3 mL) in a sealed vessel at 200° C. for 45 min in a microwave reactor. The reaction was concentrated in vacuo, and the products were purified by reverse HPLC.

### Example 41

### Procedure for the Synthesis of 6-(cis-4-fluoro-4-(3-(trifluoromethyl)phenyl sulfonyl)cyclohexyl)-2-(trifluoromethyl)-6,7-dihydro-5H-pyrrolo[3,4-b]pyridin-5-one (207)

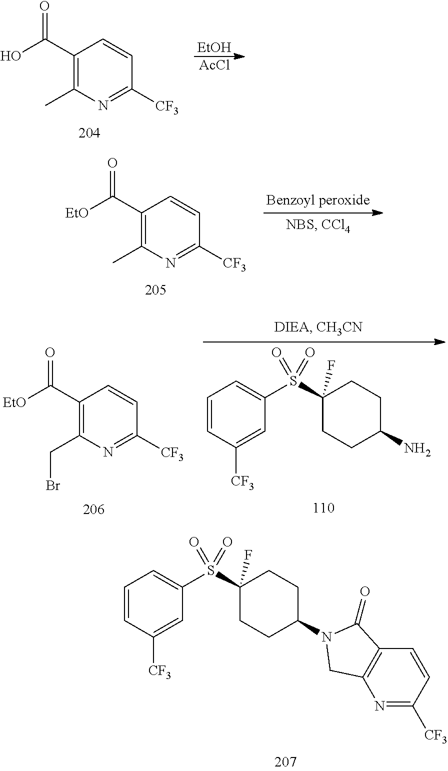

### A. Preparation of ethyl 2-methyl-6-(trifluoromethyl)nicotinate (205)

2-Methyl-6-(trifluoromethyl)nicotinic acid (204) (3.58 g, 17.5 mmol) was stirred in EtOH (50 mL) at room temperature. AcCl (2.48 mL, 34.9 mmol) was added dropwise, and the reaction was then heated to reflux for 6 hours. The reaction was concentrated in vacuo, the residue was taken up in EtOAc, washed with saturated NaHCO₃ solution (twice), dried (Na₂SO₄), and the solvent removed in vacuo to give ethyl 2-methyl-6-(trifluoromethyl)nicotinate (205) (3.33 g, 82%). ¹H NMR (300 mHz, CDCl₃) δ 1.42 (t, 3H, J=7.26 Hz), 2.89 (s, 3H), 4.24 (q, 2H, J=7.26 Hz), 7.59 (d, 1H, J=8.58 Hz), 8.34 (d, 1H, J=8.14 Hz). The product was used without further purification.

### B. Preparation of ethyl 2-(bromomethyl)-6-(trifluoromethyl)nicotinate (206)

Ethyl 2-methyl-6-(trifluoromethyl)nicotinate (205) (3.33 g, 14.3 mmol), NBS (2.54 g, 14.3 mmol), and benzoyl peroxide (0.59 g, 4.3 mmol) were stirred under argon in dry CCl₄ (80 mL) at reflux for 16 hours. The reaction was washed with saturated NaHCO₃ solution, dried (Na₂SO₄), and the solvent was removed in vacuo to provide a 3:1 mixture of ethyl 2-(bromomethyl)-6-(trifluoromethyl)nicotinate (206) with starting material (4.07 g); ¹H NMR (300 mHz, CDCl₃) δ 1.26 (t, 3H, J=7.48 Hz), 4.29 (q, 2H, J=7.26 Hz), 4.85 (s, 2H), 7.51 (d, 1H, J=8.58 Hz), 8.25 (d, 1H, J=8.58 Hz). The crude product was used without purification or isolation.

### C. Preparation of 6-(cis-4-fluoro-4-(3-(trifluoromethyl)phenylsulfonyl)cyclohexyl)-2-(trifluoro-methyl)-6,7-dihydro-5H-pyrrolo[3,4-b]pyridin-5-one (207)

Crude ethyl 2-(bromomethyl)-6-(trifluoromethyl)nicotinate (206) (120 mg, 0.384 mmol), DIEA (0.17 mL, 0.96 mmol), and cis-4-fluoro-4-(3-(trifluoromethyl)phenyl sulfonyl)cyclohexanamine (110) (62 mg, 0.19 mmol) were heated in CH₃CN at 120° C. for 25 minutes, then 130° C. for 30 minutes in a microwave reactor. The reaction was concentrated and purified by reverse phase HPLC to give 6-(cis-4-fluoro-4-(3-(trifluoromethyl)phenyl sulfonyl)cyclohexyl)-2-(trifluoro-methyl)-6,7-dihydro-5H-pyrrolo[3,4-b]pyridin-5-one (207).

### Example 42

### Procedure for the Synthesis of 2-(trans-4-fluoro-4-(3-(trifluoromethyl)phenylsulfonyl)cyclohexyl)-5-(trifluoromethyl)isoindoline-1,3-dione (209)

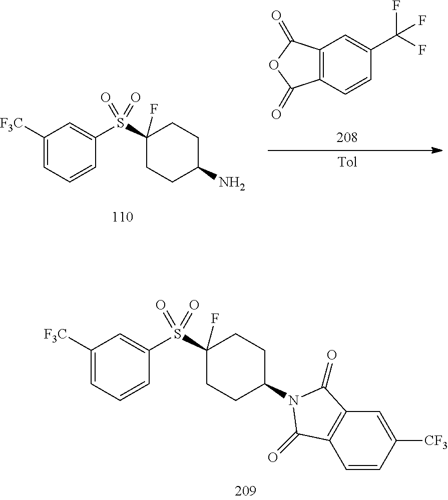

Trans-4-fluoro-4-(3-(trifluoromethyl)phenylsulfonyl)cyclo-hexanamine (110) (206 mg, 0.63 mmol) and 5-trifluoromethyl-phthalic anhydride (208) (136 mg, 0.63 mmol) were stirred under argon in toluene (5 mL) at reflux for 2 hours. The reaction was concentrated in vacuo and purified by reverse phase HPLC to give 2-(trans-4-fluoro-4-(3-(trifluoromethyl)phenylsulfonyl)cyclohexyl)-5-(trifluoromethyl)isoindoline-1,3-dione (209).

### Example 43

### Procedure for the Synthesis of N-(2-amino-2-methylpropyl)-2,2,2-trifluoro-N-trans-4-fluoro-4-(3-(trifluoromethyl)phenylsulfonyl)cyclohexyl)acetamide hydrochloride (212)

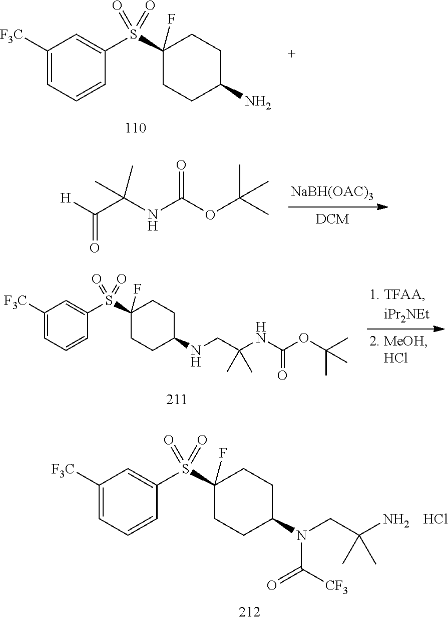

### A. Preparation of tert-butyl 1-trans-4-fluoro-4-(3-(trifluoromethyl)phenylsulfonyl)cyclohexyl amino)-2-methylpropan-2-ylcarbamate (211)

Trans-4-fluoro-4-(3-(trifluoromethyl)phenylsulfonyl)cyclohexanamine (110) (309 mg, 1.2 mmol), tert-butyl 2-methyl-1-oxopropan-2-ylcarbamate (210) (224 mg, 1.2 mmol), and NaBH(OAc)₃ (374 mg, 1.68 mmol) were stirred under argon in DCM (10 mL) under argon at room temperature for 3 hours. The reaction was quenched with NaHCO₃ saturated solution (30 mL) and extracted with EtOAc. The organics washed with brine, dried (Na₂SO₄) and concentrated in vacuo to give tert-butyl 1-trans-4-fluoro-4-(3-(trifluoromethyl)phenylsulfonyl)cyclohexylamino)-2-methylpropan-2-ylcarbamate (211) (547 g, 91); ¹H NMR (300 mHz CDCl₃) δ 1.18 (s 9H), 1.69 (m, 6H), 2.23 (m, 2H), 2.58 (s, 2H), 2.79 (m, 1H), 4.66 (s, 1H), 7.67 (t, 1 H, J=7.85 Hz), 7.89 (d, 1H, J=7.71 Hz), 8.05 (d, 1H, J=7.95 Hz), 8.12 (s, 1H).

### B. Preparation of N-(2-amino-2-methylpropyl)-2,2,2-trifluoro-N-trans-4-fluoro-4-(3-(trifluoro methyl)phenylsulfonyl)cyclohexyl)acetamide hydride chloride (211)

tert-butyl-1-trans-4-fluoro-4-(3-(trifluoromethyl)phenylsulfonyl)cyclohexylamino)-2-methyl propan-2-ylcarbamate (211) (788 mg, 1.59 mmol) and i-Pr₂NEt (0.58 mL, 3.17 mmol) were stirred under argon in DCM (10 mL) at 0° C. TFAA (0.33 mL, 2.37 mmol) was added dropwise and the reaction mixture stirred for 3 hours allowing to warm to room temperature. The reaction was quenched (saturated NaHCO₃ solution (30 mL)), and the aqueous layer was extracted with EtOAc. The organics were washed with brine, dried (Na₂SO₄), and concentrated in vacuo. The residue was treated with HCl in MeOH to give N-(2-amino-2-methylpropyl)-2,2,2-trifluoro-N-trans-4-fluoro-4-(3-(trifluoromethyl)phenylsulfonyl)cyclohexyl)acetamide hydride chloride (212). The product was purified by reverse phase HPLC.

### Example 44

### Procedure for the Synthesis of 6-isopropoxy-5-methylpyrimidine-4-carboxylic acid (216)

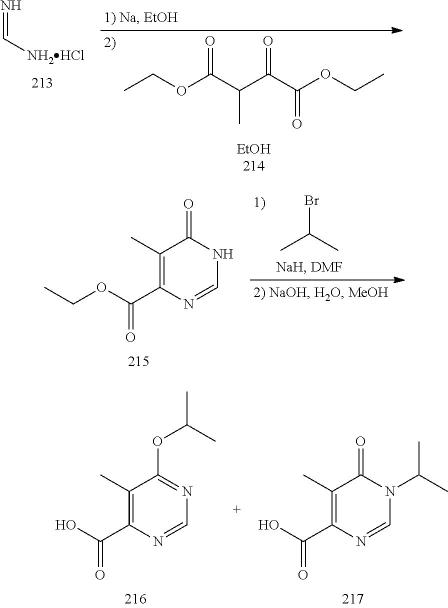

### A. Preparation of ethyl 5-methyl-6-oxo-1,6-dihydropyrimidine-4-carboxylate (215)

Sodium chips (2.85 g, 124 mmol) were added to absolute EtOH (100 mL) under argon at room temperature. The resultant solution was added to formamidine hydrochloride (213) (10.0 g, 124 mmol), and the reaction stirred at room temperature for 45 minutes. The resultant precipitate was removed by filtration, diethyl oxapropionate (214) (24 g, 119 mmol) added, and the reaction heated at 85° C. for 16 hours. The reaction was diluted with EtOAc (300 mL), warmed to 70° C., filtered through a silica plug (eluting with hot EtOAc) and the combined organics concentrated in vacuo. The reaction was repeated twice and all product combined to give 5-methyl-6-oxo-1,6-dihydropyrimidine-4-carboxylate (215) (11.0 g, 18%). ¹H NMR (300 mHz —CD₃OD) δ 1.28 (t, 3H, J=7.08 Hz), 2.09 (s, 3H), 4.29 (q, 2H, J=7.14 Hz), 7.99 (s, 1H).

### B. Preparation of 6-isopropoxy-5-methylpyrimidine-4-carboxylic acid (216)

5-Methyl-6-oxo-1,6-dihydropyrimidine-4-carboxylate (215) (1.87 g, 10.0 mmol) was stirred under argon in DMF (15 mL) at room temperature. NaH ((60% dispersion in mineral oil), 288 mg, 12.0 mmol) was added, and the reaction stirred for 30 minutes. 2-Bromopropane (1.5 mL, 16 mmol) was added, the reaction stirred at 60° C. for 16 hours, quenched with H₂O, and concentrated in vacuo. The residue was heated at 50° C. in 2N NaOH/MeOH (30 mL/30 mL) for 5 hours, concentrated in vacuo, acidified with 6N HCl, and extracted with EtOAc. The organics were dried and concentrated in vacuo to give a mixture of 6-isopropoxy-5-methylpyrimidine-4-carboxylic acid (216) and 1-isopropyl-5-methyl-6-oxo-1,6-dihydropyrimidine-4-carboxylic acid (217) (750 mg, 3/1), which was used without additional purification.

### Example 45

### Procedure for the Synthesis of N,3-dimethyl-3-(3-(trifluoromethyl)phenyl sulfonyl)butan-1-amine hydrochloride (227)

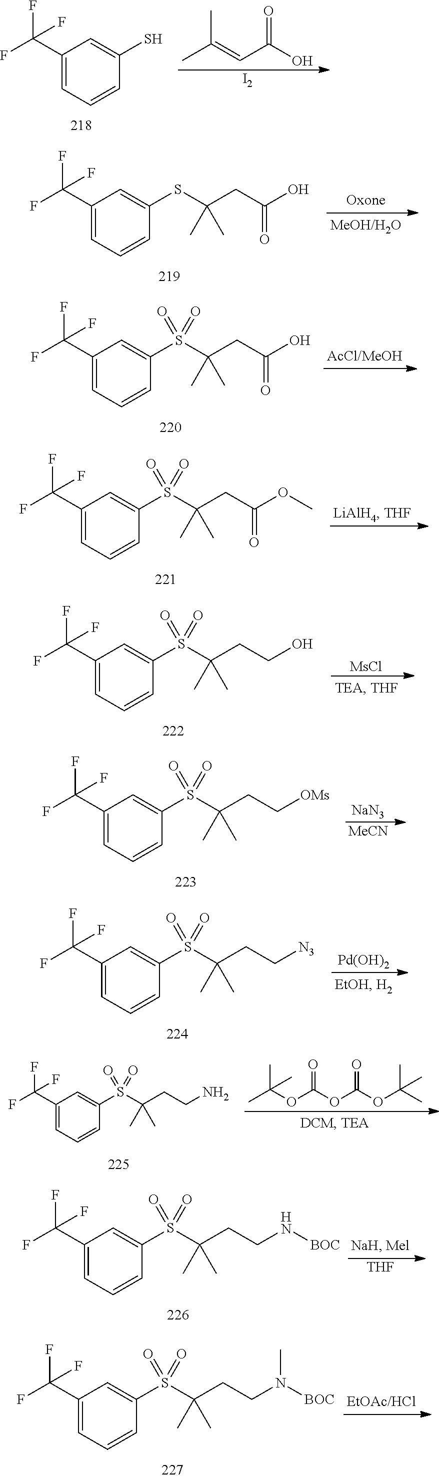

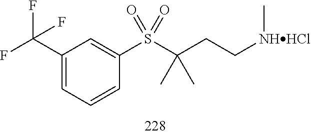

### A. Preparation of 3-methyl-3-(3-(trifluoromethyl)phenylthio)butanoic acid (219)

3-(Trifluoromethyl)benzenethiol (218) (25 g, 140.3 mmol), 3,3-dimethylacrylic acid (14 g, 140 mmol) and iodine (6.9 g, 27 mmol) were heated under argon at 100° C. for 4 hours. After cooling the reaction mixture was taken up in EtOAc, washed with saturated sodium metabisulphite solution. The organics were separated, dried, and concentrated in vacuo. The residue was purified by automated column chromatography (5% PE/EtOAc) to give 3-methyl-3-(3-(trifluoromethyl)phenylthio)butanoic (219) (30.6 g, 79%). ¹H NMR (300 mHz —CD₃Cl) δ 1.43 (s, 6H), 2.55 (s, 2H), 7.49 (t, 1H, J=7.74 Hz), 7.65 (d, 1H, J=7.8 Hz), 7.78 (d, 1H, J=7.71 Hz), 7.84 (s, 1H).

### B. Preparation of 3-methyl-3-(3-(trifluoromethyl)phenylsulfonyl)butanoic acid (220)

3-Methyl-3-(3-(trifluoromethyl)phenylthio)butanoic (219) (13.5 g, 48.6 mmol) and oxone (89 g, 145.7 mmol) were stirred in MeOH/H₂O (450 ml, 2:1) at room temperature for 16 hours. The reaction was filtered, the filtrate concentrated in vacuo, and the residue was taken up in EtOAc, washed repeatedly with H₂O, dried and concentrated in vacuo to give 3-methyl-3-(3-(trifluoromethyl)phenylthio)butanoic (220) (14.1 g, 94%). ¹H NMR (300 mHz —CD₃Cl) δ 1.50 (s, 6H), 2.77 (s, 2H), 7.77 (t, 1H, J=7.83 Hz), 7.97 (d, 1H, J=7.6 Hz), 8.11 (d, 1H, J=7.8 Hz), 8.17 (s, 1H). The product was used without further purification.

### C. Preparation of methyl 3-methyl-3-(3-(trifluoromethyl)phenylsulfonyl)butanoate (221)

3-Methyl-3-(3-(trifluoromethyl)phenylthio)butanoic (220) (10 g, 32.3 mmol) was stirred in MeOH (100 mL) at room temperature. AcCl (4.6 mL, 64.5 mmol) was added dropwise with stirring, and the reaction heated at relux for 2 hours. The reaction was then concentrated in vacuo, taken up in EtOAc, washed with H₂O, dried and concentrated in vacuo to give methyl 3-methyl-3-(3-(trifluoromethyl)phenylsulfonyl)butanoate (221) (9.55 g, 91%). ¹H NMR (300 mHz —CD₃Cl) δ 1.49 (s, 6H), 2.72 (s, 2H), 3.69 (s, 3H), 7.78 (t, 1H, J=7.8 Hz), 7.96 (d, 1H, J=7.71 Hz), 8.10 (d, 1H, J=7.8 Hz), 8.16 (s, 1H). The product was used without further purification.

### D. Preparation of 3-methyl-3-(3-(trifluoromethyl)phenylsulfonyl)butan-1-ol (222)

Methyl 3-methyl-3-(3-(trifluoromethyl)phenylsulfonyl)butanoate (9.55 g, 29.5 mmol) (221) was stirred under argon in THF at room temperature. LAH (1.3 g, 35.4 mmol) was added, the reaction stirred for 2 hours and quenched with 1M NaOH solution. The resultant solid was removed by filtration through Celite, the filtrate was concentrated in vacuo, and the residue partitioned between EtOAc and H₂O. The organics were dried and concentrated in vacuo to give 3-methyl-3-(3-(trifluoromethyl)phenylsulfonyl)butan-1-ol (222) (8.8 g, 94%). ¹H NMR (300 mHz —CD₃Cl) δ 1.38 (s, 6H), 2.03 (m, 2H), 3.86 (m, 2H), 7.75 (t, 1H, J=7.8 Hz), 7.94 (d, 1H, J=7.71 Hz), 8.10 (d, 1H, J=7.8 Hz), 8.16 (s, 1H). The product was used without further purification.

### E. Preparation of 3-methyl-3-(3-(trifluoromethyl)phenylsulfonyl)butyl methanesulfonate (223)

3-Methyl-3-(3-(trifluoromethyl)phenylsulfonyl)butan-1-ol (8.2 g, 27.6 mmol) (222) and TEA (5.0 mL, 36 mmol) were stirred under argon in THF at room temperature. MsCl was added in portions and the reaction stirred for 45 min. The resultant precipitate was removed by filtration. The filtrate was concentrated in vacuo, and the residue was taken up in DCM and washed sequentially with NH₄Cl saturated solution and NaHCO₃ solution. The organics were dried and concentrated in vacuo to give 3-methyl-3-(3-(trifluoromethyl)phenylsulfonyl)butyl methanesulfonate (223) (10.08 g, 100%). ¹H NMR (300 mHz —CD₃Cl) δ 1.38 (s, 6H), 2.22 (t, 2H, J=6.9 Hz), 3.03 (s, 3H), 4.47 (t, 2H, J=6.81 Hz), 7.76 (t, 1H, J=7.8 Hz), 7.95 (d, 1 H, J=7.71 Hz), 8.09 (m, 2H). The product was used without further purification.

### F. Preparation of 1-(4-azido-2-methylbutan-2-ylsulfonyl)-3-(trifluoromethyl)benzene (224)

3-Methyl-3-(3-(trifluoromethyl)phenylsulfonyl)butan-1-ol (10.08 g, 29.5 mmol) (223) and NaN₃ (9.55 g, 147 mmol) were stirred in MeCN at reflux for 16 hours. The reaction was concentrated in vacuo, and the residue was taken up in EtOAc and washed sequentially with NH₄Cl saturated solution and NaHCO₃ solution. The organics were dried, concentrated in vacuo, and the residue purified by automated column chromatography (20% EtOAc/PE) to give 1-(4-azido-2-methylbutan-2-ylsulfonyl)-3-(trifluoromethyl)benzene (224) (6.31 g, 66.6%). ¹H NMR (300 mHz —CD₃Cl) δ 1.36 (s, 6H), 2.02 (m, 2H), 3.52 (t, 1H, J=7.29 Hz), 7.75 (t, 1H, J=7.74 Hz), 7.95 (d, 1H, J=7.74 Hz), 8.08 (d, 1H, J=7.83 Hz), 8.13 (s, 1H).

### G. Preparation of 3-methyl-3-(3-(trifluoromethyl)phenylsulfonyl)butan-1-amine (225)

1-(4-azido-2-methylbutan-2-ylsulfonyl)-3-(trifluoromethyl)benzene (224) (6.31 g, 20.0 mmol) and Pd(OH)₂ (600 mg, cat) were taken up in EtOH (150 mL) and placed in a Parr hydrogenator. The reaction was agitated under H₂ (50 PSI) for 1 hour at room temperature, filtered through Celite, and the filtrate concentrated in vacuo to give 3-methyl-3-(3-(trifluoromethyl)phenylsulfonyl)butan-1-amine (225) (3.96 g, 62%) which was used without further purification.

### H. Preparation of tert-butyl 3-methyl-3-(3-(trifluoromethyl)phenylsulfonyl)butylcarbamate (226)

3-Methyl-3-(3-(trifluoromethyl)phenylsulfonyl)butan-1-amine (225) (750 mg, 2.5 mmol), TEA (0.7 mL, 5.0 mmol) and BOC carbonate (522 mg, 3.0 mmol) were stirred in DCM (30 mL) at room temperature for 1 hour. The reaction was concentrated in vacuo and the residue purified by automated column chromatography (20% EtOAc/PE) to give tert-butyl 3-methyl-3-(3-(trifluoromethyl)phenylsulfonyl)butylcarbamate (226) (680 mg, 75%). ¹H NMR (300 mHz —CD₃Cl) δ 1.36 (s, 6H), 1.43 (s, 9H), 1.94 (m, 2H), 3.31 (m, 2H), 4.65 (bs, 1H), 7.74 (t, 1H, J=7.8 Hz), 7.94 (d, 1H, J=7.89 Hz), 8.09 (d, 1H, J=7.89 Hz), 8.14 (s, 1H).

### I. Preparation of tert-butyl methyl(3-methyl-3-(3-(trifluoromethyl)phenyl sulfonyl)butyl)carbamate (227)

tert-Butyl 3-methyl-3-(3-(trifluoromethyl)phenylsulfonyl)butylcarbamate (226) (680 mg, 1.87 mmol) was stirred under argon in THF at room temperature. NaH ((60% dispersion in mineral oil), 90 mg, 2.25 mmol) was added, and the reaction stirred for 30 minutes. MeI (140 μL, 2.25 mmol) was added, the reaction stirred at room temperature for 16 hours, quenched with H₂O, and concentrated in vacuo. The residue was taken up in DCM, washed sequentially with NH₄Cl saturated solution and NaHCO₃ solution, dried, and concentrated in vacuo to give tert-butyl methyl(3-methyl-3-(3-(trifluoromethyl)phenylsulfonyl)butyl)carbamate (227) (690 mg, 90%). ¹H NMR (300 mHz —CD₃Cl) δ 1.27 (s, 6H), 1.34 (s, 9H), 1.86 (m, 2H), 2.78 (s, 3H), 3.29 (m, 2H), 7.67 (t, 1H, J=7.8 Hz), 7.87 (d, 1H, J=7.71 Hz), 8.02 (d, 1H, J=7.86 Hz), 8.07 (s, 1H).

### J. Preparation of N,3-dimethyl-3-(3-(trifluoromethyl)phenylsulfonyl)butan-1-amine hydrochloride (228)

tert-Butyl methyl(3-methyl-3-(3-(trifluoromethyl)phenylsulfonyl)butyl)carbamate (227) (690 mg, 1.68 mmol) was stirred in EtOAc (30 mL) at room temperature. Gaseous HCl was bubbled through the solution for 1 minute, and the reaction stirred at room temperature for 20 minutes. The solvent was removed in vacuo to give N,3-dimethyl-3-(3-(trifluoromethyl)phenylsulfonyl)butan-1-amine hydrochloride (228) (1.18 g, 82%). The product was used without further purification.

### Example 46

### Procedure for the Synthesis of 3-(3-fluoro-5-(trifluoromethyl)phenyl sulfonyl)-3-methylbutan-1-amine (229)

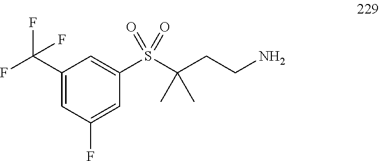

### A. Preparation of N-3-(3-fluoro-5-(trifluoromethyl)phenylthio)-3-methylbutanoic acid (232)

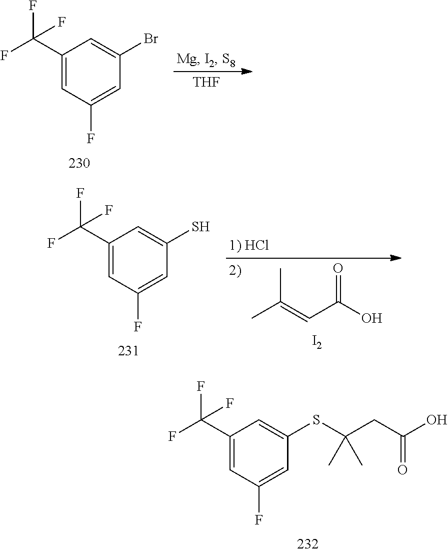

### A1. Preparation of 3-fluoro-5-(trifluoromethyl)benzenethiol magnesium bromide (231)

Mg ribbon (1.09 g, 44.9 mmol; cleaned with hexane/Et₂O) and I₂ (initiator) was stirred in dry THF (75 mL) at room temperature. 1-Bromo-3-fluoro-5-(trifluoromethyl)benzene (230) (10.0 g, 41.2 mmol) was added dropwise and the reaction stirred for 2 hours at room temperature (Reaction initiated with heat gun). Sulfur (1.32 g, 41.2 mmol) was added and the reaction stirred at room temperature for 2 hours. The reaction was filtered and the filtrate concentrated in vacuo to give crude 3-fluoro-5-(trifluoromethyl)benzenethiol magnesium bromide (231) which was used without confirmation or purification.

### A2. Preparation of 3-(3-fluoro-5-(trifluoromethyl)phenylthio)-3-methylbutanoic acid (232)

Crude 3-fluoro-5-(trifluoromethyl)benzenethiol magnesium bromide (231) (5.03 g, 26.7 mmol) was partitioned between 1 M HCl and Et₂O. The organics were separated, dried, and concentrated in vacuo. 3,3-Dimethylacyrlic acid (2.67 g, 26.7 mmol) and I₂ (2.25 g, 8.9 mmol) were added and the reaction heated at 100° C. for 3 hours. After cooling, the reaction mixture was taken up in EtOAc, washed with saturated sodium metabisulphite solution until decolouored and the organics separated, dried and concentrated in vacuo. The residue was purified by automated column chromatography (8% PE/EtOAc) to give 3-(3-fluoro-5-(trifluoromethyl)phenylthio)-3-methylbutanoic acid (232) (2.0 g, 25%). ¹H NMR (300 mHz —CD₃Cl) δ 1.46 (s, 6H), 2.58 (s, 2H), 7.37 (d, 1H, J=8.01 Hz), 7.53 (d, 1 H, J=8.07 Hz), 7.65 (s, 1H).

### B. Preparation of 3-(3-fluoro-5-(trifluoromethyl)phenylsulfonyl)-3-methylbutan-1-amine (229)

3-(3-Fluoro-5-(trifluoromethyl)phenylsulfonyl)-3-methylbutan-1-amine (229) was prepared in analogous fashion to 3-methyl-3-(3-(trifluoromethyl)phenylsulfonyl)butan-1-amine (228) using 3-(3-fluoro-5-(trifluoromethyl)phenylthio)-3-methylbutanoic acid (232).

### Example 47

### Procedure for the Synthesis of 2-methyl-2-(3-(trifluoromethyl)phenyl sulfonyl)propan-1-amine (238)

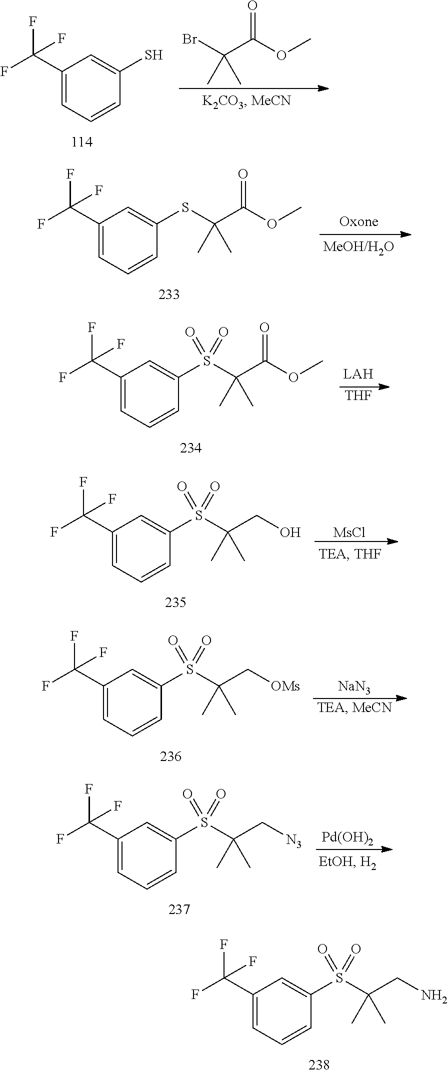

### A. Preparation of methyl 2-methyl-2-(3-(trifluoromethyl)phenylthio)propanoate (233)

3-(Trifluoromethyl)benzenethiol (114) (22.2 g, 124.6 mmol), methyl 2-bromo-2-methylpropanoate (17.1 mL, 137.06 mmol), and K₂CO₃ were heated at reflux for 16 hours. The reaction was filtered, the filtrate concentrated in vacuo, and the residue partitioned between EtOAc and H₂O. The organics were dried, concentrated in vacuo, and the residue purified by automated column chromatography (5% EtOAc/PE) to give methyl 2-methyl-2-(3-(trifluoromethyl)phenylthio)propanoate (233) (32.77 g, 95%). ¹H NMR (300 mHz —CD₃Cl) δ 1.49 (s, 6H), 3.65 (s, 3H), 7.44 (d, 2H, J=7.77 Hz), 7.62 (m, 2H), 7.07 (s, 1H).

### B. Preparation of methyl 2-methyl-2-(3-(trifluoromethyl)phenylsulfonyl)propanoate (234)

Methyl 2-methyl-2-(3-(trifluoromethyl)phenylthio)propanoate (233) (32.77 g, 118 mmol), and oxone (217.4 g, 354 mmol) were stirred in MeOH/H₂O (600 ml, 2:1) at room temperature for 16 hours. The reaction was filtered and the filtrate concentrated in vacuo. The residue was taken up in EtOAc, washed repeatedly with H₂O, dried, and concentrated in vacuo to give methyl 2-methyl-2-(3-(trifluoromethyl)phenylsulfonyl)propanoate (234) (32.19 g, 88%). ¹H NMR (300 mHz —CD₃Cl) δ 1.64 (s, 6H), 3.70 (s, 3H), 7.72 (t, 1H, J=7.86 Hz), 7.95 (d, 1H, J=7.83 Hz), 8.06 (d, 1H, J=7.98 Hz), 8.11 (s, 1H). The product was used with further purification.

### C. Preparation of 2-methyl-2-(3-(trifluoromethyl)phenylsulfonyl)propan-1-amine (238)

### 2-Methyl-2-(3-(trifluoromethyl)phenylsulfonyl)propan-1-amine (238) was prepared in an analogous fashion to 3-methyl-3-(3-(trifluoromethyl)phenylsulfonyl)butan-1-amine (228) using methyl 2-methyl-2-(3-(trifluoromethyl)phenylsulfonyl)propanoate (234).

### Example 48

### General Synthetic Protocols

### A. General Coupling Protocol for the Synthesis of Compounds with General Structure (239)

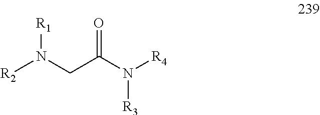

### Exemplified by the Synthesis of N-cyclohexyl-2-(4-methyl-4-(3-(trifluoromethyl)phenyl sulfonyl)piperidin-1-yl)acetamide (240)

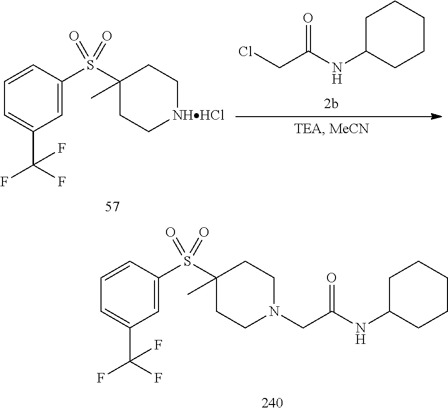

### 4-Methyl-4-(3-(trifluoromethyl)phenylsulfonyl)piperidine hydrochloride (57) (81 mg, 0.26 mmol), TEA (109 μL, 0.78 mmol) and 2-chloro-N-cyclohexylacetamide (2b) (46 mg, 0.26 mmol) were stirred in MeCN (2 mL) at room temperature for 16 hours. The reaction was concentrated in vacuo and purified by reverse phase HPLC to give N-cyclohexyl-2-(4-methyl-4-(3-(trifluoromethyl)phenylsulfonyl)piperidin-1-yl)acetamide (240).

### B. General Coupling Protocol for the Synthesis of Compounds with General

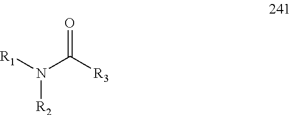

### Exemplified by the Synthesis of 3-fluoro-5-methoxy-N-(4-(3-(trifluoromethyl)phenyl sulfonyl)cyclohexyl)benzamide (243)

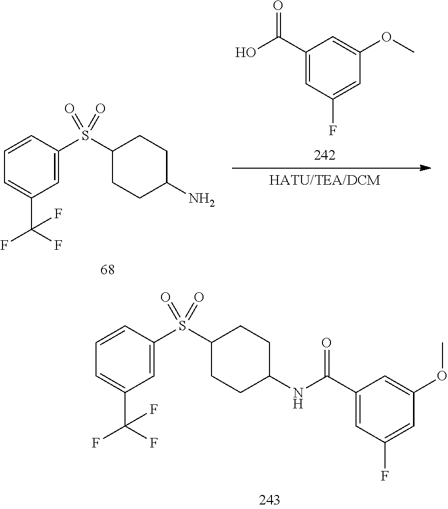

4-(3-(Trifluoromethyl)phenylsulfonyl)cyclohexanamine (68) (100 mg, 0.32 mmol), HATU (167 mg, 0.44 mmol), TEA (167 μL, 1.2 mmol), and 3-fluoro-5-methoxy benzoic acid (242) (54 mg, 0.32 mmol) were stirred in DCM (2 mL) at room temperature for 16 hours. The reaction was concentrated in vacuo and the residue purified by reverse phase HPLC to give 3-fluoro-5-methoxy-N-(4-(3-(trifluoromethyl)phenylsulfonyl)cyclohexyl)benzamide (243).

Stoichiometries given above are to be considered exemplary but may be varied. Suitable organic bases may be used as alternates to TEA (e.g. DIPEA). For HCl salts, at least one additional equivalent of base to that described must be employed. DMF may be substituted for DCM as solvent.

### C. General Coupling Protocol for the Synthesis of Compounds with General Structure (244)

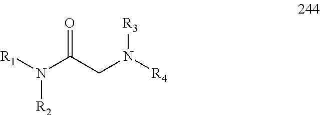

### Exemplified by the Synthesis of 2-(cyclohexylamino)-1-(4-(3-(trifluoromethyl)phenyl sulfonyl)piperidin-1-yl)ethanone (246)

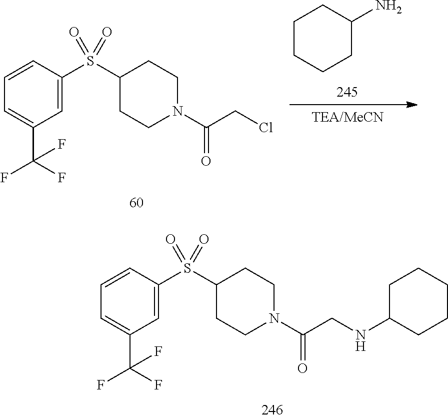

2-chloro-1-(4-(3-(trifluoromethyl)phenylsulfonyl)piperidin-1-yl)ethanone (60) (70 mg, 0.2 mmol), TEA (83 μL, 0.6 mmol) and cyclohexylamine (245) (23 μL, 0.2 mmol) were stirred in MeCN (2 mL) at room temperature for 16 hours. The reaction was concentrated in vacuo, and the residue purified by reverse phase HPLC to give 2-(cyclohexylamino)-1-(4-(3-(trifluoromethyl)phenylsulfonyl)piperidin-1-yl)ethanone (246).

Stoichiometries given above are to be considered exemplary but may be varied. Suitable organic bases may be used as alternates to TEA (e.g. DIPEA). For HCl salts, at least one additional equivalent of base to that described must be employed.

### D. General Coupling Protocol for the Synthesis of Compounds with General Structure (247)

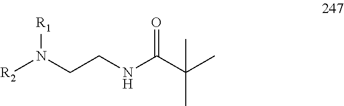

### Exemplified by the Synthesis of N-(2-(4-(3-(trifluoromethyl)phenylsulfonyl)piperidin-1-yl)ethyl)pivalamide (248)

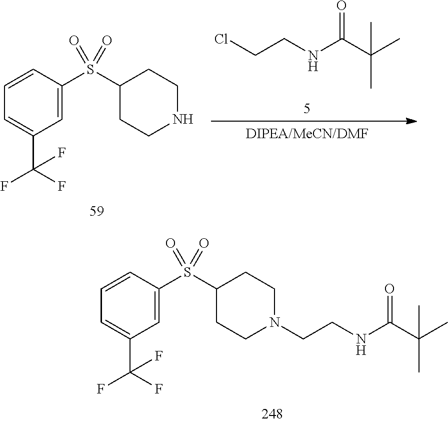

N-(2-chloroethyl)pivalamide (5) (30 mg, 0.19 mmol), DIPEA (107 μL, 0.62 mmol), and 4-(3-(trifluoromethyl)phenylsulfonyl)piperidine (59) (38 mg, 0.12 mmol) were heated in DMF/CH₃CN (1/1, 2 mL) in a sealed tube for at 100° C. for 168 hours. The reaction was diluted with EtOAc (4 mL), washed with saturated aqueous NaHCO₃, and concentrated in vacuo. The crude material was purified by reverse phase HPLC to give N-(2-(4-(3-(trifluoromethyl)phenylsulfonyl)piperidin-1-yl)ethyl)pivalamide (248).

### E. General Coupling Protocol for the Synthesis of Compounds with General Structure (249)

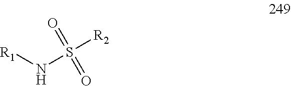

### Exemplified by the Synthesis of N-((1s,4s)-4-fluoro-4-(3-(trifluoromethyl)phenyl sulfonyl)cyclohexyl)-1,3,5-trimethyl-1H-pyrazole-4-sulfonamide (251)

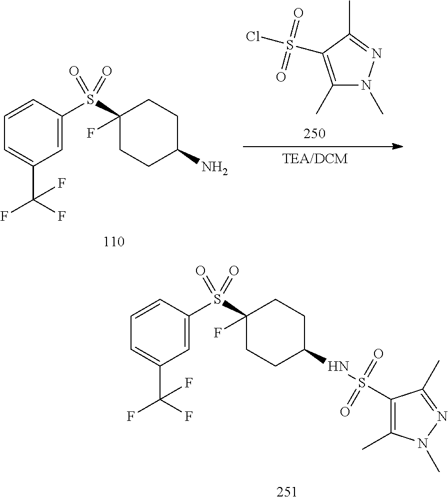

Cis-4-fluoro-4-(3-(trifluoromethyl)phenylsulfonyl)cyclohexanamine (110) (75 mg, 0.21 mmol), TEA (111 μL, 0.8 mmol), and 1,3,5-trimethyl-1H-pyrazole-4-sulfonyl chloride (250) (0.21 mmol) were stirred at room temperature for 16 hours. The reaction was concentrated in vacuo and the crude material purified by reverse phase HPLC to give N—(Cis-4-fluoro-4-(3-(trifluoromethyl)phenylsulfonyl)cyclohexyl-1,3,5-trimethyl-1H-pyrazole-4-sulfonamide (251).

### F. General Coupling Protocol for the Synthesis of Compounds with General Structure (252)

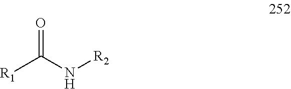

### Exemplified by the Synthesis of trans-N-(4-(3-chloro-4-fluorophenoxy)phenyl)-3-(3-(trifluoromethyl)phenylsulfonyl)cyclobutanecarboxamide (253)

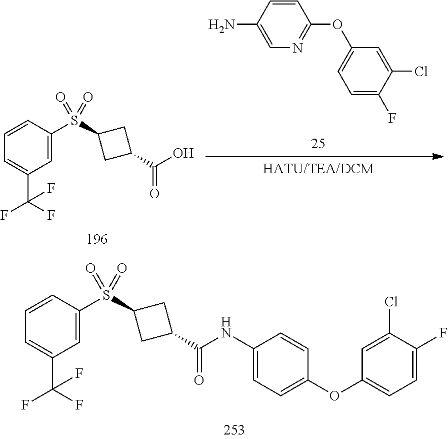

Cis-3-(3-(trifluoromethyl)phenylsulfonyl)cyclobutanecarboxylic acid (196) (60 mg, 0.19 mmol), HATU (100 mg, 0.27 mmol), TEA (111 μL, 0.8 mmol), and 6-(3-chloro-4-fluorophenoxy)pyridin-3-amine (25) (0.19 mmol) were stirred in DCM at room temperature for 16 hours. The reaction was concentrated in vacuo and the crude material purified by reverse phase HPLC to give trans-N-(4-(3-chloro-4-fluorophenoxy)phenyl)-3-(3-(trifluoromethyl)phenylsulfonyl)cyclobutane-carboxamide (253).

### G. General Coupling Protocol for the Synthesis of Amides Containing an Alkyl Amine Exemplified by the Synthesis of 1-amino-N-(cis)-4-fluoro-4-(3-(trifluoromethyl)phenylsulfonyl)cyclohexyl)cyclohexanecarboxamide (254)

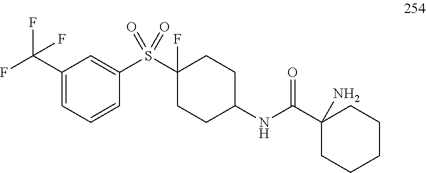

The compounds were prepared using a HATU coupling protocol with Boc protected-aminocarboxylic acids and DCM as solvent. The reaction solution was washed with saturated NaHCO₃, dried (Na₂SO₄) and concentrated in vacuo. The product was treated with 2 M HCl in Et₂O, concentrated in vacuo and the crude material purified by reverse phase HPLC.

### H. General Cyclization Protocol for the Synthesis of Compounds with General

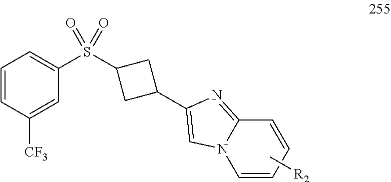

### Exemplified by the Synthesis of 6-(trifluoromethyl)-3-(3-(3-(trifluoromethyl)phenylsulfonyl)cyclobutyl)imidazo[1,2-a]pyridine (257)

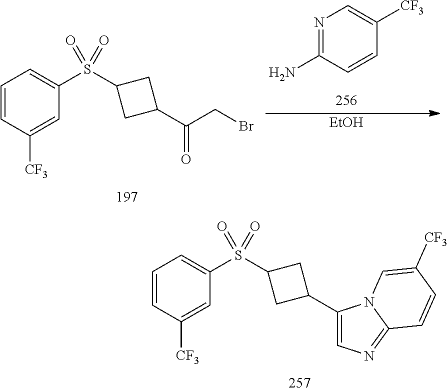

2-Bromo-1-(3-(3-(trifluoromethyl)phenylsulfonyl)cyclobutyl)ethanone (197) (133 mg, 0.345 mmol) and 5-(trifluoromethyl)pyridin-2-amine (256) (56 mg, 0.35 mmol) were heated in a sealed vessel in EtOH (2 mL) at 125° C. for 50 minutes in a microwave reactor. The reaction was concentrated in vacuo and purified by reverse phase HPLC to give 6-(trifluoromethyl)-3-(3-(3-(trifluoromethyl)phenylsulfonyl)cyclobutyl)imidazo[1,2-a]pyridine (257).

### I. General Cyclization Protocol for the Synthesis of Compounds with General Structure (258)

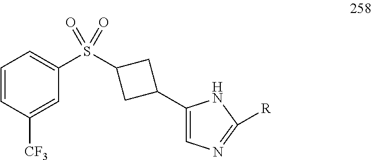

### Exemplified by the Synthesis of 2-(trifluoromethyl)-5-(3-(3-(trifluoromethyl)phenylsulfonyl)cyclobutyl)-1H-imidazole (260)

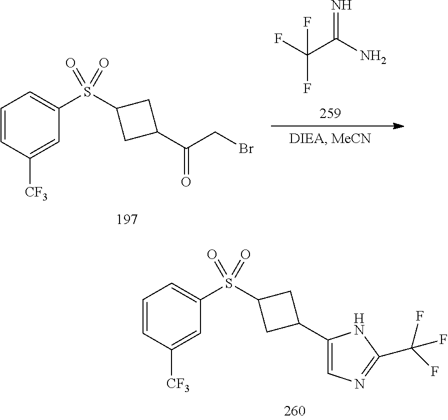

2-Bromo-1-(3-(3-(trifluoromethyl)phenylsulfonyl)cyclobutyl)ethanone (197) (100 mg, 0.260 mmol), DIEA (226 μL, 1.30 mmol), and 2,2,2-trifluoroacetimidamide (259) (58 mg, 0.52 mmol) were stirred in CH₃CN (3 mL) at room temperature for 4 days. The reaction was concentrated in vacuo and purified by reverse phase HPLC to give 2-(trifluoromethyl)-5-(3-(3-(trifluoromethyl)phenylsulfonyl)cyclobutyl)-1H-imidazole (260).

### J. General Reductive Amination Protocol for the Synthesis of Compounds with the General Structure (262)

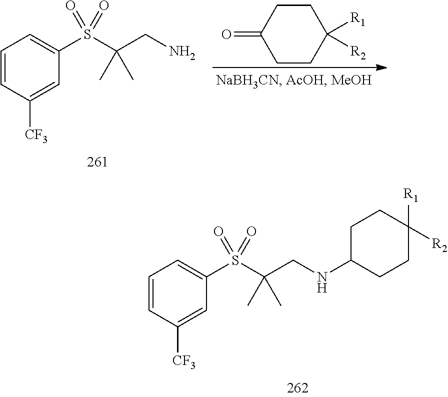

2-Methyl-2-(3-(trifluoromethyl)phenylsulfonyl)propan-1-amine (1 eq), 4-substituted piperidinone (4 eq), NaBH₃CN (3 eq) and HOAc (cat) were heated in MeOH at 60° C. for 16 hours. The reaction was concentrated in vacuo and the residue purified by reverse phase HPLC.

### K. General Synthetic Protocol for Compounds with General Structure (263)

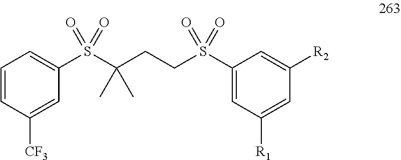

### Exemplified by the Synthesis of 1-(3-methyl-3-(3-(trifluoromethyl)phenylsulfonyl)butyl-sulfonyl)-3-(trifluoromethoxy)benzene (266)

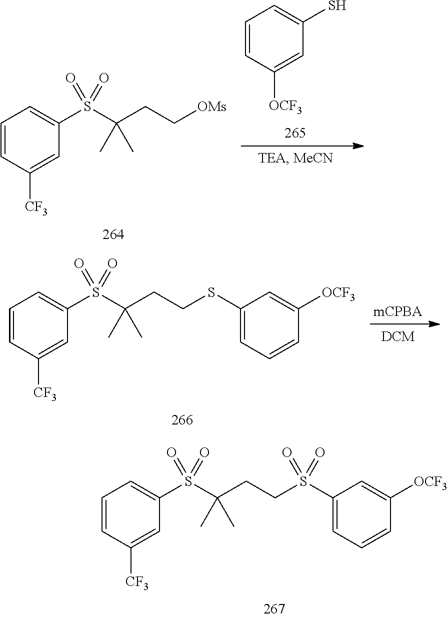

### K1. Preparation of (3-methyl-3-(3-(trifluoromethyl)phenylsulfonyl)butyl)(3-(trifluoromethoxy)-phenyl)sulfane (266)

3-Methyl-3-(3-(trifluoromethyl)phenylsulfonyl)butyl methanesulfonate (264) (250 mg, 0.75 mmol), 3-trifluoromethoxythiophenol (142 mg, 0.75 mmol) and TEA (152 μL, 1.5 mmol) were heated at reflux in MeCN for 16 hours. The reaction was concentrated in vacuo and the residue purified by automated column chromatography (5% EtOAc/PE) to give (3-methyl-3-(3-(trifluoromethyl)phenylsulfonyl)butyl)(3-(trifluoromethoxy)-phenyl)sulfane (280 mg, 79%). The product was confirmed by LCMS.

### K2. Preparation of 1-(3-methyl-3-(3-(trifluoromethyl)phenylsulfonyl)butyl-sulfonyl)-3-(trifluoro-methoxy)benzene (267)

(3-Methyl-3-(3-(trifluoromethyl)phenylsulfonyl)butyl)(3-(trifluoromethoxy)-phenyl)sulfane (266) (200 mg, 0.42 mmol) and mCPBA (213 mg, 1.2 mmol) were stirred in DCM at room temperature for 16 hours. The organics were washed with 2 M NaOH, separated, dried and concentrated in vacuo to give 1-(3-methyl-3-(3-(trifluoromethyl)phenylsulfonyl)butyl-sulfonyl)-3-(trifluoro-methoxy)benzene (267) (116 mg, 55%). ¹H NMR (300 mHz —CD₃Cl) δ 1.24 (s, 6 H), 2.08 (m, 2H), 3.36 (m, 2H), 7.49 (d, 1H, J=8.22 Hz), 7.64 (m, 3H), 7.75 (d, 1 H, J=7.87 Hz), 7.86 (m, 2H), 7.93 (s, 1H)

### Example 49

### Synthesis of Exemplary Compounds and Mass Spectrometry

Following the general procedures set forth in Examples 1-48, the following compounds listed in Table 1 below were prepared. Mass spectrometry was employed with the final compound and at various stages throughout the synthesis as a confirmation of the identity of the product obtained (M+1). For the mass spectrometric analysis, samples were prepared at an approximate concentration of 1 μg/mL in methanol:water (50:50 v/v) with 0.1% formic acid. Samples were then analyzed by a Waters 3100 Applied Biosystems API3000 single quadrupole mass spectrometer and scanned in the range of 250 to 700 m/z.

TABLE 1

| | | | Mass |
|---|---|---|---|
| | | | Spec. |
| No. | Structure | Name | (m/z) |
| 1 | 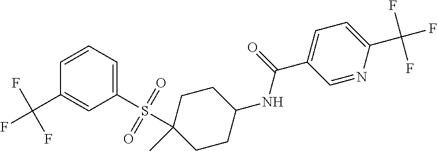 | N-(4-methyl-4-(3- (trifluoromethyl)phenylsulfonyl) cyclohexyl)-6-(trifluoromethyl) nicotinamide | 495.27 |
| 2 | 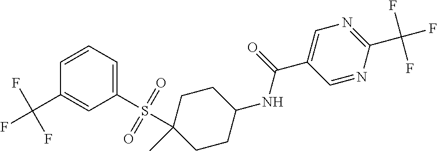 | N-(4-methyl-4-(3- (trifluoromethyl)phenylsulfonyl) cyclohexyl)-2-(trifluoromethyl) pyrimidine-5-carboxamide | 496.26 |
| 3 | 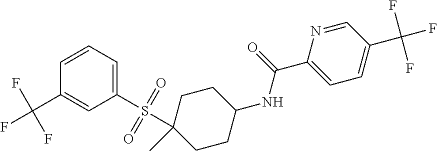 | N-(4-methyl-4-(3- (trifluoromethyl)phenylsulfonyl) cyclohexyl)-5-(trifluoromethyl) picolinamide | 495.27 |
| 4 | 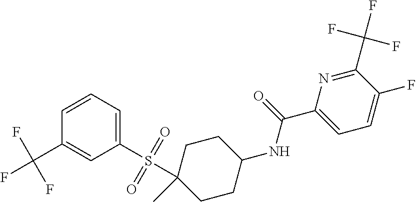 | 4-fluoro-N-(4-methyl-4-(3- (trifluoromethyl)phenylsulfonyl) cyclohexyl)-3-(trifluoromethyl) benzamide | 512.27 |
| 5 | 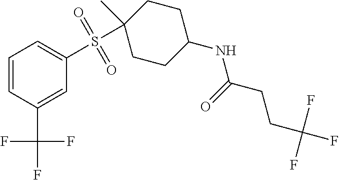 | 4,4,4-trifluoro-N-(4-methyl-4-(3- (trifluoromethyl)phenylsulfonyl) cyclohexyl)butanamide | 446.28 |
| 6 | 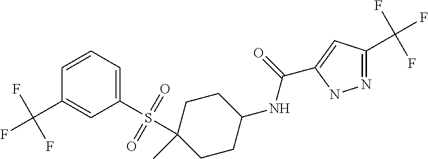 | 1-methyl-N-(4-methyl-4-(3- (trifluoromethyl)phenylsulfonyl) cyclohexyl)-3-(trifluoromethyl)-1H- pyrazole-5-carboxamide | 498.29 |
| 7 | 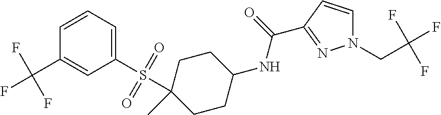 | N-(4-methyl-4-(3- (trifluoromethyl)phenylsulfonyl) cyclohexyl)-l-(2,2,2-trifluoroethyl)- 1H-pyrazole-3-carboxamide | 498.29 |
| 8 | 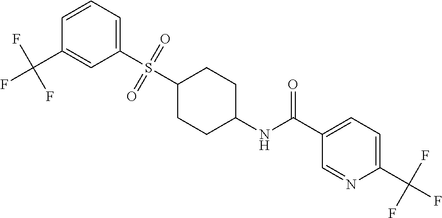 | 6-(trifluoromethyl)-N-(4-(3- (trifluoromethyl)phenylsulfonyl) cyclohexyl)nicotinamide | 481.25 |
| 9 | 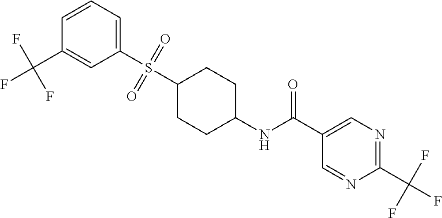 | 2-(trifluoromethyl)-N-(4-(3- (trifluoromethyl)phenylsulfonyl) cyclohexyl)pyrimidine-5- carboxamide | 482.25 |
| 10 | 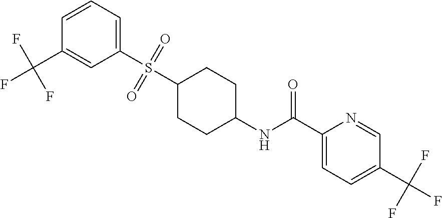 | 5-(trifluoromethyl)-N-(4-(3- (trifluoromethyl)phenylsulfonyl) cyclohexyl)picolinamide | 481.26 |
| 11 | 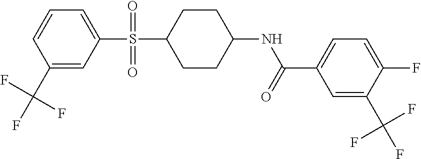 | 4-fluoro-3-(trifluoromethyl)-N-(4- (3-(trifluoromethyl)phenylsulfonyl) cyclohexyl)benzamide | 435.33 |
| 12 | 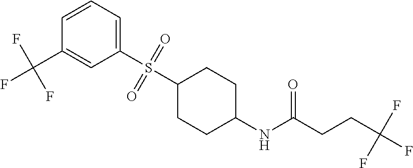 | 4,4,4-trifluoro-N-(4-(3- (trifluoromethyl)phenylsulfonyl) cyclohexyl)butanamide | 498.22 |
| 13 | 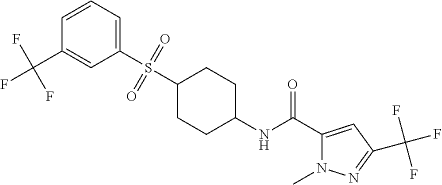 | 1-methyl-3-(trifluoromethyl)-N-(4- (3-(trifluoromethyl)phenylsulfonyl) cyclohexyl)-1H-pyrazole-5- carboxamide | 432.23 |
| 14 | 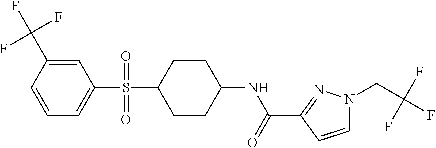 | 1-(2,2,2-trifluoroethyl)-N-(4-(3- (trifluoromethyl)phenylsulfonyl) cyclohexyl)-1H-pyrazole-3- carboxamide | 484.24 |
| 15 | 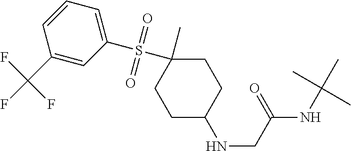 | N-tert-butyl-2-(4-methyl-4-(3- (trifluoromethyl)phenylsulfonyl) cyclohexylamino)acetamide | 484.24 |
| 16 | 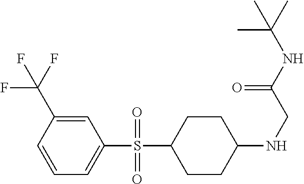 | N-tert-butyl-2-(4-(3- (trifluoromethyl)phenylsulfonyl) cyclohexylamino)acetamide | 421.1 |
| 17 | 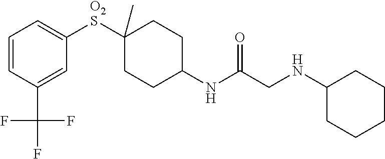 | 2-(cyclohexylamino)-N-(4-methyl- 4-(3- (trifluoromethyl)phenylsulfonyl) cyclohexyl)acetamide | 461.37 |
| 18 | 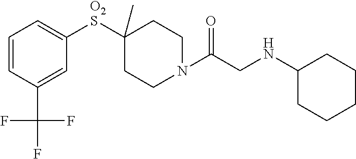 | 2-(cyclohexylamino)-l-(4-methyl- 4-(3- (trifluoromethyl)phenylsulfonyl) piperidin-l-yl)ethanone | 447.36 |
| 19 | 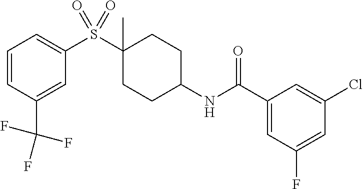 | 3-chloro-5-fluoro-N-(4-methyl-4- (3-(trifluoromethyl)phenylsulfonyl) cyclohexyl)benzamide | 478.21 |
| 20 | 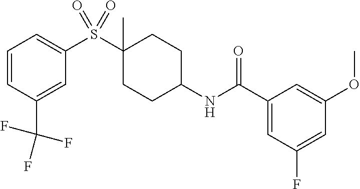 | 3-fluoro-5-methoxy-N-(4-methyl-4- (3-(trifluoromethyl)phenylsulfonyl) cyclohexyl)benzamide | 474.27 |
| 21 | 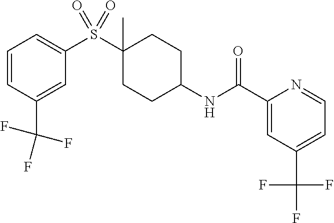 | N-(4-methyl-4-(3- (trifluoromethyl)phenylsulfonyl) cyclohexyl)-4- (trifluoromethyl)picolinamide | 495.24 |
| 22 | 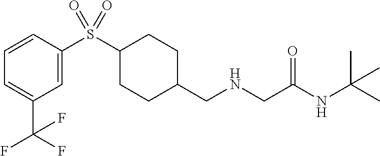 | N-tert-butyl-2-((4-(3- (trifluoromethyl)phenylsulfonyl) cyclohexyl)methylamino)acetamide | 435.3 |
| 23 | 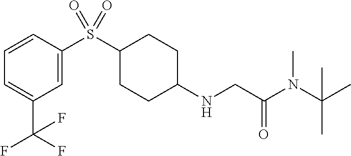 | N-tert-butyl-N-methyl-2-(4-(3- (trifluoromethyl)phenylsulfonyl) cyclohexylamino)acetamide | 435.3 |
| 24 | 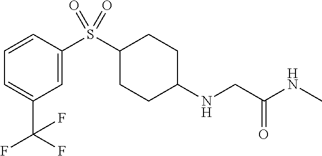 | N-methyl-2-(4-(3- (trifluoromethyl)phenylsulfonyl) cyclohexylamino)acetamide | 379.24 |
| 25 | 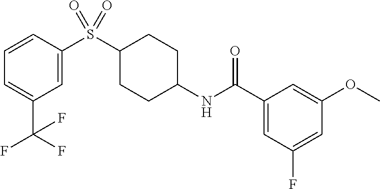 | 3-fluoro-5-methoxy-N-(4-(3- (trifluoromethyl)phenylsulfonyl) cyclohexyl)benzamide | 460.21 |
| 26 | 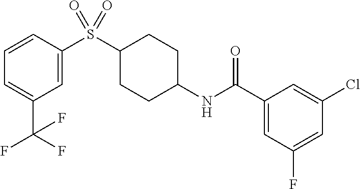 | 3-chloro-5-fluoro-N-(4-(3- (trifluoromethyl)phenylsulfonyl) cyclohexyl)benzamide | 464.15 |
| 27 | 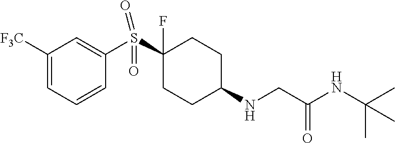 | N-tert-butyl-2-((1S,4S)-4-fluoro-4- (3-(trifluoromethyl)phenylsulfonyl) cyclohexylamino)acetamide | 439.26 |
| 28 | 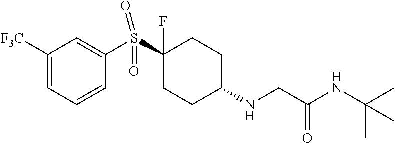 | N-tert-butyl-2-((1R,4R)-4-fluoro-4- (3-(trifluoromethyl)phenylsulfonyl) cyclohexylamino)acetamide | 439.26 |
| 29 | 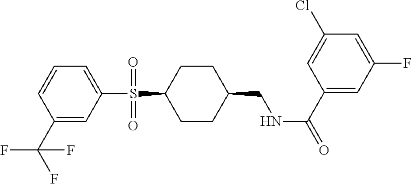 | 3-chloro-5-fluoro-N-(((1S,4S)-4-(3- (trifluoromethyl)phenylsulfonyl) cyclohexyl)methyl)benzamide | 478.17 |
| 30 | 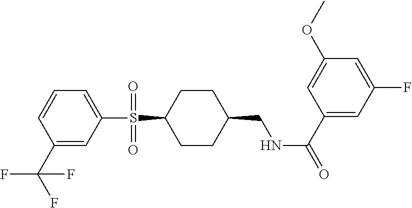 | 3-fluoro-5-methoxy-N-(((1S,4S)-4- (3-(trifluoromethyl)phenylsulfonyl) cyclohexyl)methyl)benzamide | 474.24 |
| 31 | 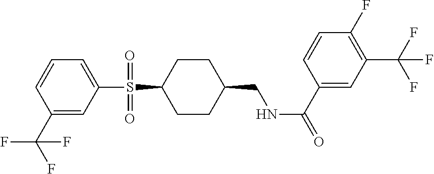 | 4-fluoro-3-(trifluoromethyl)-N- (((1S,4S)-4-(3- (trifluoromethyl)phenylsulfonyl) cyclohexyl)methyl)benzamide | 512.23 |
| 32 | 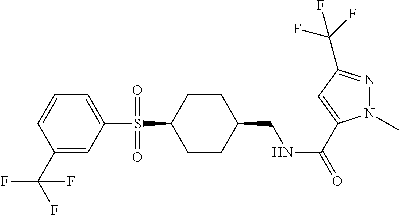 | 1-methyl-3-(trifluoromethyl)-N- (((1S,4S)-4-(3- (trifluoromethyl)phenylsulfonyl) cyclohexyl)methyl)-1H-pyrazole-5- carboxamide | 498.23 |
| 33 | 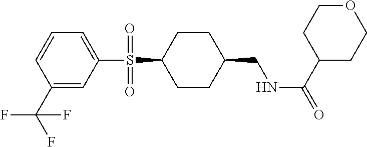 | N-(((1S,4S)-4-(3- (trifluoromethyl)phenylsulfonyl) cyclohexyl)methyl)tetrahydro-2H- pyran-4-carboxamide | 434.25 |
| 34 | 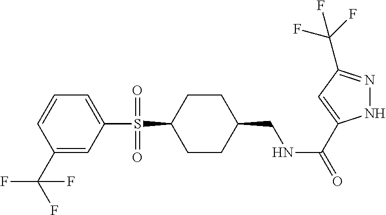 | 3-(trifluoromethyl)-N-(((1S,4S)-4- (3-(trifluoromethyl)phenylsulfonyl) cyclohexyl)methyl)-1H-pyrazole-5- carboxamide | 484.22 |
| 35 | 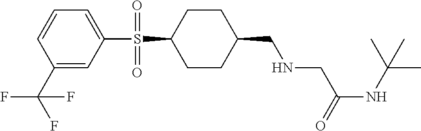 | N-tert-butyl-2-(((1S,4S)-4-(3- (trifluoromethyl)phenylsulfonyl) cyclohexyl)methylamino)acetamide | 435.29 |
| 36 | 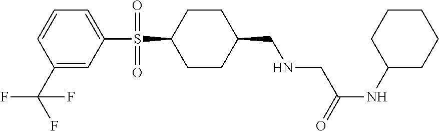 | N-cyclohexyl-2-(((1S,4S)-4-(3- (trifluoromethyl)phenylsulfonyl) cyclohexyl)methylamino)acetamide | 461.3 |
| 37 | 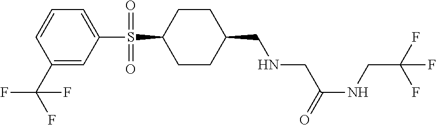 | N-(2,2,2-trifluoroethyl)-2-(((1S,4S)- 4-(3- (trifluoromethyl)phenylsulfonyl) cyclohexyl)methylamino)acetamide | 461.24 |
| 38 | 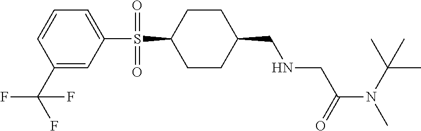 | N-tert-butyl-N-methyl-2-(((1S,4S)- 4-(3- (trifluoromethyl)phenylsulfonyl) cyclohexyl)methylamino)acetamide | 449.31 |
| 39 | 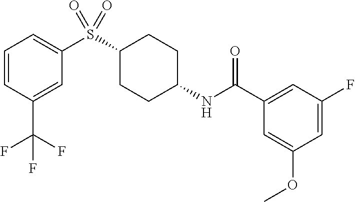 | 3-fluoro-5-methoxy-N-((1S,4S)-4- (3-(trifluoromethyl)phenylsulfonyl) cyclohexyl)benzamide | 460.21 |
| 40 | 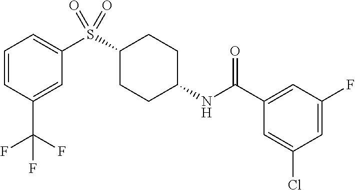 | 3-chloro-5-fluoro-N-((1S,4S)-4- (3-(trifluoromethyl)phenylsulfonyl) cyclohexyl)benzamide | 464.15 |
| 41 | 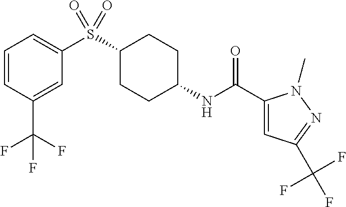 | 1-methyl-3-(trifluoromethyl)-N- ((1S,4S)-4-(3- (trifluoromethyl)phenylsulfonyl) cyclohexyl)-1H-pyrazole-5- carboxamide | 484.21 |
| 42 | 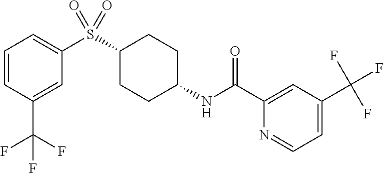 | 4-(trifluoromethyl)-N-((1S,4S)-4- (3-(trifluoromethyl)phenylsulfonyl) cyclohexyl)picolinamide | 481.19 |
| 43 | 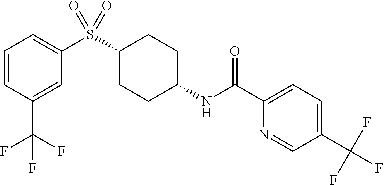 | 5-(trifluoromethyl)-N-((1S,4S)-4- (3-(trifluoromethyl)phenylsulfonyl) cyclohexyl)picolinamide | 481.19 |
| 44 | 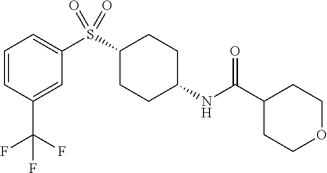 | N-((1S,4S)-4-(3- (trifluoromethyl)phenylsulfonyl) cyclohexyl)tetrahydro-2H-pyran-4- carboxamide | 420.24 |
| 45 | 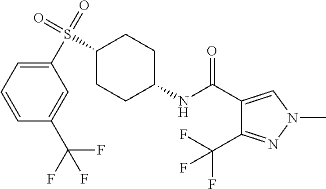 | 1-methyl-3-(trifluoromethyl)-N- ((1S,4S)-4-(3- (trifluoromethyl)phenylsulfonyl) cyclohexyl)-1H-pyrazole-4- carboxamide | 484.22 |
| 46 | 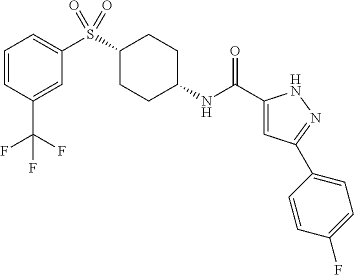 | 3-(4-fluorophenyl)-N-((1S,4S)-4-(3- (trifluoromethyl)phenylsulfonyl) cyclohexyl)-1H-pyrazole-5- carboxamide | 496.24 |
| 47 | 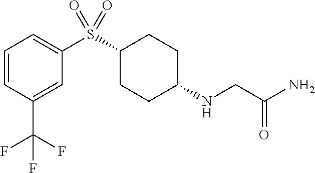 | 2-((1S,4S)-4-(3- (trifluoromethyl)phenylsulfonyl) cyclohexylamino)acetamide | 365.21 |
| 48 | 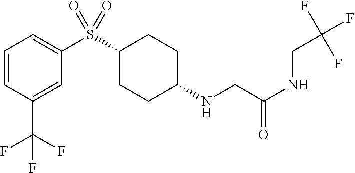 | N-(2,2,2-trifluoroethyl)-2-((1S,4S)- 4-(3- (trifluoromethyl)phenylsulfonyl) cyclohexylamino)acetamide | 447.22 |
| 49 | 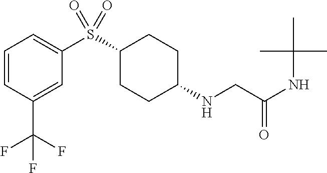 | N-tert-butyl-2-((1S,4S)-4-(3- (trifluoromethyl)phenylsulfonyl) cyclohexylamino)acetamide | 421.28 |
| 50 | 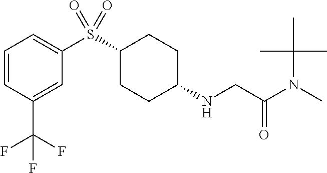 | N-tert-butyl-N-methyl-2-((1S,4S)- 4-(3- (trifluoromethyl)phenylsulfonyl) cyclohexylamino)acetamide | 435.29 |
| 51 | 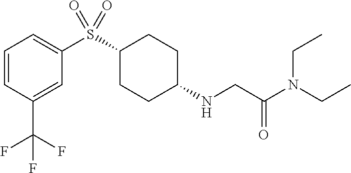 | N,N-diethyl-2-((1S,4S)-4-(3- (trifluoromethyl)phenylsulfonyl) cyclohexylamino)acetamide | 421.27 |
| 52 | 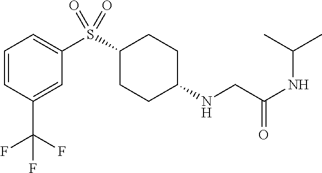 | N-isopropyl-2-((1S,4S)-4-(3- (trifluoromethyl)phenylsulfonyl) cyclohexylamino)acetamide | 407.26 |
| 53 | 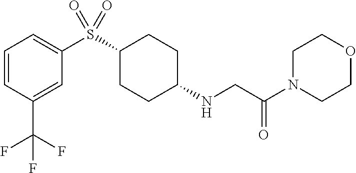 | 1-morpholino-2-((1S,4S)-4-(3- (trifluoromethyl)phenylsulfonyl) cyclohexylamino)ethanone | 435.24 |
| 54 | 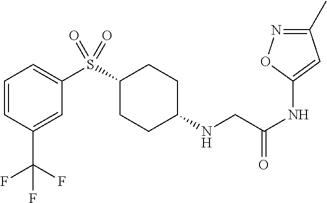 | N-(3-methylisoxazol-5-yl)-2- ((1S,4S)-4-(3- (trifluoromethyl)phenylsulfonyl) cyclohexylamino)acetamide | 446.21 |
| 55 | 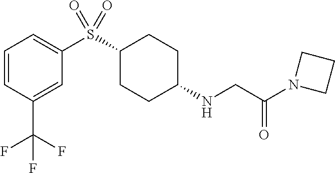 | 1-(azetidin-1-yl)-2-((1S,4S)-4-(3- (trifluoromethyl)phenylsulfonyl) cyclohexylamino)ethanone) | 405.25 |
| 56 | 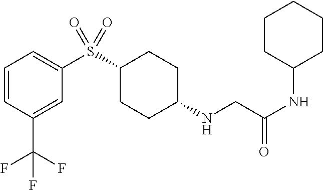 | N-cyclohexyl-2-((1S,4S)-4-(3- (trifluoromethyl)phenylsulfonyl) cyclohexylamino)acetamide | 447.29 |
| 57 | 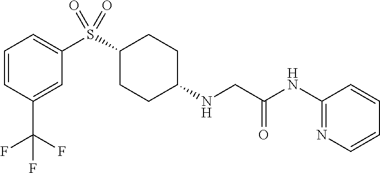 | N-(pyridin-2-yl)-2-((1S,4S)-4-(3- (trifluoromethyl)phenylsulfonyl) cyclohexylamino)acetamide | 442.23 |
| 58 | 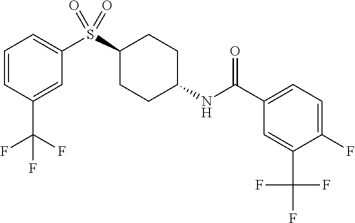 | 4-fluoro-3-(trifluoromethyl)-N- ((1R,4R)-4-(3- (trifluoromethyl)phenylsulfonyl) cyclohexyl)benzamide | 498.21 |
| 59 | 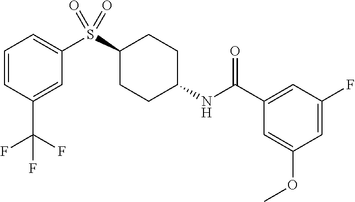 | 3-fluoro-5-methoxy-N-((1R,4R)-4- (3-(trifluoromethyl)phenylsulfonyl) cyclohexyl)benzamide | 460.22 |
| 60 | 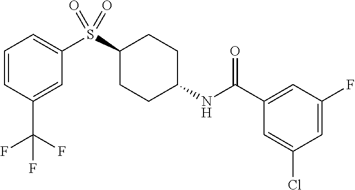 | 3-chloro-5-fluoro-N-((1R,4R)-4-(3- (trifluoromethyl)phenylsulfonyl) cyclohexyl)benzamide | 464.16 |
| 61 | 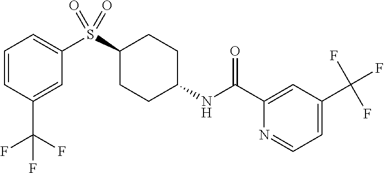 | 4-(trifluoromethyl)-N-((1R,4R)-4- (3-(trifluoromethyl)phenylsulfonyl) cyclohexyl)picolinamide | 481.19 |
| 62 | 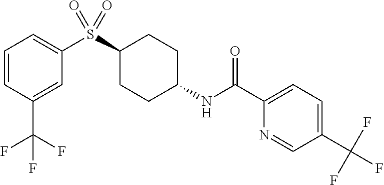 | 5-(trifluoromethyl)-N-((1R,4R)-4- (3-(trifluoromethyl)phenylsulfonyl) cyclohexyl)picolinamide | 481.19 |
| 63 | 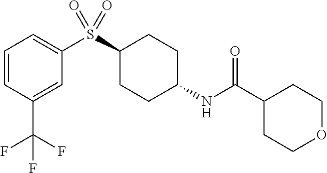 | N-((1R,4R)-4-(3- (trifluoromethyl)phenylsulfonyl) cyclohexyl)tetrahydro-2H-pyran-4- carboxamide | 420.24 |
| 64 | 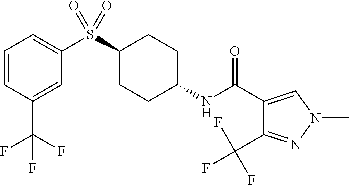 | 1-methyl-3-(trifluoromethyl)-N- ((1R,4R)-4-(3- (trifluoromethyl)phenylsulfonyl) cyclohexyl)-1H-pyrazole-4- carboxamide | 484.22 |
| 65 | 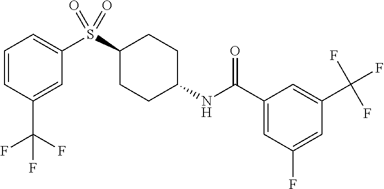 | 3-fluoro-5-(trifluoromethyl)-N- ((1R,4R)-4-(3- (trifluoromethyl)phenylsulfonyl) cyclohexyl)benzamide | 498.2 |
| 66 | 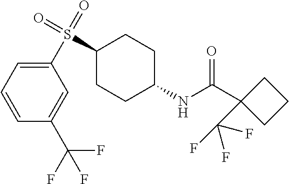 | 1-(trifluoromethyl)-N-((1R,4R)-4- (3-(trifluoromethyl)phenylsulfonyl) cyclohexyl)cyclobutanecarboxamide | 458.21 |
| 67 | 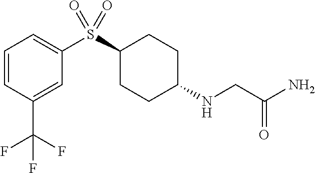 | 2-((1R,4R)-4-(3- (trifluoromethyl)phenylsulfonyl) cyclohexylamino)acetamide | 365.21 |
| 68 | 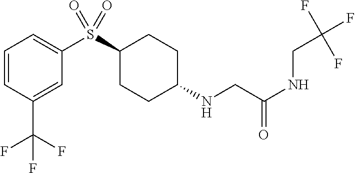 | N-(2,2,2-trifluoroethyl)-2-((1R,4R)- 4-(3- (trifluoromethyl)phenylsulfonyl) cyclohexylamino)acetamide | 447.2 |
| 69 | 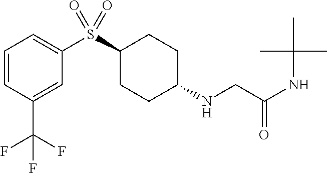 | N-tert-butyl-2-((1R,4R)-4-(3- (trifluoromethyl)phenylsulfonyl) cyclohexylamino)acetamide | 421.25 |
| 70 | 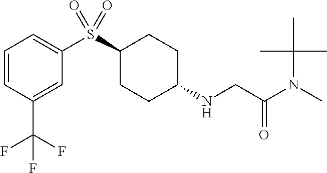 | N-tert-butyl-N-methyl-2-((1R,4R)- 4-(3- (trifluoromethyl)phenylsulfonyl) cyclohexylamino)acetamide | 435.28 |
| 71 | 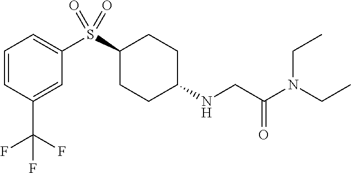 | N,N-diethyl-2-((1R,4R)-4-(3- (trifluoromethyl)phenylsulfonyl) cyclohexylamino)acetamide | 421.25 |
| 72 | 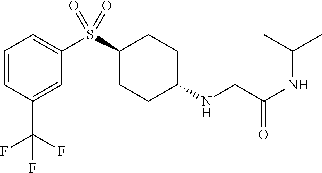 | N-isopropyl-2-((1R,4R)-4-(3- (trifluoromethyl)phenylsulfonyl) cyclohexylamino)acetamide | 407.25 |
| 73 | 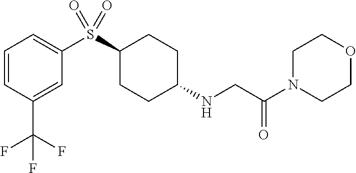 | 1-morpholino-2-((1R,4R)-4-(3- (trifluoromethyl)phenylsulfonyl) cyclohexylamino)ethanone | 435.23 |
| 74 | 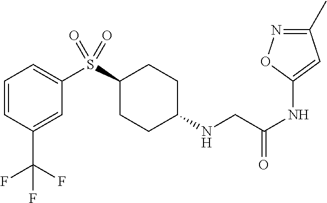 | N-(3-methylisoxazol-5-yl)-2- ((1R,4R)-4-(3- (trifluoromethyl)phenylsulfonyl) cyclohexylamino)acetamide | 446.21 |
| 75 | 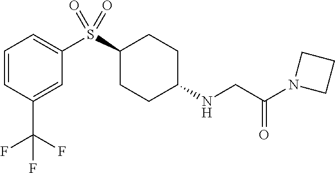 | 1-(azetidin-1-yl)-2-((1R,4R)-4-(3- (trifluoromethyl)phenylsulfonyl) cyclohexylamino)ethanone | 405.24 |
| 76 | 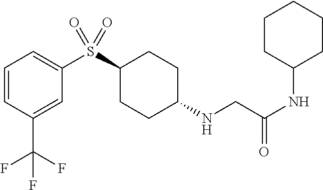 | N-cyclohexyl-2-((1R,4R)-4-(3- (trifluoromethyl)phenylsulfonyl) cyclohexylamino)acetamide | 447.27 |
| 77 | 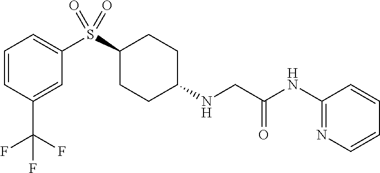 | N-(pyridin-2-yl)-2-((1R,4R)-4-(3- (trifluoromethyl)phenylsulfonyl) cyclohexylamino)acetamide | 442.21 |
| 78 | 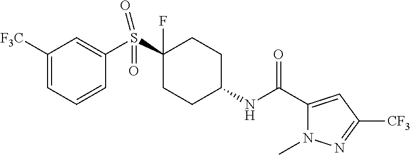 | N-((1R,4R)-4-fluoro-4-(3- (trifluoromethyl)phenylsulfonyl) cyclohexyl)-1-methyl-3- (trifluoromethyl)-1H-pyrazole-5- carboxamide | 502.2 |
| 79 | 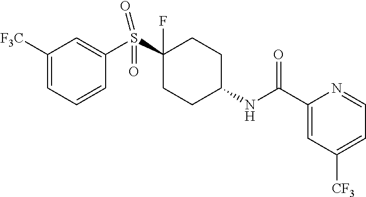 | N-((1R,4R)-4-fluoro-4-(3- (trifluoromethyl)phenylsulfonyl) cyclohexyl)-4- (trifluoromethyl)picolinamide | 499.17 |
| 80 | 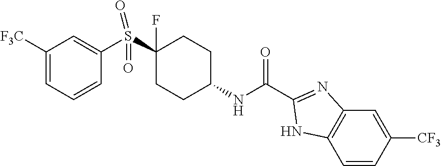 | N-((1R,4R)-4-fluoro-4-(3- (trifluoromethyl)phenylsulfonyl) cyclohexyl)-5-(trifluoromethyl)-1H- benzo[d]imidazole-2-carboxamide | 538.22 |
| 81 | 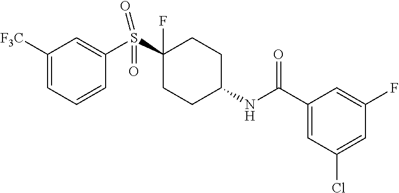 | 3-chloro-5-fluoro-N-((1R,4R)-4- fluoro-4-(3- (trifluoromethyl)phenylsulfonyl) cyclohexyl)benzamide | 482.14 |
| 82 | 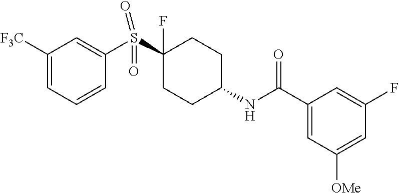 | 3-fluoro-N-((1R,4R)-4-fluoro-4-(3- (trifluoromethyl)phenylsulfonyl) cyclohexyl)-5-methoxybenzamide | 478.19 |
| 83 | 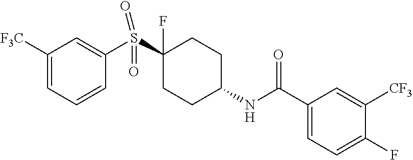 | 4-fluoro-N-((1R,4R)-4-fluoro-4-(3- (trifluoromethyl)phenylsulfonyl) cyclohexyl)-3- (trifluoromethyl)benzamide | 516.18 |
| 84 | 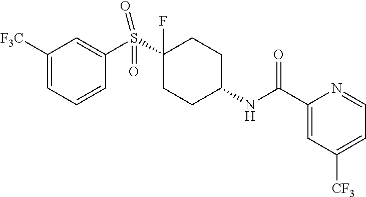 | N-((1S,4S)-4-fluoro-4-(3- (trifluoromethyl)phenylsulfonyl) cyclohexyl)-4- (trifluoromethyl)picolinamide | 499.17 |
| 85 | 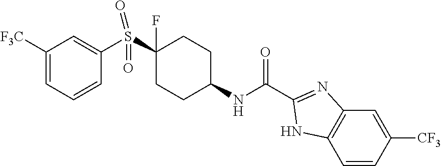 | N-((1S,4S)-4-fluoro-4-(3- (trifluoromethyl)phenylsulfonyl) cyclohexyl)-5-(trifluoromethyl)-1H- benzo[d]imidazole-2-carboxamide | 538.17 |
| 86 | 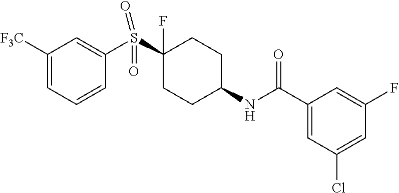 | 3-chloro-5-fluoro-N-((1S,4S)-4- fluoro-4-(3- (trifluoromethyl)phenylsulfonyl) cyclohexyl)benzamide | 482.09 |
| 87 | 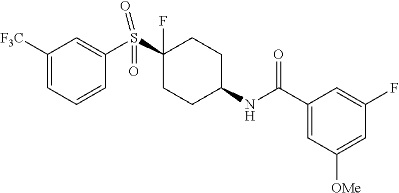 | 3-fluoro-N-((1S,4S)-4-fluoro-4-(3- (trifluoromethyl)phenylsulfonyl) cyclohexyl)-5-methoxybenzamide | 478.16 |
| 88 | 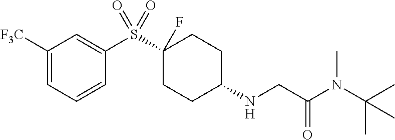 | N-tert-butyl-2-((1S,4S)-4-fluoro-4- (3-(trifluoromethyl)phenylsulfonyl) cyclohexylamino)-N- methylacetamide | 453.2 |
| 89 | 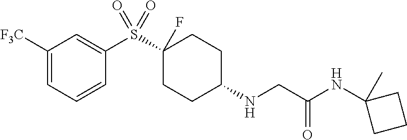 | 2-((1S,4S)-4-fluoro-4-(3- (trifluoromethyl)phenylsulfonyl) cyclohexylamino)-N-(1- methylcyclobutyl)acetamide | 451.19 |
| 90 | 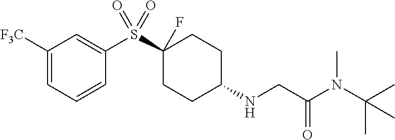 | N-tert-butyl-2-((1R,4R)-4-fluoro-4- (3-(trifluoromethyl)phenylsulfonyl) cyclohexylamino)-N- methylacetamide | 453.2 |
| 91 | 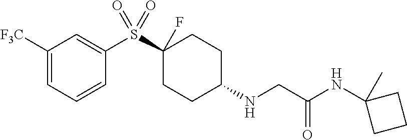 | 2-((1R,4R)-4-fluoro-4- (3- (trifluoromethyl)phenylsulfonyl) cyclohexylamino)-N-(1- methylcyclobutyl)acetamide | 451.19 |
| 92 | 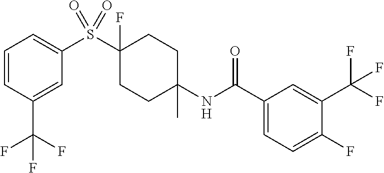 | 4-fluoro-N-(4-fluoro-1-methyl-4-(3- (trifluoromethyl)phenylsulfonyl) cyclohexyl)-3- (trifluoromethyl)benzamide | 530.15 |
| 93 | 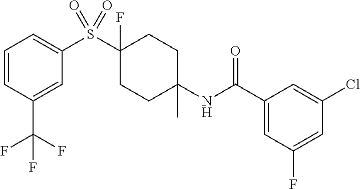 | 3-chloro-5-fluoro-N-(4-fluoro-1- methyl-4-(3- (trifluoromethyl)phenylsulfonyl) cyclohexyl)benzamide | 496.11 |
| 94 | 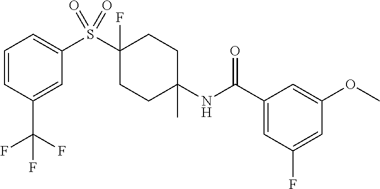 | 3-fluoro-N-(4-fluoro-1-methyl-4-(3- (trifluoromethyl)phenylsulfonyl) cyclohexyl)-5-methoxybenzamide | 492.16 |
| 95 | 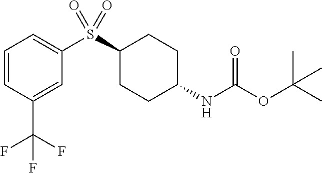 | tert-butyl(1R,4R)-4-(3- (trifluoromethyl)phenylsulfonyl) cyclohexylcarbamate | 408.15 |
| 96 | 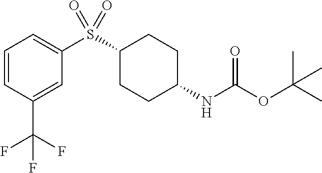 | tert-butyl(1S,4S)-4-(3- (trifluoromethyl)phenylsulfonyl) cyclohexylcarbamate | 408.17 |
| 97 | 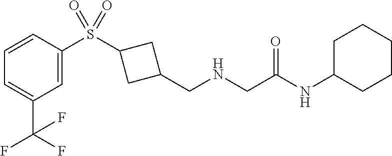 | N-cyclohexyl-2-((3-(3- (trifluoromethyl)phenylsulfonyl) cyclobutyl)methylamino)acetamide | 524.17 |
| 98 | 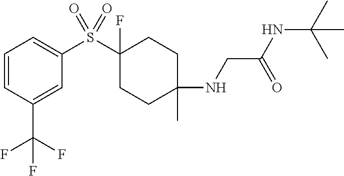 | N-tert-butyl-2-(4-fluoro-1-methyl- 4-(3- (trifluoromethyl)phenylsulfonyl) cyclohexylamino)acetamide | 524.17 |
| 99 | 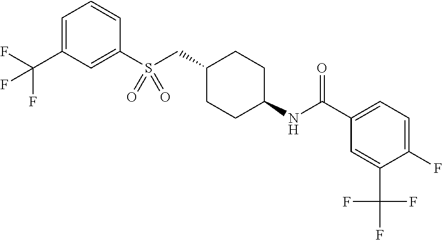 | 4-fluoro-3-(trifluoromethyl)-N- ((1R,4R)-4-((3- (trifluoromethyl)phenylsulfonyl) methyl)cyclohexyl)benzamide | 464.15 |
| 100 | 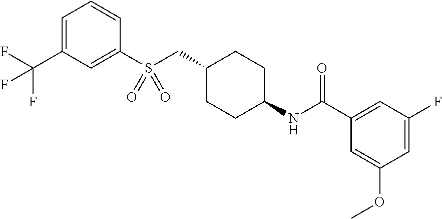 | 3-fluoro-5-methoxy-N-((1R,4R)-4- ((3- (trifluoromethyl)phenylsulfonyl) methyl)cyclohexyl)benzamide | 464.15 |
| 101 | 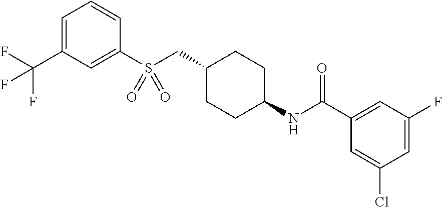 | 3-chloro-5-fluoro-N-((1R,4R)-4- ((3- (trifluoromethyl)phenylsulfonyl) methyl)cyclohexyl)benzamide | 514.18 |
| 102 | 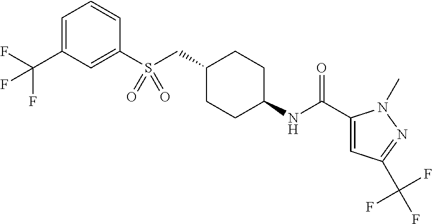 | 1-methyl-3-(trifluoromethyl)-N- ((1R,4R)-4-((3- (trifluoromethyl)phenylsulfonyl) methyl)cyclohexyl)-1H-pyrazole- 5-carboxamide | 530.18 |
| 103 | 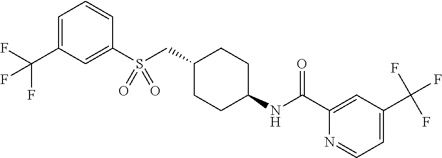 | 4-(trifluoromethyl)-N-((1R,4R)-4- ((3- (trifluoromethyl)phenylsulfonyl) methyl)cyclohexyl)picolinamide | 476.18 |
| 104 | 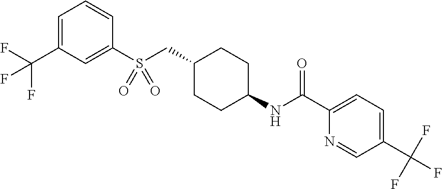 | 5-(trifluoromethyl)-N-((1R,4R)-4- ((3- (trifluoromethyl)phenylsulfonyl) methyl)cyclohexyl)picolinamide | 524.17 |
| 105 | 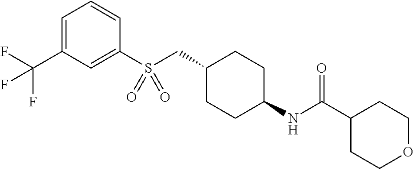 | N-((1R,4R)-4-((3- (trifluoromethyl)phenylsulfonyl) methyl)cyclohexyl)tetrahydro-2H- pyran-4-carboxamide | 524.17 |
| 106 | 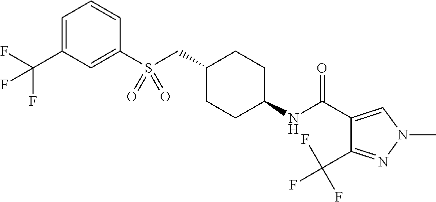 | 1-methyl-3-(trifluoromethyl)-N- ((1R,4R)-4-((3- (trifluoromethyl)phenylsulfonyl) methyl)cyclohexyl)-1H-pyrazole-4- carboxamide | 464.15 |
| 107 | 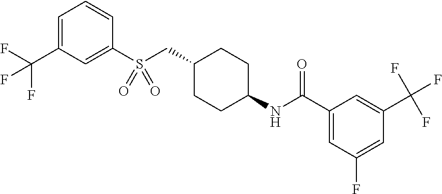 | 3-fluoro-5-(trifluoromethyl)-N- ((1R,4R)-4-((3- (trifluoromethyl)phenylsulfonyl) methyl)cyclohexyl)benzamide | 464.15 |
| 108 | 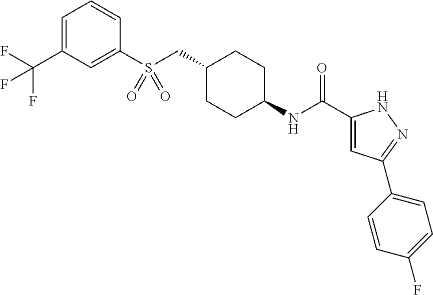 | 3-(4-fluorophenyl)-N-((1R,4R)-4- ((3- (trifluoromethyl)phenylsulfonyl) methyl)cyclohexyl)-1H-pyrazole- 5-carboxamide | 514.16 |
| 109 | 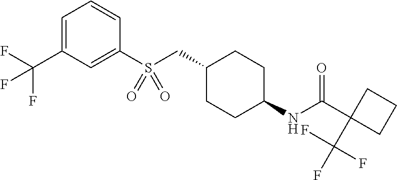 | 1-(trifluoromethyl)-N-((1R,4R)-4- ((3- (trifluoromethyl)phenylsulfonyl) methyl)cyclohexyl) cyclobutanecarboxamide | 530.18 |
| 110 | 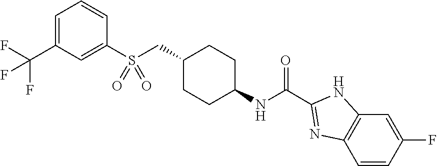 | 6-fluoro-N-((1R,4R)-4-((3- (trifluoromethyl)phenylsulfonyl) methyl)cyclohexyl)-1H- benzo[d]imidazole-2-carboxamide | 476.18 |
| 111 | 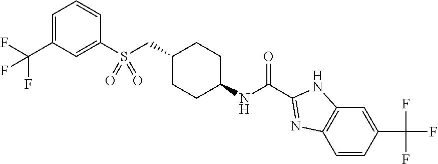 | 6-(trifluoromethyl)-N-((1R,4R)-4- ((3- (trifluoromethyl)phenylsulfonyl) methyl)cyclohexyl)-1H- benzo[d]imidazole-2-carboxamide | 474.24 |
| 112 | 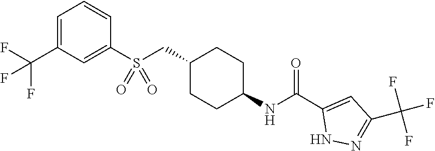 | 3-(trifluoromethyl)-N-((lR,4R)-4- ((3- (trifluoromethyI)phenylsulfonyl) methyl)cyclohexyl)-1H-pyrazole-5- carboxamide | 478.17 |
| 113 | 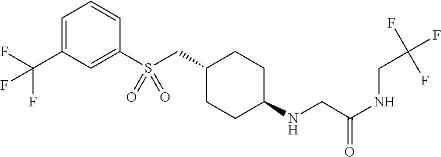 | N-(2,2,2-trifluoroethyl)-2-((1R,4R)- 4-((3-(trifluoromethyl)phenylsulfonyl) methyl)cyclohexylamino)acetamide | 512.21 |
| 114 | 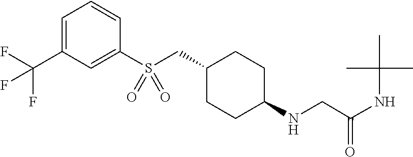 | N-tert-butyl-2-((1R,4R)-4-((3- (trifluoromethyl)phenylsulfonyl) methyl)cyclohexylamino)acetamide | 544.22 |
| 115 | 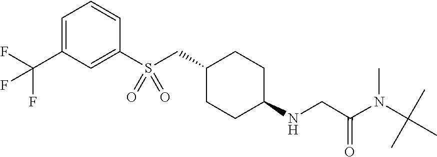 | N-tert-butyl-N-methyl-2-((1R,4R)- 4-((3- (trifluoromethyl)phenylsulfonyl) methyl)cyclohexylamino)acetamide | 490.2 |
| 116 | 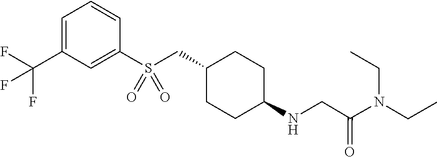 | N,N-diethyl-2-((1R,4R)-4-((3- (trifluoromethyl)phenylsulfonyl) methyl)cyclohexylamino)acetamide | 478.17 |
| 117 | 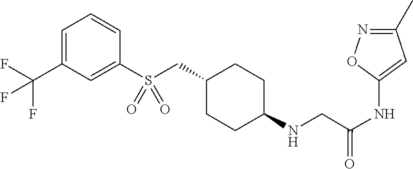 | N-(3-methylisoxazol-5-yl)-2- ((1R,4R)-4-((3- (trifluoromethyl)phenylsulfonyl) methyl)cyclohexylamino)acetamide | 478.17 |
| 118 | 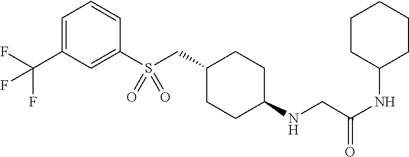 | N-cyclohexyl-2-((1R,4R)-4-((3- (trifluoromethyl)phenylsulfonyl) methyl)cyclohexylamino)acetamide | 495.21 |
| 119 | 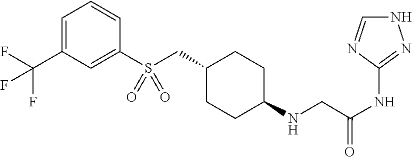 | N-(1H-1,2,4-triazol-3-yl)-2- ((1R,4R)-4-((3- (trifluoromethyl)phenylsulfonyl) methyl)cyclohexylamino)acetamide | 526.25 |
| 120 | 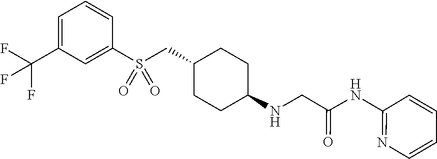 | N-(pyridin-2-yl)-2-((1R,4R)-4-((3- (trifluoromethyl)phenylsulfonyl) methyl)cyclohexylamino)acetamide | 512.24 |
| 121 | 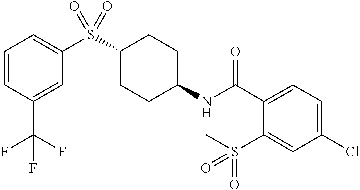 | 4-chloro-2-(methylsulfonyl)-N- ((1R,4R)-4-(3- (trifluoromethyl)phenylsulfonyl) cyclohexyl)benzamide | 474.24 |
| 122 | 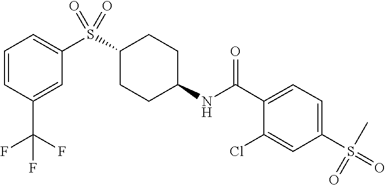 | 2-chloro-4-(methylsulfonyl)-N- ((1R,4R)-4-(3- (trifluoromethyl)phenylsulfonyl) cyclohexyl)benzamide | 478.19 |
| 123 | 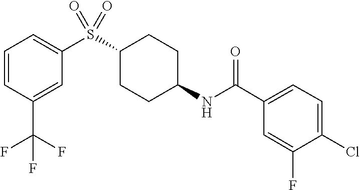 | 4-chloro-3-fluoro-N-((1R,4R)-4-(3- (trifluoromethyl)phenylsulfonyl) cyclohexyl)benzamide | 498.24 |
| 124 | 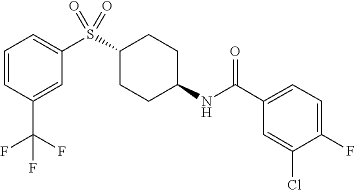 | 3-chloro-4-fluoro-N-((1R,4R)4-(3- (trifluoromethyl)phenylsulfonyl) cyclohexyl)benzamide | 495.23 |
| 125 | 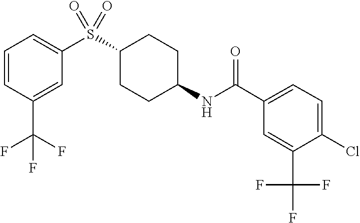 | 4-chloro-3-(trifluoromethyl)-N- ((1R,4R)-4-(3- (trifluoromethyl)phenylsulfonyl) cyclohexyl)benzamide | 495.23 |
| 126 | 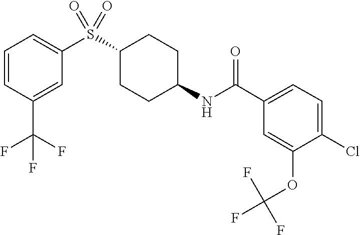 | 4-chloro-3-(trifluoromethoxy)-N- ((1R,4R)-4-(3- (trifluoromethyl)phenylsulfonyl) cyclohexyl)benzamide | 434.25 |
| 127 | 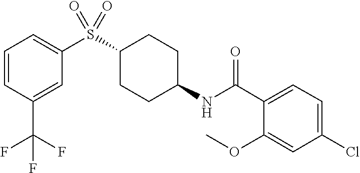 | 4-chloro-2-methoxy-N-((1R,4R)-4- (3-(trifluoromethyl)phenylsulfonyl) cyclohexyl)benzamide | 498.23 |
| 128 | 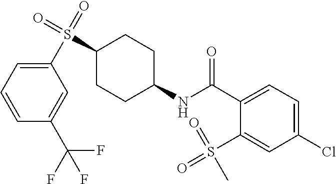 | 4-chloro-2-(methylsulfonyl)-N- ((1S,4S)-4-(3- (trifluoromethyl)phenylsulfonyl) cyclohexyl)benzamide | 512.24 |
| 129 | 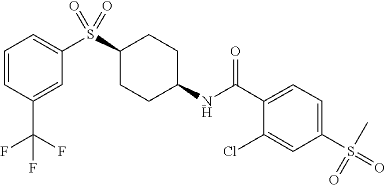 | 2-chloro-4-(methylsulfonyl)-N- ((1S,4S)-4-(3- (trifluoromethyl)phenylsulfonyl) cyclohexyl)benzamide | 510.26 |
| 130 | 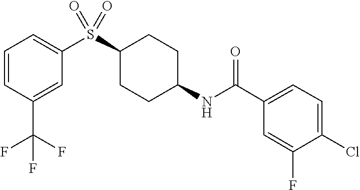 | 4-chloro-3-fluoro-N-((1S,4S)-4-(3- (trifluoromethyl)phenylsulfonyl) cyclohexyl)benzamide | 472.23 |
| 131 | 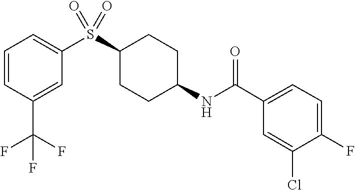 | 3-chloro-4-fluoro-N-((1S,4S)-4-(3- (trifluoromethyl)phenylsulfonyl) cyclohexyl)benzamide | 484.23 |
| 132 | 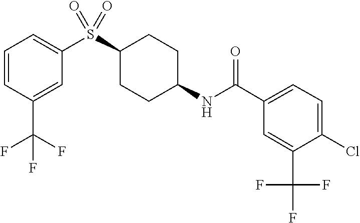 | 4-chloro-3-(trifluoromethyl)-N- ((1S,4S)-4-(3- (trifluoromethyl)phenylsulfonyl) cyclohexyl)benzamide | 534.26 |
| 133 | 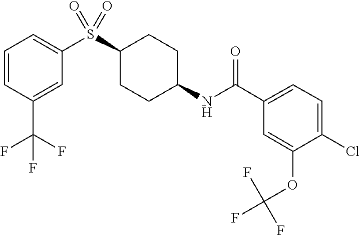 | 4-chloro-3-(trifluoromethoxy)-N- ((1S,4S)-4-(3- (trifluoromethyl)phenylsulfonyl) cyclohexyl)benzamide | 484.21 |
| 134 | 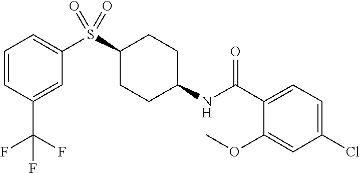 | 4-chloro-2-methoxy-N-((1S,4S)-4- (3-(trifluoromethyl)phenylsulfonyl) cyclohexyl)benzamide | 461.24 |
| 135 | 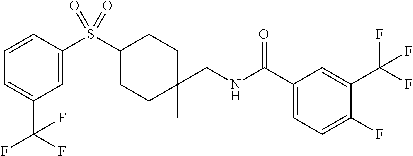 | 4-fluoro-N-((1-methyl-4-(3- (trifluoromethyl)phenylsulfonyl) cyclohexyl)methyl)-3- (trifluoromethyl)benzamide | 435.3 |
| 136 | 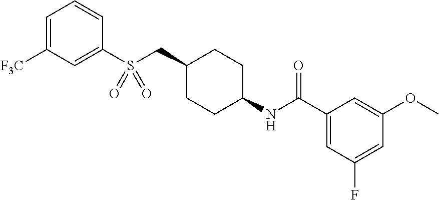 | 3-fluoro-5-methoxy-N-((1S,4S)-4- ((3- (trifluoromethyl)phenylsulfonyl) methyl)cyclohexyl)benzamide | 449.31 |
| 137 | 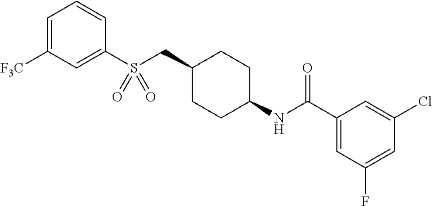 | 3-chloro-5-fluoro-N-((1S,4S)-4-((3- (trifluoromethyl)phenylsulfonyl) methyl)cyclohexyl)benzamide | 435.3 |
| 138 | 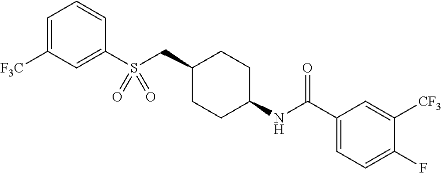 | 4-fluoro-3-(trifluoromethyl)-N- ((1S,4S)-4-((3- (trifluoromethyl)phenylsulfonyl) methyl)cyclohexyl)benzamide | 460.23 |
| 139 | 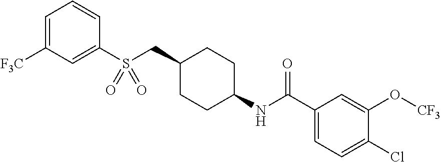 | 4-chloro-3-(trifluoromethoxy)-N- ((1S,4S)-4-((3- (trifluoromethyl)phenylsulfonyl) methyl)cyclohexyl)benzamide | 461.31 |
| 140 | 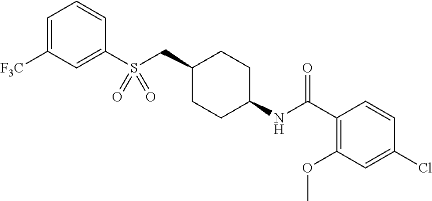 | 4-chloro-2-methoxy-N-((1S,4S)-4- ((3- (trifluoromethyl)phenylsulfonyl) methyl)cyclohexyl)benzamide | 446.25 |
| 141 | 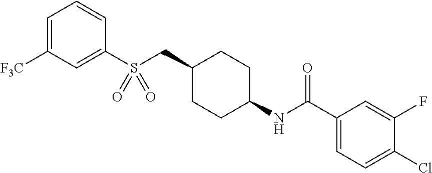 | 4-chloro-3-fluoro-N-((1S,4S)-4-((3- (trifluoromethyl)phenylsulfonyl) methyl)cyclohexyl)benzamide | 456.26 |
| 142 | 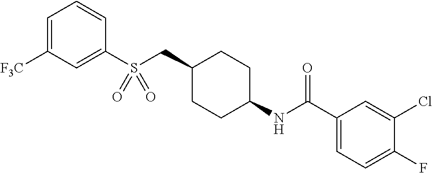 | 3-chloro-4-fluoro-N-((1S,4S)-4-((3- (trifluoromethyl)phenylsulfonyl) methyl)cyclohexyl)benzamide | 435.26 |
| 143 | 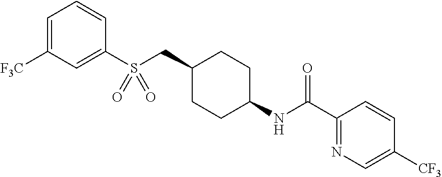 | 5-( trifluoromethyl)-N-((1S,4S)-4- ((3- (trifluoromethyl)phenylsulfonyl) methyl)cyclohexyl)picolinamide | 449.28 |
| 144 | 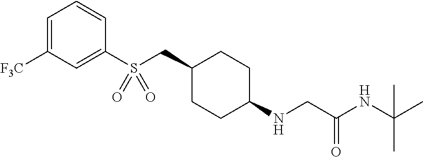 | N-tert-butyl-2-((1S,4S)-4-((3- (trifluoromethyl)phenylsulfonyl) methyl)cyclohexylamino)acetamide | 453.27 |
| 145 | 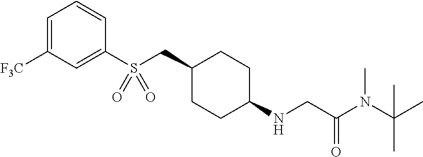 | N-tert-butyl-N-methyl-2-((1S,4S)- 4-((3- (trifluoromethyl)phenylsulfonyl) methyl)cyclohexylamino)acetamide | 463.9 |
| 146 | 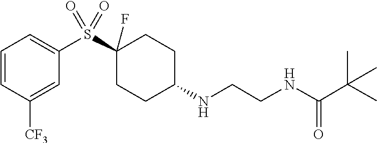 | N-(2-((1R,4R)-4-fluoro-4-(3- (trifluoromethyl)phenylsulfonyl) cyclohexylamino)ethyl)pivalamide | 453.24 |
| 147 | 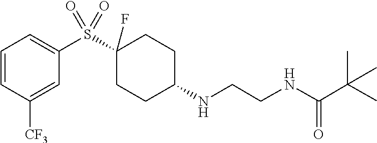 | N-(2-((1S,4S)-4-fluoro-4-(3- (trifluoromethyl)phenylsulfonyl) cyclohexylamino)ethyl)pivalamide | 453.24 |
| 148 | 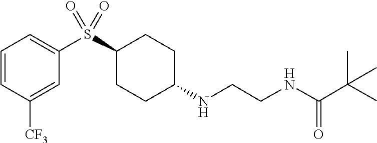 | N-(2-((1R,4R)-4-(3- (trifluoromethyl)phenylsulfonyl) cyclohexylamino)ethyl)pivalamide | 435.26 |
| 149 | 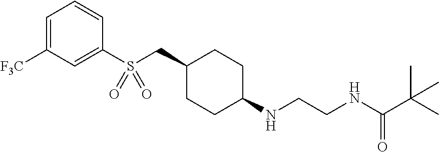 | N-(2-((1S,4S)-4-((3- (trifluoromethyl)phenylsulfonyl) methyl)cyclohexylamino)ethyl) pivalamide | 449.28 |
| 150 | 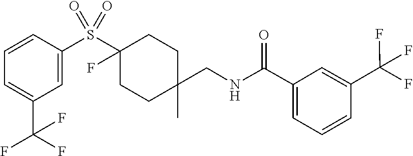 | 4-fluoro-N-((4-fluoro-1-methyl-4- (3-(trifluoromethyl)phenylsulfonyl) cyclohexyl)methyl)-3- (trifluoromethyl)benzamide | 467.26 |
| 151 | 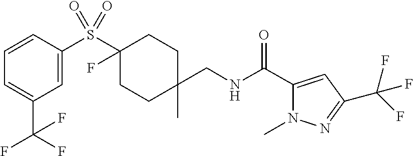 | N-((4-fluoro-1-methyl-4-(3- (trifluoromethyl)phenylsulfonyl) cyclohexyl)methyl)-l-methyl-3- (trifluoromethyl)-1H-pyrazole-5- carboxamide | 544.22 |
| 152 | 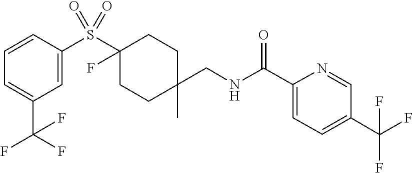 | N-((4-fluoro-1-methyl-4-(3- (trifluoromethyl)phenylsulfonyl) cyclohexyl)methyl)-5- (trifluoromethyl)picolinamide | 530.22 |
| 153 | 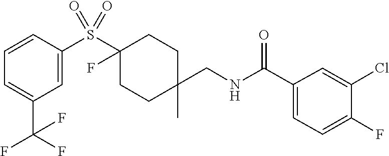 | 3-chloro-4-fluoro-N-((4-fluoro-1- methyl-4-(3- (trifluoromethyl)phenylsulfonyl) cyclohexyl)methyl)benzamide | 527.23 |
| 154 | 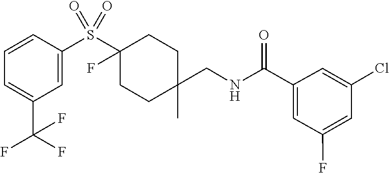 | 3-chloro-5-fluoro-N-((4-fluoro-1- methyl-4-(3- (trifluoromethyl)phenylsulfonyl) cyclohexyl)methyl)benzamide | 510.17 |
| 155 | 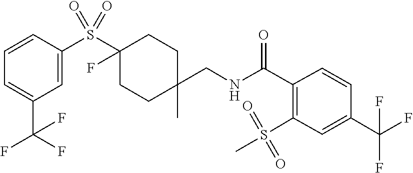 | N-((4-fluoro-1-methyl-4-(3- (trifluoromethyl)phenylsulfonyl) cyclohexyl)methyl)-2- (methylsulfonyl)-4- (trifluoromethyl)benzamide | 510.17 |
| 156 | 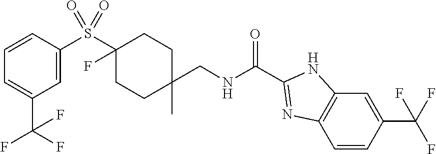 | N-((4-fluoro-1-methyl-4-(3- (trifluoromethyl)phenylsulfonyl) cyclohexyl)methyl)-6- (trifluoromethyl)-1H- benzo[d]imidazole-2-carboxamide | 604.2 |
| 157 | 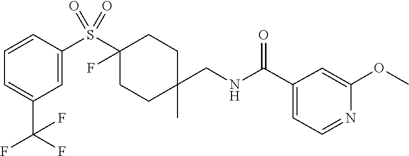 | N-((4-fluoro-1-methyl-4-(3- (trifluoromethyl)phenylsulfonyl) cyclohexyl)methyl)-2- methoxyisonicotinamide | 566.24 |
| 158 | 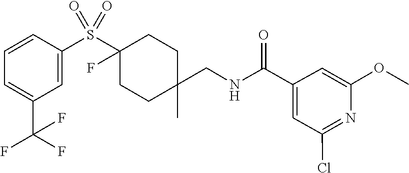 | 2-chloro-N-((4-fluoro-1-methyl-4- (3-(trifluoromethyl)phenylsulfonyl) cyclohexyl)methyl)-6- methoxyisonicotinamide | 489.23 |
| 159 | 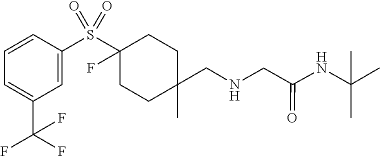 | N-tert-butyl-2-((4-fluoro-1-methyl- 4-(3- (trifluoromethyl)phenylsulfonyl) cyclohexyl)methylamino)acetamide | 523.2 |
| 160 | 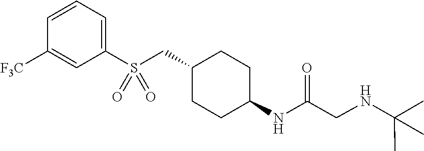 | 2-(tert-butylamino)-N-((1R,4R)-4- ((3- (trifluoromethyl)phenylsulfonyl) methyl)cyclohexyl)acetamide | 463.8 |
| 161 | 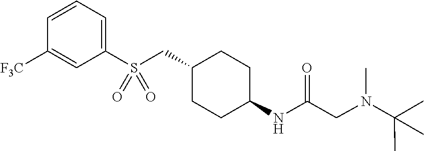 | 2-(tert-butyl(methyl)amino)-N- ((1R,4R)-4-((3- (trifluoromethyl)phenylsulfonyl) methyl)cyclohexyl)acetamide | 463.9 |
| 162 | 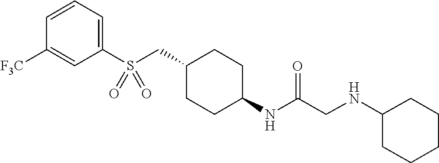 | 2-(cyclohexylamino)-N-((1R,4R)-4- ((3- (trifluoromethyl)phenylsulfonyl) methyl)cyclohexyl)acetamide | 435.26 |
| 163 | 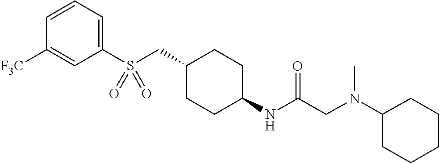 | 2-(cyclohexyl(methyl)amino)-N- (1R,4R)-4-((3- (trifluoromethyl)phenylsulfonyl) methyl)cyclohexyl)acetamide | 461.27 |
| 164 | 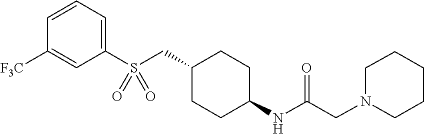 | 2-(piperidin-1-yl)-N-((1R,4R)-4- ((3- (trifluoromethyl)phenylsulfonyl) methyl)cyclohexyl)acetamide | 483.25 |
| 165 | 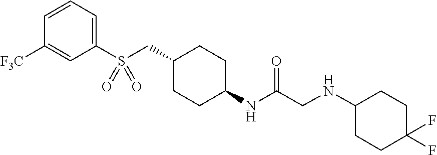 | 2-(4,4-difluorocyclohexylamino)-N- ((1R,4R)-4-((3- (trifluoromethyl)phenylsulfonyl) methyl)cyclohexyl)acetamide | 435.26 |
| 166 | 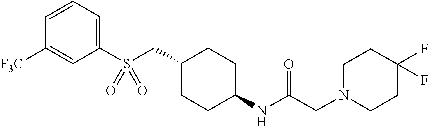 | 2-(4,4-difluoropiperidin-1-yl)-N- ((1R,4R)-4-((3- (trifluoromethyl)phenylsulfonyl) methyl)cyclohexyl)acetamide | 449.28 |
| 167 | 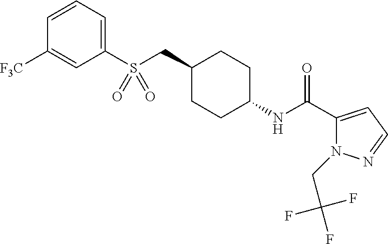 | l-(2,2,2-trifluoroethyl)-N-((1R,4R)- 4-((3- (trifluoromethyl)phenylsulfonyl) methyl)cyclohexyl)-1H-pyrazole-5- carboxamide | 461.27 |
| 168 | 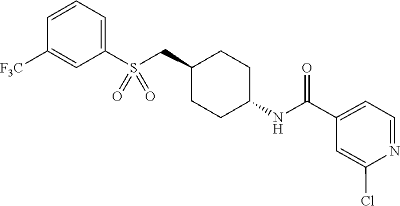 | 2-chloro-N-((1R.4R)-4-((3- (trifluoromethyl)phenylsulfonyl) methyl)cyclohexyl)isonicotinamide | 475.31 |
| 169 | 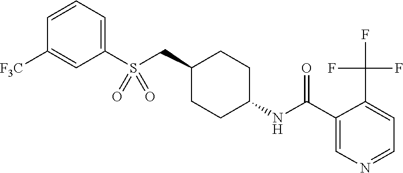 | 4-(trifluoromethyl)-N-((1R,4R)-4- ((3- (trifluoromethyl)phenylsulfonyl) methyl)cyclohexyl)nicotinamide | 447.26 |
| 170 | 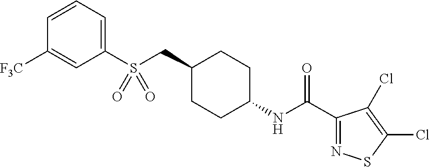 | 4,5-dichloro-N-((1R,4R)-4-((3- (trifluoromethyl)phenylsulfonyl) methyl)cyclohexyl)isothiazole-3- carboxamide | 497.26 |
| 171 | 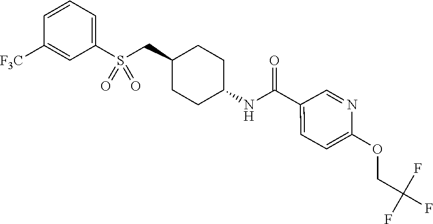 | 6-(2,2,2-trifluoroethoxy)-N- ((1R,4R)-4-((3- (trifluoromethyl)phenylsulfonyl) methyl)cyclohexyl)nicotinamide | 483.25 |
| 172 | 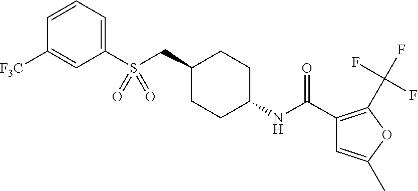 | 5-methyl-2-(trifluoromethyl)-N- ((1R,4R)-4-((3- (trifluoromethyl)phenylsulfonyl) methyl)cyclohexyl)furan-3- carboxamide | 494.8 |
| 173 | 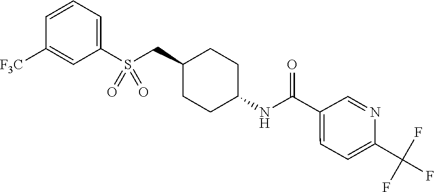 | 6-(trifluoromethyl)-N-((1R,4R)-4- ((3- (trifluoromethyl)phenylsulfonyl) methyl)cyclohexyl)nicotinamide | 498.23 |
| 174 | 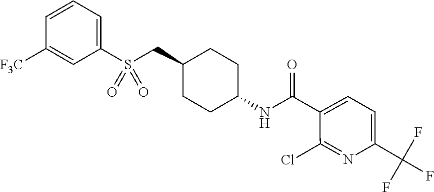 | 2-chloro-6-(trifluoromethyl)-N- ((1R,4R)-4-((3- (trifluoromethyl)phenylsulfonyl) methyl)cyclohexyl)nicotinamide | 461.16 |
| 175 | 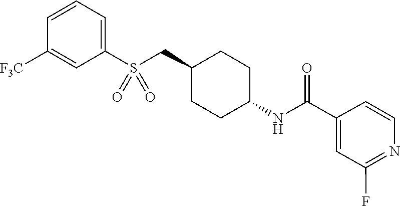 | 2-fluoro-N-((1R,4R)-4-((3- (trifluoromethyl)phenylsulfonyl) methyl)cyclohexyl)isonicotinamide | 495.21 |
| 176 | 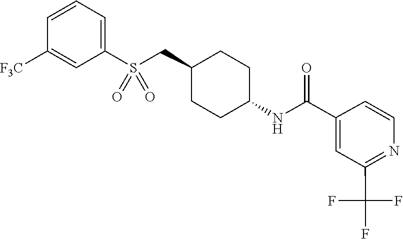 | 2-(trifluoromethyl)-N-((1R,4R)-4- ((3- (trifluoromethyl)phenylsulfonyl) methyl)cyclohexyl)isonicotinamide | 501.11 |
| 177 | 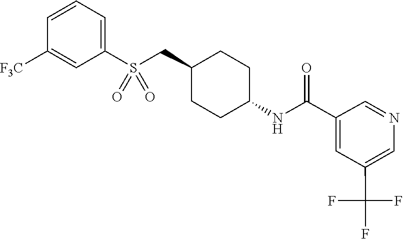 | 5-(trifluoromethyl)-N-((1R,4R)-4- ((3- (trifluoromethyl)phenylsulfonyl) tmehyl)cyclohexyl)nicotinamide | 525.23 |
| 178 | 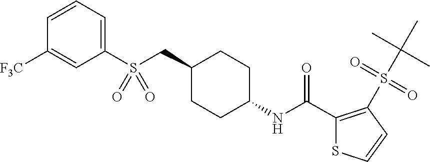 | 3-(tert-butylsulfonyl)-N-((1R.4R)- 4-((3- (trifluoromethyl)phenylsulfonyl) methyl)cyclohexyl)thiophene-2- carboxamide | 498.2 |
| 179 | 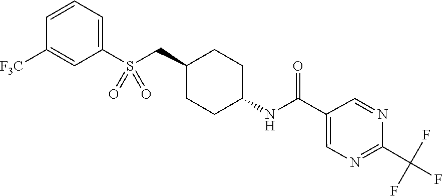 | 2-(trifluoromethyl)-N-((1R.4R)-4- ((3- (trifluoromethyl)phenylsulfonyl) methyl)cyclohexyl)pyrimidine-5- carboxamide | 495.21 |
| 180 | 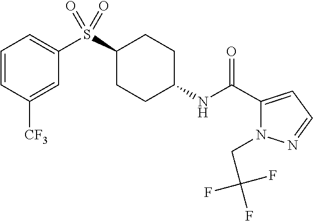 | 1-(2,2,2-trifluoroethyl)-N-((1R,4R)- 4-(3- (trifluoromethyl)phenylsulfonyl) cyclohexyl)-1H-pyrazole-5- carboxamide | 529.18 |
| 181 | 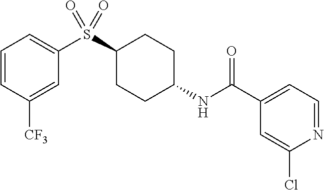 | 2-chloro-N-((1R,4R)-4-(3- (trifluoromethyl)phenylsulfonyl) cyclohexyl)isonicotinamide | 445.18 |
| 182 | 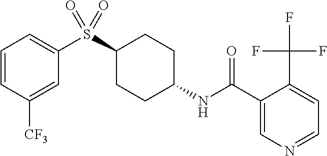 | 4-(trifluoromethyl)-N-((1R,4R)-4- (3-(trifluoromethyl)phenylsulfonyl) cyclohexyl)nicotinamide | 495.21 |
| 183 | 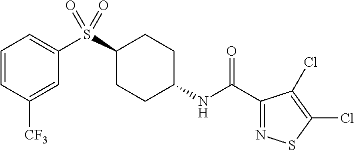 | 4,5-dichloro-N-((1R,4R)-4-(3- (trifluoromethyl)phenylsulfonyl) cyclohexyl)isothiazole-3- carboxamide | 495.21 |
| 184 | 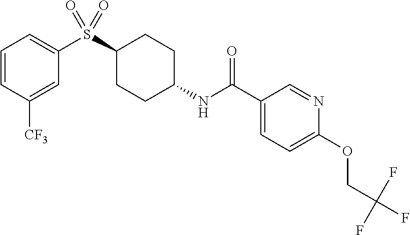 | 6-(2,2,2-trifluoroethoxy)-N- (1R,4R)-4-(3- (trifluoromethyl)phenylsulfonyl) cyclohexyl)nicotinamide | 552.23 |
| 185 | 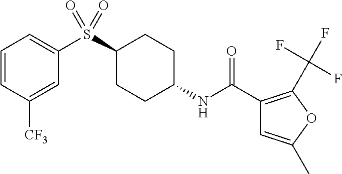 | 5-methyl-2-(trifluoromethyl)-N- ((1R,4R)-4-(3- (trifluoromethyl)phenylsulfonyl) cyclohexyl)furan-3-carboxamide | 496.2 |
| 186 | 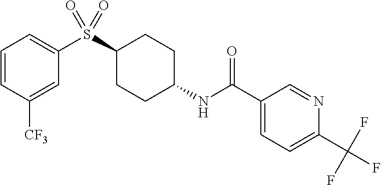 | 6-(trifluoromethyl)-N-((1R,4R)-4- (3-(trifluoromethyl)phenylsulfonyl) cyclohexyl)nicotinamide | 484.18 |
| 187 | 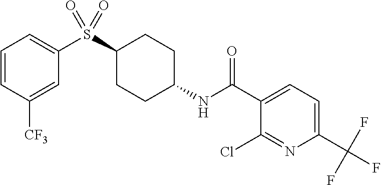 | 2-chloro-6-(trifluoromethyl)-N- ((1R,4R)-4-(3- (tritluoromethyl)phenylsulfonyl) cyclohexyl)nicotinamide | 447.15 |
| 188 | 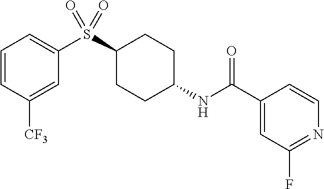 | 2-fluoro-N-((1R,4R)-4-(3- (trifluoromethyl)phenylsulfonyl) cyclohexyl)isonicotinamide | 481.17 |
| 189 | 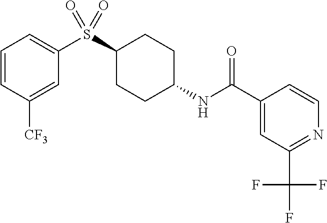 | 2-(trifluoromethyl)-N-((1R,4R)-4- (3-(trifluoromethyl)phenylsulfonyl) cyclohexyl)isonicotinamide | 487.07 |
| 190 | 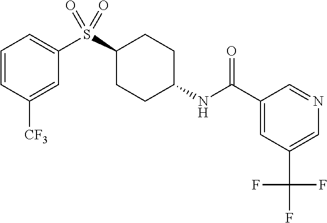 | 5-(trifluoromethyl)-N-((1R,4R)-4- (3-(trifluoromethyl)phenylsulfonyl) cyclohexyl)nicotinamide | 511.2 |
| 191 | 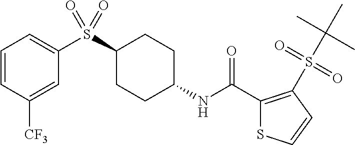 | 3-(tert-butylsulfonyl)-N-((1R,4R)- 4-(3- (trifluoromethyl)phenylsulfonyl) cyclohexyl)thiophene-2- carboxamide | 484.15 |
| 192 | 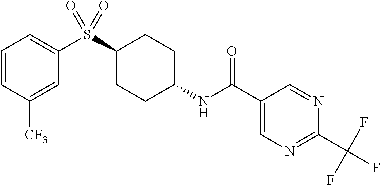 | 2-(trifluoromethyl)-N-((1R,4R)-4- (3-(trifluoromethyl)phenylsulfonyl) cyclohexyl)pyrimidine-5- carboxamide | 481.16 |
| 193 | 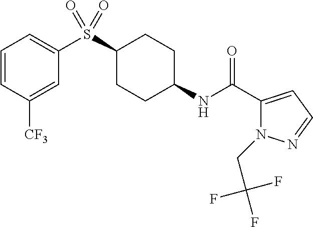 | l-(2,2,2-trifluoroethyl)-N-((1S,4S)- 4-(3- (trifluoromethyl)phenylsulfonyl) cyclohexyl)-1H-pyrazole-5- carboxamide | 515.14 |
| 194 | 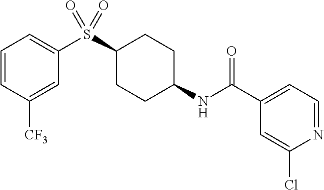 | 2-chloro-N-((1S,4S)-4-(3- (trifluoromethyl)phenylsulfonyl) cyclohexyl)isonicotinamide | 431.15 |
| 195 | 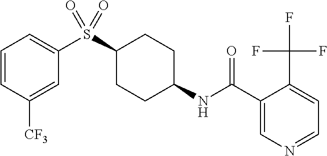 | 4-(trifluoromethyl)-N-((1S,4S)-4- (3-(trifluoromethyl)phenylsulfonyl) cyclohexyl)nicotinamide | 481.16 |
| 196 | 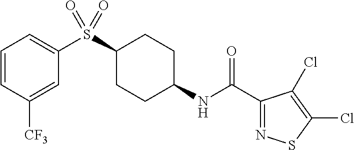 | 4,5-dichloro-N-((1S,4S)-4-(3- (trifluoromethyl)phenylsulfonyl) cyclohexyl)isothiazole-3- carboxamide | 481.16 |
| 197 | 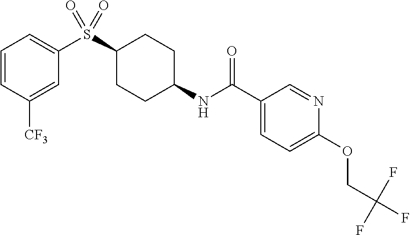 | 6-(2,2,2-trifluoroethoxy)-N- ((1S,4S)-4-(3- (trifluoromethyl)phenylsulfonyl) cyclohexyl)nicotinamide | 538.18 |
| 198 | 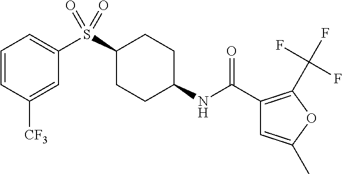 | 5-methyl-2-(trifluoromethyl)-N- ((1S,4S)-4-(3- (trifluoromethyl)phenylsulfonyl) cyclohexyl)furan-3-carboxamide | 482.13 |
| 199 | 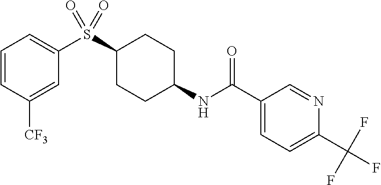 | 6-(trifluoromethyl)-N-((1S,4S)-4- (3-(trifluoromethyl)phenylsulfonyl) cyclohexyl)nicotinamide | 484.18 |
| 200 | 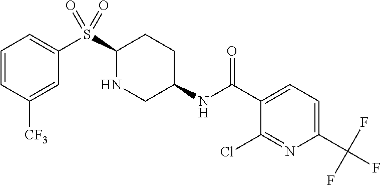 | 2-chloro-6-(trifluoromethyl)-N- ((1S,4S)-4-(3- (trifluoromethyl)phenylsulfonyl) cyclohexyl)nicotinamide | 447.11 |
| 201 | 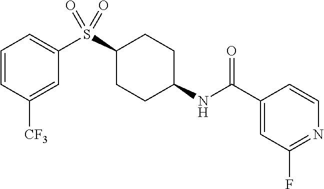 | 2-fluoro-N-((1S,4S)-4-(3- (trifluoromethyl)phenylsulfonyl) cyclohexyl)isonicotinamide | 481.16 |
| 202 | 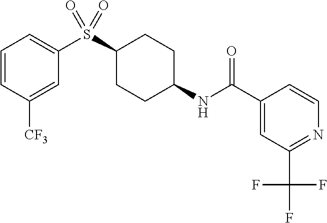 | 2-(trifluoromethyl)-N-((1S,4S)-4- (3-(trifluoromethyl)phenylsulfonyl) cyclohexyl)isonicotinamide | 487.06 |
| 203 | 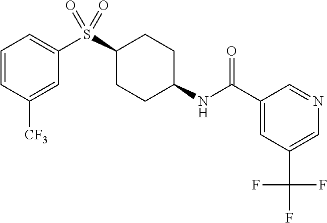 | 5-(trifluoromethyl)-N-((1S,4S)-4- (3-(trifluoromethyl)phenylsulfonyl) cyclohexyl)nicotinamide | 511.17 |
| 204 | 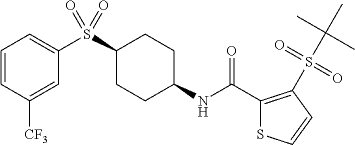 | 3-(tert-butylsulfonyl)-N-((1S,4S)-4- (3-(trifluoromethyl)phenylsulfonyl) cyclohexyl)thiophene-2- carboxamide | 484.15 |
| 205 | 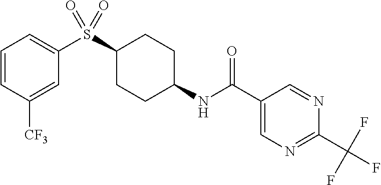 | 2-(trifluoromethyl)-N-((1S,4S)-4- (3-(trifluoromethyl)phenylsulfonyl) cyclohexyl)pyrimidine-5- carboxamide | 481.16 |
| 206 | 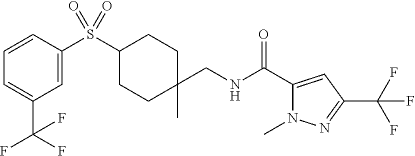 | 1-methyl-N-((1-methyl-4-(3- (trifluoromethyl)phenylsulfonyl) cyclohexyl)methyl)-3- (trifluoromethyl)-1H-pyrazole-5- carboxamide | 515.13 |
| 207 | 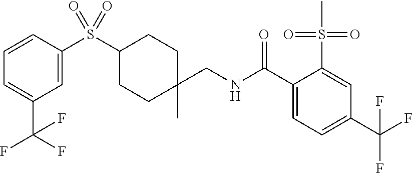 | N-((1-methyl-4-(3- (trifluoromethyl)phenylsulfonyl) cyclohexyl)methyl)-2- (methylsulfonyl)-4- (trifluoromethyl)benzamide | 431.15 |
| 208 | 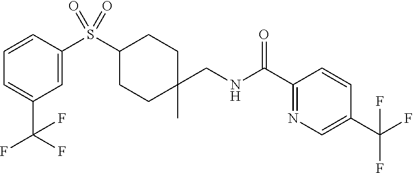 | N-((1-methyl-4-(3- (trifluoromethyl)phenylsulfonyl) cyclohexyl)methyl)-5- (trifluoromethyl)picolinamide | 481.16 |
| 209 | 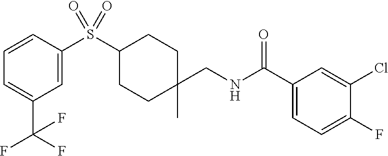 | 3-chloro-4-fluoro-N-((1-methyl-4- (3-(trifluoromethyl)phenylsulfony]) cyclohexyl)methyl)benzamide | 481.16 |
| 210 | 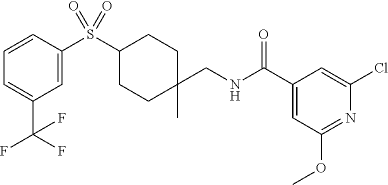 | 2-chloro-6-methoxy-N-((1-methyl- 4-(3- (trifluoromethyl)phenylsulfonyl) cyclohexyl)methyl)isonicotinamide | 481.16 |
| 211 | 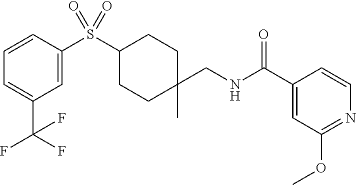 | 2-methoxy-N-((1-methyl-4-(3- (trifluoromethyl)phenylsulfonyl) cyclohexyl)methyl)isonicotinamide | 538.18 |
| 212 | 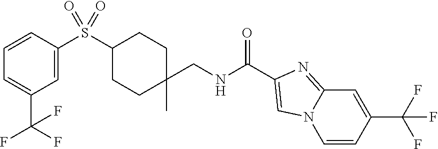 | N-((1-methyl-4-(3- (trifluoromethyl)phenylsulfonyl) cyclohexyl)methy])-7- (trifluoromethyl)imidazo[1,2- a]pyridine-2-carboxamide | 482.16 |
| 213 | 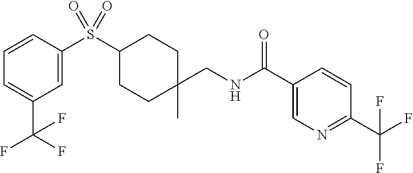 | N-((1-methyl-4-(3- (trifluoromethyl)phenylsulfonyl) cyclohexyl)methyl)-6- (trifluoromethyl)nicotinamide | 512.21 |
| 214 | 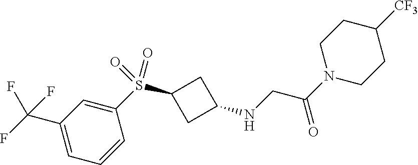 | 2-(trans-3-(3- (trifluoromethyl)phenylsulfonyl) cyclobutylamino)-1-(4- (trifluoromethyl)piperidin-1- yl)ethanone | 473.2 |
| 215 | 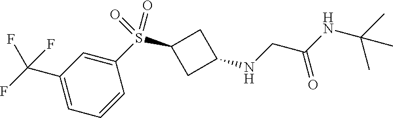 | N-tert-butyl-2-(trans-3-(3- (trifluoromethyl)phenylsulfonyl) cyclobutylamino)acetamide | 393.2 |
| 216 | 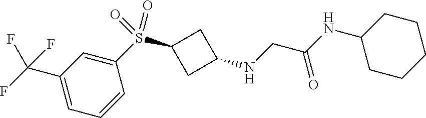 | N-cyclohexyl-2-(trans-3-(3- (trifluoromethyl)phenylsulfonyl) cyclobutylamino)acetamide | 419.2 |
| 217 | 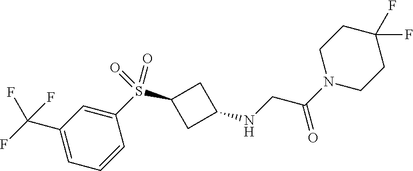 | 1-(4,4-difluoropiperidin-1-yl)-2- (trans-3-(3- (trifluoromethyl)phenylsulfonyl) cyclobutylamino)ethanone | 441.2 |
| 218 | 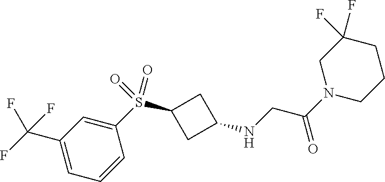 | 1-(3,3-difluoropiperidin-1-yl)-2- (trans-3-(3- (trifluoromethyl)phenylsulfonyl) cyclobutylamino)ethanone | 441.2 |
| 219 | 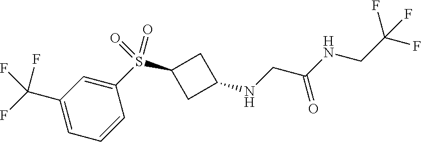 | N-(2,2,2-trifluoroethyl)-2-(trans-3- (3- (trifluoromethyl)phenylsulfonyl) cyclobutylamino)acetamide | 419.2 |
| 220 | 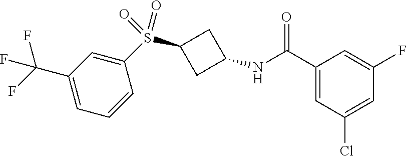 | 3-chloro-5-fluoro-N-(trans-3-(3- (trifluoromethyl)phenylsulfonyl) cyclobutyl)benzamide | 436.1 |
| 221 | 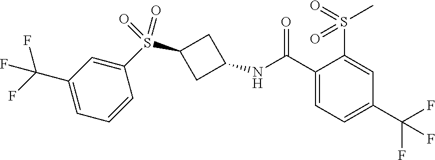 | 2-(methylsulfonyl)-4- (trifluoromethyl)-N-(trans-3-(3- (trifluoromethyl)phenylsulfonyl) cyclobutyl)benzarnide | 530.1 |
| 222 | 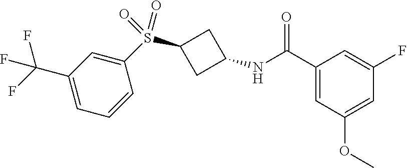 | 3-fluoro-5-methoxy-N-(trans-3-(3- (trifluoromethyl)phenylsulfonyl) cyclobutyl)benzamide | 432.1 |
| 223 | 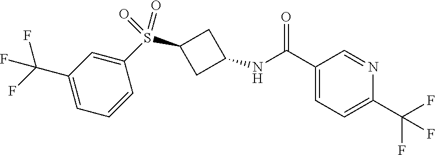 | 6-(trifluoromethyl)-N-(trans-3-(3- (trifluoromethyl)phenylsulfonyl) cyclobutyl)nicotinamide | 453.1 |
| 224 | 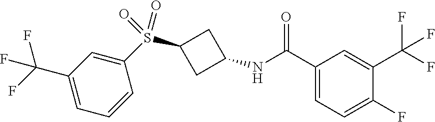 | 4-fluoro-3-(trifluoromethyl)-N- (trans-3-(3- (trifluoromethyl)phenylsulfonyl) cyclobutyl)benzamide | 470.1 |
| 225 | 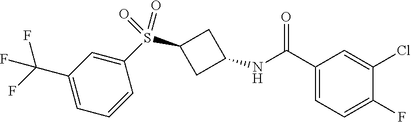 | 3-chloro-4-fluoro-N-(trans-3-(3- (trifluoromethyl)phenylsulfonyl) cyclobutyl)benzamide | 436.1 |
| 226 | 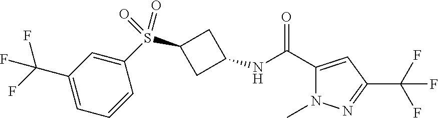 | 1-methyl-3-(trifluoromethyl)-N- (trans-3-(3- (trifluoromethyl)phenylsulfonyl) cyclobutyl)-1H-pyrazole-5- carboxamide | 456.1 |
| 227 | 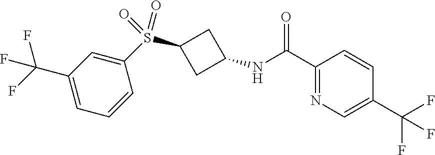 | 5-(trifluoromethyl)-N-(trans-3-(3- (trifluoromethyl)phenylsulfonyl) cyclobutyl)picolinamide | 453.1 |
| 228 | 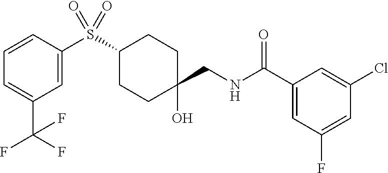 | 3-chloro-5-fluoro-N-(((1S,4S)-1- hydroxy-4-(3- (trifluoromethyl)phenylsulfonyl) cyclohexyl)methyl)benzamide | 494.2 |
| 229 | 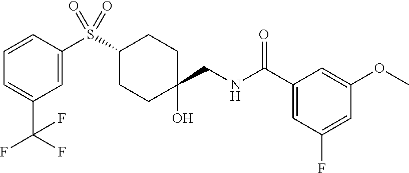 | 3-fluoro-N-(((1S,4S)-1-hydroxy-4- (3-(trifluoromethyl)phenylsulfonyl) cyclohexyl)methyl)-5- methoxybenzamide | 490.2 |
| 230 | 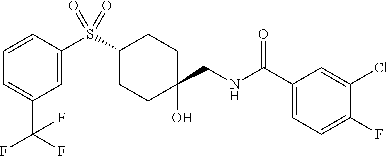 | 3-chloro-4-fluoro-N-(((1S,4S)-1- hydroxy-4-(3- (trifluoromethyl)phenylsulfonyl) cyclohexyl)methyl)benzamide | 494.2 |
| 231 | 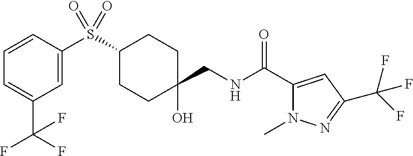 | N-(((1S,4S)-1-hydroxy-4-(3- (trifluoromethyl)phenylsulfonyl) cyclohexyl)methyl)-1-methyl-3- (trifluoromethyl)-1H-pyrazole-5- carboxamide | 514.2 |
| 232 | 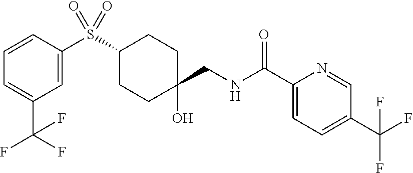 | N-(((1S,4S)-1-hydroxy-4-(3- (trifluoromethyl)phenylsulfonyl) cyclohexyl)methyl)-5- (trifluoromethyl)picolinamide | 511.2 |
| 233 | 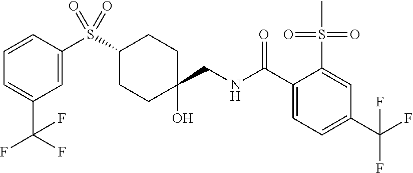 | N-(((1S,4S)-1-hydroxy-4-(3- (trifluoromethyl)phenylsulfonyl) cyclohexyl)methyl)-2- (methylsulfonyl)-4- (trifluoromethyl)benzamide | 588.2 |
| 234 | 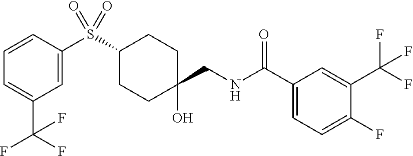 | 4-fluoro-N-(((1S,4S)-1-hydroxy-4- (3-(trifluoromethyl)phenylsulfonyl) cyclohexyl)methyl)-3- (trifluoromethyl)benzamide | 528.2 |
| 235 | 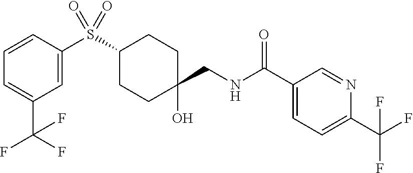 | N-(((1S,4S)-1-hydroxy-4-(3- (trifluoromethyl)phenylsulfonyl) cyclohexyl)methyl)-6- (trifluoromethyl)nicotinamide | 511.2 |
| 236 | 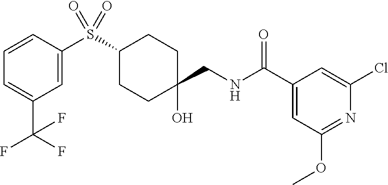 | 2-chloro-N-(((1S,4S)-1-hydroxy-4- (3-(trifluoromethyl)phenylsulfonyl) cyclohexyl)methyl)-6- methoxyisonicotinamide | 507.2 |
| 237 | 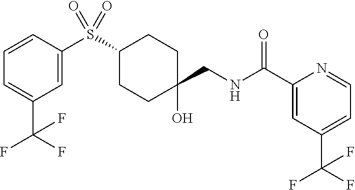 | N-(((1S,4S)-1-hydroxy-4-(3- (trifluoromethyl)phenylsulfonyl) cyclohexyl)methyl)-4- (trifluoromethyl)picolinamide | 511.2 |
| 238 | 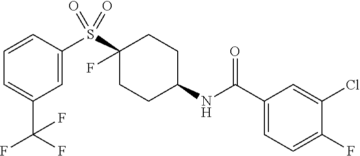 | 3-chloro-4-fluoro-N-((1S,4S)-4- fluoro-4-(3- (trifluoromethyl)phenylsulfonyl) cyclohexyl)benzamide | 482.2 |
| 239 | 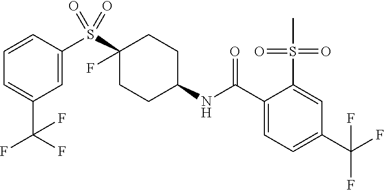 | N-((1S,4S)-4-fluoro-4-(3- (trifluoromethyl)phenylsulfonyl) cyclohexyl)-2-(methylsulfonyl)-4- (trifluoromethyl)benzamide | 576.2 |
| 240 | 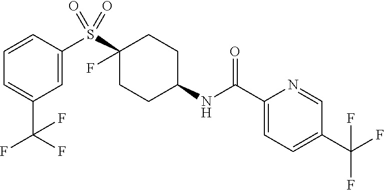 | N-((1S,4S)-4-fluoro-4-(3- (trifluoromethyl)phenylsulfonyl) cyclohexyl)-5- (trifluoromethyl)picolinamide | 499.2 |
| 241 | 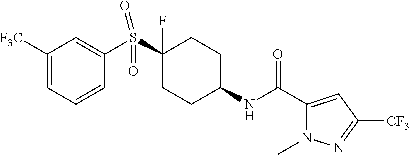 | N-((1S,4S)-4-fluoro-4-(3- (trifluoromethyl)phenylsulfonyl) cyclohexyl)-1-methyl-3- (trifluoromethyl)-1H-pyrazole-5- carboxamide | 502.2 |
| 242 | 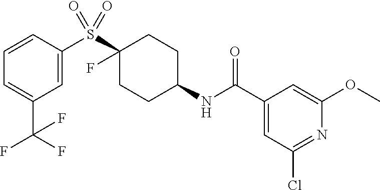 | 2-chloro-N-((1S,4S)-4-fluoro-4-(3- (trifluoromethyl)phenylsulfonyl) cyclohexyl)-6- methoxyisonicotinamide | 495.2 |
| 243 | 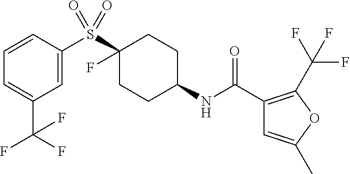 | N-((1S,4S)-4-fluoro-4-(3- (trifluoromethyl)phenylsulfonyl) cyclohexyl)-5-methyl-2- (trifluoromethyl)furan-3- carboxamide | 502.2 |
| 244 | 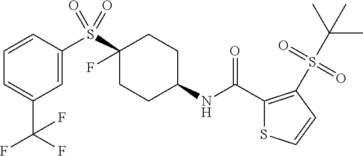 | 3-(tert-butylsulfonyl)-N-((1S,4S)-4- fluoro-4-(3- (trifluoromethyl)phenylsulfonyl) cyclohexyl)thiophene-2- carboxamide | 556.2 |
| 245 | 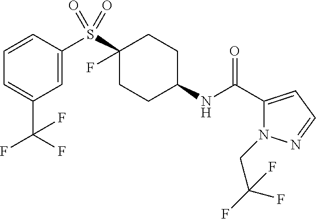 | N-((1S,4S)-4-fluoro-4-(3- (trifluoromethyl)phenylsulfonyl) cyclohexyl)-1-(2,2,2-trifluoroethyl)- 1H-pyrazole-5-carboxamide | 502.2 |
| 246 | 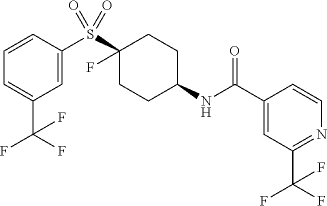 | N-((1S,4S)-4-fluoro-4-(3- (trifluoromethyl)phenylsulfonyl) cyclohexyl)-2- (trifluoromethyl)isonicotinamide | 499.2 |
| 247 | 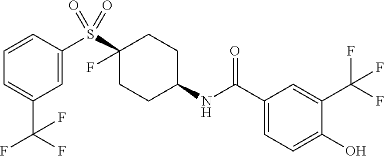 | N-((1S,4S)-4-fluoro-4-(3- (trifluoromethyl)phenylsulfonyl) cyclohexyl)-4-hydroxy-3- (trifluoromethyl)benzamide | 514.2 |
| 248 | 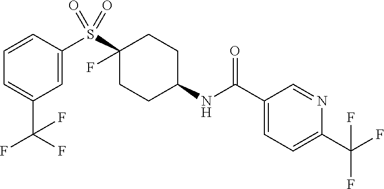 | N-((1S,4S)-4-fluoro-4-(3- (trifluoromethyl)phenylsulfonyl) cyclohexyl)-6- (trifluoromethyl)nicotinamide | 499.2 |
| 249 | 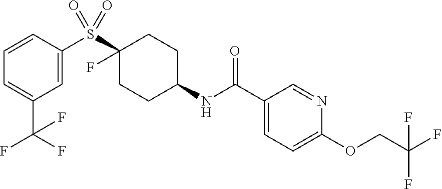 | N-((1S,4S)-4-fluoro-4-(3- (trifluoromethyl)phenylsulfonyl) cyclohexyl)-6-(2,2,2- trifluoroethoxy)nicotinamide | 529.2 |
| 250 | 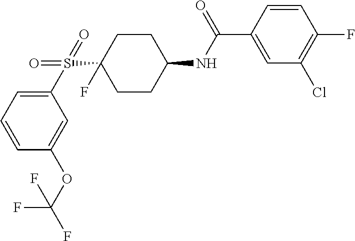 | 3-chloro-4-fluoro-N-((1R,4R)-4- fluoro-4-(3- (trifluoromethoxy)phenylsulfonyl) cyclohexyl)benzamide | 497.87 |
| 251 | 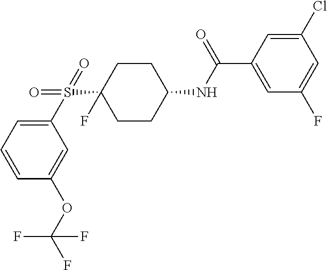 | 3-chloro-5-fluoro-N-((1S,4S)-4- fluoro-4-(3- (trifluoromethoxy)phenylsulfonyl) cyclohexyl)benzamide | 497.87 |
| 252 | 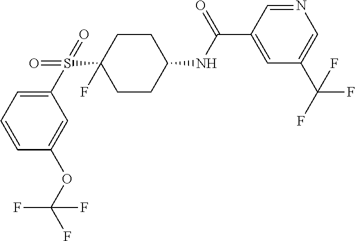 | N-((1S,4S)-4-fluoro-4-(3- (trifluoromethoxy)phenylsulfonyl) cyclohexyl)-5- (trifluoromethyl)nicotinamide | 514.42 |
| 253 | 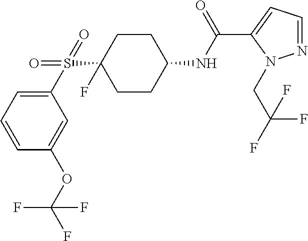 | N-((1S,4S)-4-fluoro-4-(3- (trifluoromethoxy)phenylsulfonyl) cyclohexyl)-1-(2,2,2-trifluoroethyl)- 1H-pyrazole-5-carboxamide | 517.42 |
| 254 | 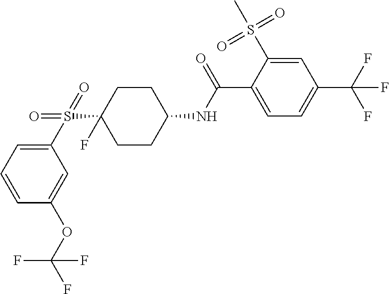 | N-((1S,4S)-4-fluoro-4-(3- (trifluoromethoxy)phenylsulfonyl) cyclohexyl)-2-(methylsulfonyl)-4- (trifluoromethyl)benzamide | 591.52 |
| 255 | 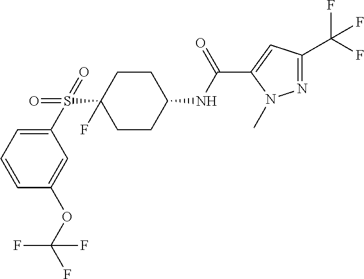 | N-((1S,4S)-4-fluoro-4-(3- (trifluoromethoxy)phenylsulfonyl) cyclohexyl)-1-methyl-3- (trifluoromethyl)-1H-pyrazole-5- carboxamide | 517.42 |
| 256 | 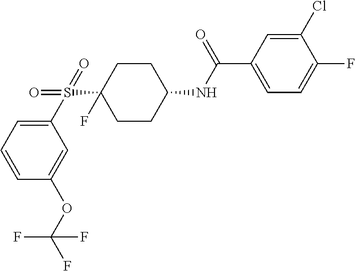 | 3-chloro-4-fluoro-N-((1S,4S)-4- fluoro-4-(3- (trifluoromethoxy)phenylsulfonyl) cyclohexyl)benzamide | 497.87 |
| 257 | 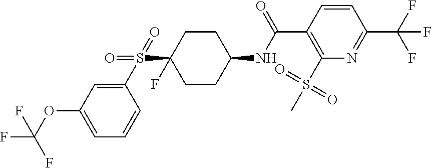 | N-((1S,4S)-4-fluoro-4-(3- (trifluoromethoxy)phenylsulfonyl) cyclohexyl)-2-(methylsulfonyl)-6- (trifluoromethyl)nicotinamide | 592.50 |
| 258 | 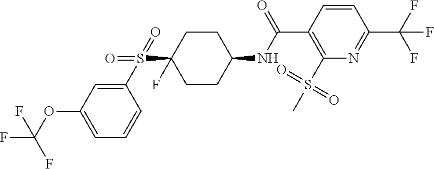 | N-((1S,4S)-4-fluoro-4-(3- (trifluoromethoxy)phenylsulfonyl) cyclohexyl)-2-(methylsulfonyl)-6- (trifluoromethyl)nicotinamide | 592.50 |
| 259 | 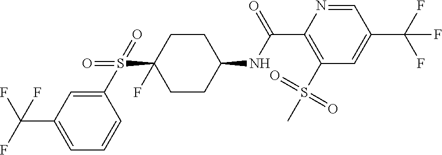 | N-((1S,4S)-4-fluoro-4-(3- (trifluoromethyl)phenylsulfonyl) cyclohexyl)-3-(methylsulfonyl)-5- (trifluoromethyl)picolinamide | 576.51 |
| 260 | 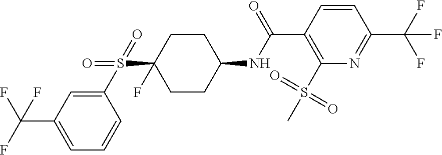 | N-((1S,4S)-4-fluoro-4-(3- (trifluoromethyl)phenylsulfonyl) cyclohexyl)-2-(methylsulfonyl)-6- (trifluoromethyl)nicotinamide | 576.51 |
| 261 | 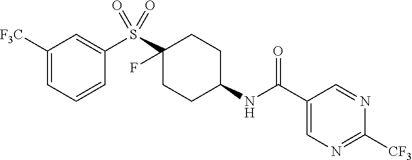 | N-((1S,4S)-4-fluoro-4-(3- (trifluoromethyl)phenylsulfonyl) cyclohexyl)-2- (trifluoromethyl)pyrimidine-5- carboxamide | 499.40 |
| 262 | 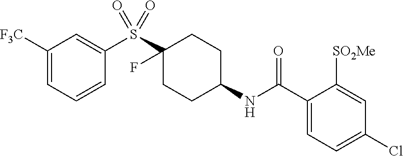 | 4-chloro-N-((1S,4S)-4-fluoro-4-(3- (trifluoromethyl)phenylsulfonyl) cyclohexyl)-2- (methylsulfonyl)benzamide | 541.97 |
| 263 | 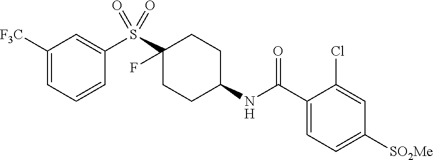 | 2-chloro-N-((1S,4S)-4-fluoro-4-(3- (trifluoromethyl)phenylsulfonyl) cyclohexyl)-4- (methylsulfonyl)benzamide | 541.97 |
| 264 | 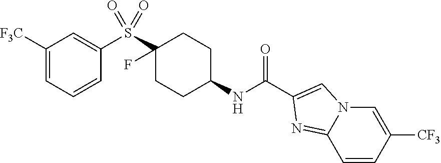 | N-((1S,4S)-4-fluoro-4-(3- (trifluoromethyl)phenylsulfonyl) cyclohexyl)-6- (trifluoromethyl)imidazo[1,2- a]pyridine-2-carboxamide | 537.45 |
| 265 | 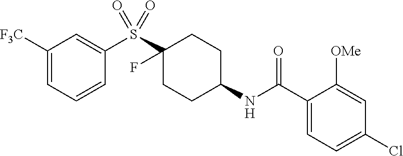 | 4-chloro-N-((1S,4S)-4-fluoro-4-(3- (trifluoromethyl)phenylsulfonyl) cyclohexyl)-2-methoxybenzamide | 493.90 |
| 266 | 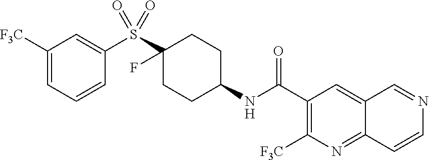 | N-((1S,4S)-4-fluoro-4-(3- (trifluoromethyl)phenylsulfonyl) cyclohexyl)-2-(trifluoromethyl)-1,6- naphthyridine-3-carbosamide | 549.46 |
| 267 | 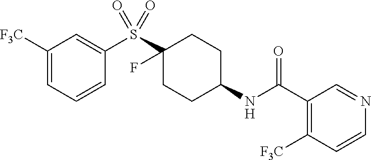 | N-((1S,4S)-4-fluoro-4-(3- (trifluoromethyl)phenylsulfonyl) cyclohexyl)-4- (trifluoromethyl)nicotinamide | 498.42 |
| 368 | 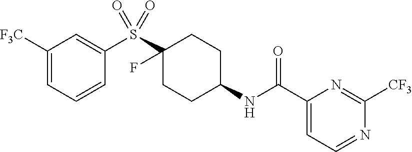 | N-((1S,4S)-4-fluoro-4-(3- (trifluoromethyl)phenylsulfonyl) cyclohexyl)-2- (trifluoromethyl)pyrimidine-4- carboxamide | 499.40 |
| 269 | 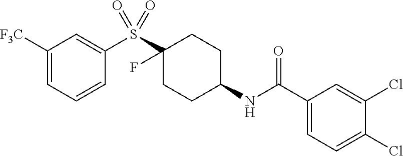 | 3,4-dichloro-N-((1S,4S)-4-fluoro-4- (3-(trifluoromethyl)phenylsulfonyl) cyclohexyl)benzamide | 498.32 |
| 270 | 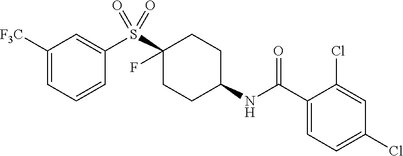 | 2,4-dichloro-N-((1S,4S)-4- fluoro-4-(3-(trifluoromethyl) phenylsulfonyl)cyclohexyl) benzamide | 498.32 |
| 271 | 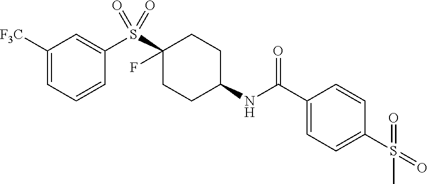 | N-((1S,4S)-4-fluoro-4-(3- (trifluoromethyl) phenylsulfonyl) cyclohexyl)-4- (methylsulfonyl)benzamide | 507.52 |
| 272 | 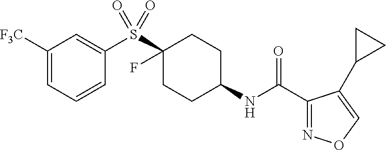 | 4-cyclopropyl-N-((1S,4S)-4- fluoro-4-(3-(trifluoromethyl) phenylsulfonyl)cyclohexyl) isoxazole-3-carboxamide | 460.45 |
| 273 | 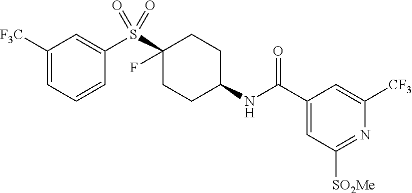 | N-((1R,4R)-4-fluoro-4-(3- (trifluoromethyl) phenylsulfonyl)cyclohexyl)-2-(methylsulfonyl)-6-(trifluoromethyl)isonicotinamide | 576.51 |
| 274 | 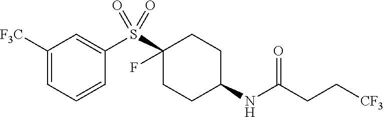 | 4,4,4-trifluoro-N-((1S,4S)-4- fluoro-4-(3-(trifluoromethyl) phenylsulfonyl)cyclohexyl)butanamide | 449.38 |
| 275 | 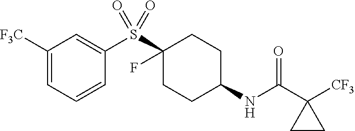 | N-((1S,4S)-4-fluoro-4-(3-(trifluoromethyl) phenylsulfonyl)cyclohexyl)-1-(trifluoromethyl) cyclopropaneCarboxamide | 461.40 |
| 276 | 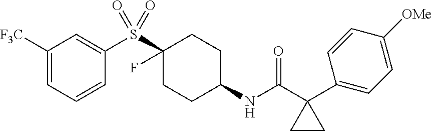 | N-((1S,4S)-4-fluoro-4-(3-(trifluoromethyl) phenylsulfonyl)cyclohexyl)-1-(4-methoxyphenyl) cyclopropanecarboxamide | 499.52 |
| 277 | 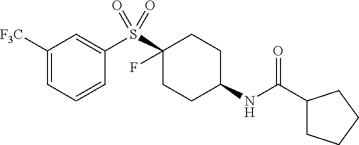 | N-((1S,4S)-4-fluoro-4-(3-(trifluoromethyl) phenylsulfonyl) cyclohexyl) cyclopentanecarboxamide | 421.45 |
| 278 | 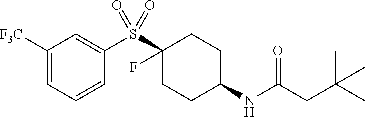 | N-((1S,4S)-4-fluoro-4-(3- (trifluoromethyl) phenylsulfonyl)cyclohexyl)- 3,3-dimethylbutanamide | 423.47 |
| 279 | 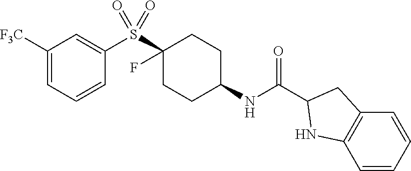 | N-((1S,4S)-4-fluoro-4-(3- (trifluoromethyl) phenylsulfonyl)cyclohexyl) indoline-2-carboxamide | 470.48 |
| 280 | 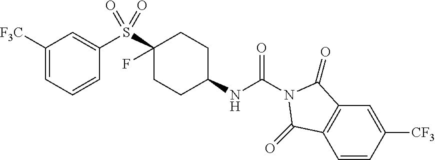 | 2-((1S,4S)-4-fluoro-4-(3-(trifluoromethyl) phenylsulfonyl)cyclohexyl)-5-(trifluoromethyl)isoindoline- 1,3-dione | 523.42 |
| 281 | 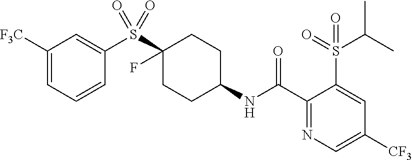 | N-((1S,4S)-4-fluoro-4-(3- (trifluoromethyl) phenylsulfonyl)cyclohexyl)-3- (isopropylsulfonyl)-5- (trifluoromethyl)picolinamide | 604.56 |
| 282 | 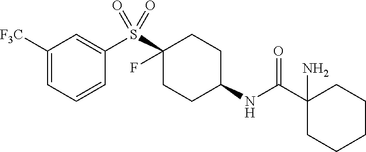 | 1-amino-N-((1S,4S)-4-fluoro- 4-(3- (trifluoromethyl) phenylsulfonyl)cyclohexyl) cyclohexanecarboxamide | 450.49 |
| 283 | 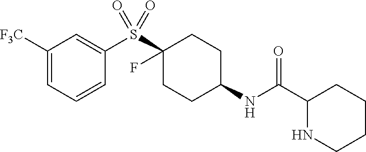 | N-((1S,4S)-4-fluoro-4-(3- (trifluorornethyl) phenylsulfonyl)cyclohexyl) piperidine-2-carboxamide | 436.47 |
| 284 | 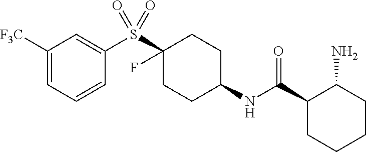 | (1R,2R)-2-amino-N-((1S,4S)- 4-fluoro-4- (3-(trifluoromethyl) phenylsulfonyl)cyclohexyl) cyclohexanecarboxamide | 450.49 |
| 285 | 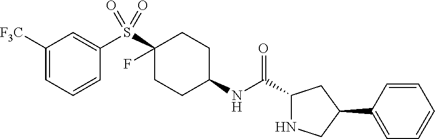 | (2S,4S)-N-((1S,4R)-4-fluoro-4-(3- (trifluoromethyl) phenylsulfonyl)cyclohexyl)-4-phenylpyrrolidine-2-carboxamide | 498.54 |
| 286 | 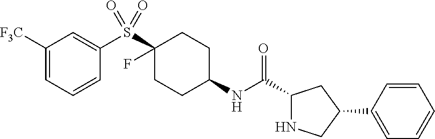 | (2S,4R)-N-((1S,4R)-4-fluoro- 4-(3-(trifluoromethyl) phenylsulfonyl)cyclohexyl)-4- phenylpyrrolidine-2- carboxamide | 498.54 |
| 287 | 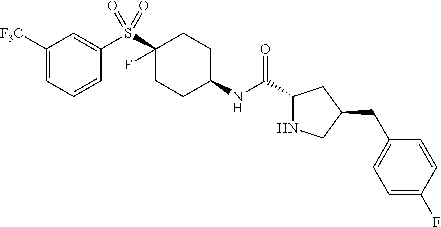 | (2S,4R)-N-((1S,4R)-4-fluoro- 4-(3-(trifluoromethyl) phenylsulfonyl)cyclohexyl)-4- (4-fluorobenzyl)pyrrolidine-2- carboxamide | 530.56 |
| 288 | 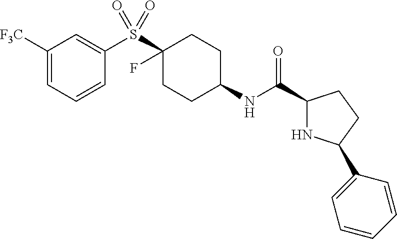 | (2R,5S)-N-((1S,4S)-4-fluoro- 4-(3-(trifluoromethyl) phenylsulfonyl)cyclohexyl)-5- phenylpyrrolidine-2- carboxamide | 498.54 |
| 289 | 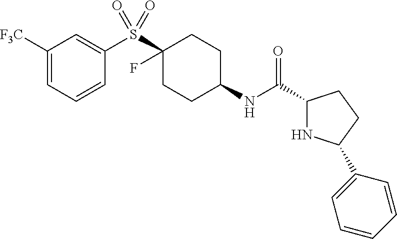 | (2S,5R)-N-((1S,4R)-4-f]uoro- 4-(3-(trifluoromethyl) phenylsulfonyl)cyclohexyl)-5- phenylpyrrolidine-2- carboxamide | 498.54 |
| 290 | 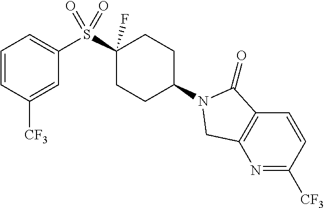 | 6-((1S,4S)-4-f1uoro-4-(3- (trifluoromethyl) phenylsulfonyl)cyclohexyl)-2- (trifluoromethyl)-6,dihydro- 5Hpyrrolo[3,4-b]pyridin-5-one | 510.43 |
| 291 | 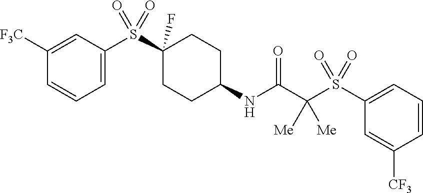 | N-((1S,4S)-4-fluoro-4-(3- (trifluoromethyl) phenylsulfonyl)cyclohexyl)-2- methyl-2-(3-(trifluoromethyl) phenylsulfonyl)propanamide | 603.57 |
| 292 | 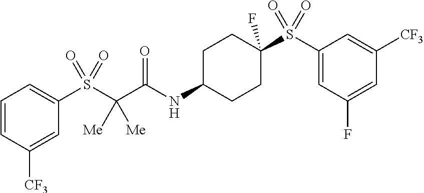 | N-((1S,4S)-4-fluoro-4-(3- fluoro-5-(trifluoromethyl) phenylsulfonyl)cyclohexyl)-2- methyl-2-(3- (trifluoromethyl)phenylsulfonyl) propanamide | 621.56 |
| 293 | 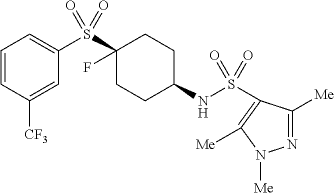 | N-((1S,4S)-4-fluoro-4-(3- (trifluoromethyl) phenylsulfonyl)cyclohexyl)- 1,3,5-trimethyl-1H-pyrazole-4- sulfonamide | 497.53 |
| 294 | 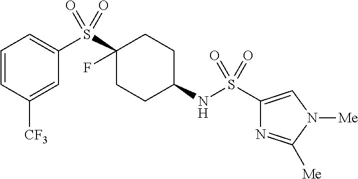 | N-((1S,4S)-4-fluoro-4-(3- (trifluoromethyl) phenylsulfonyl)cyclohexyl)- 1,2-dimethyl-1H-pyrazole-4- sulfonamide | 483.50 |
| 295 | 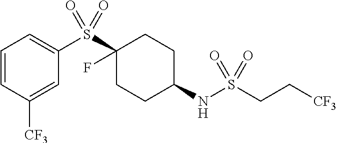 | 3,3,3-trifluoro-N-((1S,4S)-4- fluoro-4-(3-(trifluoromethyl) phenylsulfonyl)cyclohexyl) propane-1-sulfonamide | 485.44 |
| 296 | 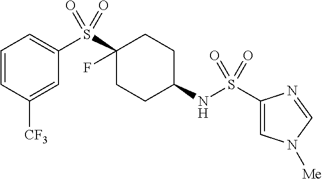 | N-((1S,4S)-4-fluoro-4-(3- (trifluoromethyl) phenylsulfonyl)cyclohexyl)-1- methyl-1H-imidazole-4- sulfonamide | 469.48 |
| 297 | 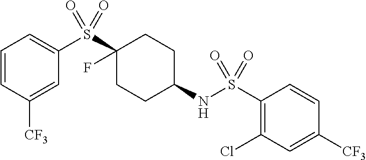 | 2-chloro-N-((1S,4S)-4-fluoro- 4-(3-(trifluoromethyl) phenylsulfonyl)cyclohexyl)-4- (trifluoromethyl) benzenesulfonamide | 567.93 |
| 298 | 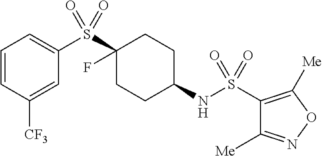 | N-((1S,4S)-4-fluoro-4-(3- (trifluoromethyl) phenylsulfonyl)cyclohexyl)- 3,5-dimethylisoxazole-4- sulfonamide | 484.49 |
| 299 | 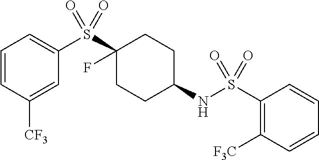 | N-((1S,4S)-4-fluoro-4-(3- (trifluoromethyl) phenylsulfonyl)cyclohexyl)-2- (trifluoromethyl) benzenesulfonamide | 533.48 |
| 300 | 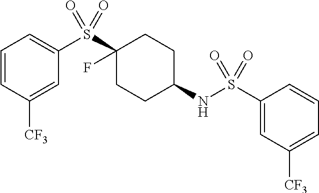 | N-((1S,4S)-4-fluoro-4-(3- (trifluoromethyl) phenylsulfonyl)cyclohexyl)-3- (trifluoromethyl) benzenesulfonamide | 533.48 |
| 301 | 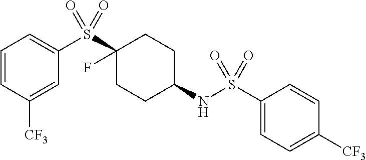 | N-((1S,4S)-4-fluoro-4-(3- (trifluoromethyl) phenylsulfonyl)cyclohexyl)-4- (trifluoromethyl) benzenesulfonamide | 533.48 |
| 302 | 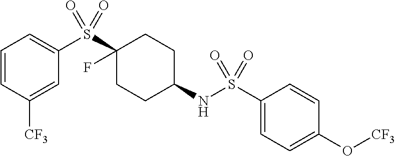 | N-((1S,4S)-4-fluoro-4-(3- (trifluoromethyl) phenylsultonyl)cyclohexyl)-4- (trifluoromethoxy) benzenesulfonamide | 549.48 |
| 303 | 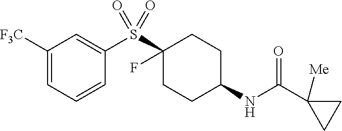 | N-((1S,4S)-4-fluoro-4-(3-(tritluoromethyl) phenylsulfonyl)cyclohexyl)-1- methylcyclopropane carboxamide | 407.43 |
| 304 | 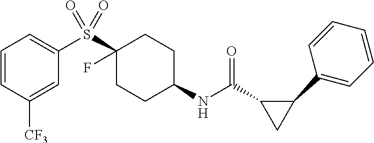 | (1S,2S)-N-((1S,4R)-4-fluoro- 4-(3-(trifluoromethyl) phenylsulfonyl)cyclohexyl)-2- phenylcyclopropane carboxamide | 469.50 |
| 305 | 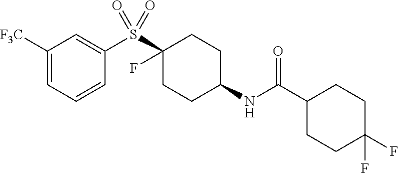 | 4,4-difluoro-N-((1S,4S)-4- fluoro-4-(3-(trifluoromethyl) phenylsulfonyl)cyclohexyl) cyclohexanecarboxamide | 471.46 |
| 306 | 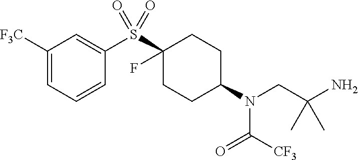 | N-(2-amino-2-methylpropyl)- 2,2,2-trifluoro-N-((1S,4S)-4- tluoro-4-(3-(trifIuoromethyl) phenylsulfonyl)cyclohexyl) acetamide | 492.45 |
| 307 | 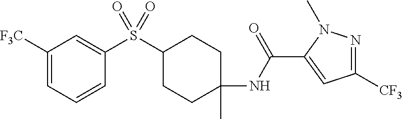 | 1-methyl-N-(1-methyl-4-(3- (trifluoromethyl) phenylsulfonyl)cyclohexyl)-3- (tritluoromethyl)-1H-pyrazole- 5-carboxamide | 497.46 |
| 308 | 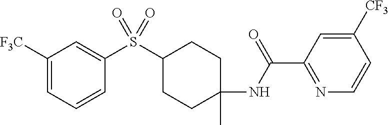 | N-(1-methyl-4-(3- (trifluoromethyl) phenylsulfonyl)cyclohexyl)-4- (trifluoromethyl)picolinamide | 494.45 |
| 309 | 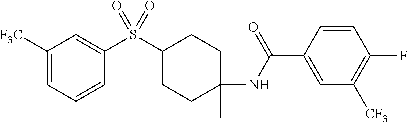 | 4-fluoro-N-(1-methyl-4-(3- (trifluoromethyl) phenylsulfonyl)cyclohexyl)-3- (trifluoromethyl)benzamide | 511.46 |
| 310 | 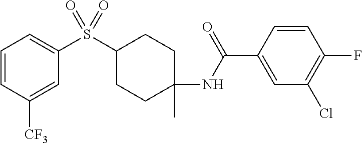 | 3-chloro-4-fluoro-N-(1- methyl-4-(3-(trifluoromethyl) phenylsulfonyl)cyclohexyl) benzamide | 477.90 |
| 311 | 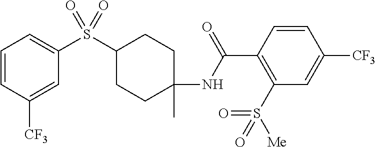 | N-(1-methyl-4-(3- (trifluoromethyl) phenylsulfonyl)cyclohexyl)-2- (methylsulfonyl)-4- (trifluoromethyl)benzamide | 571.55 |
| 312 | 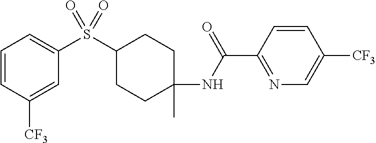 | N-(1-methyl-4-(3- (trifluoromethyl) phenylsulfonyl)cyclohexyl)-5- (trifluoromethyl)picolinamide | 494.45 |
| 313 | 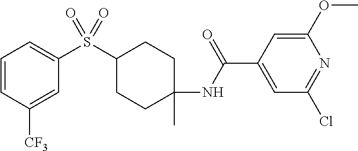 | 2-chloro-6-methoxy-N-(1- methyl-4-(3-(trifluoromethyl) phenylsulfonyl)cyclohexyl) isonicotinamide | 490.93 |
| 314 | 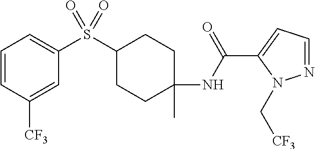 | N-(1-methyl-4-(3- (trifluoromethyl) phenylsulfonyl)cyclohexyl)-1- (2,2,2-trifluoroethyl)- 1Hpyrazole-5-carboxamide | 497.46 |
| 315 | 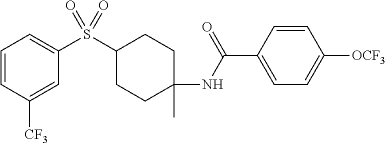 | N-(1-methyl-4-(3- (trifluoromethyl) phenylsulfonyl)cyclohexyl)-4- (trifluoromethoxy)benzamide | 509.46 |
| 316 | 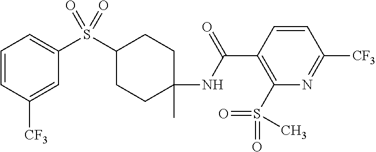 | N-(1-methyl-4-(3-(trifluoromethyl) phenylsulfonyl)cyclohexyl)-2- (methylsulfonyl)-6- (trifluoromethyl)nicotinamide | 572.54 |
| 317 | 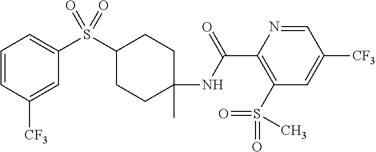 | N-(1-methyl-4-(3- (trifluoromethyl) phenylsulfonyl)cyclohexyl)-3- (methylsulfonyl)-5- (trifluoromethyl)picolinamide | 572.54 |
| 318 | 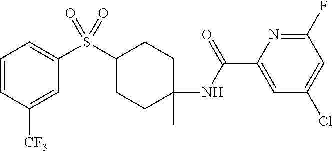 | 3-chloro-5-fluoro-N-(1- methyl-4-(3-(trifluoromethyl) phenylsulfonyl)cyclohexyl) benzamide | 477.90 |
| 319 | 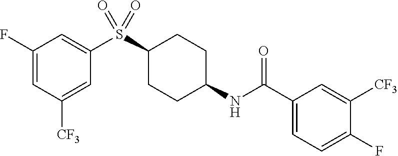 | 4-fluoro-N-((1S,4S)-4-(3- fluoro-5-(trifluoromethyl) phenylsulfonyl)cyclohexyl)-3- (trifluoromethyl)benzamide | 515.42 |
| 320 | 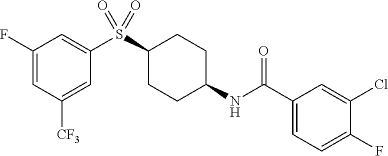 | 3-chloro-4-fluoro-N-((1S,4S)- 4-(3-fluoro-5-(trifluoromethyl) phenylsulfonyl)cyclohexyl) benzamide | 481.87 |
| 321 | 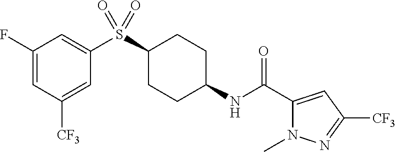 | N-((1S,4S)-4-(3-fluoro-5- (trifluoromethyl) phenylsulfonyl)cyclohexyl)-1- methyl-3-(trifluoromethyl)- 1Hpyrazole-5-carboxamide | 501.42 |
| 322 | 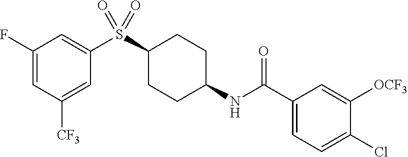 | 4-chloro-N-((1S,4S)-4-(3- fluoro-5-(trifluoromethyl) phenylsulfonyl)cyclohexyl)-3- (trifluoromethoxy)benzamide | 547.87 |
| 323 | 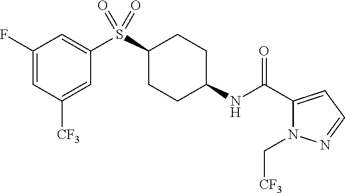 | N-((1S,4S)-4-(3-fluoro-5- (trifluoromethyl) phenylsulfonyl)cyclohexyl)-1- (2,2,2-trifluoroethyl)- 1Hpyrazole-5-carboxamide | 501.42 |
| 324 | 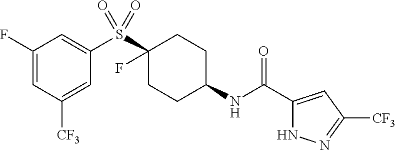 | N-((1S,4S)-4-fluoro-4-(3- fluoro-5-(trifluoromethyl) phenylsulfonyl)cyclohexyl)-3- (trifluoromethyl)-1H-pyrazole- 5-carboxamide | 505.38 |
| 325 | 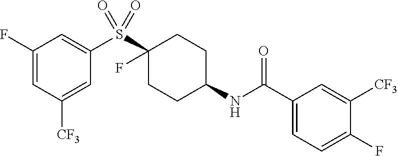 | 4-fluoro-N-((1S,4S)-4-fluoro- 4-(3-fluoro-5-(trifluoromethyl) phenylsulfonyl)cyclohexyl)-3- (trifluoromethyl)benzamide | 533.41 |
| 326 | 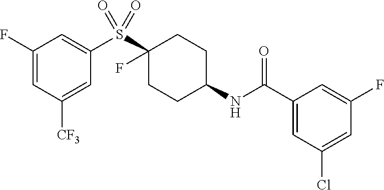 | 3-chloro-5-fluoro-N-((1S,4S)- 4-fluoro-4-(3-fluoro-5- (trifluoromethyl) phenylsulfonyl)cyclohexyl) benzamide | 499.86 |
| 327 | 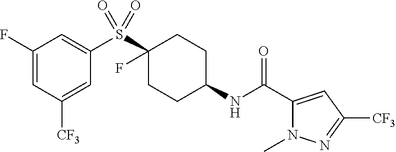 | N-((1S,4S)-4-fluoro-4-(3- fluoro-5-(trifluoromethyl) phenylsulfonyl)cyclohexyl)-1- methyl-3-(trifluoromethyl)- 1Hpyrazole-5-carboxamide | 519.41 |
| 328 | 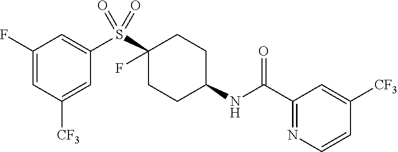 | N-((1S,4S)-4-fluoro-4-(3- fluoro-5-(trifluoromethyl) phenylsulfonyl)cyclohexyl)-4- (trifluoromethyl)picolinamide | 516.41 |
| 329 | 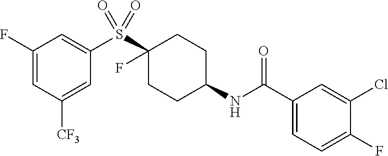 | 3-chloro-4-fluoro-N-((1S,4S)- 4-fluoro-4-(3-fluoro-5- (trifluoromethyl)phenyl sulfonyl)cyclohexyl) benzamide | 499.86 |
| 330 | 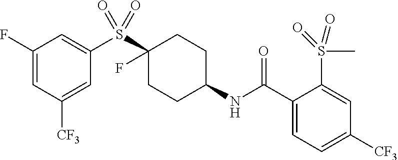 | N-((1S,4S)-4-fluoro-4-(3- fluoro-5-(trifluoromethyl) phenylsulfonyl)cyclohexyl)-2- (methylsulfonyl)-4- (trifluoromethyl)benzamide | 593.51 |
| 331 | 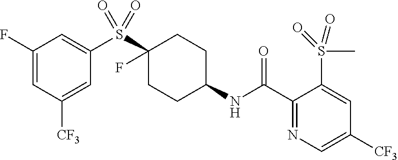 | N-((1S,4S)-4-fluoro-4-(3- fluoro-5-(trifluoromethyl) phenylsulfonyl)cyclohexyl)-3- (methylsulfonyl)-5- (trifluoromethyl)picolinamide | 594.50 |
| 332 | 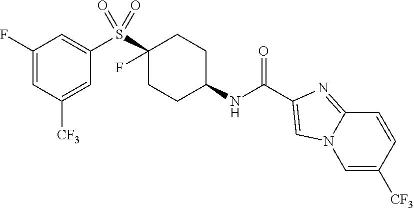 | N-((1S,4S)-4-fluoro-4-(3- fluoro-5-(trifluoromethyl) phenylsulfonyl)cyclohexyl)-6- (trifluoromethyl)imidazo[l,2- a]pyridine-2-carboxamide | 555.44 |
| 333 | 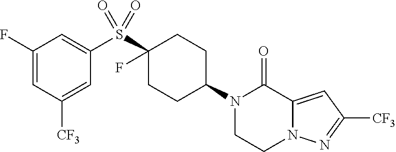 | 5-((1S,4S)-4-fluoro-4- (3-fluoro-5-(trifluoromethyl) phenylsulfonyl)cyclohexyl)-2- (trifluoromethyl)-6,7- dihydropyrazolo[l,5- a]pyrazin-4(5H)-one | 531.42 |
| 334 | 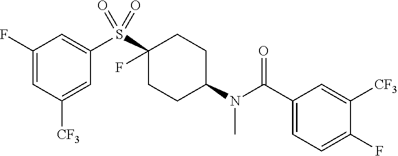 | 4-fluoro-N-((1S,4S)-4-fluoro- 4-(3-fluoro-5- (trifluoromethyl)phenyl sulfonyl)cyclohexyl)-N- methyl-3-(trifluorornethyl) benzamide | 547.44 |
| 335 | 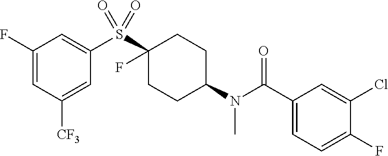 | 3-chloro-4-fluoro-N-((1S,4S)- 4-fluoro-4-(3-fluoro-5-(trifluoromethyl) phenylsulfonyl)cyclohexyl)-N- methylbenzamide | 513.88 |
| 336 | 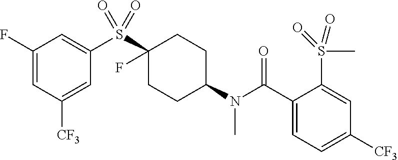 | N-((1S,4S)-4-fluoro-4-(3- fluoro-5-(trifluoromethyl) phenylsulfonyl)cyclohexyl)-N- methyl-2-(methylsulfonyl)-4- (trifluoromethyl)benzamide | 607.53 |
| 337 | 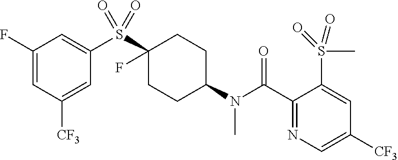 | N-((1S,4S)-4-fluoro-4-(3- fluoro-5-(trifluoromethyl) phenylsulfonyl)cyclohexyl)-N- methyl-3-(methylsulfonyl)-5- (trifluoromethyl)picolinarnide | 608.52 |
| 338 | 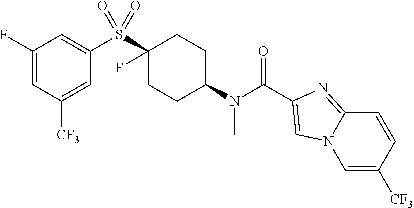 | N-((1S,4S)-4-fluoro-4-(3- fluoro-5-(trifluoromethyl) phenylsulfonyl)cyclohexyl)-N- methyl-6-(trifluoromethyl) imidazo[1,2-a]pyridine- 2-carboxamide | 569.47 |
| 339 | 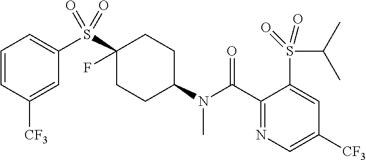 | 3-(isopropylsulfonyl)-5- (trifluoromethyl)- N-((1S,4S)-4-(3- (trifluoromethyl) phenylsulfonyl)cyclohexyl) picolinamide | 586.57 |
| 340 | 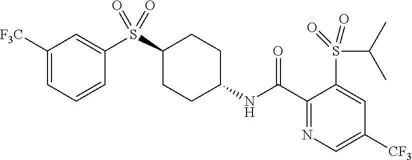 | 3-(isopropylsulfonyl)-5- (trifluoromethyl)- N-((1R,4R)-4-(3- (trifluoromethyl) phenylsulfonyl)cyclohexyl) picolinamide | 586.57 |
| 341 | 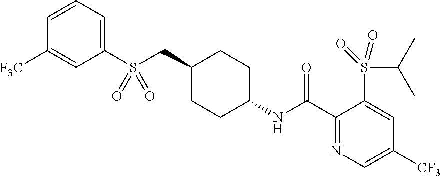 | 3-(isopropylsulfonyl)-5- (trifluoromethyl)- N-((1R,4R)-4-((3- (trifluoromethyl) phenylsulfonyl)methyl) cyclohexyl)picolinamide | 600.60 |
| 342 | 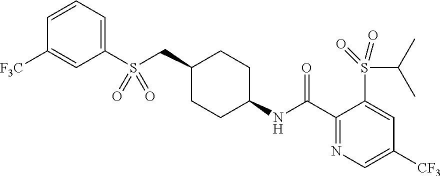 | 3-(isopropylsulfonyl)-5- (trifluoromethyl)- N-((1S,4S)-4-((3- (trifluoromethyl) phenylsulfonyl)methyl) cyclohexyl)picolinamide | 600.60 |
| 343 | 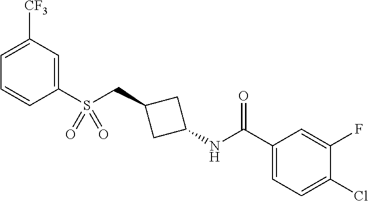 | 4-chloro-3-fluoro-N-((1R,3R)- 3-((3-(trifluoromethyl) phenylsulfonyl)methyl) cyclobutyl)benzamide | 449.85 |
| 344 | 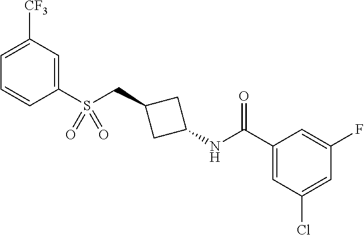 | 3-chloro-5-fluoro-N-((1R,3R)- 3-((3-(trifluoromethyl) phenylsulfonyl)methyl) cyclobutyl)benzamide | 449.85 |
| 345 | 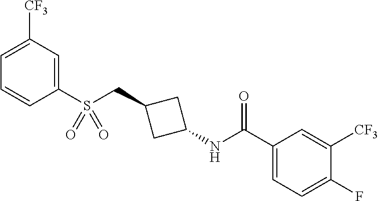 | 4-fluoro-3-(trifluoromethyl)- N-((1R,3R)-3- ((3-(trifluoromethyl) phenylsulfonyl)methyl) cyclobutyl)benzamide | 583.40 |
| 346 | 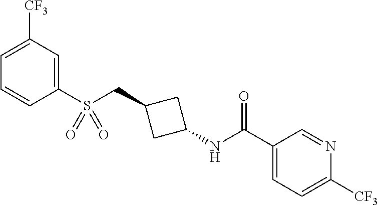 | 6-(trifluoromethyl)-N- ((1R,3R)-3-((3- (trifluoromethyl) phenylsulfonyl)methyl) cyclobutyl)nicotinamide | 466.40 |
| 347 | 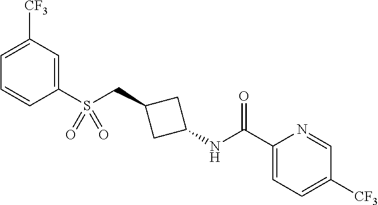 | 5-(trifluoromethyl)-N- ((1R,3R)-3-((3- (trifluoromethyl) phenylsulfonyl)methyl) cyclobutyl)picolinamide | 466.40 |
| 348 | 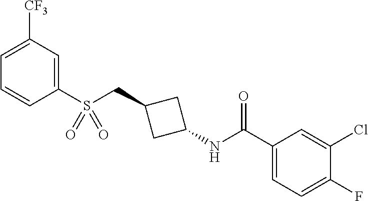 | 3-chloro-4-fluoro-N-((1R,3R)- 3-((3- (trifluoromethyl) phenylsulfonyl)methyl) cyclobutyl)benzamide | 449.85 |
| 349 | 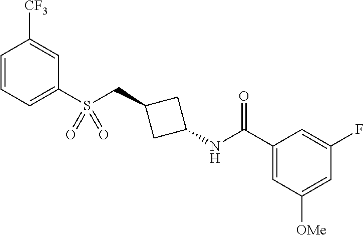 | 3-fluoro-5-methoxy-N- ((1R,3R)-3-((3- (trifluoromethyl) phenylsulfonyl)methyl) cyclobutyl)benzamide | 445.43 |
| 350 | 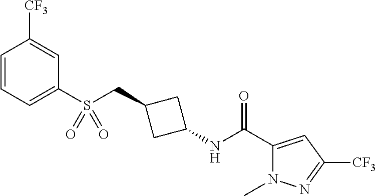 | 1-methyl-3-(trifluoromethyl)- N-((1R,3R)- 3-((3- (trifluoromethyl) phenylsulfonyl)methyl) cyclobutyl)-1H-pyrazole-5- carboxamide | 469.40 |
| 351 | 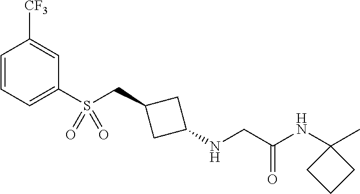 | N-(1-methylcyclobutyl)-2- ((1R,3R)-3-((3- (trifluoromethyl) phenylsulfonyl)methyl) cyclobutylamino)acetamide | 418.48 |
| 352 | 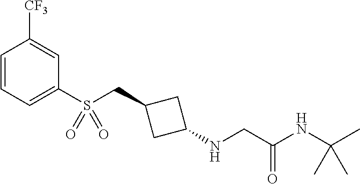 | N-tert-butyl-2-((1R,3R)-3-((3- (trifluoromethyl) phenylsulfonyl)methyl) cyclobutylamino)acetamide | 406.47 |
| 353 | 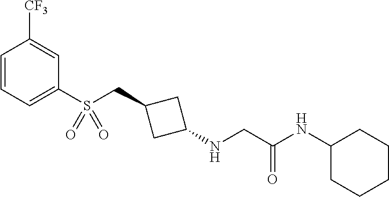 | N-cyclohexyl-2-((1R,3R)-3- ((3-(trifluoromethyl) phenylsulfonyl)methyl) cyclobutylamino)acetamide | 432.50 |
| 354 | 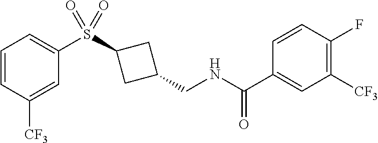 | 4-fluoro-3-(trifluoromethyl)- N-(((1R,3R)- 3-(3-(trifluoromethyl) phenylsulfonyl)cyclobut yl)methyl)benzamide | 483.40 |
| 355 | 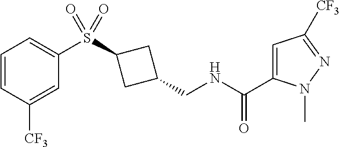 | 1-methyl-3-(trifluoromethyl)- N-(((1R,3R)- 3-(3-(trifluoromethyl) phenylsulfonyl)cyclobutyl) methyl)-1H-pyrazole-5- carboxamide | 469.40 |
| 356 | 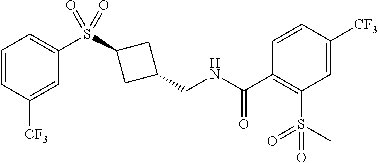 | 2-(methylsulfonyl)-4- (trifluoromethyl)-N- (((1R,3R)-3-(3- (trifluoromethyl) phenylsulfonyl)cyclobutyl) methyl)benzamide | 543.50 |
| 357 | 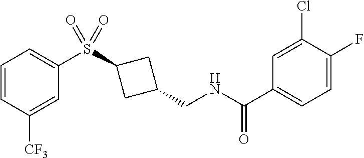 | 3-chloro-4-fluoro-N- (((1R,3R)-3-(3- (trifluorornethyl) phenylsulfonyl)cyclobutyl) methyl)benzamide | 449.85 |
| 358 | 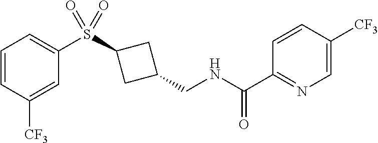 | 5-(trifluoromethyl)-N- (((1R,3R)-3-(3- (trifluoromethyl) phenylsulfonyl)cyclobutyl) methyl)picolinamide | 466.40 |
| 359 | 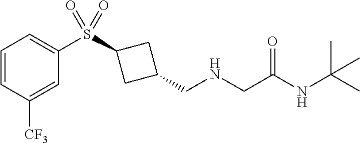 | N-tert-butyl-2-(((1R,3R)-3-(3- (trifluoromethyl) phenylsulfonyl)cyclobutyl) methylamino)acetamide | 406.47 |
| 360 | 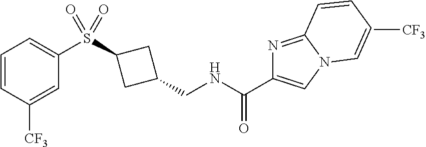 | 6-(trifluoromethyl)-N- (((1R,3R)-3-(3- (trifluoromethyl) phenylsulfonyl)cyclobutyl) methyl)imidazo[l,2-a]pyridine-2- carboxamide | 505.44 |
| 361 | 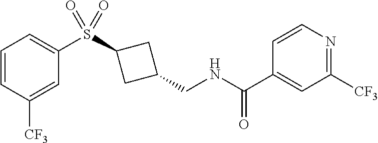 | 2-(trifluoromethyl)-N- (((1R,3R)-3-(3- (trifluoromethyl) phenylsulfonyl)cyclobutyl) methyl)isonicotinamide | 466.40 |
| 362 | 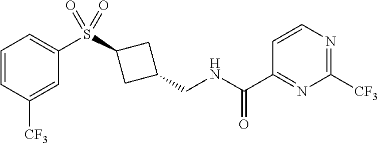 | 2-(trifluoromethyl)-N- (((1R,3R)-3-(3- (trifluoromethyl) phenylsulfonyl)cyclobutyl) methyl)pyrimidine-4- carboxamide | 467.39 |
| 363 | 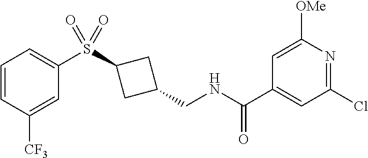 | 2-chloro-6-methoxy-N- (((1R,3R)-3-(3- (trifluoromethyl) phenylsulfonyl)cyclobutyl) methyl)isonicotinamide | 462.87 |
| 364 | 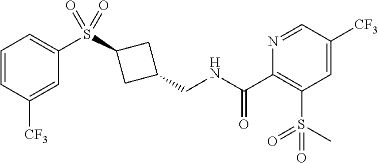 | 3-(methylsulfonyl)-5- (trifluoromethyl)-N-(((1R,3R)- 3-(3-(trifluoromethyl) phenylsulfonyl)cyclobutyl) methyl)picolinamide | 544.49 |
| 365 | 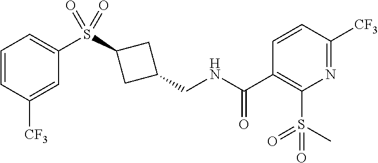 | 2-(methylsulfonyl)-6- (trifluoromethyl)-N-(((1R,3R)- 3-(3-(trifluoromethyl) phenylsulfonyl)cyclobutyl) methyl)nicotinamide | 544.49 |
| 366 | 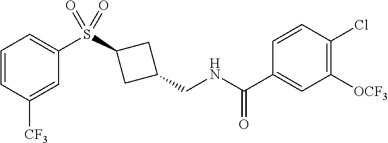 | 4-chloro-3-(trifluoromethoxy)- N-(((1R,3R)-3-(3- (trifluoromethyl) phenylsulfonyl)cyclobutyl) methyl)benzamide | 515.86 |
| 367 | 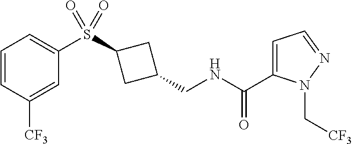 | 1-(2,2,2-trifluoroethyl)-N- (((1R,3R)-3-(3- (trifluoromethyl) phenylsulfonyl)cyclobutyl) methyl)-1H-pyrazole-5- carboxamide | 469.40 |
| 368 | 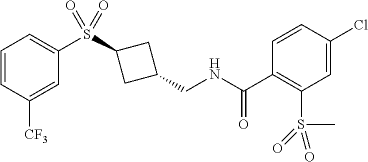 | 4-chloro-2-(methylsulfonyl)- N-(((1R,3R)3-(3- (trifluoromethyl) phenylsulfonyl)cyclobutyl) methyl)benzamide | 509.95 |
| 369 | 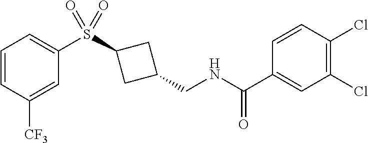 | 3,4-dichloro-N-(((1R,3R)-3-(3- (trifluoromethyl) phenylsulfonyl)cyclobutyl) methyl)benzamide | 466.30 |
| 370 | 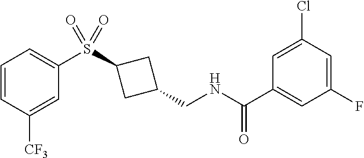 | 3-chloro-5-fluoro-N- (((1R,3R)-3-(3- (trifluoromethyl) phenylsulfonyl)cyclobutyl) methyl)benzamide | 449.85 |
| 371 | 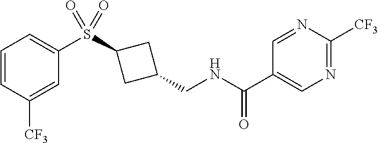 | 2-(trifluoromethyl)-N- (((1R,3R)-3-(3- (trifluoromethyl) phenylsulfonyl)cyclobutyl) methyl)pyrimidine-5- carboxamide | 467.39 |
| 372 | 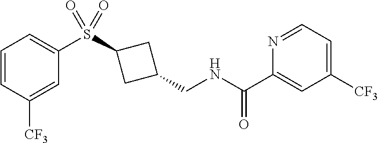 | 4-(trifluoromethyl)-N- (((1R,3R)-3-(3- (trifluoromethyl) phenylsulfonyl)cyclobutyl) methyl)picolinamide | 466.40 |
| 373 | 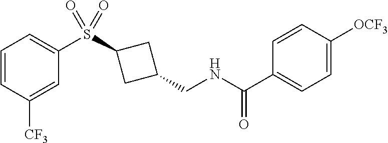 | 4-(trifluoromethoxy)-N- (((1R,3R)-3-(3- (trifluoromethyl) phenylsulfonyl)cyclobutyl) methyl)benzamide | 481.41 |
| 374 | 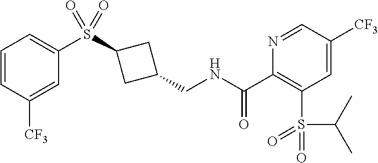 | 3-(isopropylsulfonyl)-5- (trifluoromethyl)-N-(((1R,3R)- 3-(3-(trifluoromethyl) phenylsulfonyl)cyclobutyl) methyl)picolinamide | 572.54 |
| 375 | 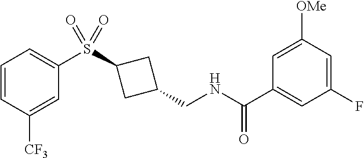 | 3-fluoro-5-methoxy-N- (((1R,3R)-3-(3- (trifluoromethyl) phenylsulfonyl)cyclobutyl) methyl)benzamide | 445.43 |
| 376 | 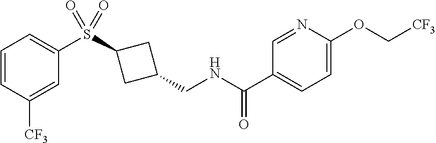 | 6-(2,2,2-trifluoroethoxy)-N- (((1R,3R)-3-(3-(trifluoromethyl) phenylsulfonyl)cyclobutyl) methyl)nicotinamide | 496.43 |
| 377 | 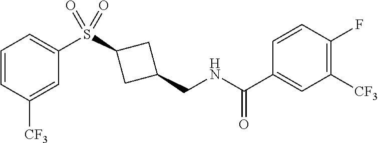 | 4-fluoro-3-(trifluoromethyl)-N- (((1S,3S)-3-(3- (trifluoromethyl) phenylsulfonyl)cyclobutyl) methyl)benzamide | 483.40 |
| 378 | 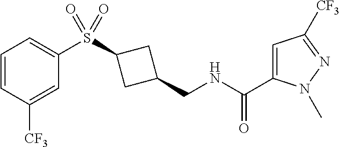 | 1-methyl-3-(trifluoromethyl)- N-(((1S,3S)-3-(3- (trifluoromethyl) phenylsulfonyl)cyclobutyl) methyl)-1H-pyrazole-5- carboxamide | 469.40 |
| 379 | 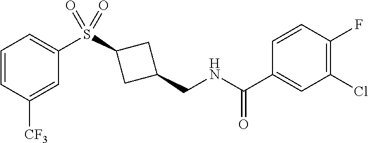 | 3-chloro-4-fluoro-N-(((1S,3S)- 3-(3-(trifluoromethyl) phenylsulfonyl)cyclobutyl) methyl)benzamide | 449.85 |
| 380 | 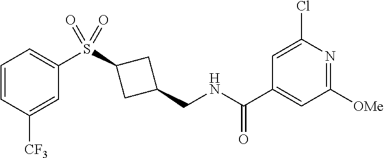 | 2-chloro-6-methoxy-N- (((1S,3S)-3-(3- (trifluoromethyl) phenylsulfonyl)cyclobutyl) methyl)isonicotinamide | 462.87 |
| 381 | 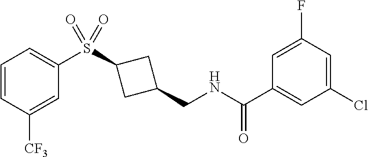 | 3-chloro-5-fluoro-N-(((1S,3S)- 3-(3-(trifluoromethyl) phenylsulfonyl)cyclobutyl) methyl)benzamide | 449.85 |
| 382 | 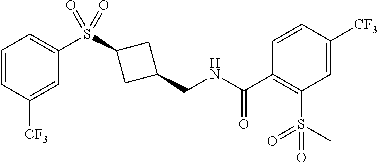 | 2-(methylsulfonyl)-6-(trifluoromethyl)- N-(((1S,3S)-3- (3-(trifluoromethyl) phenylsulfonyl)cyclobutyl) methyl)nicotinamide | 554.49 |
| 383 | 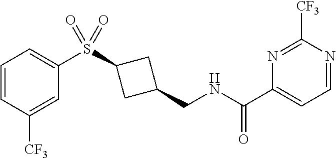 | 2-(trifluoromethyl)-N- (((1S,3S)-3-(3- (trifluoromethyl) phenylsulfonyl)cyclobutyl) methyl)pyrimidine-4- carboxamide | 467.39 |
| 384 | 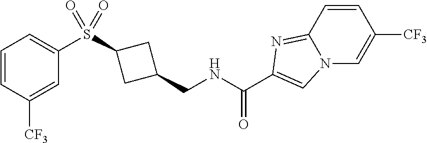 | 6-(trifluoromethyl)-N- (((1S,3S)-3-(3- (trifluoromethyl) phenylsulfonyl)cyclobutyl) methyl)imidazo[l,2-a]pyridine-2- carboxamide | 505.44 |
| 385 | 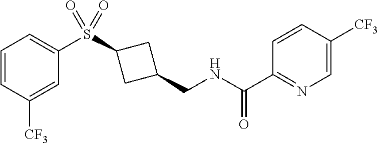 | 5-(trifluoromethyl)-N- (((1S,3S)-3-(3- (trifluoromethyl) phenylsulfonyl)cyclobutyl) methyl)picolinamide | 466.40 |
| 386 | 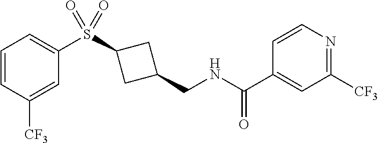 | 2-(trifluoromethyl)-N- (((1S,3S)-3-(3- (trifluoromethyl) phenylsulfonyl)cyclobutyl) methyl)isonicotinamide | 466.40 |
| 387 | 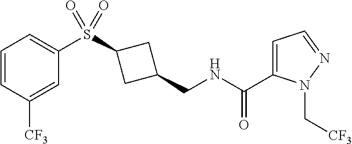 | 1-(2,2,2-trifluoroethyl)-N- (((1S,3S)-3-(3- (trifluoromethyl) phenylsulfonyl)cyclobutyl) methyl)-1H-pyrazole-5- carboxamide | 469.40 |
| 388 | 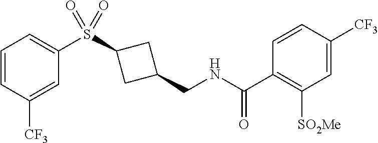 | 3-(methylsulfonyl)-5-(trifluoromethyl)- N-(((1S,3S)- 3-(3-(trifluoromethyl) phenylsulfonyl)cyclobutyl) methyl)picolinamide | 544.49 |
| 389 | 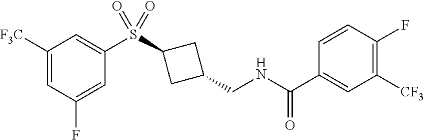 | 4-fluoro-N-(((1R,3R)-3-(3- fluoro-5-(trifluoromethyl) phenylsulfonyl)cyclobutyl) methyl)-3-(trifluoromethyl) benzamide | 501.39 |
| 390 | 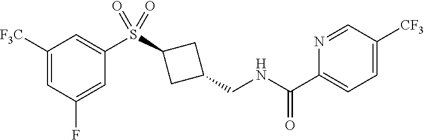 | N-(((1R,3R)-3-(3-fluoro-5- (trifluoromethyl) phenylsulfonyl)cyclobutyl) methyl)-5-(trifluoromethyl) picolinamide | 484.39 |
| 391 | 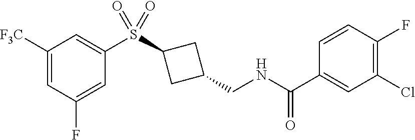 | 3-chloro-4-fluoro-N- (((1R,3R)-3-(3-fluoro-5- (trifluoromethyl) phenylsulfonyl)cyclobutyl) rnethyl)benzamide | 467.84 |
| 392 | 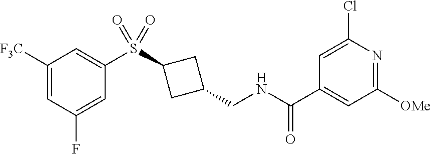 | 2-chloro-N-(((1R,3R)-3-(3- fluoro-5-(trifluoromethyl) phenylsulfonyl)cyclobutyl) rnethyl)-6-methoxy isonicotinamide | 480.86 |
| 393 | 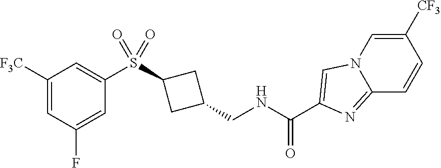 | N-(((1R,3R)-3-(3-fluoro-5- (trifluoromethyl) phenylsulfonyl)cyclobutyl) methyl)-6-(trifluoromethyl) imidazo[1,2-a]pyridine-2-carboxamide | 523.43 |
| 394 | 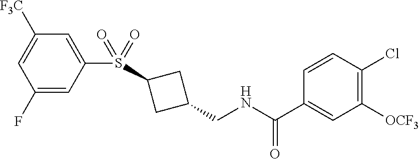 | 4-chloro-N-(((1R,3R)-3-(3- fluoro-5-(trifluoromethyl) phenylsulfonyl)cyclobutyl) methyl)-3-(trifluoromethoxy)benzamide | 533.85 |
| 395 | 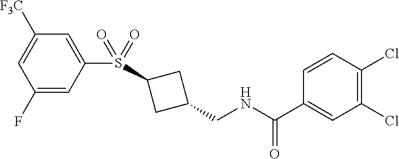 | 3,4-dichloro-N-(((1R,3R)-3-(3- fluoro-5-(trifluoromethyl) phenylsulfonyl)cyclobutyl) methyl)benzamide | 484.29 |
| 396 | 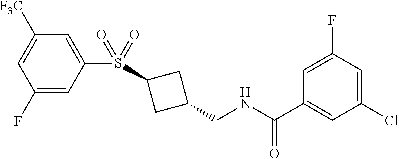 | 3-chloro-5-fluoro-N- (((1R,3R)-3-(3-fluoro-5- (trifluoromethyl) phenylsulfonyl)cyclobutyl) methyl)benzamide | 467.84 |
| 397 | 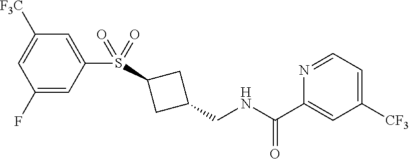 | N-(((1R,3R)-3-(3-fluoro-5- (trifluoromethyl) phenylsulfonyl)cyclobutyl) methyl)-4- (trifluoromethyl)picolinamide | 484.39 |
| 398 | 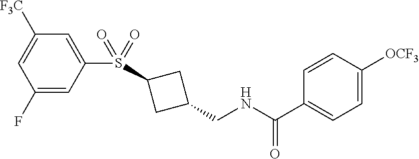 | N-(((1R,3R)-3-(3-fluoro-5- (trifluoromethyl) phenylsulfonyl)cyclobutyl) methyl)-4- (trifluoromethoxy)benzamide | 499.40 |
| 399 | 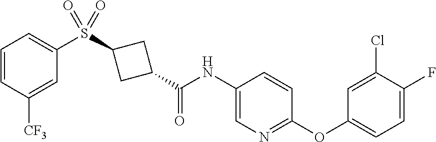 | (1R,3R)-N-(6-(3-chloro-4- fluorophenoxy)pyridin-3-yl)-3- (3-(trifluoromethyl) phenylsulfonyl)cyclobutane carboxamide | 528.91 |
| 400 | 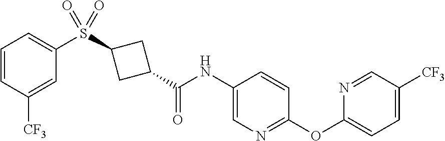 | (1R,3R)-3-(3-(trifluoromethyl) phenylsulfonyl)-N-(6-(5- (trifluoromethyl)pyridin-2- yloxy)pyridin-3-yl) cyclobutanecarboxamide | 545.46 |
| 401 | 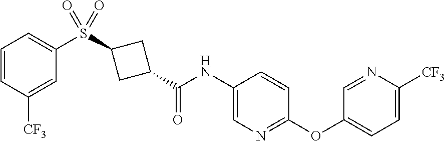 | (1R,3R)-3-(3-(trifluoromethyl) phenylsulfonyl)-N-(6-(6- (trifluoromethyl)pyridin-3- yloxy)pyridin-3-yl) cyclobutanecarboxamide | 545.46 |
| 402 | 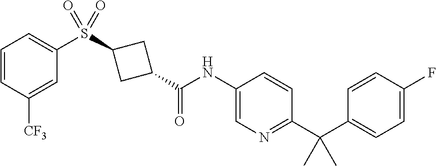 | (1R,3R)-N-(2-(4-fluorophenyl)propan- 2-yl)-3-(3-(trifluoromethyl) phenylsulfonyl)cyclobutane carboxamide | 443.46 |
| 403 | 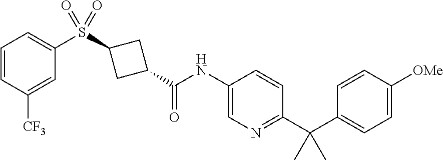 | (1R,3R)-N-(2-(4- methoxyphenyl)propan-2-yl)- 3-(3-(trifluoromethyl) phenylsulfonyl)cyclobutane carboxamide | 455.49 |
| 404 | 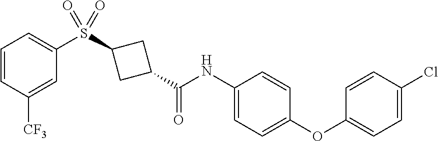 | (1R,3R)-N-(4-(4- chlorophenoxy)phenyl)-3-(3- (trifluoromethyl) phenylsulfonyl)cyclobutane carboxamide | 509.93 |
| 405 | 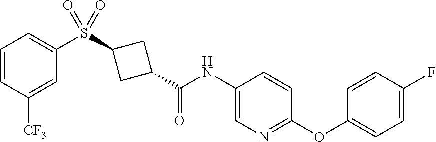 | (1R,3R)-N-(6-(4- fluorophenoxy)pyridin-3-yl)-3- (3-(trifluoromethyl) phenylsulfonyl)cyclobutane carboxamide | 494.46 |
| 406 | 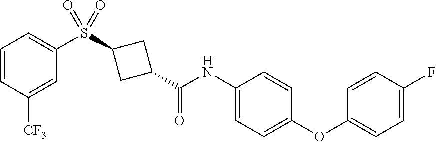 | (1R,3R)-N-(4-(4- fluorophenoxy)phenyl)-3-(3- (trifluoromethyl) phenylsulfonyl)cyclobutane carboxamide | 493.47 |
| 407 | 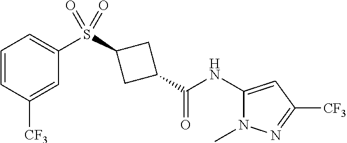 | (1R,3R)-N-(1-methyl-3- (trifluoromethyl)-1H-pyrazol- 5-yl)-3-(3-(trifluoromethyl) phenylsulfonyl) cyclobutanecarboxamide | 455.38 |
| 408 | 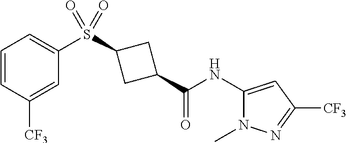 | (1S,3S)-N-(1-methyl-3- (trifluoromethyl)-1H-pyrazol- 5-yl)-3-(3-(trifluoromethyl) phenylsulfonyl)cyclobutane carboxamide | 455.38 |
| 409 | 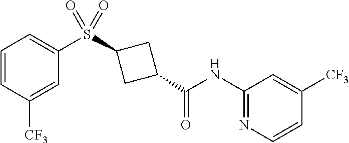 | (1R,3R)-3-(3-(trifluoromethyl) phenylsulfonyl)-N-(4- (trifluoromethyl)pyridin-2- yl)cyclobutanecarboxamide | 452.37 |
| 410 | 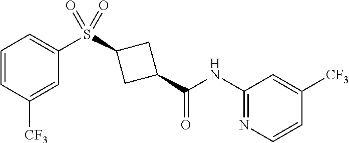 | (1S,3S)-3-(3-(trifluoromethyl) phenylsulfonyl)-N-(4- (trifluoromethyl)pyridin-2- yl)cyclobutanecarboxamide | 452.37 |
| 411 | 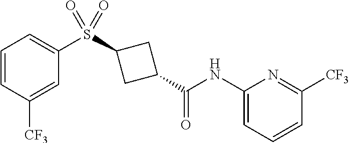 | (1R,3R)-3-(3-(trifluoromethyl) phenylsulfonyl)-N-(6- (trifluoromethyl)pyridin-2- yl)cyclobutanecarboxamide | 452.37 |
| 412 | 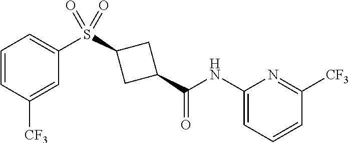 | (1S,3S)-3-(3-(trifluoromethyl) phenylsulfonyl)-N-(6-(trifluoromethyl)pyridin-2-yl)cyclobutanecarboxamide | 452.37 |
| 413 | 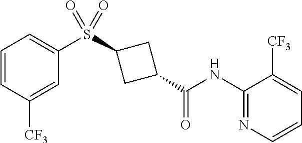 | (1R,3R)-3-(3-(trifluoromethyl) phenylsulfonyl)-N-(3-(trifluoromethyl)pyridin-2-yl)cyclobutanecarboxamide | 452.37 |
| 414 | 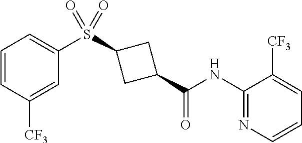 | (1S,3S)-3-(3-(trifluoromethyl) phenylsulfonyl)-N-(3-(trifluoromethyl)pyridin-2-yl)cyclobutanecarboxamide | 452.37 |
| 415 | 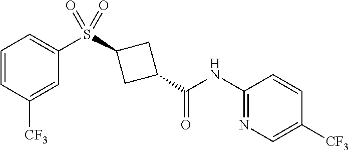 | (1R,3R)-3-(3-(trifluoromethyl) phenylsulfonyl)-N-(5-(trifluoromethyl)pyridin-2-yl)cyclobutanecarboxamide | 452.37 |
| 416 | 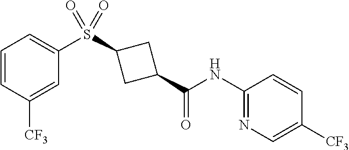 | (1S,3S)-3-(3-(trifluoromethyl) phenylsulfonyl)-N-(5-(trifluoromethyl)pyridin-2-yl)cyclobutanecarboxamide | 452.37 |
| 417 | 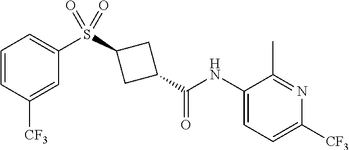 | (1R,3R)-N-(2-methyl-6- (trifluoromethyl)pyridin-3-yl)- 3-(3-(trifluoromethyl) phenylsulfonyl)cyclobutane carboxamide | 466.40 |
| 418 | 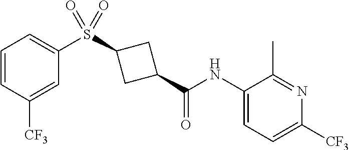 | (1S,3S)-N-(2-methyl-6- (trifluoromethyl)pyridin-3-yl)- 3-(3-(trifluoromethyl) phenylsulfonyl)cyclobutane carboxamide | 466.40 |
| 419 | 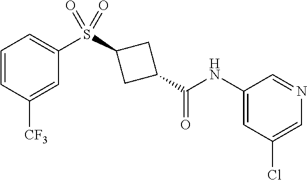 | (1R,3R)-N-(5-chloropyridin-3- yl)-3-(3-(trifluoromethyl) phenylsulfonyl)cyclobutane carboxamide | 418.82 |
| 420 | 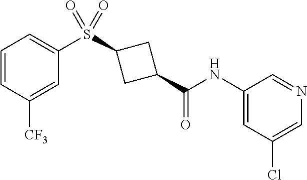 | (1S,3S)-N-(5-chloropyridin-3- yl)-3-(3-(trifluoromethyl) phenylsuIfonyl)cyclobutane carboxamide | 418.82 |
| 421 | 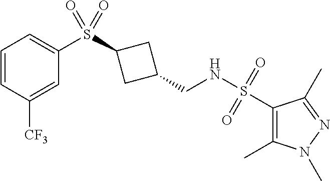 | 1,3,5-trimethyl-N-(((1R,3R)-3- (3-(trifluoromethyl) phenylsulfonyl)cyclobutyl) methyl)-1H-pyrazole-4- sulfonamide | 465.51 |
| 422 | 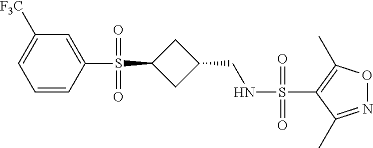 | 3,5-dimethyl-N-(((1R,3R)-3- (3-(trifluoromethyl) phenylsulfonyl)cyclobutyl) methyl)isoxazole-4- sulfonamide | 452.47 |
| 423 | 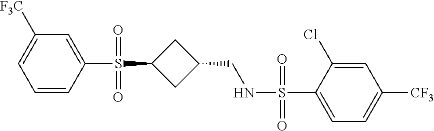 | 2-chloro-4-(trifluoromethyl)- N-(((1R,3R)-3-(3- (trifluoromethyl) phenylsulfonyl)cyclobutyl) methyl)benzenesulfonamide | 535.91 |
| 424 | 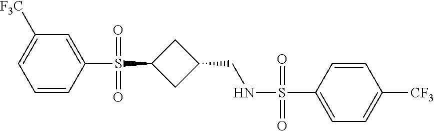 | 4-(trifluoromethyl)-N- (((1R,3R)-3-(3- (trifluoromethyl) phenylsulfonyl)cyclobutyl) methyl)benzenesulfonamide | 501.46 |
| 425 | 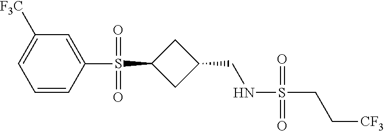 | 3,3,3-trifluoro-N-(((1R,3R)-3- (3-(trifluoromethyl) phenylsulfonyl)cyclobutyl) methyl)propane-1-sulfonamide | 453.42 |
| 426 | 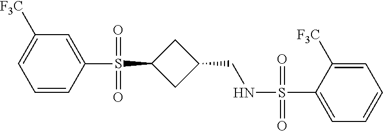 | 2-(trifluoromethyl)-N- (((1R,3R)-3-(3- (trifluoromethyl) phenylsulfonyl)cyclobutyl) methyl)benzenesulfonamide | 501.46 |
| 427 | 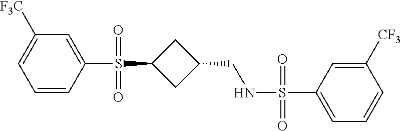 | 3-(trifluoromethyl)-N- (((1R,3R)-3-(3- (trifluoromethyl) phenylsulfonyl)cyclobutyl) methyl)benzenesulfonamide | 501.46 |
| 428 | 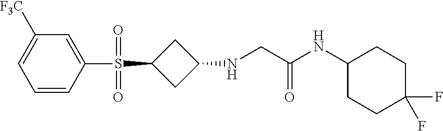 | N-(4,4-ditluorocyclohexyl)-2-((1R,3R)- 3-(3- (trifluoromethyl)phenyl sulfonyl)cyclobutylamino) acetamide | 454.46 |
| 429 | 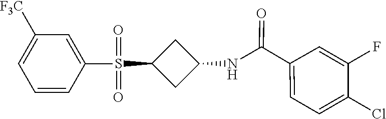 | 4-chloro-3-fluoro-N-((1S,3S)- 3-(3-(trifluoromethyl) phenylsulfonyl) cyclobutyl)benzamide | 435.82 |
| 430 | 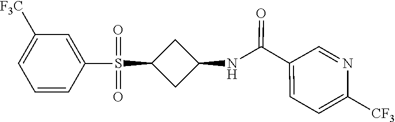 | 6-(trifluoromethyl)-N- ((1S,3S)-3-(3-(triffuoromethyl) phenylsulfonyl)cyclobutyl) nicotinamide | 452.37 |
| 431 | 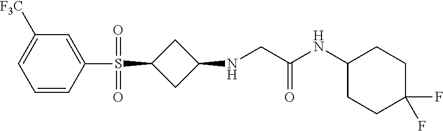 | N-(4,4-difluorocyclohexyl)-2-((1S,3S)- 3-(3-(trifluoromethyl) phenylsulfonyl)cyclobutylamino) acetamide | 454.46 |
| 432 | 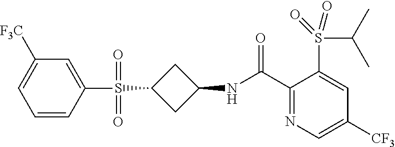 | 3-(isopropylsulfonyl)-5- (trifluoromethyl)-N-((1R,3R)-3-(3- (trifluoromethyl) phenylsulfonyl)cyclobutyl) picolinamide | 558.52 |
| 433 | 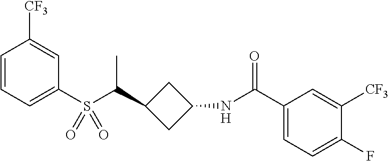 | 4-fluoro-3-(trifluoromethyl)- N-((1R,3R)-3-((R)-1-(3- (trifluoromethyl) phenylsulfonyl)ethyl) cyclobutyl)benzamide | 497.43 |
| 434 | 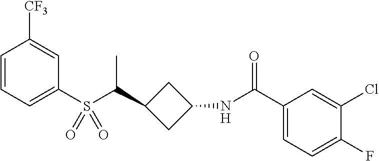 | 3-chloro-4-fluoro-N-((1R,3R)- 3-( (R)-1-(3-(trifluoromethyl) phenylsulfonyl)ethyl) cyclobutyl)benzamide | 463.88 |
| 435 | 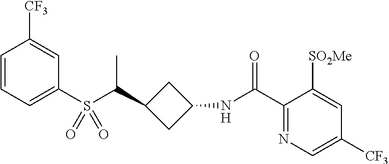 | 3-(methylsulfonyl)-5-(trifluoromethyl)- N-((1R,3R)- 3-((R)-1-(3-(trifluoromethyl) phenylsulfonyl)ethyl)cyclobutyl) picolinamide | 558.52 |
| 436 | 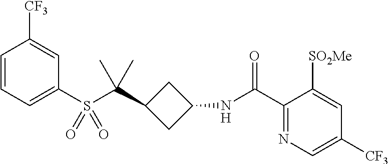 | 3-(methylsulfonyl)-5-(trifluoromethyl)- N-((1R,3R)- 3-(2-(3-(trifluoromethyl) phenylsulfonyl)propan-2- yl)cyclobutyl)picolinamide | 572.54 |
| 437 | 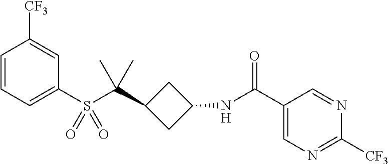 | 2-(trifluoromethyl)-N- ((1R,3R)-3-(2-(3- (trifluoromethyl) phenylsulfonyl)propan-2- yl)cyclobutyl)pyrimidine-5- carboxamide | 495.44 |
| 438 | 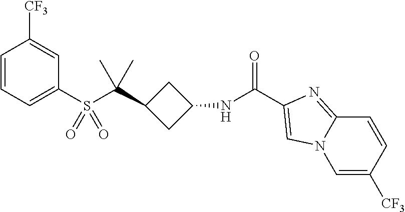 | 6-(trifluoromethyl)-N- ((1R,3R)-3-(2-(3- (trifluoromethyl) phenylsulfonyl)propan-2- yl)cyclobutyl)imidazo[1,2- a]pyridine-2-carboxamide | 533.49 |
| 439 | 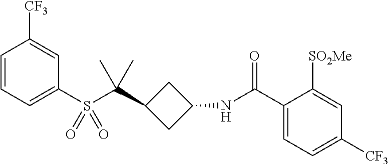 | 2-(methylsulfonyl)-4-(trifluoromethyl)- N-((1R,3R)- 3-(2-(3-(tritluoromethyl) phenylsulfonyl)propan-2- yl)cyclobutyl)benzamide | 571.55 |
| 440 | 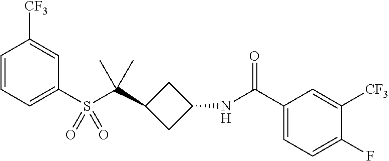 | 4-fluoro-3-(trifluoromethyl)- N-((1R,3R)-3-(2-(3- (trifluoromethyl) phenylsulfonyl)propan-2- yl)cyclobutyl)benzamide | 511.46 |
| 441 | 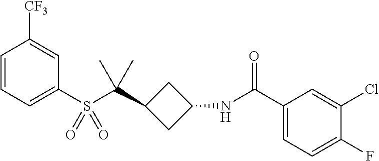 | 3-chloro-4-fluoro-N-((1R,3R)- 3-(2-(3-(trifluoromethyl) phenylsulfonyl)propan-2- yl)cyclobutyl)benzamide | 477.90 |
| 442 | 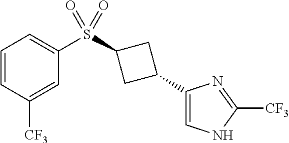 | 2-(trifluoromethyl)-4- ((1R,3R)-3-(3- (trifluoromethyl) phenylsulfonyl)cyclobutyl)- 1H-imidazole | 398.32 |
| 443 | 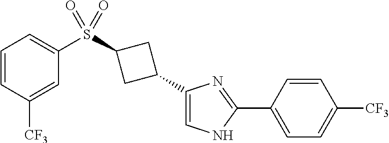 | 2-(4-(trifluoromethyl)phenyl)- 4-((1R,3R)-3-(3- (trifluoromethyl) phenylsulfonyI)cyclobutyl)- 1H-imidazole | 474.42 |
| 444 | 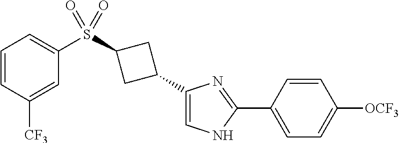 | 2-(4- (trifluoromethoxy)phenyl)-4- ((1R,3R)-3-(3- (trifluoromethyl) phenylsulfonyl)cyclobutyl)- 1H-imidazole | 490.42 |
| 445 | 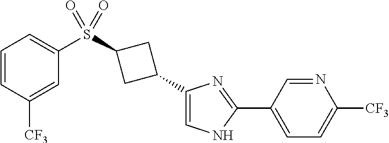 | 2-(trifluoromethyl)-5-(4- ((1R,3R)-3-(3- (trifluoromethyl) phenylsulfonyl)cyclobutyl)- 1H-imidazol-2-yl)pyridine | 475.41 |
| 446 | 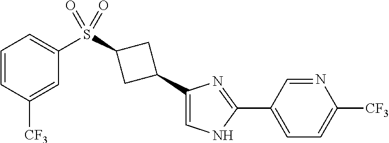 | 2-(trifluoromethyl)-5-(4- ((1S,3S)-3-(3-(trifluoromethyl) phenylsulfonyl)cyclobutyl)- 1H-imidazoI-2-yl)pyridine | 475.41 |
| 447 | 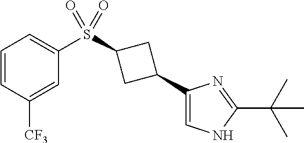 | 2-tert-butyl-4-((1S,3S)-3-(3- (trifluoromethyl) phenylsulfonyl)cyclobutyl)- 1H-imidazole | 386.44 |
| 448 | 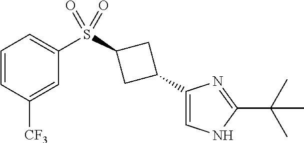 | 2-tert-butyl-4-((1R,3R)-3-(3- (trifluoromethyl) phenylsulfonyl)cyclobutyl)- 1H-imidazole | 386.44 |
| 449 | 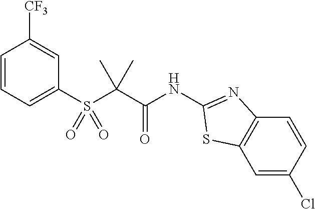 | N-(6-chlorobenzo[d]thiazol-2- yl)-2-methyl-2-(3- (trifluoromethyl) phenylsulfonyl)propanamide | 462.89 |
| 450 | 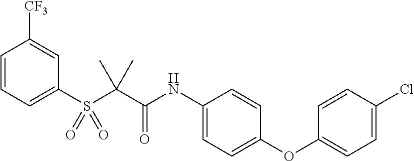 | N-(6-chlorobenzo[d]thiazol-2- yl)-2-methyl-2-(3- (trifluoromethyl) phenylsulfonyl)propanamide | 497.91 |
| 451 | 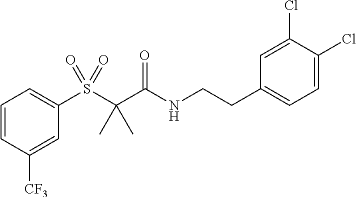 | N-(3,4-dichlorophenethyl)-2- methyl-2-(3-(trifluoromethyl) phenylsulfonyl)propanamide | 468.32 |
| 452 | 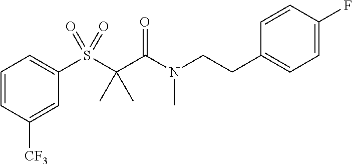 | N-(4-fluorophenethyl)-N,2- dimethyl-2-(3- (trifluoromethyl) phenylsulfonyl)propanamide | 431.44 |
| 453 | 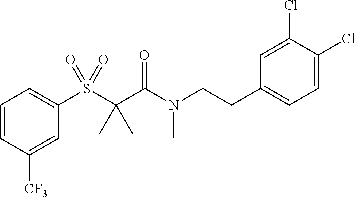 | N-(3,4-dichlorophenethyl)- N,2-dimethyl-2-(3- (trifluoromethyl) phenylsulfonyl)propanamide | 482.34 |
| 454 | 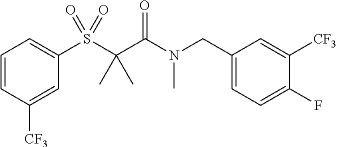 | N-(4-fluoro-3-(trifluoromethyl)benzyl)- N,2-dimethyl-2-(3- (trifluoromethyl) phenylsulfonyl)propanamide | 485.42 |
| 455 | 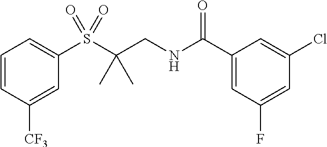 | 3-chloro-5-fluoro-N-(2- methyl-2-(3-(trifluoromethyl) phenylsulfonyl)propyl)benzamide | 437.84 |
| 456 | 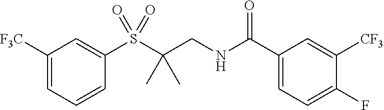 | 4-fluoro-N-(2-methyl-2-(3- (trifluoromethyl) phenylsulfonyl)propyl)-3- (trifluoromethyl)benzamide | 471.39 |
| 457 | 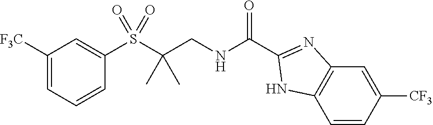 | N-(2-methyl-2-(3- (trifluoromethyl) phenylsulfonyl)propyl)-5- (trifluoromethyl)-1H- benzo[d]imidazole-2- carboxamide | 493.42 |
| 458 | 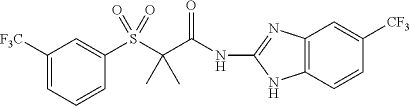 | 2-methyl-N-(5- (trifluoromethyl)-1H-benzo[d]imidazol- 2-yl)-2-(3-(trifluoromethyl) phenylsulfonyl)propanamide | 479.40 |
| 459 | 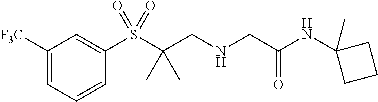 | 2-(2-methyl-2-(3- (trifluoromethyl) phenylsulfonyl)propylamino)- N-(l- methylcyclobutyl)acetamide | 406.46 |
| 460 | 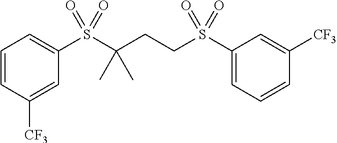 | | 488.46 |
| 461 | 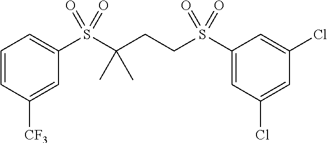 | 1,3-dichloro-5-(3-methyl-3-(3- (trifluoromethyl) phenylsulfonyl)butyl sulfonyl)benzene | 489.36 |
| 462 | 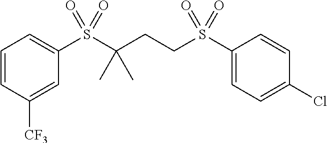 | 1-(4-(4-chlorophenylsulfonyl)- 2-methylbutan-2-ylsulfonyl)-3-(trifluoromethyl)benzene | 454.91 |
| 463 | 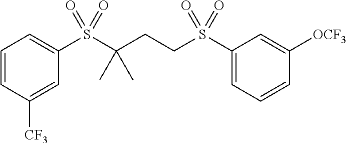 | 1-(3-methyl-3-(3- (trifluoromethyl) phenylsulfonyl)butylsulfonyl)- 3-(trifluoromethoxy)benzene | 504.46 |
| 464 | 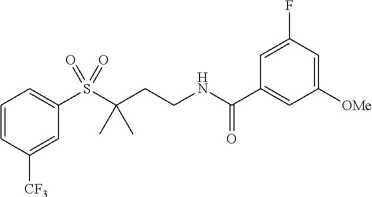 | 3-fluoro-5-methoxy-N-(3- methyl-3-(3-(trifluoromethyl) phenylsulfonyl)butyl) benzamide | 447.44 |
| 465 | 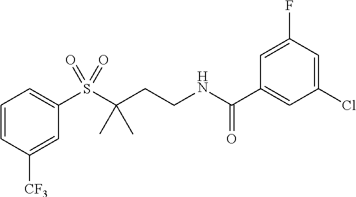 | 3-chloro-5-tluoro-N-(3- methyl-3-(3-(trifluoromethyl) phenylsulfonyl)butyl) benzamide | 451.86 |
| 466 | 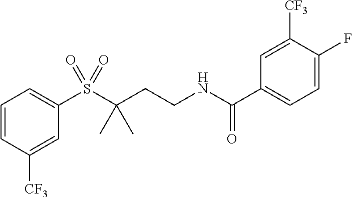 | 4-fluoro-N-(3-methyl-3-(3- (trifluoromethyl) phenylsulfonyl)butyl)-3- (trifluoromethyl)benzamide | 485.42 |
| 467 | 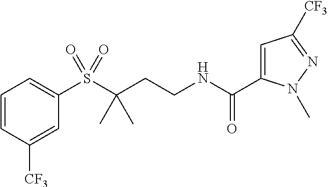 | 1-methyl-N-(3-methyl-3-(3- (trifluoromethyl) phenylsulfonyl)butyl)-3- (trifluoromethyl)-1H-pyrazole- 5-carboxamide | 471.42 |
| 468 | 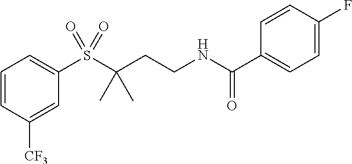 | 4-fluoro-N-(3-methyl-3-(3- (trifluoromethyl) phenylsulfonyl)butyl) benzamide | 417.42 |
| 469 | 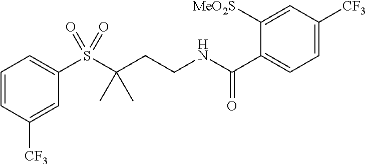 | N-(3-methyl-3-(3- (trifluoromethyl) phenylsulfonyl)butyl)-2- (methylsulfonyl)-4- (trifluoromethyl)benzamide | 545.52 |
| 470 | 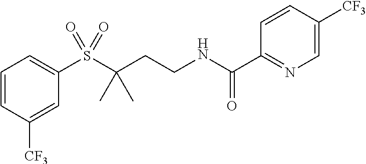 | N-(3-methyl-3-(3- (trifluoromethyl) phenylsulfonyl)butyl)-5- (trifluoromethyl)picolinamide | 468.41 |
| 471 | 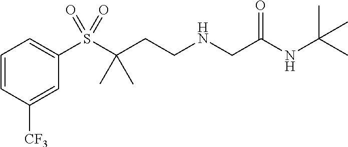 | N-tert-butyl-2-(3-methyl-3-(3- (trifluoromethyl) phenylsulfonyl)butylamino) acetamide | 408.48 |
| 472 | 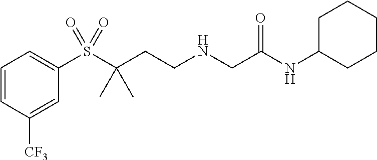 | N-cyclohexyl-2-(3-methyl-3-(3- (trifluoromethyl) phenylsulfonyl)butylamino) acetamide | 434.52 |
| 473 | 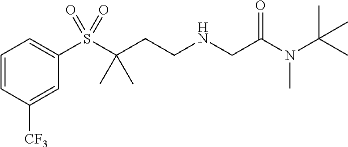 | N-tert-butyl-N-methyl-2-(3- methyl-3-(3-(trifluoromethyl) phenylsulfonyl)butylamino) acetamide | 422.51 |
| 474 | 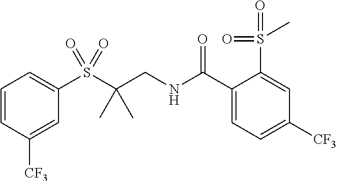 | N-(2-methyl-2-(3- (trifluoromethyl) phenylsulfonyl)propyl)-2- (methylsulfonyl)-4- (tritluoromethyl)benzamide | 531.49 |
| 475 | 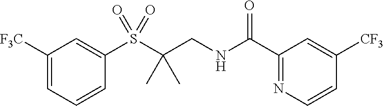 | N-(2-methyl-2-(3- (trifluoromethyl) phenylsulfonyl)propyl)-4- (trifluoromethyl)picolinamide | 454.38 |
| 476 | 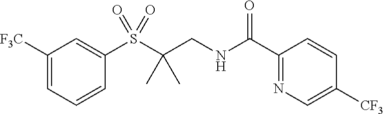 | N-(2-methyl-2-(3- (trifluoromethyl) phenylsulfonyl)propyl)-5- (trifluoromethyl)picolinamide | 454.38 |
| 477 | 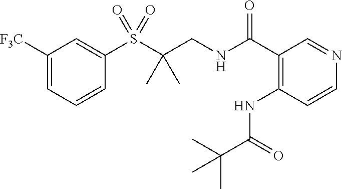 | N-(2-methyl-2-(3- (trifluoromethyl) phenylsulfonyl)propyl)-4- pivalamidonicotinamide | 485.52 |
| 478 | 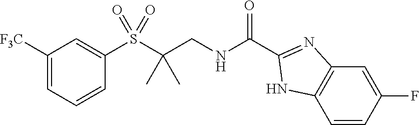 | 5-fluoro-N-(2-methyl-2-(3- (trifluoromethyl) phenylsulfonyl)propyl)-1H- benzo[d]imidazole-2- carboxamide | 443.42 |
| 479 | 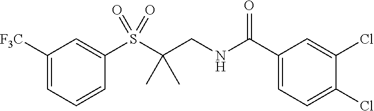 | 3,4-dichloro-N-(2-methyl-2-(3- (trifluoromethyl) phenylsulfonyl)propyl) benzamide | 454.29 |
| 480 | 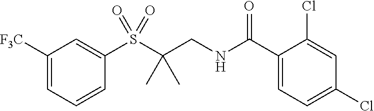 | 2,4-dichloro-N-(2-methyl-2-(3- (trifluoromethyl) phenylsulfonyl)propyl) benzamide | 454.29 |
| 481 | 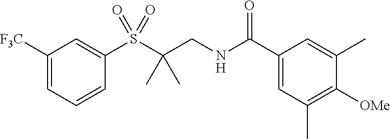 | 4-methoxy-3,5-dimethyl-N-(2- methyl-2-(3-(trifluoromethyl) phenylsulfonyl)propyl) benzamide | 443.48 |
| 482 | 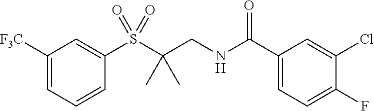 | 3-chloro-4-fluoro-N-(2- methyl-2-(3-(trifluoromethyl) phenylsulfonyl)propyl) benzamide | 437.84 |
| 483 | 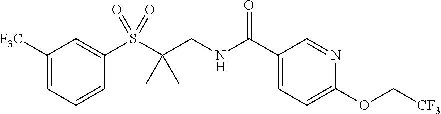 | N-(2-methyl-2-(3- (trifluoromethyl) phenylsulfonyl)propyl)-6- (2,2,2-trifluoroethoxy) nicotinamide | 484.41 |
| 484 | 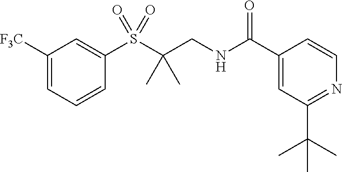 | 2-tert-butyl-N-(2-methyl-2-(3- (trifluoromethyl) phenylsulfonyl)propyl) isonicotinamide | 442.50 |
| 485 | 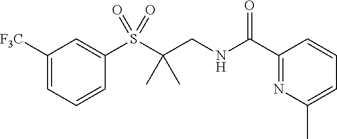 | 6-methyl-N-(2-methyl-2-(3- (trifluoromethyl) phenylsulfonyl)propyl) picolinamide | 400.42 |
| 486 | 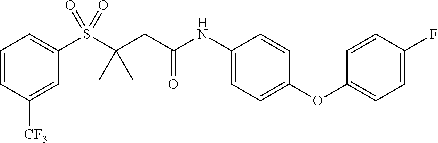 | N-(4-(4- fluorophenoxy)phenyl)-3- methyl-3-(3-(trifluoromethyl) phenylsulfonyl)butanamide | 495.49 |
| 487 | 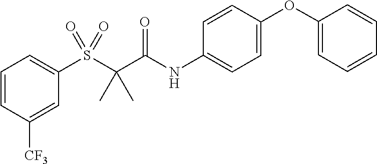 | 2-methyl-N-(4- phenoxyphenyl)-2-(3-(trifluoromethyl) phenylsulfonyl)propanamide | 463.47 |
| 488 | 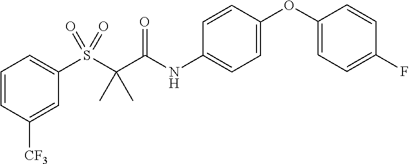 | N-(4-(4- fluorophenoxy)phenyl)-2- methyl-2-(3-(trifluoromethyl) phenylsulfonyl)propanamide | 481.46 |
| 489 | 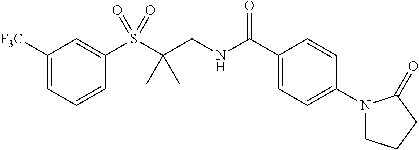 | N-(2-methyl-2-(3- (trifluoromethyl) phenylsulfonyl)propyl)-4-(2- oxopyrrolidin-1-yl)benzenesulfonamide | 504.54 |
| 490 | 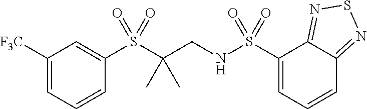 | N-(2-methyl-2-(3- (trifluoromethyl) phenylsulfonyl)propyl)benzo [c]][1,2,5]thiadiazole-4- sulfonamide | 479.52 |
| 491 | 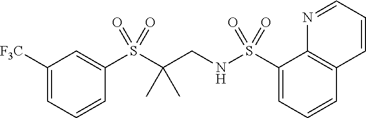 | N-(2-methyl-2-(3- (trifluoromethyl) phenylsulfonyl)propyl)quinoline- 8-sulfonamide | 472.50 |
| 492 | 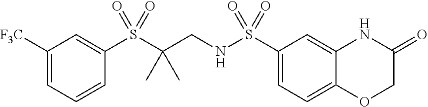 | N-(2-methyl-2-(3- (trifluoromethyl) phenylsulfonyl)propyl)-3-oxo- 3,4-dihydro-2H- benzo[b][1,4]oxazine-6- sulfonamide | 492.49 |
| 493 | 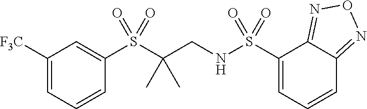 | N-(2-methyl-2-(3- (trifluoromethyl) phenylsulfonyl)propyl)benzo [c]][1,2,5]oxadiazole-4- sulfonamide | 463.45 |
| 494 | 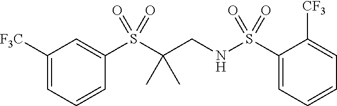 | N-(2-methyl-2-(3- (trifluoromethyl) phenylsulfonyl)propyl)-2- (trifluoromethyl) benzenesulfonamide | 489.45 |
| 495 | 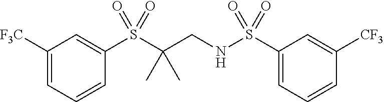 | N-(2-methyl-2-(3- (trifluoromethyl) phenylsulfonyl)propyl)-3- (trifluoromethyl) benzenesulfonamide | 489.45 |
| 496 | 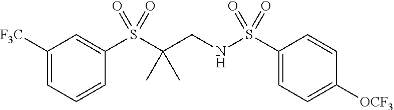 | N-(2-methyl-2-(3- (trifluoromethyl) phenylsulfonyl)propyl)-4- (trifluoromethoxy) benzenesulfonamide | 505.45 |
| 497 | 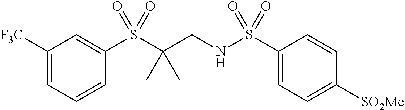 | N-(2-methyl-2-(3- (trifluoromethyl) phenylsulfonyl)propyl)-4- (methylsulfonyl) benzenesulfonamide | 499.54 |
| 498 | 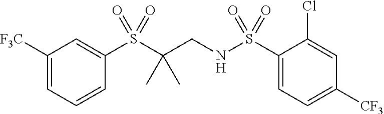 | 2-chloro-N-(2-methyl-2-(3- (trifluoromethyl) phenylsulfonyl)propyl)-4- (trifluoromethyl) benzenesulfonamide | 523.90 |
| 499 | 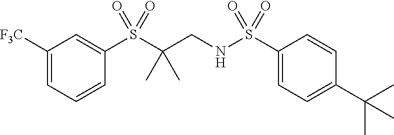 | 4-tert-butyl-N-(2-methyl-2-(3- (trifluoromethyl) phenylsulfonyl)propyl) benzenesulfonamide | 477.56 |
| 500 | 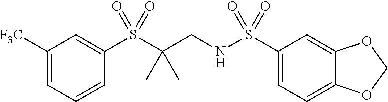 | N-(2-methyl-2-(3- (trifluoromethyl) phenylsulfonyl)propyl)benzo[d] [1,3]dioxole-5-sulfonamide | 465.46 |
| 501 | 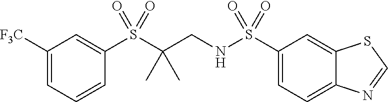 | N-(2-methyl-2-(3- (trifluoromethyl) phenylsulfonyl)propyl)benzo [d]thiazole-6-sulfonamide | 478.53 |
| 502 | 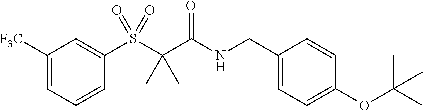 | N-(4-tert-butoxybenzyl)-2- methyl-2-(3-(trifluoromethyl) phenylsulfonyl)propanamide | 457.51 |
| 503 | 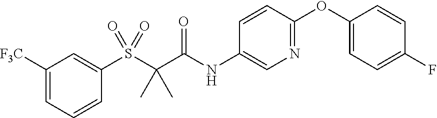 | N-(6-(4-fluorophenoxy) pyridin-3-yl)-2-methyl-2-(3- (trifluoromethyl) phenylsulfonyl)propanamide | 482.45 |
| 504 | 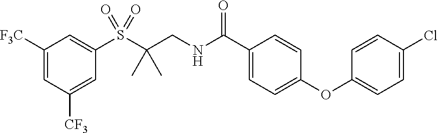 | 4-(4-chlorophenoxy)-N-(2- methyl-2-(3-(trifluoromethyl) phenylsulfonyl)propyl)benzamide | 511.94 |
| 505 | 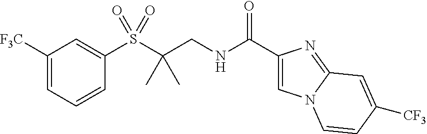 | N-(2-methyl-2-(3- (trifluoromethyl) phenylsulfonyl)propyl)-7- (trifluoromethyl)imidazo[1,2- a]pyridine-2-carboxamide | 493.42 |
| 506 | 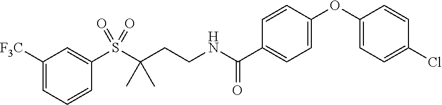 | 4-(4-chlorophenoxy)-N-(3- methyl-3-(3-(trifluoromethyl) phenylsulfonyl)butyl) benzamide | 525.97 |
| 507 | 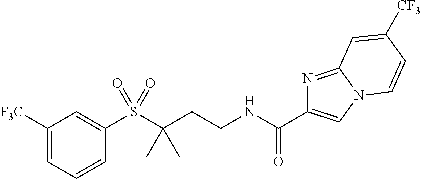 | N-(3-methyl-3-(3- (trifluoromethyl) phenylsulfonyl)butyl)-7- (trifluoromethyl)imidazo[1,2- a]pyridine-2-carboxamide | 507.45 |
| 508 | 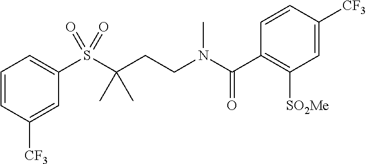 | N-methyl-N-(3-methyl-3-(3- (trifluoromethyl) phenylsulfonyl)butyl)-2- (methylsulfonyl)-4- (trifluoromethyl)benzamide | 55.54 |
| 509 | 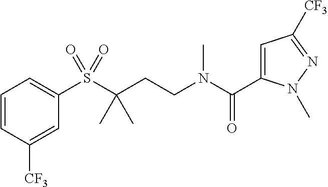 | N,1-dimethyl-N-(3-methyl-3- (3-(trifluoromethyl) phenylsulfonyl)butyl)-3- (trifluoromethyl)-1H-pyrazole- 5-carboxamide | 485.44 |
| 510 | 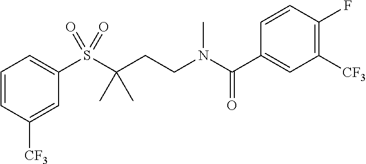 | 4-fluoro-N-methyl-N-(3- methyl-3-(3-(trifluoromethyl) phenylsulfonyl)butyl)-3- (trifluoromethyl)benzamide | 499.44 |
| 511 | 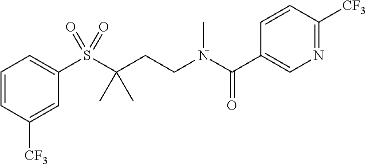 | N-methyl-N-(3-methyl-3-(3- (trifluoromethyl) phenylsulfonyl)butyl)-6- (trifluoromethyl)nicotinamide | 482.44 |
| 512 | 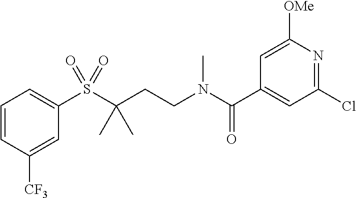 | 2-chloro-6-methoxy-N-methyl- N-(3-methyl-3-(3- (trifluoromethyl) phenylsulfonyl)butyl) isonicotinamide | 478.91 |
| 513 | 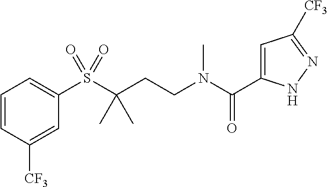 | N-methyl-N-(3-methyl-3-(3- (trifluoromethyl) phenylsulfonyl)butyl)-3- (trifluoromethyl)-1H-pyrazole- 5-carboxamide | 471.42 |
| 514 | 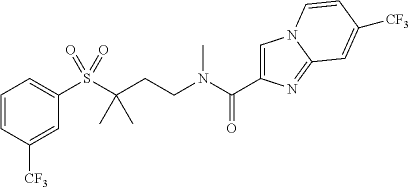 | N-methyl-N-(3-methyl-3-(3- (trifluoromethyl) phenylsulfonyl)butyl)-7- (trifluoromethyl)imidazo[1,2- a]pyridine-2-carboxamide | 521.48 |
| 515 | 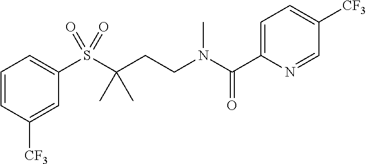 | N-methyl-N-(3-methyl-3-(3- (trifluoromethyl) phenylsulfonyl)bulyl)-5- (trifluoromethyl)picolinamide | 482.44 |
| 516 | 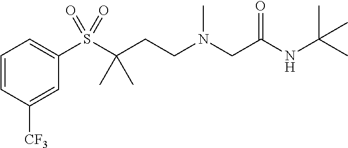 | N-tert-butyl-2-(methyl(3- methyl-3-(3-(trifluoromethyl) phenylsulfonyl)butyl) amino)acetamide | 422.51 |
| 517 | 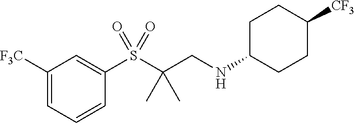 | (1R,4R)-N-(2-methyl-2-(3- (trifluoromethyl) phenylsulfonyl)propyl)-4- (trifluoromethyl)cyclo hexanamine | 431.44 |
| 518 | 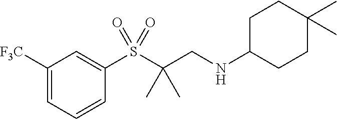 | 4,4-dimethyl-N-(2-methyl-2- (3-(trifluoromethyl) phenylsulfonyl)propyl) cyclohexanamine | 391.49 |
| 519 | 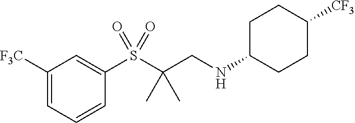 | (1S,4S)-N-(2-methyl-2-(3- (trifluoromethyl) phenylsulfonyl)propyl)-4- (trifluoromethyl) cyclohexanamine | 431.44 |
| 520 | 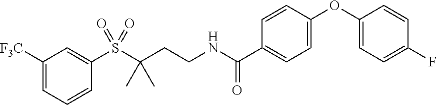 | 4-(4-fluorophenoxy)-N-(3- methyl-3-(3-(trifluoromethyl) phenylsulfonyl)butyl) benzamide | 509.51 |
| 521 | 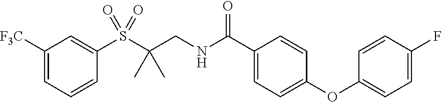 | 4-(4-fluorophenoxy)-N-(2- methyl-2-(3-(trifluoromethyl) phenylsulfonyl)propyl) benzamide | 495.49 |
| 522 | 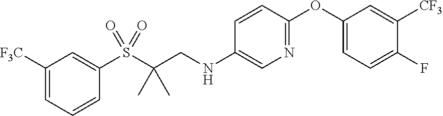 | N-(6-(4-fluoro-3- (trifluoromethyl)phenoxy) pyridin-3-yl)-2-methyl-2-(3- (trifluoromethyl) phenylsulfonyl)propanamide | 550.45 |
| 523 | 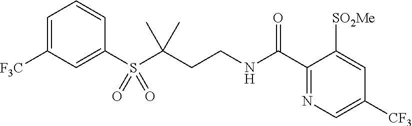 | N-(3-methyl-3-(3-(trifluoromethyl) phenylsulfonyl)butyl)-3- (methylsulfonyl)-5- (trifluorornethyl)picolinamide | 546.50 |
| 524 | 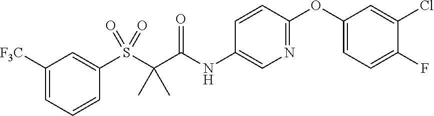 | N-(6-(3-chloro-4- tluorophenoxy)pyridin-3-yl)-2-methyl- 2-(3-(trifluoromethyl) phenylsulfonyl)propanamide | 516.80 |
| 525 | 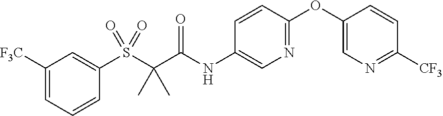 | 2-methyl-2-(3- (trifluoromethyl) phenylsulfonyl)-N-(6-(6- (trifluoromethyl)pyridin-3- yloxy)pyridin-3-yl) propanamide | 533.44 |
| 526 | 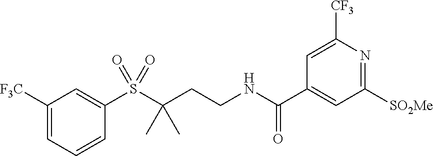 | N-(3-methyl-3-(3- (trifluoromethyl) phenylsulfonyl)butyl)-2- (methylsulfonyl)-6- (trifluoromethyl)isonicotinamide | 546.50 |
| 527 | 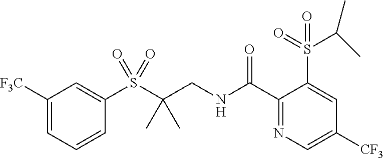 | 3-(isopropylsulfonyl)-N-(2- methyl-2-(3-(trifluoromethyl) phenylsulfonyl)propyl)-5- (trifluoromethyl)picolinamide | 560.53 |
| 528 | 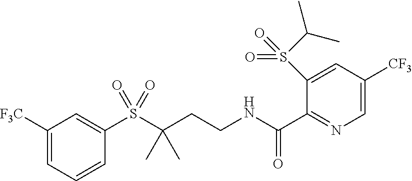 | 3-(isopropylsulfonyl)-N-(3- methyl-3-(3-(trifluoromethyl) phenylsulfonyl)butyl)-5- (trifluoromethyl)picolinamide | 574.56 |
| 529 | 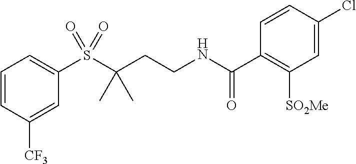 | 4-chloro-N-(3-methyl-3-(3- (trifluoromethyl) phenylsulfonyl)butyl)-2- (methylsulfonyl)benzamide | 511.96 |
| 530 | 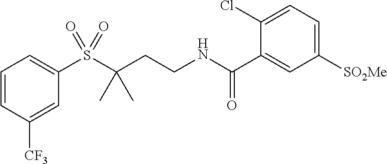 | 2-chloro-N-(3-methyl-3-(3- (trifluoromethyl) phenylsulfonyl)butyl)-5- (methylsulfonyl)benzamide | 511.96 |
| 531 | 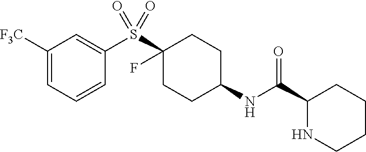 | (R)-N-((1S,4S)-4-fluoro-4-(3- (trifluoromethyl) phenylsulfonyl)cyclohexyl) piperidine-2-carboxamide | 436.46 |
| 532 | 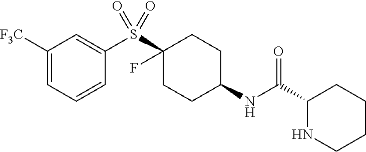 | (S)-N-((1S,4R)-4-fluoro-4-(3- (trifluoromethyl) phenylsulfonyl)cyclohexyl) piperidine-2-carboxamide | 436.46 |
| 533 | 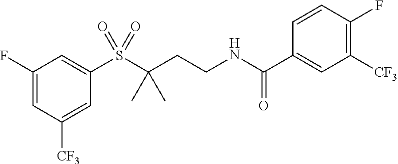 | 4-fluoro-N-(3-(3-fluoro-5- (trifluoromethyl) phenylsulfonyl)-3- methylbutyl)-3- (trifluoromethyl)benzamide | 503.41 |
| 534 | 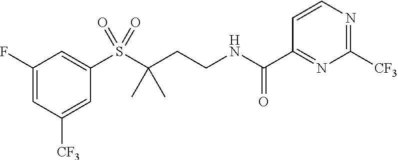 | N-(3-(3-fluoro-5- (trifluoromethyl) phenylsulfonyl)-3- methylbutyl)-2- (trifluoromethyl)pyrimidine-4- carboxamide | 487.39 |
| 535 | 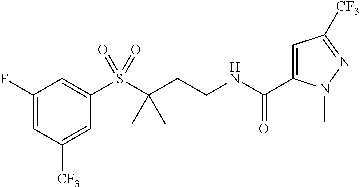 | N-(3-(3-fluoro-5- (trifluoromethyl) phenylsulfonyl)-3- methylbutyl)-l-methyl-3- (trifluoromcthyl)-1H-pyrazole- 5-carboxamide | 489.41 |
| 536 | 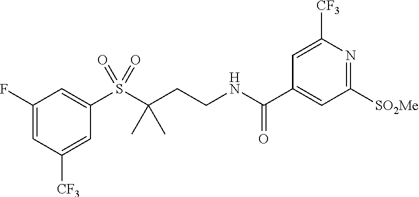 | N-(3-(3-fluoro-5- (trifluoromethyl) phenylsulfonyl)-3- methylbutyl)-2- (methylsulfonyl)-6- (trifluoromethyl) isonicotinamide | 564.49 |
| 537 | 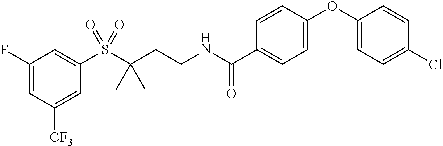 | 4-(4-chlorophenoxy)-N-(3-(3- fluoro-5-(trifluoromethyl) phenylsulfonyl)-3- methylbutyl)benzamide | 543.96 |
| 538 | 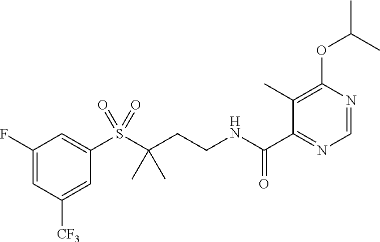 | 6-isopropoxy-5-methyl-N-(3- methyl-3-(3-(trifluoromethyl) phenylsulfonyl)butyl)pyrimidine- 4-carboxamide | 473.51 |
| 539 | 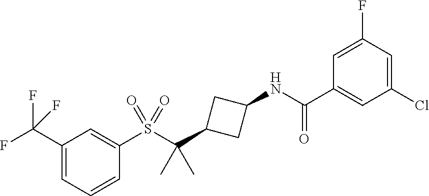 | | |
| 540 | 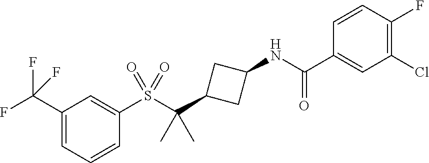 | | |
| 541 | 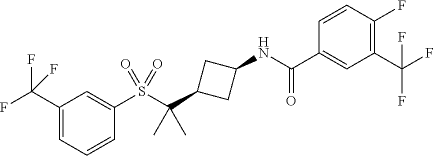 | | |
| 542 | 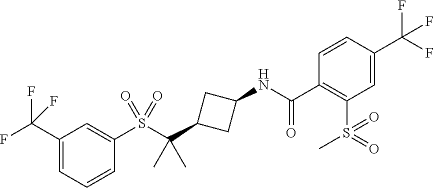 | | |
| 543 | 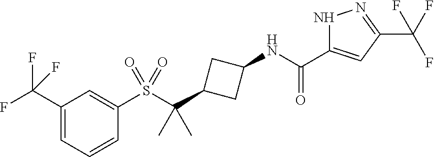 | | |
| 544 | 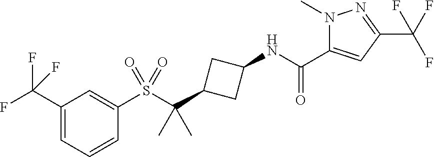 | | |
| 545 | 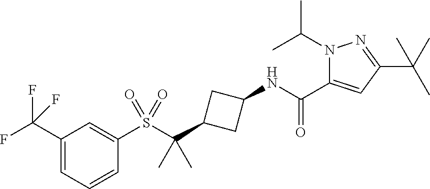 | | |
| 546 | 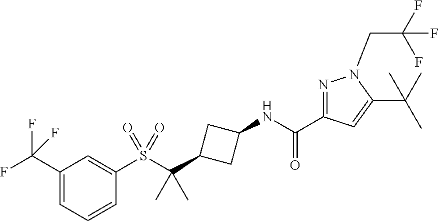 | | |
| 547 | 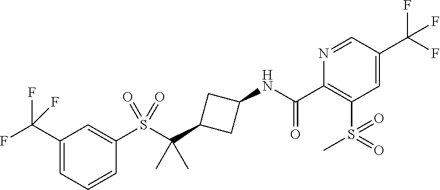 | | |
| 548 | 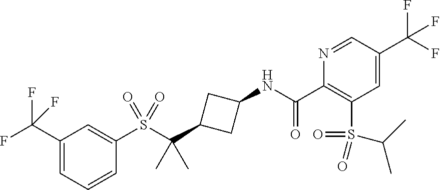 | | |
| 549 | 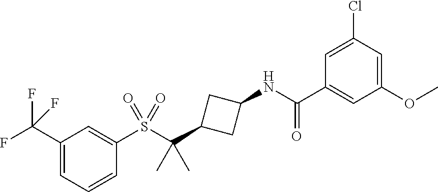 | | |
| 550 | 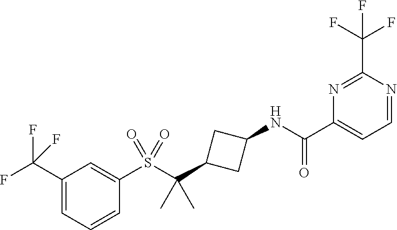 | | |
| 551 | 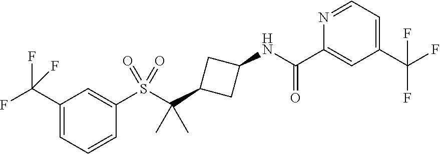 | | |
| 552 | 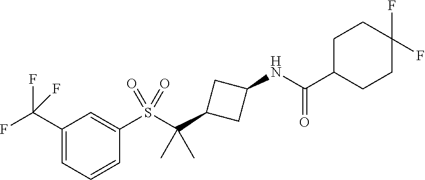 | | |
| 553 | 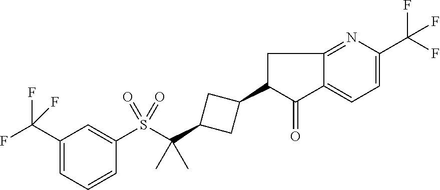 | | |
| 554 | 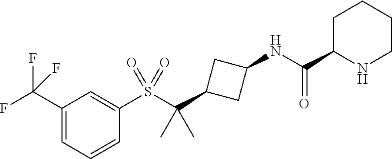 | | |
| 555 | 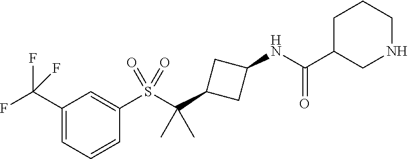 | | |
| 556 | 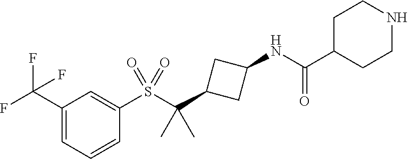 | | |
| 557 | 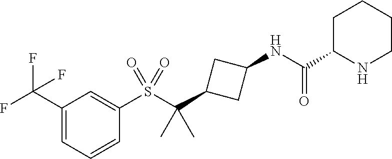 | | |
| 558 | 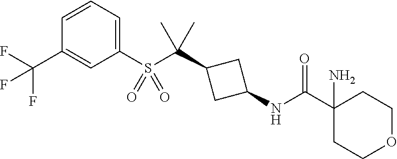 | | |
| 559 | 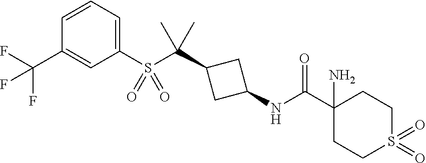 | | |
| 560 | 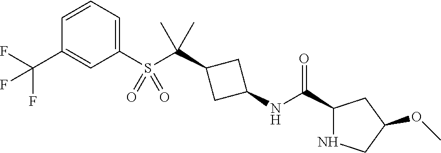 | | |
| 561 | 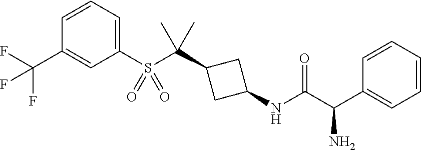 | | |
| 562 | 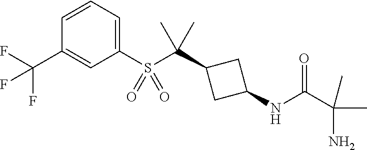 | | |
| 563 | 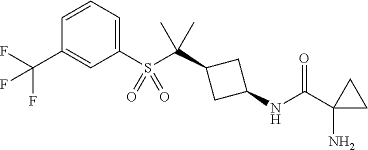 | | |
| 564 | 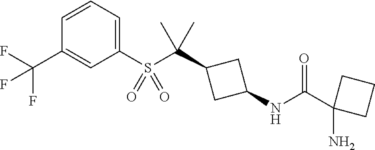 | | |
| 565 | 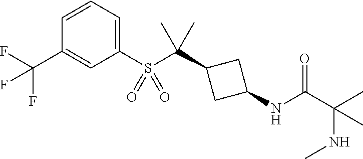 | | |
| 566 | 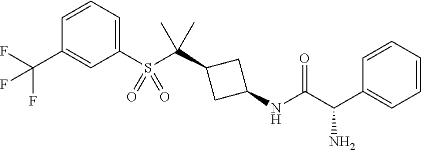 | | |
| 567 | 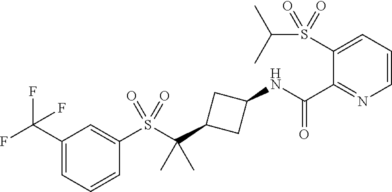 | | |
| 568 | 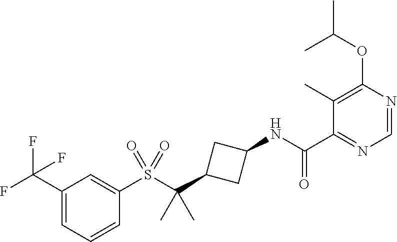 | | |
| 569 | 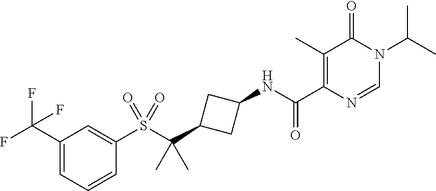 | | |
| 570 | 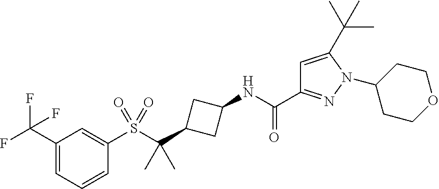 | | |
| 571 | 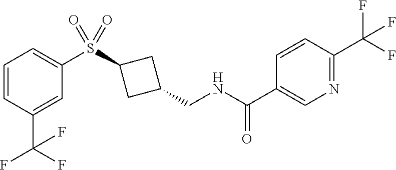 | | |
| 572 | 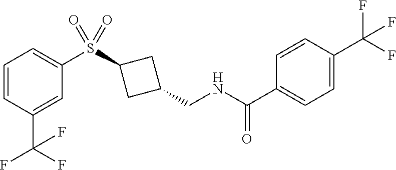 | | |
| 573 | 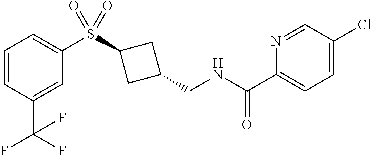 | | |
| 574 | 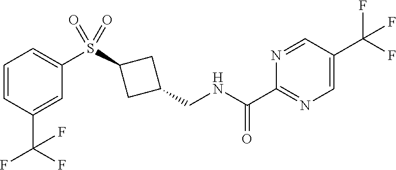 | | |
| 575 | 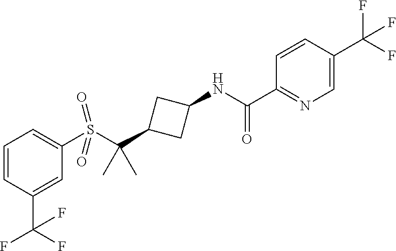 | | |
| 576 | 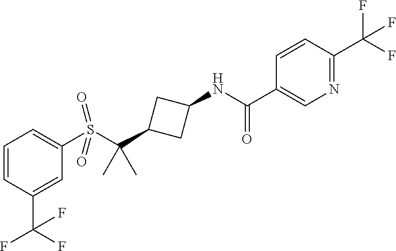 | | |
| 577 | 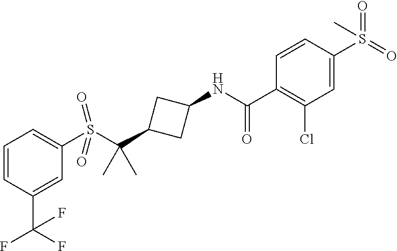 | | |
| 578 | 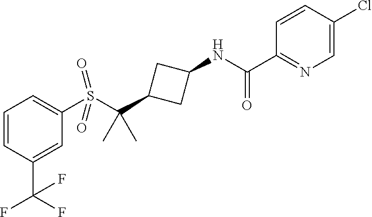 | | |
| 579 | 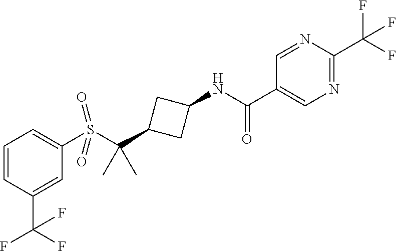 | | |
| 580 | 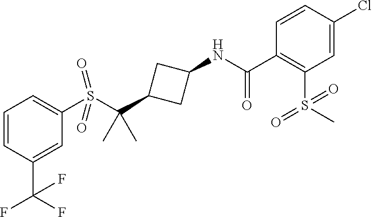 | | |
| 581 | 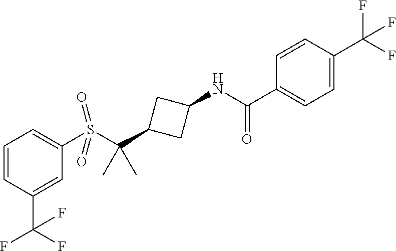 | | |
| 582 | 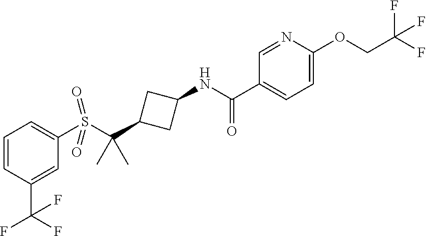 | | |
| 583 | 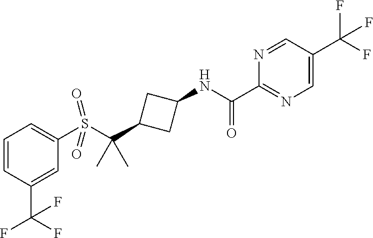 | | |
| 584 | 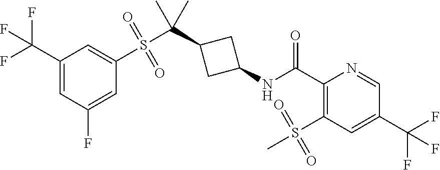 | | |
| 585 | 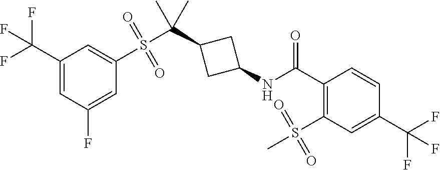 | | |
| 586 | 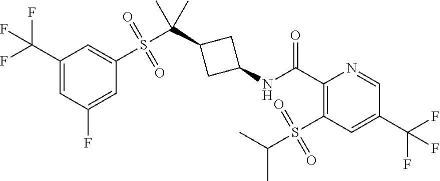 | | |
| 587 | 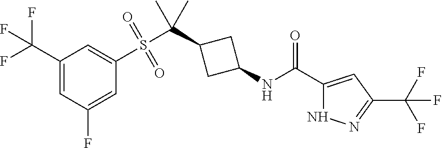 | | |
| 588 | 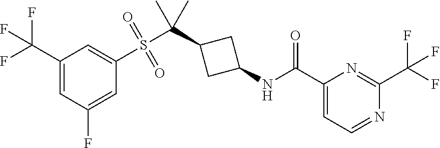 | | |
| 589 | 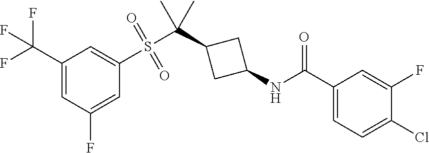 | | |
| 590 | 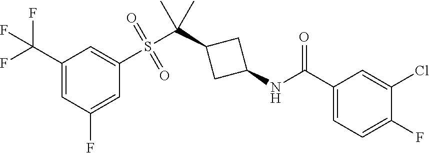 | | |
| 591 | 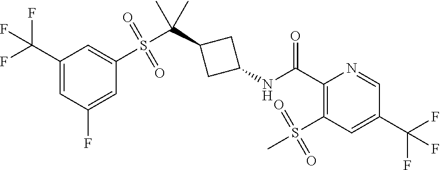 | | |
| 592 | 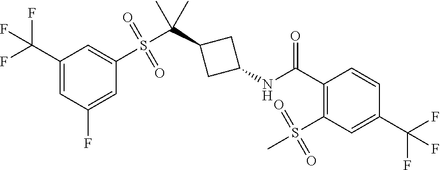 | | |
| 593 | 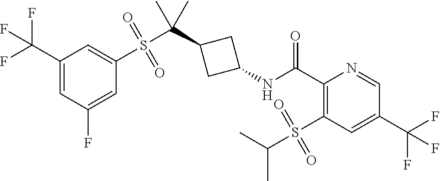 | | |
| 594 | 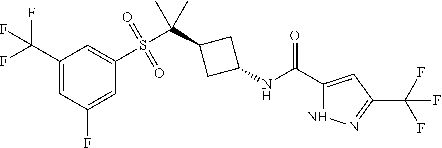 | | |
| 595 | 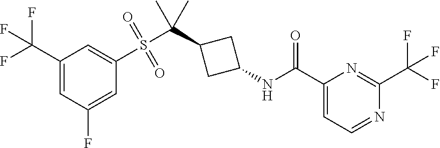 | | |
| 596 | 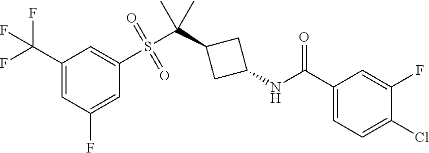 | | |
| 597 | 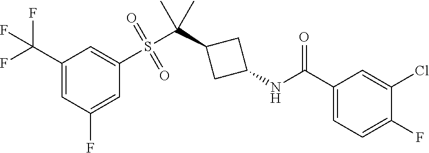 | | |
| 598 | 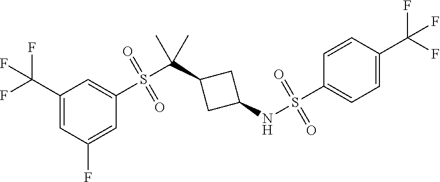 | | |
| 599 | 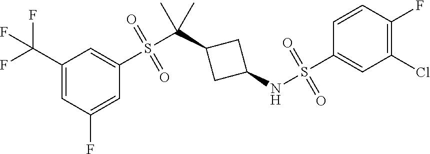 | | |
| 600 | 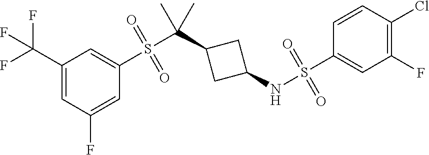 | | |
| 601 | 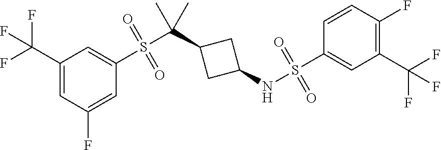 | | |
| 602 | 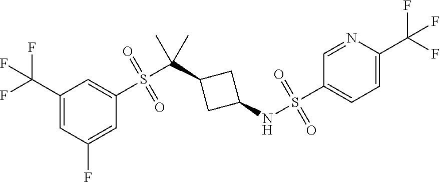 | | |
| 603 | 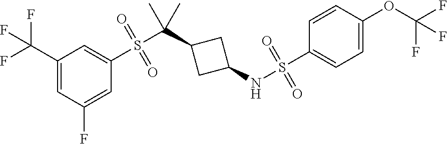 | | |
| 604 | 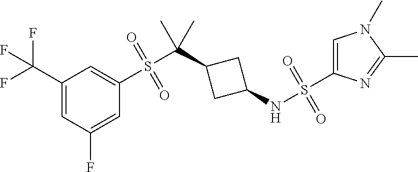 | | |
| 605 | 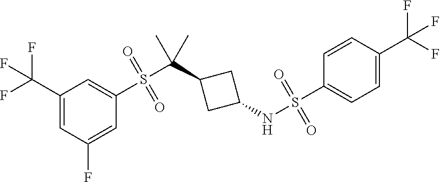 | | |
| 606 | 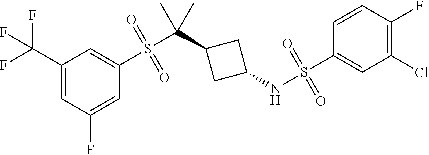 | | |
| 607 | 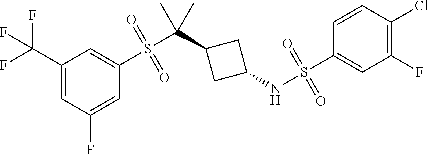 | | |
| 608 | 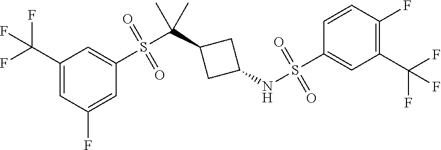 | | |
| 609 | 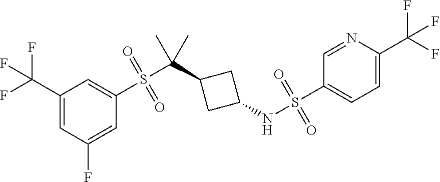 | | |
| 610 | 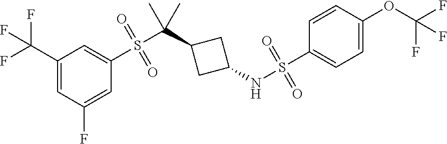 | | |
| 611 | 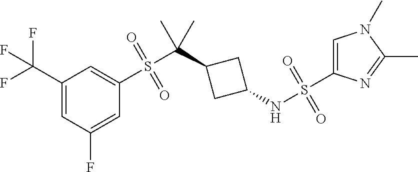 | | |
| 612 | 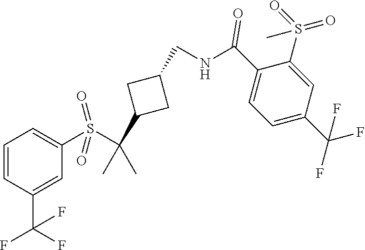 | | |
| 613 | 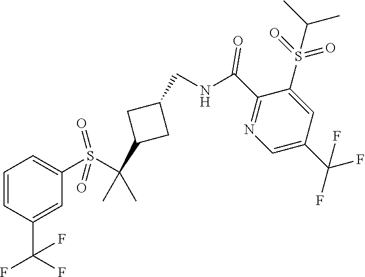 | | |
| 614 | 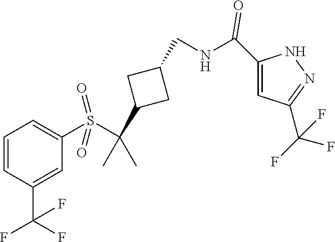 | | |
| 615 | 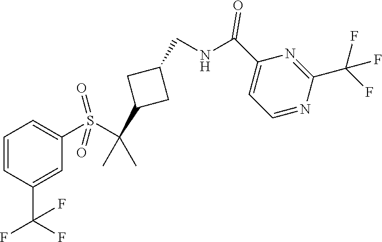 | | |
| 616 | 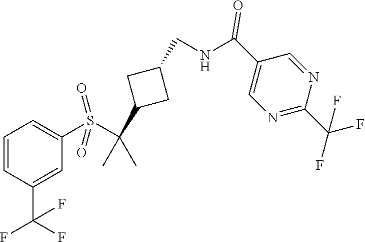 | | |
| 617 | 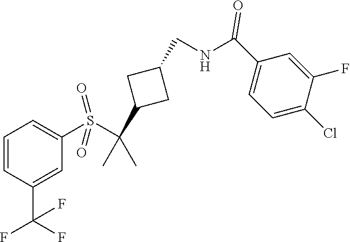 | | |
| 618 | 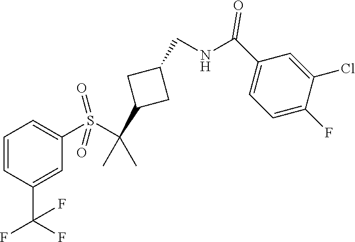 | | |
| 619 | 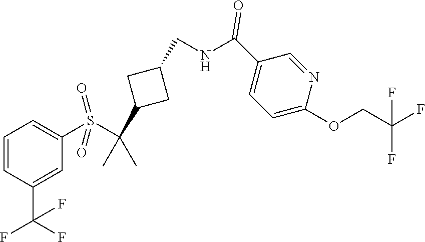 | | |
| 620 | 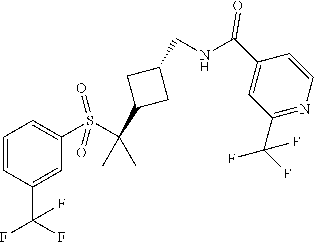 | | |
| 621 | 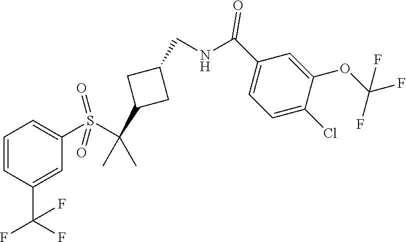 | | |
| 622 | 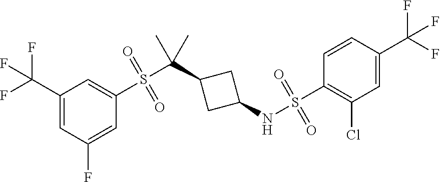 | | |
| 623 | 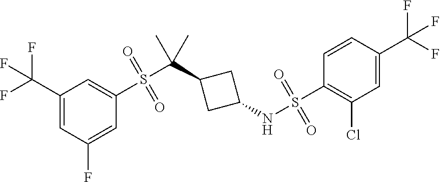 | | |
| 624 | 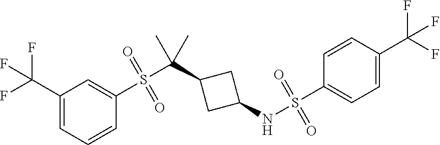 | | |
| 625 | 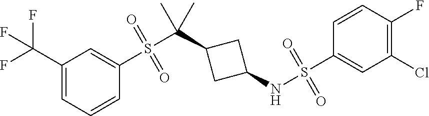 | | |
| 626 | 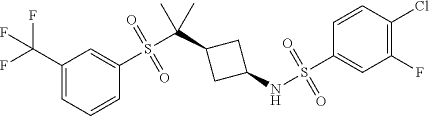 | | |
| 627 | 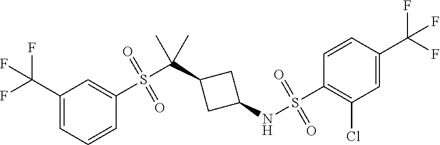 | | |
| 628 | 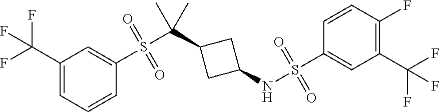 | | |
| 629 | 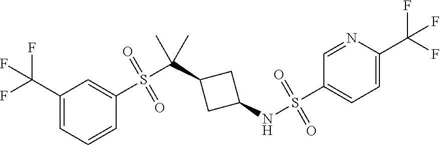 | | |
| 630 | 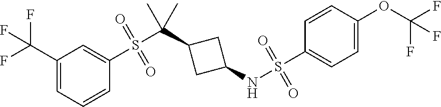 | | |
| 631 | 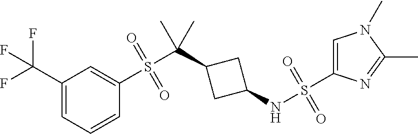 | | |
| 632 | 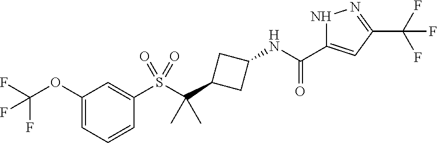 | | |
| 633 | 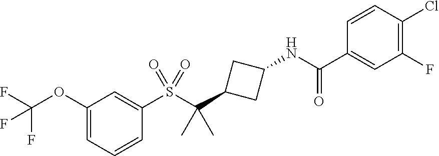 | | |
| 634 | 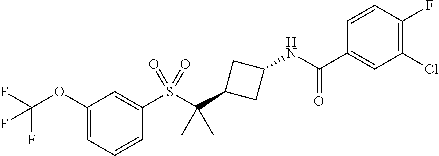 | | |
| 635 | 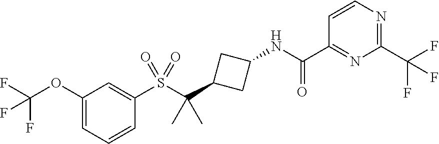 | | |
| 636 | 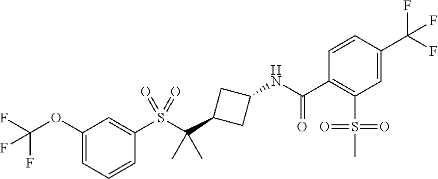 | | |
| 637 | 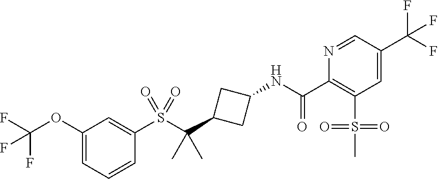 | | |
| 638 | 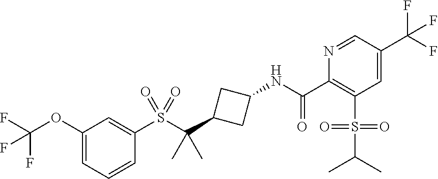 | | |
| 639 | 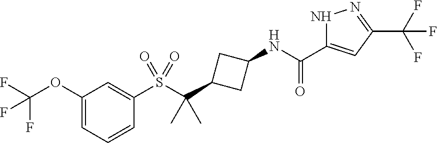 | | |
| 640 | 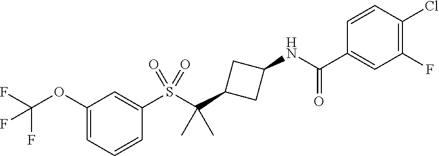 | | |
| 641 | 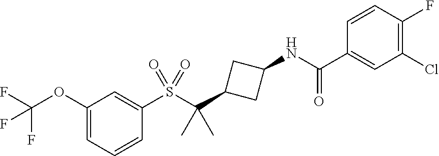 | | |
| 642 | 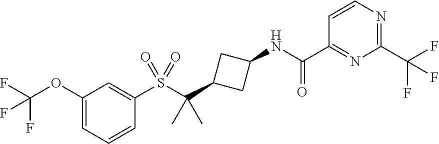 | | |
| 643 | 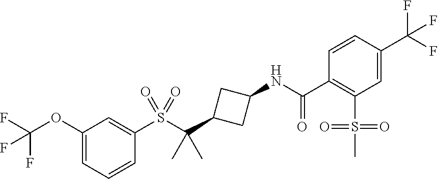 | | |
| 644 | 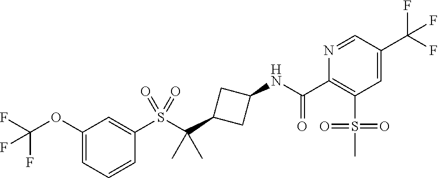 | | |
| 645 | 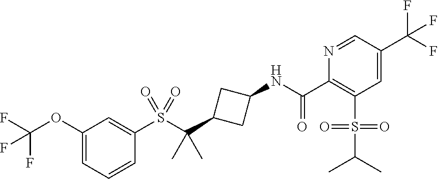 | | |
| 646 | 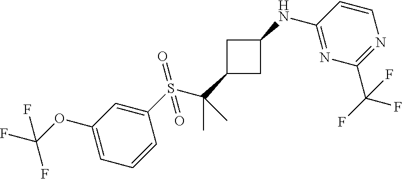 | | |
| 647 | 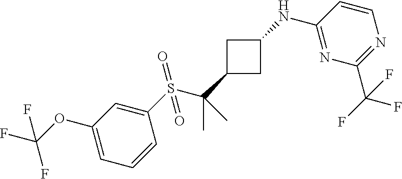 | | |
| 648 | 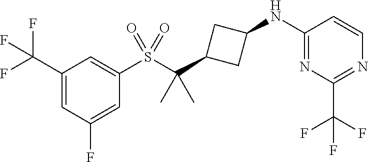 | | |
| 649 | 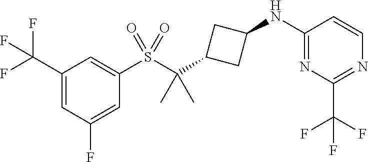 | | |
| 650 | 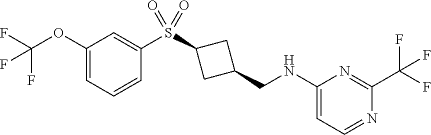 | | |
| 651 | 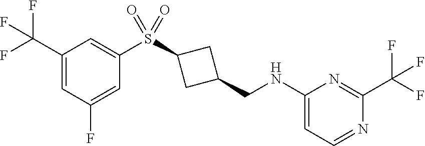 | | |
| 652 | 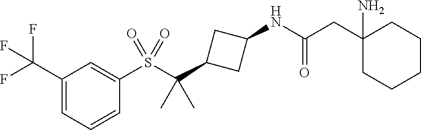 | | |
| 653 | 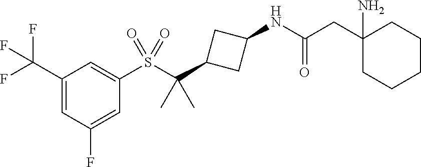 | | |
| 654 | 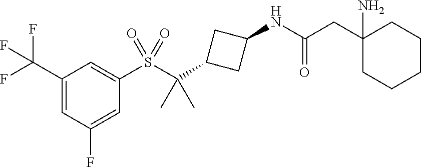 | | |
| 655 | 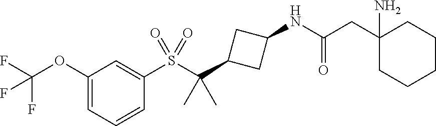 | | |
| 656 | 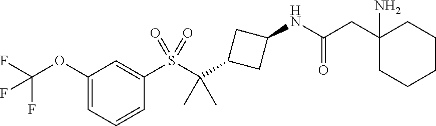 | | |
| 657 | 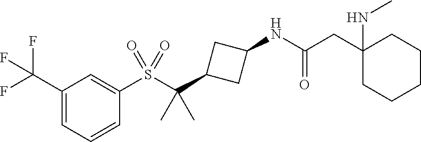 | | |
| 658 | 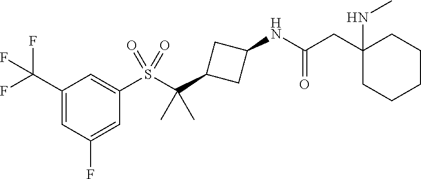 | | |
| 659 | 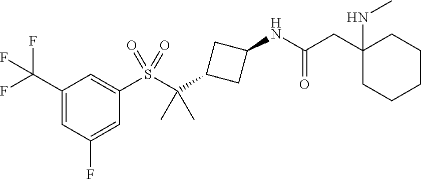 | | |
| 660 | 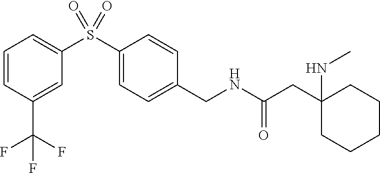 | | |
| 661 | 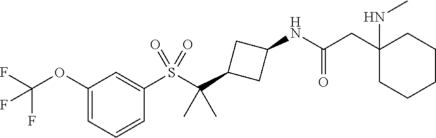 | | |
| 662 | 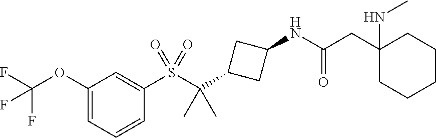 | | |
| 663 | 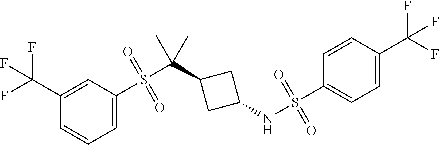 | | |
| 664 | 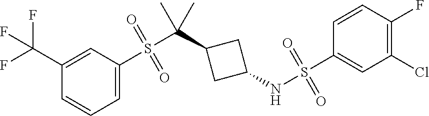 | | |
| 665 | 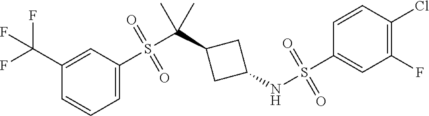 | | |
| 666 | 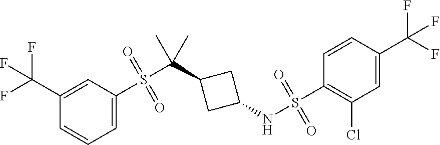 | | |
| 667 | 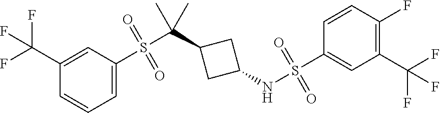 | | |
| 668 | 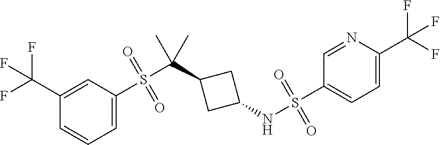 | | |
| 669 | 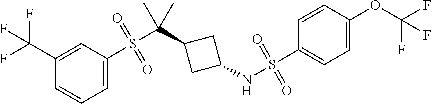 | | |
| 670 | 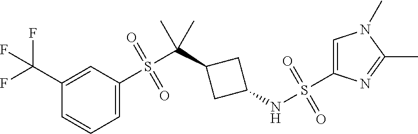 | | |
| 671 | 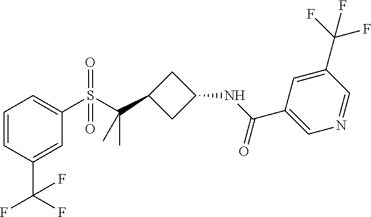 | | |
| 672 | 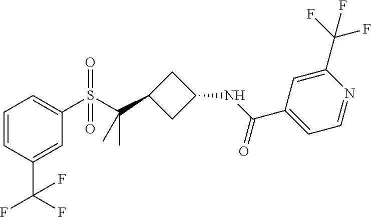 | | |
| 673 | 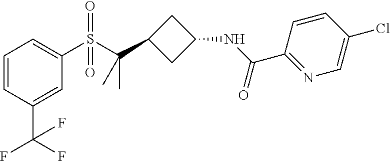 | | |
| 674 | 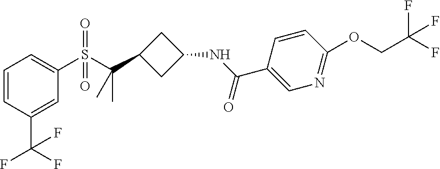 | | |
| 675 | 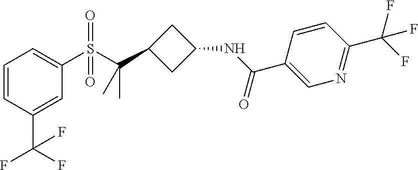 | | |
| 676 | 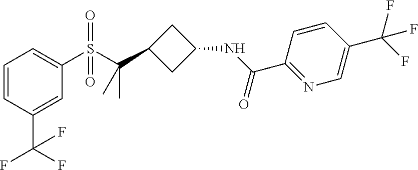 | | |
| 677 | 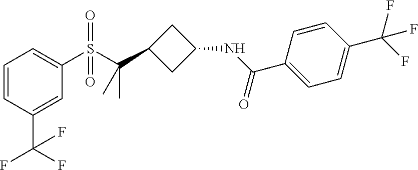 | | |
| 678 | 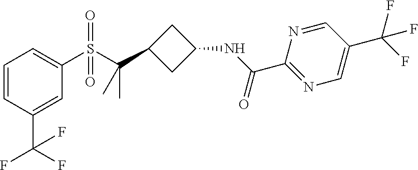 | | |
| 679 | 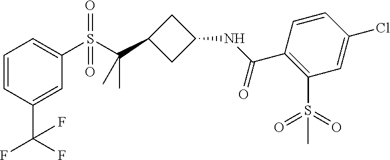 | | |
| 680 | 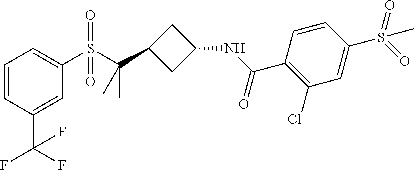 | | |
| 681 | 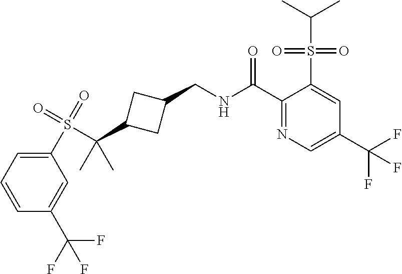 | | |
| 682 | 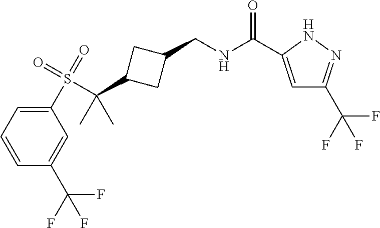 | | |
| 683 | 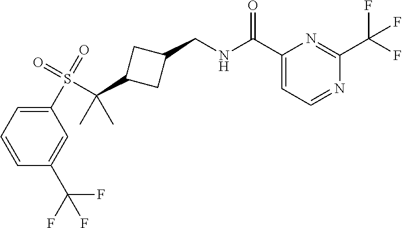 | | |
| 684 | 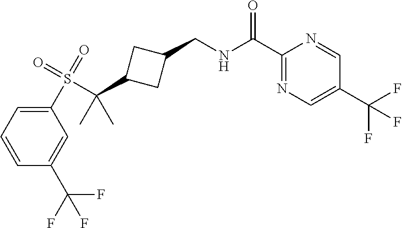 | | |
| 685 | 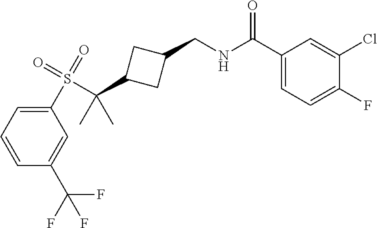 | | |
| 686 | 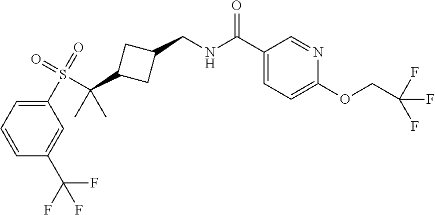 | | |
| 687 | 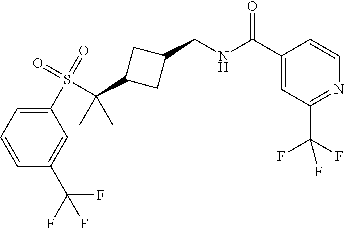 | | |
| 688 | 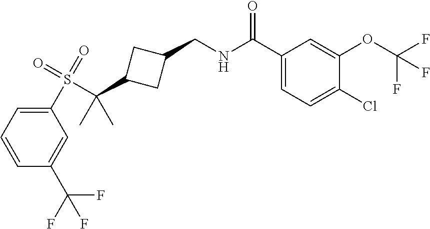 | | |
| 689 | 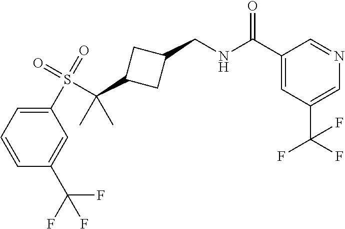 | | |
| 690 | 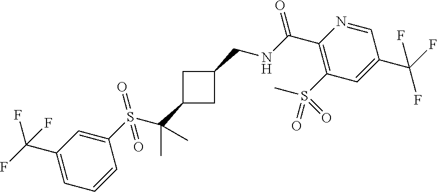 | | |
| 691 | 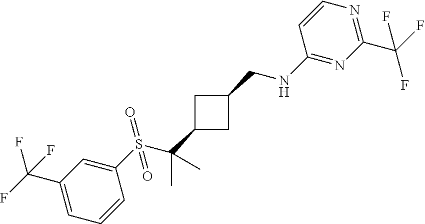 | | |
| 692 | 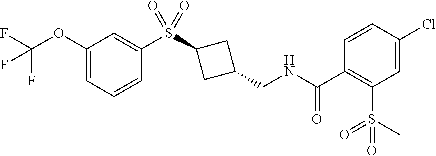 | | |
| 693 | 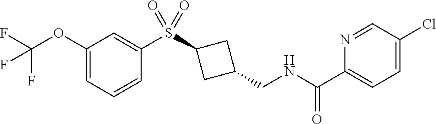 | | |
| 694 | 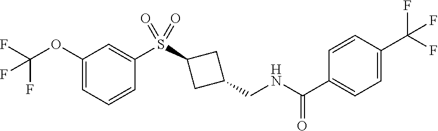 | | |
| 695 | 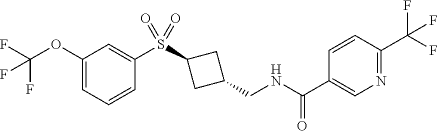 | | |
| 696 | 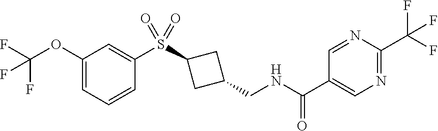 | | |
| 697 | 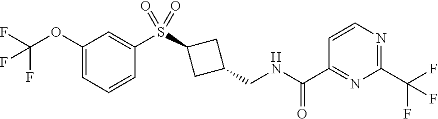 | | |
| 698 | 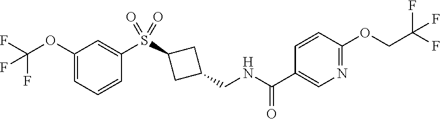 | | |
| 699 | 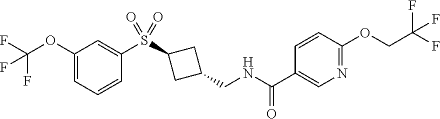 | | |
| 700 | 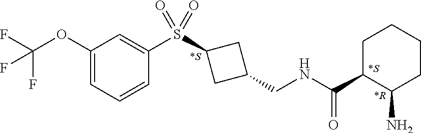 | | |
| 701 | 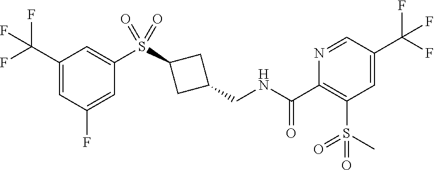 | | |
| 702 | 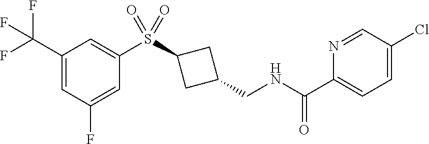 | | |
| 703 | 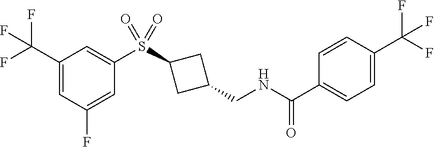 | | |
| 704 | 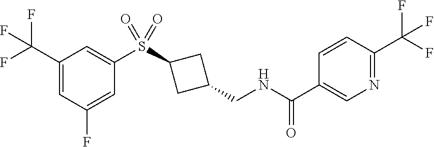 | | |
| 705 | 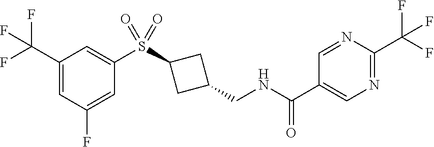 | | |
| 706 | 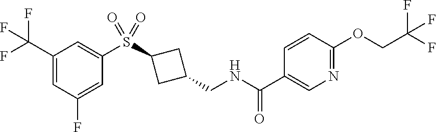 | | |
| 707 | 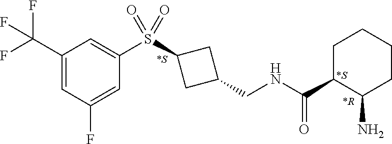 | | |
| 708 | 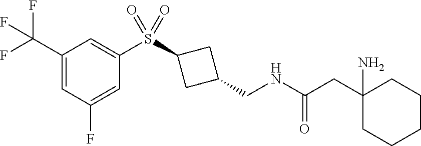 | | |
| 709 | 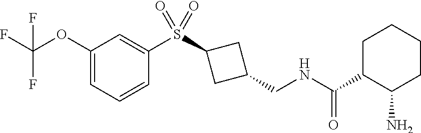 | | |
| 710 | 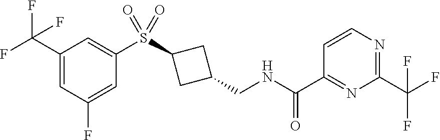 | | |
| 711 | 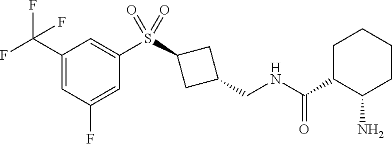 | | |
| 712 | 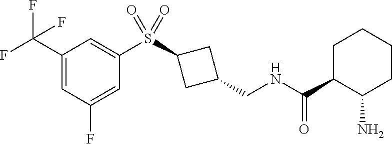 | | |
| 713 | 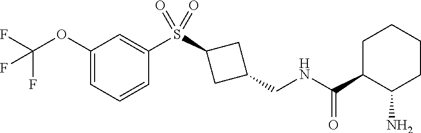 | | |
| 714 | 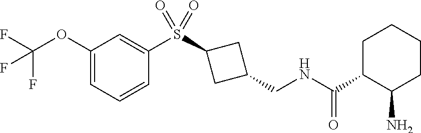 | | |
| 715 | 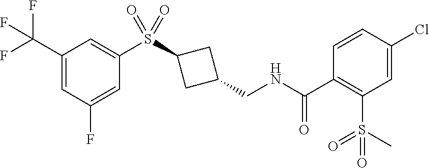 | | |
| 716 | 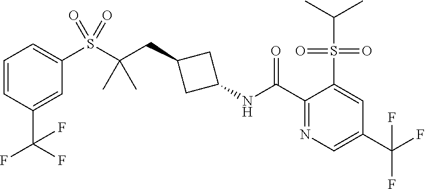 | | |
| 717 | 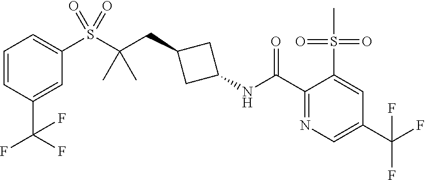 | | |
| 718 | 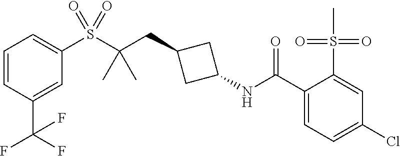 | | |
| 719 | 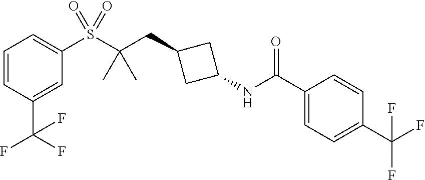 | | |
| 720 | 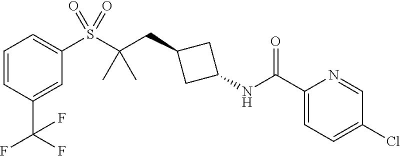 | | |
| 721 | 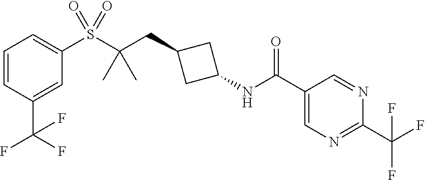 | | |
| 722 | 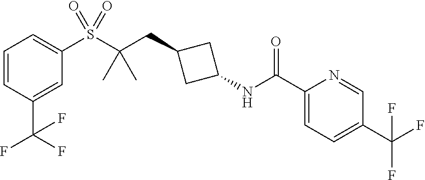 | | |
| 723 | 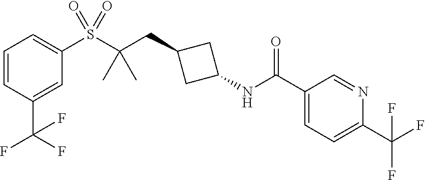 | | |
| 724 | 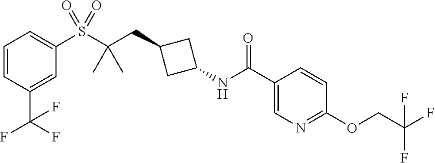 | | |
| 725 | 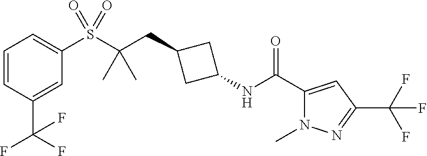 | | |
| 726 | 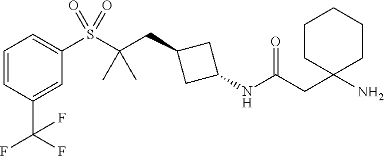 | | |
| 727 | 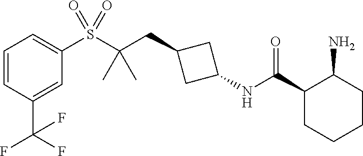 | | |
| 728 | 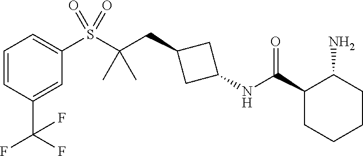 | | |
| 729 | 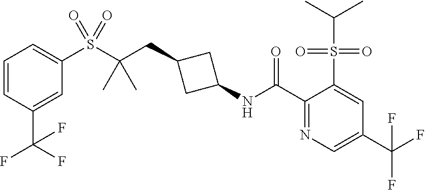 | | |
| 730 | 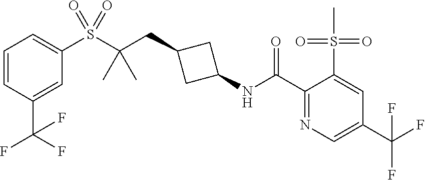 | | |
| 731 | 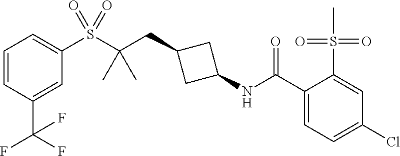 | | |
| 732 | 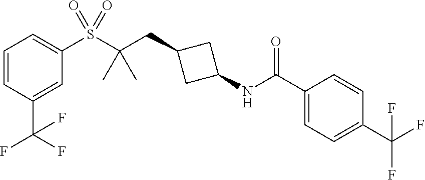 | | |
| 733 | 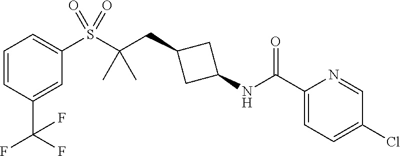 | | |
| 734 | 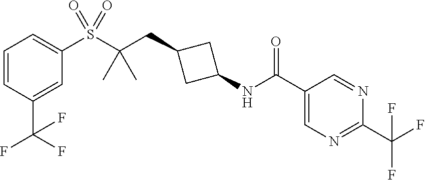 | | |
| 735 | 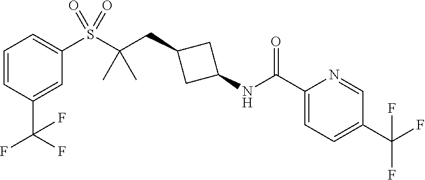 | | |
| 736 | 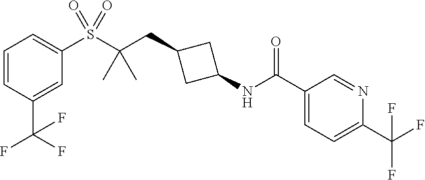 | | |
| 737 | 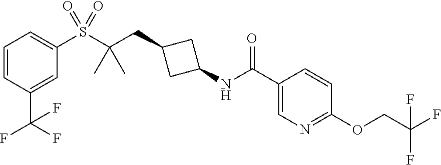 | | |
| 738 | 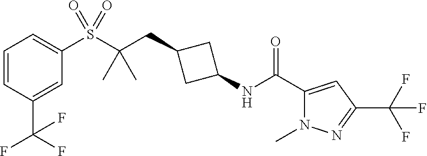 | | |
| 739 | 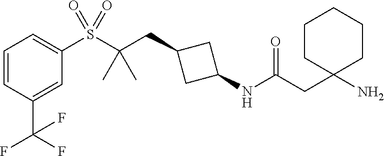 | | |
| 740 | 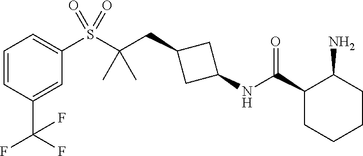 | | |
| 741 | 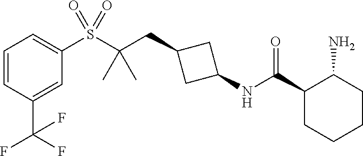 | | |
| 742 | 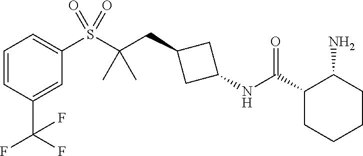 | | |
| 743 | 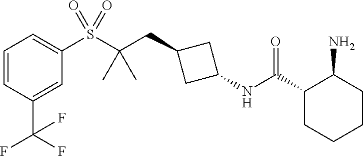 | | |
| 744 | 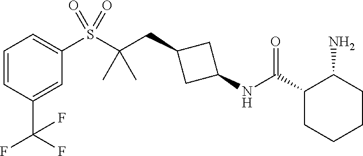 | | |
| 745 | 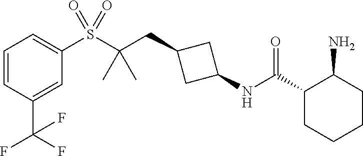 | | |
| 746 | 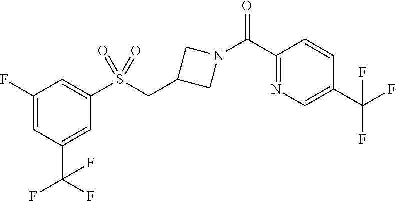 | | |
| 747 | 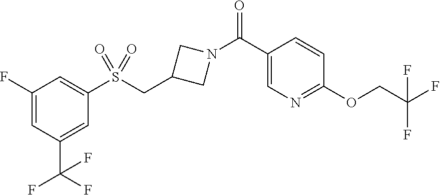 | | |
| 748 | 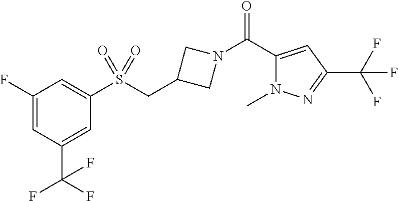 | | |
| 749 | 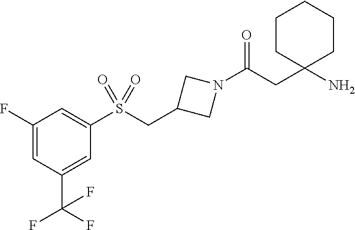 | | |
| 750 | 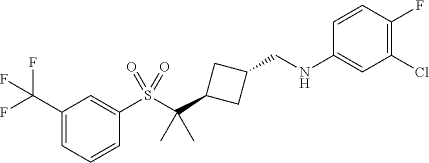 | | |
| 751 | 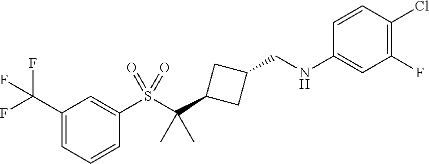 | | |
| 752 | 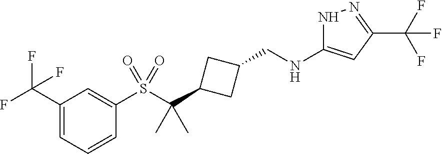 | | |
| 753 | 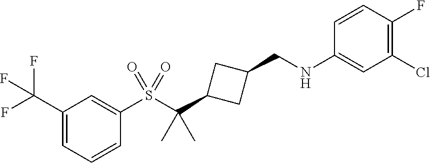 | | |
| 754 | 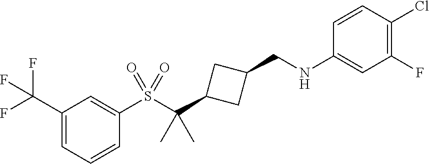 | | |
| 755 | 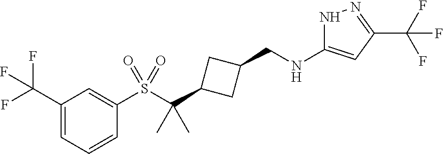 | | |
| 756 | 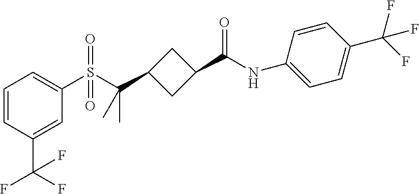 | | |
| 757 | 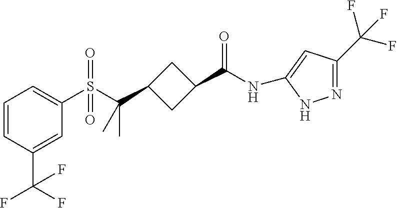 | | |
| 758 | 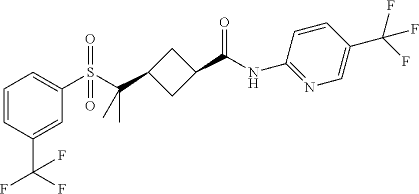 | | |
| 759 | 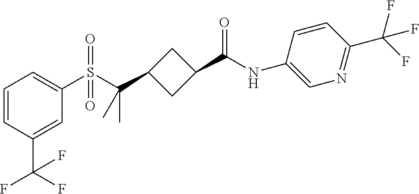 | | |
| 760 | 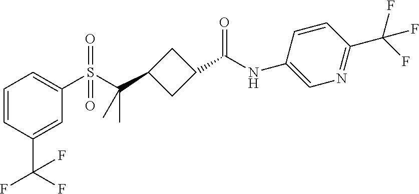 | | |
| 761 | 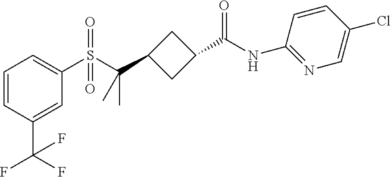 | | |
| 762 | 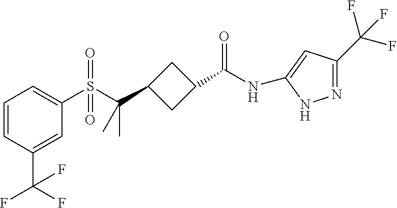 | | |
| 763 | 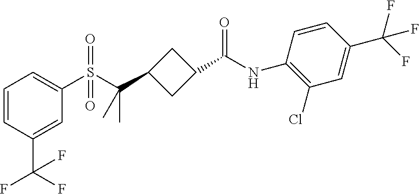 | | |
| 764 | 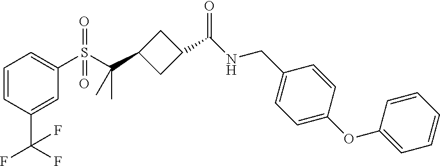 | | |
| 765 | 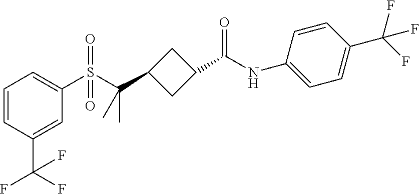 | | |
| 766 | 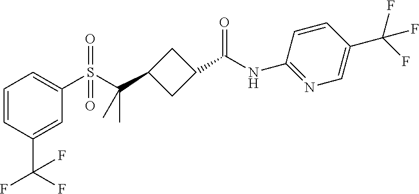 | | |
| 767 | 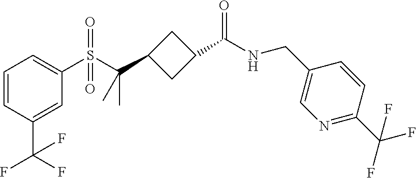 | | |
| 768 | 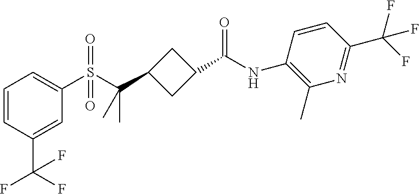 | | |
| 769 | 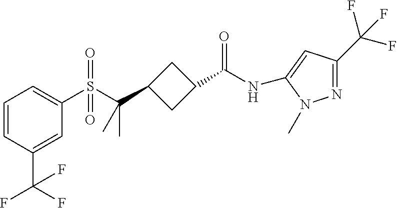 | | |
| 770 | 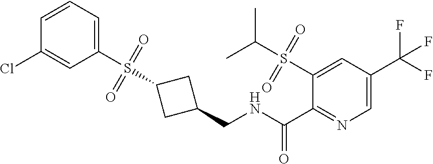 | | |
| 771 | 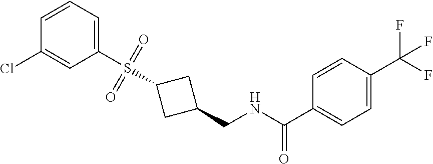 | | |
| 772 | 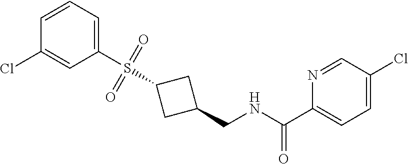 | | |
| 773 | 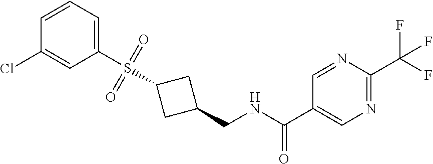 | | |
| 774 | 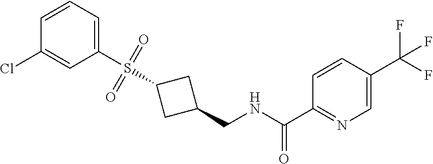 | | |
| 775 | 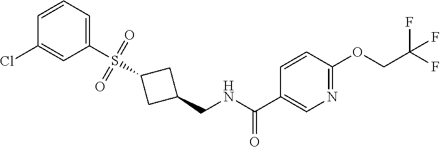 | | |
| 776 | 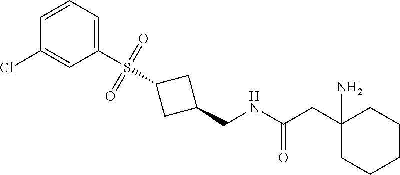 | | |
| 777 | 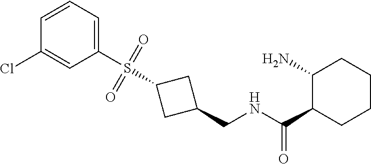 | | |
| 778 | 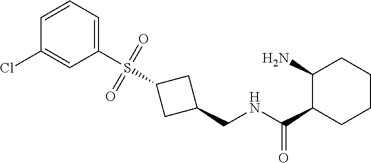 | | |
| 779 | 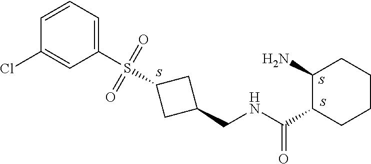 | | |
| 780 | 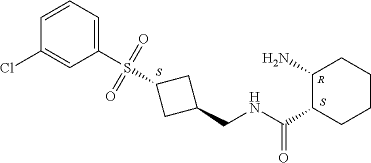 | | |

### Example 50

### Channel Blocking Activities

### T-Type Channel Blocking

A. Transformation of HEK cells:

T-type calcium channel blocking activity was assayed in human embryonic kidneycells, HEK 293 (Invitrogen), stably transfected with the T-type calcium channel subunits. Briefly, cells were cultured in Dulbecco's modified eagle medium (DMEM) supplemented with 10% fetal bovine serum, 200 U/ml penicillin, and 0.2 mg/mL streptomycin at 37° C. with 5% CO₂. At 85% confluency, cells were split with 0.25% trypsin/1 mM EDTA and plated at 10% confluency on glass coverslips. At 12 hours, the medium was replaced, and the cells stably transfected using a standard calcium phosphate protocol and the appropriate calcium channel cDNA's. Fresh DMEM was supplied, and the cells transferred to 28° C./5% CO₂. Cells were incubated for 1 to 2 days prior to whole cell recording.

Standard patch-clamp techniques were employed to identify blockers of T-type currents. Briefly, previously described HEK cell lines stably expressing human α_{1G}, α_{1H}, and α_{1I} T-type channels were used for all the recordings (passage #: 4-20, 37° C., 5% CO₂). Whole cell patch clamp experiments were performed using an Axopatch 200B amplifier (Axon Instruments, Burlingame, Calif.) linked to a personal computer equipped with pCLAMP software. Data were analyzed using Clampfit (Axon Instruments) and SigmaPlot 4.0 (Jandel Scientific). To obtain T-type currents, plastic dishes containing semi-confluent cells were positioned on the stage of a ZEISS AXIOVERT S100 microscope after replacing the culture medium with external solution (Table 2). Whole-cell patches were obtained using pipettes (borosilicate glass with filament, O.D.: 1.5 mm, I.D.: 0.86 mm, 10 cm length), fabricated on a SUTTER P-97 puller with resistance values of ˜5 MΩ (Table 3).

TABLE 2
External Solution 500 ml - pH 7.4, 265.5 mOsm

| Salt | Final mM | Stock M | Final ml |
|---|---|---|---|
| CsCl | 142 | 1 | 71 |
| CaCl₂ | 2 | 1 | 1 |
| MgCl₂ | 1 | 1 | 0.5 |
| HEPES | 10 | 0.5 | 10 |
| glucose | 10 | — | 0.9 grams |

TABLE 3
Internal Solution 50 ml - pH 7.3 with CsOH, 270 mOsm

| Salt | Final mM | Stock M | Final ml |
|---|---|---|---|
| Cs-Methanesulfonate | 126.5 | — | 1.442 gr/50 ml |
| MgCl2 | 2 | 1 | 0.1 |
| HEPES | 10 | 0.5 | 1 |
| EGTA-Cs | 11 | 0.25 | 2.2 |
| ATP | 2 | 0.2 | 0.025 |
| | | | (1 aliquot/2.5 ml) |

T-type currents were reliably obtained by using two voltage protocols:
“non-inactivating,” and
“inactivation.”

In the non-inactivating protocol, the holding potential is set at −110 mV and with a pre-pulse at −100 mV for 1 second prior to the test pulse at −40 mV for 50 ms. In the inactivation protocol, the pre-pulse is at approximately −85 mV for 1 second, which inactivates about 15% of the T-type channels (Scheme 1).

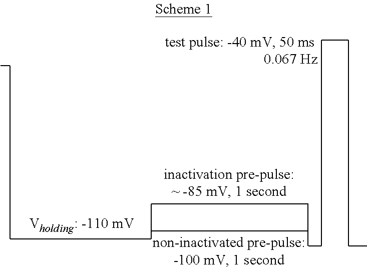

Test compounds were dissolved in external solution, 0.1-0.01% DMSO. After ˜10 minutes rest, they were applied by gravity close to the cell using a WPI microfil tubing. The “non-inactivated” pre-pulse was used to examine the resting block of a compound. The “inactivated” protocol was employed to study voltage-dependent block. However, the initial data shown below were mainly obtained using the non-inactivated protocol only. IC₅₀ values are shown for various compounds of the invention in Table 4. Values are shown in nM, with values above 10,000 nM represented as “10000 nM.” The data show that each of compounds 1-3, 5-9, 17, 19, 20, 29-33, 40-42, 45, 50-52, 54 and 56-64 exhibited activity at less than 1 μM. Further, compounds 3, 54, and 59 exhibited activity at less than 0.01 μM, with compound 59 demonstrating the lowest IC₅₀.

TABLE 4
T-Type Calcium Channel Block

| Compound | α_{1G} (nM) | α_{1H} (nM) |
|---|---|---|
| 1 | 10000 | 7400 |
| 2 | 1380 | 1170 |
| 3 | | 10000 |
| 4 | 8540 | 6740 |
| 5 | | 10000 |
| 6 | 10000 | 10000 |
| 7 | 10000 | 10000 |
| 8 | 10000 | 6710 |
| 9 | 1130 | 1030 |
| 10 | 4200 | 3820 |
| 11 | 10000 | 10000 |
| 12 | | |
| 13 | 10000 | 10000 |
| 14 | 1320 | 330 |
| 15 | 10000 | 4620 |
| 16 | 10000 | 9180 |
| 17 | 2330 | 1420 |
| 18 | 3060 | 2720 |
| 19 | 5240 | 3020 |
| 20 | 1970 | 1380 |
| 21 | 6560 | 4020 |
| 22 | — | — |
| 23 | 2790 | 1190 |
| 24 | — | — |
| 25 | 2790 | 4660 |
| 26 | 2790 | 10000 |
| 27 | 760 | 720 |
| 28 | 1230 | 1270 |
| 29 | 260 | 430 |
| 30 | 1290 | 1410 |
| 31 | 1150 | 5540 |
| 32 | 500 | 2690 |
| 33 | — | — |
| 34 | — | — |
| 35 | 1000 | 10000 |
| 36 | 5250 | 5110 |
| 37 | 9280 | 3620 |
| 38 | | |
| 39 | — | 10000 |
| 40 | — | — |
| 41 | — | — |
| 42 | 1510 | 2860 |
| 43 | — | |
| 44 | — | |
| 45 | | 10000 |
| 46 | 1250 | 910 |
| 47 | — | — |
| 48 | 1430 | 2330 |
| 49 | 10000 | 10000 |
| 50 | 10000 | 10000 |
| 51 | — | 10000 |
| 52 | 860 | 790 |
| 53 | — | — |
| 54 | 10000 | 7290 |
| 55 | — | — |
| 56 | 820 | 480 |
| 57 | 4940 | 1530 |
| 58 | 1850 | 1480 |
| 59 | 9130 | 8090 |
| 60 | 6440 | 3900 |
| 61 | — | 10000 |
| 62 | 1080 | 550 |
| 63 | — | — |
| 64 | — | 10000 |
| 65 | 10000 | 10000 |
| 66 | 10000 | 10000 |
| 67 | — | — |
| 68 | | 10000 |
| 69 | 10000 | 10000 |
| 70 | 8810 | 2580 |
| 71 | — | — |
| 72 | — | — |
| 73 | — | — |
| 74 | 1530 | 1210 |
| 75 | — | — |
| 76 | 1030 | 510 |
| 77 | — | — |
| 78 | — | — |
| 79 | 1190 | 440 |
| 80 | 900 | 460 |
| 81 | 380 | 220 |
| 82 | 7490 | 1810 |
| 83 | 2850 | 960 |
| 84 | 2870 | 650 |
| 85 | 2150 | 1230 |
| 86 | 10000 | 1930 |
| 87 | 6440 | 2100 |
| 88 | 10000 | 3030 |
| 89 | 1710 | 990 |
| 90 | — | — |
| 91 | 2450 | 940 |
| 92 | 2230 | 640 |
| 93 | — | 10000 |
| 94 | 10000 | 10000 |
| 95 | 6970 | 2150 |
| 96 | 10000 | 3820 |
| 97 | 2140 | 10000 |
| 98 | 10000 | 10000 |
| 99 | 10000 | 10000 |
| 100 | 5390 | 1790 |
| 101 | 3670 | 3500 |
| 102 | 10000 | 10000 |
| 103 | 10000 | |
| 104 | 10000 | 10000 |
| 105 | 10000 | 2480 |
| 106 | 2180 | 10000 |
| 107 | | 10000 |
| 108 | 6300 | 10000 |
| 109 | 10000 | 10000 |
| 110 | | 10000 |
| 111 | — | — |
| 112 | 10000 | 8360 |
| 113 | 10000 | 5350 |
| 114 | 10000 | 10000 |
| 115 | 10000 | 10000 |
| 116 | 8320 | 4240 |
| 117 | 3100 | 3370 |
| 118 | 10000 | 10000 |
| 119 | — | — |
| 120 | — | 10000 |
| 121 | 720 | 930 |
| 122 | 690 | 990 |
| 123 | 30 | 440 |
| 124 | 350 | 540 |
| 125 | 2910 | 2970 |
| 126 | 1300 | 2120 |
| 127 | 1140 | 1560 |
| 128 | — | — |
| 129 | — | — |
| 130 | 430 | 940 |
| 131 | 160 | 610 |
| 132 | 3670 | 4550 |
| 133 | 350 | 400 |
| 134 | 2630 | 1820 |
| 135 | 780 | 760 |
| 136 | 540 | 550 |
| 137 | 430 | 330 |
| 138 | 9880 | 2840 |
| 139 | 1880 | 1020 |
| 140 | 1100 | 700 |
| 141 | 4250 | 3610 |
| 142 | 8230 | 5880 |
| 143 | 10000 | 6750 |
| 144 | 1350 | 670 |
| 145 | 5830 | 1340 |
| 146 | — | — |
| 147 | — | — |
| 148 | — | — |
| 149 | — | — |
| 150 | 6470 | 2050 |
| 151 | 2900 | |
| 152 | 3600 | |
| 153 | 5230 | |
| 154 | 3600 | |
| 155 | 960 | |
| 156 | — | — |
| 157 | — | — |
| 158 | 10000 | 7200 |
| 159 | 10000 | 10000 |
| 160 | — | — |
| 161 | — | — |
| 162 | 10000 | 10000 |
| 163 | 10000 | — |
| 164 | — | — |
| 165 | 4370 | 1390 |
| 166 | 10000 | 8470 |
| 167 | — | — |
| 168 | — | — |
| 169 | — | — |
| 170 | — | — |
| 171 | — | — |
| 172 | 10000 | 6730 |
| 173 | — | — |
| 174 | — | — |
| 175 | — | — |
| 176 | — | — |
| 177 | — | — |
| 178 | — | 10000 |
| 179 | — | — |
| 180 | — | — |
| 181 | — | — |
| 182 | — | — |
| 183 | — | — |
| 184 | — | — |
| 185 | — | — |
| 186 | — | — |
| 187 | — | — |
| 188 | — | — |
| 189 | — | — |
| 190 | 5560 | 2530 |
| 191 | — | — |
| 192 | — | — |
| 193 | 6970 | 1590 |
| 194 | — | — |
| 195 | — | — |
| 196 | — | — |
| 197 | 10000 | 5660 |
| 198 | 10000 | 10000 |
| 199 | — | — |
| 200 | — | — |
| 201 | — | — |
| 202 | 10000 | 10000 |
| 203 | 2570 | 1890 |
| 204 | 3550 | 6460 |
| 205 | — | — |
| 206 | 3520 | 2550 |
| 207 | 1340 | 1130 |
| 208 | 1140 | 1110 |
| 209 | 3410 | 2530 |
| 210 | 4950 | 4140 |
| 211 | — | — |
| 212 | 2220 | — |
| 213 | 940 | — |
| 214 | — | — |
| 215 | — | — |
| 216 | — | — |
| 217 | — | — |
| 218 | — | — |
| 219 | — | — |
| 220 | — | — |
| 221 | — | — |
| 222 | — | — |
| 223 | — | — |
| 224 | — | — |
| 225 | — | — |
| 226 | — | — |
| 227 | — | — |
| 228 | 330 | — |
| 229 | — | — |
| 230 | 1180 | — |
| 231 | — | — |
| 232 | — | — |
| 233 | — | — |
| 234 | 2120 | — |
| 235 | — | — |
| 236 | — | — |
| 237 | — | — |
| 238 | 2780 | — |
| 239 | 1610 | — |
| 240 | 1360 | — |
| 241 | 1450 | — |
| 242 | 4300 | — |
| 243 | 4450 | — |
| 244 | 3570 | — |
| 245 | 3810 | — |
| 246 | 1720 | — |
| 247 | — | — |
| 248 | 950 | 430 |
| 249 | 2650 | 360 |
| 250 | 4280 | 990 |
| 251 | 3590 | 570 |
| 252 | 450 | 670 |
| 253 | 1190 | 460 |
| 254 | 740 | 260 |
| 255 | 2010 | 3320 |
| 256 | 2010 | 3320 |
| 257 | 6900 | 9020 |
| 258 | 7340 | >10000 |
| 259 | 6110 | 9620 |
| 260 | 2300 | 3650 |
| 261 | >10000 | 7180 |
| 262 | 2890 | 1470 |
| 263 | 2230 | 1360 |
| 264 | 4480 | >10000 |
| 265 | >10000 | >10000 |
| 266 | 3670 | 5560 |
| 267 | 2630 | 490 |
| 268 | >10000 | 1910 |
| 269 | >10000 | >10000 |
| 270 | 2540 | 3580 |
| 271 | 2630 | 1650 |
| 272 | >10000 | >10000 |
| 273 | >10000 | >10000 |
| 274 | 2540 | 1050 |
| 275 | >10000 | >10000 |
| 276 | >10000 | 2300 |
| 277 | 2460 | 540 |
| 278 | 2480 | 2230 |
| 279 | >10000 | 2090 |
| 280 | — | — |
| 281 | 8530 | 1460 |
| 282 | — | — |
| 283 | — | — |
| 284 | — | — |
| 285 | >10000 | 1280 |
| 286 | 3190 | 1540 |
| 287 | 6500 | 1130 |
| 288 | >10000 | 3260 |
| 289 | 1400 | 1040 |
| 290 | 1180 | 570 |
| 291 | >10000 | 6060 |
| 292 | — | >10000 |
| 293 | 4770 | 1850 |
| 294 | — | >10000 |
| 295 | 630 | 320 |
| 296 | 2830 | 850 |
| 297 | 850 | 300 |
| 298 | 880 | 240 |
| 299 | 640 | 210 |
| 300 | 280 | 430 |
| 301 | >10000 | — |
| 302 | 970 | 850 |
| 303 | 4340 | 4150 |
| 304 | 1550 | 2440 |
| 305 | >10000 | >10000 |
| 306 | 4960 | 7350 |
| 307 | 2410 | 1540 |
| 308 | 4200 | 2270 |
| 309 | 9900 | >10000 |
| 310 | 6340 | 5710 |
| 311 | 5080 | 8500 |
| 312 | 2960 | 1940 |
| 313 | >10000 | >10000 |
| 314 | — | — |
| 315 | — | >10000 |
| 316 | 3990 | 2910 |
| 317 | 1140 | 860 |
| 318 | 2430 | 1330 |
| 319 | 4820 | 3430 |
| 320 | 290 | 190 |
| 321 | — | >10000 |
| 322 | 3260 | 630 |
| 323 | 1350 | 720 |
| 324 | 3180 | 1220 |
| 325 | 2400 | 1330 |
| 326 | >1000 | 1480 |
| 327 | 2790 | 750 |
| 328 | 10000 | 2470 |
| 329 | >10000 | >10000 |
| 330 | 8560 | 930 |
| 331 | 2650 | 1510 |
| 332 | 1560 | 780 |
| 333 | 1820 | 960 |
| 334 | >10000 | 2390 |
| 335 | >10000 | 4710 |
| 336 | 690 | 1350 |
| 337 | >10000 | 8660 |
| 338 | >10000 | 6790 |
| 339 | 5270 | 3330 |
| 340 | 9870 | 3170 |
| 341 | 2190 | 2020 |
| 342 | 1710 | 760 |
| 343 | 630 | 270 |
| 344 | >10000 | >10000 |
| 345 | 5530 | 1930 |
| 346 | 1260 | 410 |
| 347 | 2370 | 260 |
| 348 | 4520 | 540 |
| 349 | >10000 | >10000 |
| 350 | >10000 | >10000 |
| 351 | >10000 | 7430 |
| 352 | 640 | 230 |
| 353 | 5020 | 1980 |
| 354 | >10000 | 7060 |
| 355 | 1430 | 740 |
| 356 | 9510 | 3170 |
| 357 | >10000 | >10000 |
| 358 | 8370 | >10000 |
| 359 | 5420 | 20000 |
| 360 | >10000 | >10000 |
| 361 | 1620 | 200 |
| 362 | — | >10000 |
| 363 | — | >10000 |
| 364 | 520 | 210 |
| 365 | 6480 | 2190 |
| 366 | >10000 | >10000 |
| 367 | 900 | 350 |
| 368 | 950 | 400 |
| 369 | >10000 | >10000 |
| 370 | 680 | 640 |
| 371 | 1060 | 660 |
| 372 | 7170 | 9630 |
| 373 | 820 | 610 |
| 374 | 7730 | 2110 |
| 375 | — | — |
| 376 | — | — |
| 377 | 2340 | 1040 |
| 378 | 2790 | 1630 |
| 379 | 1810 | 300 |
| 380 | — | >10000 |
| 381 | — | >10000 |
| 382 | >10000 | >10000 |
| 383 | >10000 | 5950 |
| 384 | >10000 | 4740 |
| 385 | >10000 | >10000 |
| 386 | 490 | 270 |
| 387 | 720 | 710 |
| 388 | — | — |
| 389 | 6080 | 8980 |
| 390 | — | — |
| 391 | — | — |
| 392 | — | — |
| 393 | — | — |
| 394 | — | — |
| 395 | — | — |
| 396 | — | — |
| 397 | — | — |
| 398 | — | — |
| 399 | 1250 | 1060 |
| 400 | >10000 | 5920 |
| 401 | >10000 | 9260 |
| 402 | 640 | 570 |
| 403 | 1190 | 1040 |
| 404 | — | >10000 |
| 405 | >10000 | |
| 406 | — | — |
| 407 | >10000 | 1660 |
| 408 | 8000 | 420 |
| 409 | 2650 | 3620 |
| 410 | 3490 | 1790 |
| 411 | >10000 | 2430 |
| 412 | 6810 | 1550 |
| 413 | — | — |
| 414 | — | — |
| 415 | >10000 | >10000 |
| 416 | >10000 | 8180 |
| 417 | >10000 | 4690 |
| 418 | >10000 | 2870 |
| 419 | >10000 | >10000 |
| 420 | 2020 | 710 |
| 421 | — | — |
| 422 | 2250 | 1390 |
| 423 | >10000 | >10000 |
| 424 | 2290 | 1040 |
| 425 | 1550 | 1070 |
| 426 | >10000 | — |
| 427 | 3820 | 880 |
| 428 | — | >10000 |
| 429 | >10000 | 8370 |
| 430 | — | — |
| 431 | 920 | 240 |
| 432 | 1290 | 330 |
| 433 | — | >10000 |
| 434 | 1130 | 680 |
| 435 | — | — |
| 436 | — | — |
| 437 | — | — |
| 438 | — | — |
| 439 | — | — |
| 440 | — | — |
| 441 | — | — |
| 442 | — | — |
| 443 | — | — |
| 444 | — | — |
| 445 | — | — |
| 446 | — | — |
| 447 | — | — |
| 448 | — | — |
| 449 | 450 | 550 |
| 450 | 610 | 4490 |
| 451 | 260 | 2410 |
| 452 | 570 | 9810 |
| 453 | 690 | 3930 |
| 454 | 610 | 3480 |
| 455 | 400 | 2500 |
| 456 | 710 | 910 |
| 457 | 560 | 1200 |
| 458 | 900 | 1920 |
| 459 | 820 | 10000 |
| 460 | 100 | 10000 |
| 461 | 190 | 10000 |
| 462 | 560 | 10000 |
| 463 | 350 | 10000 |
| 464 | 60 | 2140 |
| 465 | 50 | 960 |
| 466 | 1000 | 550 |
| 467 | 170 | 1170 |
| 468 | 350 | 4860 |
| 469 | 430 | 5040 |
| 470 | 130 | 5840 |
| 471 | 610 | 10000 |
| 472 | 620 | 3470 |
| 473 | 680 | 9280 |
| 474 | 560 | 10000 |
| 475 | 480 | 6970 |
| 476 | 400 | 5620 |
| 477 | 540 | 10000 |
| 478 | 270 | 5340 |
| 479 | 900 | 2090 |
| 480 | 720 | 5440 |
| 481 | 720 | 10000 |
| 482 | 960 | 3690 |
| 483 | 900 | 3850 |
| 484 | 530 | 6690 |
| 485 | 710 | — |
| 486 | 510 | 860 |
| 487 | 210 | 3310 |
| 488 | 290 | 2880 |
| 489 | 900 | 10000 |
| 490 | 460 | 10000 |
| 491 | 470 | 10000 |
| 492 | 590 | 9260 |
| 493 | 680 | 10160 |
| 494 | 400 | 3530 |
| 495 | 190 | 2050 |
| 496 | 170 | 350 |
| 497 | 520 | — |
| 498 | 260 | 1380 |
| 499 | 90 | 1010 |
| 500 | 340 | 3110 |
| 501 | 420 | 10000 |
| 502 | 360 | 3060 |
| 503 | 190 | 2810 |
| 504 | 540 | 1760 |
| 505 | 610 | 9930 |
| 506 | 290 | 640 |
| 507 | 380 | 8300 |
| 508 | 850 | — |
| 509 | 650 | — |
| 510 | 170 | 5870 |
| 511 | 960 | — |
| 512 | 330 | 10000 |
| 513 | 490 | 10000 |
| 514 | 840 | 10000 |
| 515 | 610 | — |
| 516 | 620 | 10000 |
| 517 | 170 | 6140 |
| 518 | 600 | 4120 |
| 519 | 550 | 3280 |
| 520 | 110 | 470 |
| 521 | 470 | 910 |
| 522 | 370 | 1360 |
| 523 | 560 | 10000 |
| 524 | 380 | 2070 |
| 525 | 470 | 2880 |
| 526 | 240 | 2930 |
| 527 | 70 | 10000 |
| 528 | 80 | 2410 |
| 529 | 780 | 9690 |
| 530 | 940 | — |
| 531 | 3560 | 10000 |
| 532 | 1630 | 10000 |
| 533 | 350 | 1080 |
| 534 | 830 | — |
| 535 | 700 | 4140 |
| 536 | 640 | 10000 |
| 537 | 470 | 740 |
| 538 | 620 | 3080 |

### N-Type Channel Blocking Activities

### Assay Example 1

### Fluorescent Assay for CaV2.2 Channels Using Potassium Depolarization to Initiate Channel Opening

Human Caᵥ 2.2 channels were stably expressed in HEK293 cells along with alpha2-delta and beta subunits of voltage-gated calcium channels. An inwardly rectifying potassium channel (Kir2.3) was also expressed in these cells to allow more precise control of the cell membrane potential by extracellular potassium concentration. At low bath potassium concentration, the membrane potential is relatively negative, and is depolarized as the bath potassium concentration is raised. In this way, the bath potassium concentration can be used to regulate the voltage-dependent conformations of the channels. Compounds are incubated with cells in the presence of low (4 mM) potassium or elevated (12, 25, or 30 mM) potassium to determine the affinity for compound block of resting (closed) channels at 4 mM potassium or affinity for block of open and inactivated channels at 12, 25, or 30 mM potassium. After the incubation period, Cav2.2 channel opening is triggered by addition of higher concentration of potassium (70 mM final concentration) to further depolarize the cell. The degree of state-dependent block can be estimated from the inhibitory potency of compounds after incubation in different potassium concentrations.

Calcium influx through Caᵥ 2.2 channels is determined using a calcium sensitive fluorescent dye in combination with a fluorescent plate reader. Fluorescent changes were measured with either a VIPR (Aurora Instruments) or FLIPR (Molecular Devices) plate reader.

### Protocol

1. Seed cells in Poly-D-Lysine Coated 96 or 384-well plate and keep in a 3rC-IO % C02 incubator overnight.

2. Remove media, wash cells with 0.2 mL (96-well plate) or 0.05 mL (384-well plate) Dulbecco's Phosphate Buffered Saline (D-PBS) with calcium & magnesium (Invitrogen; 14040).

3. Add 0.1 mL (96-well plate) or 0.05 mL (384-well plate) of 4˜M fluor-4 (Molecular Probes; F-14202) and 0.02% Pluronic acid (Molecular Probes; P-3(00) prepared in D-PBS with calcium & magnesium (Invitrogen; 14040) supplemented with 10 mM Glucose & 10 mM HepeslNaOH; pH 7.4.

4. Incubate in the dark at 25° C. for 60-70 min.

5. Remove dye, wash cells with 0.1 mL (96-well plate) or 0.06 mL (384-well plate) of 4, 12, 25, or 30 mM Potassium Pre-polarization Buffer (PPB).

6. Add 0.1 mL (96-well plate) or 0.03 mL (384-well plate) of 4, 12, 25, 30 mM PPB with or without test compound.

7. Incubate in the dark at 25° C. for 30 min

8. Read cell plate on VIPR instrument, Excitation=480 nm, Emission=535 nm.

9. With VIPR continuously reading, add 0.1 mL (96-well plate) or 0.03 mL (384-well plate) of Depolarization Buffer (DB), which is 2× the final assay concentration, to the cell plate.

| | | | | | | | | | |
|---|---|---|---|---|---|---|---|---|---|
| 4 | mM PPB | 12 | mM PPB | 25 | mM PPB | 30 | mM PPB | 140 | mM K DB |
| 146 | mM NaCl | 138 | mM NaCl | 125 | mM NaCl | 120 | mM NaCl | 10 | mM NaCl |
| 4 | mM KCl | 12 | mM KCl | 25 | mM KCl | 30 | mM KCl | 140 | mM KCl |
| 0.8 | mM CaCl₂ | 0.8 | mM CaCl₂ | 0.8 | mM CaCl₂ | 0.8 | mM CaCl₂ | 0.8 | mM CaCl₂ |
| 1.7 | MgCl₂ | 1.7 | MgCl₂ | 1.7 | MgCl₂ | 1.7 | MgCl₂ | 1.7 | MgCl₂ |
| 10 | HEPES | 10 | HEPES | 10 | HEPES | 10 | HEPES | 10 | HEPES |

| | | | | |
|---|---|---|---|---|
| pH = 7.2 | pH = 7.2 | pH = 7.2 | pH = 7.2 | pH = 7.2 |

### Assay Example 2

### Electrophysiological Measurement of Block of Cav2.2 Channels Using Automated Electrophysiology Instruments

Blocking of N-type calcium channels is evaluated utilizing the IonWorks HT 384 well automated patch clamp electrophysiology device. T his instrument allows synchronous recording from 384 well (48 at a time). A single whole cell recording is made in each well. Whole cell recording is established by perfusion of the internal compartment with amphotericin B.

The voltage protocol is designed to detect use-dependent blocking and uses a 2 Hz train of depolarizations (twenty 25 ms steps to +20 mY). The experimental sequence consists of a control train (pre-compound), incubation of cells with compound for 5 minutes, followed by a second train (post-compound). Use dependent block by compounds is estimated by comparing the fractional block of the first pulse in the train to block of the 20^{th} pulse.

### Protocol:

Parallel patch clamp electrophysiology is performed using IonWorks HT (Molecular Devices Corp) essentially as described by Kiss and colleagues (Kiss et al. Assay and Drug Development Technologies, I: 127-135, 2003). Briefly, a stable HEK 293 cell line (referred to as CBK) expressing N-type calcium channel subunits (α_{1B}, α₂δ, and β₃ₐ) and an inwardly rectifying potassium channel (Kᵢᵣ2.3) is used to record barium current through the N-type calcium channel. Cells are grown in TI5 culture plates to 60-90% confluence before use. Cells are rinsed 3× with 10 mL PBS (CalMg-free), followed by addition of 1.0 mL I× trypsin to the flask. Cells are incubated at 37° C. until rounded and free from plate (usually 1-3 min). Cells are then transferred to a 15 mL conical tube with 13 ml of CBK media containing serum and antibiotics and spun at setting 2 on a table top centrifuge for 2 min. The supernatant is poured off, and the pellet of cells is re-suspended in external solution (in mM): 120 NaCl, 20 BaCl₂, 4.5 KCl, 0.5 MgCl₂, 10 HEPES, 10 Glucose, pH 7.4). The concentration of cells in suspension is adjusted to achieve 1000-3000 cells per well. Cells are used immediately once they have been resuspended. The internal solution is (in mM): 100 K-Gluconate, 40 KCl, 3.2 MgCl₂, 3 EGTA, 5 HEPES, pH 7.3 with KOH. Perforated patch whole cell recording is achieved by adding the perforating amphotericin B to the internal solution. A 36 mg/mL stock of amphotericin B is made fresh in DMSO for each run. 166 μL of this stock is added to 50 mL of internal solution yielding a final working solution of 120 μg/ml.

Voltage protocols and the recording of membrane currents are performed using the IonWorks HT software/hardware system. Currents are sampled at 1.25 kHz and leakage subtraction is performed using a 10 mV step from the holding potential and assuming a linear leak conductance. No correction for liquid junction potentials is employed. Cells are voltage clamped at −70 m V for 10 s followed by a 20 pulse train of 25 ms steps to +20 mV at 2 Hz. After a control train, the cells are incubated with compound for 5 minutes and a second train is applied. Use dependent block by compounds is estimated by comparing the fractional block of the first pulse to block of the 20^{th} pulse. Wells with seal resistances less than 70 MOhms or less than 0.1 nA of Ba current at the test potential (+20 mV) are excluded from analysis. Current amplitudes are calculated with the IonWorks software. Relative current, percent inhibition and IC₅₀ values are calculated with a custom Excel/Sigmaplot macro.

Compounds are added to cells with a fluidics head from a 96-well compound plate. To compensate for the dilution of compound during addition, the compound plate concentration is 3× higher than the final concentration on the patch plate.

Two types of experiments are generally performed: screens and titrations. In the screening mode, 10-20 compounds are evaluated at a single concentration (usually 3 μM). The percent inhibition is calculated from the ratio of the current amplitude in the presence and absence of compound, normalized to the ratio in vehicle control wells. For generation of IC₅₀ values, a 10-point titration is performed on 2-4 compounds per patch plate. The range of concentrations tested is generally 0.001 to 20 μM. IC₅₀ values are calculated from the fits of the Hill equation to the data. The form of the Hill equation used is:

Relative Current=(Max−Min)/((I+(conc/IC₅₀)̂slope)+Min).

Each plate for normalization purposes and to define the Max and Min.

### Assay Example 3

### Electrophysiological Measurement of Block of Cav2.2 Channel Using Whole Cell Voltage Clamp and Using PatchXpress Automated Electrophysiology Instrument

A stable HEK 293 cell line (referred to as CBK) expressing N-type calcium channel subunits (α_{1B}, α₂δ, and β₃ₐ) and an inwardly rectifying potassium channel (Kₐ2.3) is used to record barium current through then-type calcium channel. Cells are grown either on poly-D-lysine coated coverglass (manual EP) or in TIS culture plates (PatchXpress). For the PatchExpress, cells are released from the flask using trypsin. In both cases, the external solution is (in mM): 130 CsCl₂, 10 EGTA, 10 HEPES, 2 MgCl₂, 3 MgATP, pH 7.3 with CsOH.

Barium currents are measured by manual whole-cell patch clamp using standard techniques (Hamill et. al. Pfluegers Archiv 391:85-100 (1981)). Microelectrodes are fabricated from borosilicate glass and fire-polished. Electrode resistances are generally 2 to 4 MOhm when filled with the standard internal saline. The reference electrode is a silver-silver chloride pellet. Voltages are not corrected for the liquid junction potential between the internal and external solutions and leak is subtracted using the P/n procedure. Solutions are applied to cells by bath perfusion via gravity. The experimental chamber volume is—0.2 mL. and the perfusion rate is 0.5-2 mL/min. Flow of suction through the chamber is maintained at all times. Measurement of current amplitudes is performed with PULSEFIT software (HEKA Elektronik).

PatchXpress (Molecular Devices) is a 16-well whole-cell automated patch clamp device that operates asynchronously with fully integrated fluidics. High resistance (gigaohm) seals are achieved with 50-80% success. Capacitance and series resistance compensation is automated. No correction for liquid junction potentials is employed. Leak is subtracted using the P/n procedure. Compounds are added to cells with a pipettor from a 96-well compound plate. Voltage protocols and the recording of membrane currents are performed using the PatchXpress software/hardware system. Current amplitudes are calculated with DataXpress software.

In both manual and automated patch clamp, cells are voltage clamped at −4 mV or −90 m V and 50 ms pulses to +20 mV are applied every 15 sec (0.067 Hz). Compounds are added in escalating doses to measure % inhibition. Percent inhibition is calculated from the ratio of the curerent amplitude in the presence and absence of compound. When multiple doses are achieved per cell, IC₅₀ values are calculated. The range of concentrations tested is generally 0.1 to 30 ˜M. IC₅₀ values are calculated from the fits of the Hill equation to the data. The form of the Hill equation used is: Relative Current=1/(1+(conc/IC₅₀)̂slope).

TABLE 5
N-type Calcium Channel Block

| Compound | N-Type (nM) |
|---|---|
| 1 | 1030 |
| 2 | 950 |
| 3 | 640 |
| 4 | 140 |
| 5 | 380 |
| 6 | 330 |
| 7 | 1580 |
| 8 | 2300 |
| 9 | 2520 |
| 10 | 1460 |
| 11 | 150 |
| 12 | 1940 |
| 13 | 690 |
| 14 | 2210 |
| 15 | 50 |
| 16 | 140 |
| 17 | 260 |
| 18 | 390 |
| 19 | 60 |
| 20 | 70 |
| 21 | 200 |
| 22 | — |
| 23 | 240 |
| 24 | — |
| 25 | 360 |
| 26 | 210 |
| 27 | 250 |
| 28 | 940 |
| 29 | 610 |
| 30 | 720 |
| 31 | 360 |
| 32 | 440 |
| 33 | 10000 |
| 34 | 640 |
| 35 | — |
| 36 | 740 |
| 37 | 1310 |
| 38 | — |
| 39 | 410 |
| 40 | 170 |
| 41 | — |
| 42 | 480 |
| 43 | — |
| 44 | 5930 |
| 45 | 1290 |
| 46 | 1060 |
| 47 | 10000 |
| 48 | 1650 |
| 49 | 200 |
| 50 | 90 |
| 51 | 1510 |
| 52 | 1540 |
| 53 | 4720 |
| 54 | 4630 |
| 55 | 10000 |
| 56 | 260 |
| 57 | 860 |
| 58 | 800 |
| 59 | 440 |
| 60 | 370 |
| 61 | 1650 |
| 62 | 3480 |
| 63 | 9980 |
| 64 | 550 |
| 65 | 550 |
| 66 | 8990 |
| 67 | 10000 |
| 68 | 1720 |
| 69 | 1650 |
| 70 | 1220 |
| 71 | 5870 |
| 72 | 8580 |
| 73 | 10000 |
| 74 | 4950 |
| 75 | 10000 |
| 76 | 590 |
| 77 | 740 |
| 78 | |
| 79 | 630 |
| 80 | 4310 |
| 81 | 410 |
| 82 | 3000 |
| 83 | 480 |
| 84 | 310 |
| 85 | 850 |
| 86 | 190 |
| 87 | 240 |
| 88 | 70 |
| 89 | 120 |
| 90 | — |
| 91 | 410 |
| 92 | 330 |
| 93 | 240 |
| 94 | 230 |
| 95 | 1640 |
| 96 | 430 |
| 97 | 490 |
| 98 | 680 |
| 99 | 3750 |
| 100 | 2320 |
| 101 | 10000 |
| 102 | 630 |
| 103 | 460 |
| 104 | 840 |
| 105 | 10000 |
| 106 | 1940 |
| 107 | 2710 |
| 108 | 720 |
| 109 | 10000 |
| 110 | 6020 |
| 111 | — |
| 112 | 10000 |
| 113 | 130 |
| 114 | 510 |
| 115 | 650 |
| 116 | 2090 |
| 117 | 4020 |
| 118 | 790 |
| 119 | — |
| 120 | 580 |
| 121 | 3730 |
| 122 | — |
| 123 | 660 |
| 124 | 510 |
| 125 | 900 |
| 126 | 1020 |
| 127 | 1290 |
| 128 | — |
| 129 | 10000 |
| 130 | 290 |
| 131 | 160 |
| 132 | 1720 |
| 133 | 210 |
| 134 | 500 |
| 135 | 100 |
| 136 | 440 |
| 137 | 320 |
| 138 | 420 |
| 139 | 390 |
| 140 | 700 |
| 141 | 600 |
| 142 | 380 |
| 143 | 950 |
| 144 | 1250 |
| 145 | 1190 |
| 146 | — |
| 147 | — |
| 148 | — |
| 149 | — |
| 150 | 520 |
| 151 | 580 |
| 152 | 550 |
| 153 | 500 |
| 154 | 450 |
| 155 | 540 |
| 156 | 1230 |
| 157 | 1030 |
| 158 | 480 |
| 159 | 460 |
| 160 | 1990 |
| 161 | 2450 |
| 162 | 540 |
| 163 | 470 |
| 164 | 1810 |
| 165 | 540 |
| 166 | 750 |
| 167 | 10000 |
| 168 | 10000 |
| 169 | 7110 |
| 170 | 4920 |
| 171 | 480 |
| 172 | 10000 |
| 173 | — |
| 174 | 10000 |
| 175 | — |
| 176 | 9440 |
| 177 | 2060 |
| 178 | 250 |
| 179 | — |
| 180 | 3350 |
| 181 | 1830 |
| 182 | 10000 |
| 183 | 1990 |
| 184 | 3110 |
| 185 | 1630 |
| 186 | 5840 |
| 187 | 7000 |
| 188 | 5610 |
| 189 | 1540 |
| 190 | 600 |
| 191 | 1220 |
| 192 | 10000 |
| 193 | 540 |
| 194 | 1330 |
| 195 | 3150 |
| 196 | — |
| 197 | 470 |
| 198 | 270 |
| 199 | 1250 |
| 200 | 1020 |
| 201 | 1860 |
| 202 | 590 |
| 203 | 590 |
| 204 | 210 |
| 205 | 1700 |
| 206 | 420 |
| 207 | 260 |
| 208 | 590 |
| 209 | 390 |
| 210 | 380 |
| 211 | — |
| 212 | 860 |
| 213 | 620 |
| 214 | 2090 |
| 215 | 2810 |
| 216 | 1010 |
| 217 | — |
| 218 | — |
| 219 | 10000 |
| 220 | 3350 |
| 221 | — |
| 222 | 2960 |
| 223 | — |
| 224 | 1500 |
| 225 | 2350 |
| 226 | 2150 |
| 227 | 10000 |
| 228 | 740 |
| 229 | 1020 |
| 230 | 790 |
| 231 | 1870 |
| 232 | 2660 |
| 233 | 3570 |
| 234 | 870 |
| 235 | 5940 |
| 236 | 2100 |
| 237 | 4560 |
| 238 | 90 |
| 239 | 340 |
| 240 | 350 |
| 241 | 130 |
| 242 | 110 |
| 243 | 60 |
| 244 | 40 |
| 245 | 130 |
| 246 | 130 |
| 247 | 1010 |
| 248 | 770 |
| 249 | 260 |
| 250 | 800 |
| 251 | 150 |
| 252 | 350 |
| 253 | 320 |
| 254 | 370 |
| 255 | 210 |
| 256 | 150 |
| 257 | 680 |
| 258 | 680 |
| 259 | 500 |
| 260 | 1080 |
| 261 | 1720 |
| 262 | 250 |
| 263 | 2510 |
| 264 | 470 |
| 265 | 260 |
| 266 | 2440 |
| 267 | 790 |
| 268 | 30 |
| 269 | 190 |
| 270 | 210 |
| 271 | 8160 |
| 272 | 430 |
| 273 | 430 |
| 274 | 530 |
| 275 | 700 |
| 276 | 380 |
| 277 | 470 |
| 278 | 280 |
| 279 | 450 |
| 280 | >10000 |
| 281 | 30 |
| 282 | — |
| 283 | 580 |
| 284 | 1050 |
| 285 | 360 |
| 286 | 390 |
| 287 | 310 |
| 288 | 640 |
| 289 | 540 |
| 290 | 420 |
| 291 | 50 |
| 292 | 130 |
| 293 | 1630 |
| 294 | 3760 |
| 295 | 790 |
| 296 | 4230 |
| 297 | 720 |
| 298 | 850 |
| 299 | 730 |
| 300 | 700 |
| 301 | 530 |
| 302 | 400 |
| 303 | 180 |
| 304 | 230 |
| 305 | 300 |
| 306 | 530 |
| 307 | 3120 |
| 308 | 1450 |
| 309 | 1390 |
| 310 | 1160 |
| 311 | 650 |
| 312 | 1020 |
| 313 | 1980 |
| 314 | 4920 |
| 315 | 1230 |
| 316 | 3120 |
| 317 | 2690 |
| 318 | 620 |
| 319 | 260 |
| 320 | 600 |
| 321 | 1920 |
| 322 | 500 |
| 323 | 2410 |
| 324 | 1060 |
| 325 | 320 |
| 326 | 330 |
| 327 | 630 |
| 328 | 710 |
| 329 | 350 |
| 330 | 1020 |
| 331 | 1290 |
| 332 | 620 |
| 333 | 180 |
| 334 | 360 |
| 335 | 520 |
| 336 | 1060 |
| 337 | 910 |
| 338 | 770 |
| 339 | 220 |
| 340 | 2010 |
| 341 | 510 |
| 342 | 720 |
| 343 | 1450 |
| 344 | 680 |
| 345 | 580 |
| 346 | 5540 |
| 347 | 1120 |
| 348 | 1030 |
| 349 | 890 |
| 350 | 1410 |
| 351 | 2770 |
| 352 | 3760 |
| 353 | 3210 |
| 354 | 140 |
| 355 | 190 |
| 356 | 570 |
| 357 | 200 |
| 358 | 190 |
| 359 | 1610 |
| 360 | 190 |
| 361 | 460 |
| 362 | 370 |
| 363 | 310 |
| 364 | 1400 |
| 365 | 1610 |
| 366 | 190 |
| 367 | 850 |
| 368 | 940 |
| 369 | 160 |
| 370 | 290 |
| 371 | 1160 |
| 372 | 260 |
| 373 | 250 |
| 374 | 250 |
| 375 | 260 |
| 376 | 540 |
| 377 | 460 |
| 378 | 1130 |
| 379 | 830 |
| 380 | 1470 |
| 381 | 590 |
| 382 | 1710 |
| 383 | 1360 |
| 384 | 960 |
| 385 | 710 |
| 386 | 2370 |
| 387 | 4220 |
| 388 | 1060 |
| 389 | 270 |
| 390 | 1630 |
| 391 | 730 |
| 392 | — |
| 393 | 1070 |
| 394 | 510 |
| 395 | 750 |
| 396 | 940 |
| 397 | 910 |
| 398 | 740 |
| 399 | 1140 |
| 400 | 3830 |
| 401 | 1430 |
| 402 | 1700 |
| 403 | 1460 |
| 404 | 3330 |
| 405 | 1300 |
| 406 | 1540 |
| 407 | 5630 |
| 408 | 2490 |
| 409 | 1690 |
| 410 | 1240 |
| 411 | 1020 |
| 412 | 590 |
| 413 | — |
| 414 | 1360 |
| 415 | 1760 |
| 416 | 1650 |
| 417 | 5440 |
| 418 | 3500 |
| 419 | 3430 |
| 420 | 1250 |
| 421 | 5580 |
| 422 | 2670 |
| 423 | 1280 |
| 424 | 1460 |
| 425 | 3760 |
| 426 | 1140 |
| 427 | 920 |
| 428 | 960 |
| 429 | 770 |
| 430 | >10000 |
| 431 | 1840 |
| 432 | 810 |
| 433 | 220 |
| 434 | 210 |
| 435 | 1470 |
| 436 | 630 |
| 437 | 720 |
| 438 | 400 |
| 439 | 400 |
| 440 | 80 |
| 441 | 80 |
| 442 | 2840 |
| 443 | 440 |
| 444 | 290 |
| 445 | 200 |
| 446 | 810 |
| 447 | 1830 |
| 448 | 2110 |

Additional channel blocking data are shown in Table 6.

TABLE 6
N- and T-type Channel Blocking Data

| | FLIPR Data | | | | | | | | | |
|---|---|---|---|---|---|---|---|---|---|---|
| | CAᵥ2.2 IC₅₀ | | | | CAᵥ3.2 IC₅₀ | | | CAᵥ1.2 IC₅₀ | | |
| | | | Ratio | CAᵥ3.1 | | | Ratio | | | Ratio |
| No. | Closed | Inact. | closed/inact. | IC₅₀ | Closed | Inact. | closed/inact. | Closed | Inact. | closed/inact. |
| 571 | 10000 | 420 | 23.8 | 10000 | 10000 | 10000 | 1 | 10000 | 10000 | 1 |
| 572 | 3540 | 270 | 13.1 | 8160 | 2600 | 1680 | 1.5 | 7350 | 3010 | 2.4 |
| 573 | 3240 | 430 | 7.5 | | 8120 | 5830 | 1.4 | 10000 | 10000 | 1 |
| 574 | 10000 | 1290 | 7.8 | | | | | | | |
| 575 | 3210 | 280 | 11.5 | 10000 | 5720 | 4450 | 1.3 | 7520 | 5360 | 1.4 |
| 576 | 8560 | 440 | 19.5 | 10000 | 10000 | 9900 | 1 | 10000 | 9620 | 1 |
| 577 | 10000 | 640 | 15.6 | | | | | 10000 | 10000 | 1 |
| 578 | 3460 | 460 | 7.5 | 10000 | 7710 | 5300 | 1.5 | 9070 | 8380 | 1.1 |
| 579 | 10000 | 660 | 15.2 | 10000 | 10000 | 10000 | 1 | 10000 | 9950 | 1 |
| 580 | 10000 | 260 | 38.5 | 10000 | 10000 | 10000 | 1 | 10000 | 10000 | 1 |
| 581 | 2430 | 130 | 18.7 | 4840 | 3070 | 660 | 4.7 | 3870 | 1480 | 2.6 |
| 582 | 2880 | 190 | 15.2 | 10000 | 4350 | 1310 | 3.3 | 5770 | 2060 | 2.8 |
| 583 | 10000 | 1120 | 8.9 | | | 10000 | | 10000 | | |
| 584 | 10000 | 1700 | 5.9 | 10000 | | | | 10000 | 10000 | 1 |
| 585 | 10000 | 1080 | 9.3 | | 10000 | 10000 | 1 | | 10000 | |
| 586 | 10000 | 480 | 20.8 | 10000 | 10000 | 10000 | 1 | 10000 | 9370 | 1.1 |
| 587 | 5550 | 720 | 7.7 | 10000 | 6560 | 3600 | 1.8 | 10000 | 4850 | 2.1 |
| 588 | 5860 | 950 | 6.2 | | 10000 | 3270 | 3.1 | 10000 | 10000 | 1 |
| 589 | 2370 | 270 | 8.8 | 4030 | 4140 | 1070 | 3.9 | 4910 | 1800 | 2.7 |
| 590 | 2830 | 340 | 8.3 | 6850 | 4300 | 1370 | 3.1 | 5330 | 1700 | 3.1 |
| 591 | 10000 | 1150 | 8.7 | 10000 | | | | | 10000 | |
| 592 | 10000 | 460 | 21.7 | | 10000 | 10000 | 1 | 10000 | 10000 | 1 |
| 593 | 10000 | 160 | 62.5 | 10000 | 10000 | 10410 | 1 | 10000 | 7860 | 1.3 |
| 594 | 6080 | 520 | 11.7 | 10000 | 9230 | 2350 | | | | |
| 595 | 3630 | 210 | 17.3 | 10000 | 10000 | 9830 | 1 | 10000 | 10000 | 1 |
| 596 | 1730 | 260 | 6.7 | 9740 | 3130 | 1110 | 2.8 | 3630 | 1430 | 2.5 |
| 597 | 1430 | 170 | 8.4 | 3800 | 3120 | 1210 | 2.6 | 3700 | 860 | 4.3 |
| 598 | 5740 | 700 | 8.2 | 7560 | 10000 | 4560 | 2.2 | 9380 | 2830 | 3.3 |
| 599 | 6140 | 1030 | 6 | 5590 | 10000 | 4400 | 2.3 | 8850 | 3050 | 2.9 |
| 600 | 5250 | 820 | 6.4 | 3570 | 10000 | 5080 | 2 | 6930 | 2220 | 3.1 |
| 601 | 7070 | 900 | 7.9 | 3820 | 10000 | 4970 | 2 | 8840 | 2860 | 3.1 |
| 602 | 6080 | 750 | 8.1 | 6360 | 10000 | 5380 | 1.9 | 9770 | 3940 | 2.5 |
| 603 | 4170 | 610 | 6.8 | 5480 | 10000 | 6460 | 1.5 | 6920 | 2120 | 3.3 |
| 604 | 10000 | 4380 | 2.3 | | 10000 | | | 10000 | 10000 | 1 |
| 605 | 5400 | 870 | 6.2 | 4350 | 4790 | 2210 | 2.2 | 5290 | 1920 | 2.8 |
| 606 | 2510 | 920 | 2.7 | 4170 | 8530 | 3570 | 2.4 | 5650 | 2760 | 2 |
| 607 | 2970 | 1150 | 2.6 | 5430 | 10000 | 3730 | 2.7 | 7780 | 2990 | 2.6 |
| 608 | 4030 | 1100 | 3.7 | 4920 | 10000 | 2580 | 3.9 | 7750 | 2450 | 3.2 |
| 609 | 4170 | 1170 | 3.6 | 9910 | 10000 | 2540 | 3.9 | 9990 | 4320 | 2.3 |
| 610 | 3580 | 1130 | 3.2 | 5300 | 10000 | 1430 | 7 | 8930 | 2930 | 3 |
| 611 | 10000 | 9140 | 1.1 | 10000 | | 10000 | | 10000 | 10000 | 1 |
| 612 | 8540 | 540 | 15.8 | 10000 | 9280 | 3950 | 2.3 | 10000 | 4170 | 2.4 |
| 613 | 6510 | 350 | 18.6 | 10000 | 9160 | 5450 | 1.7 | 8000 | 5920 | 1.4 |
| 614 | 2870 | 490 | 5.9 | 10000 | 7200 | 3200 | 2.3 | 8030 | 2410 | 3.3 |
| 615 | 1370 | 250 | 5.5 | 10000 | 10000 | 7170 | 1.4 | 10000 | 5610 | 1.8 |
| 616 | 4810 | 610 | 7.9 | 10000 | 10000 | 6400 | 1.6 | 10000 | 2810 | 3.6 |
| 617 | 1340 | 150 | 8.9 | 8030 | 2690 | 810 | 3.3 | 4050 | 380 | 10.7 |
| 618 | 1300 | 120 | 10.8 | 2470 | 1990 | 350 | 5.7 | 4080 | 300 | 13.6 |
| 619 | 1660 | 260 | 6.4 | 5120 | 3290 | 1270 | 2.6 | 4420 | 1360 | 3.3 |
| 620 | 2870 | 390 | 7.4 | 8710 | 5520 | 1390 | 4 | 9990 | 670 | 14.9 |
| 621 | 1830 | 150 | 12.2 | 1470 | 1530 | 200 | 7.7 | 3390 | 240 | 14.1 |
| 622 | 4790 | 680 | 7 | 4860 | 10000 | 4480 | 2.2 | 9010 | 1940 | 4.6 |
| 623 | 3370 | 740 | 4.6 | 5300 | 10000 | 1540 | 6.5 | 8070 | 1540 | 5.2 |
| 624 | 10000 | 800 | 12.5 | 2960 | 2960 | 2260 | 1.3 | 10000 | 5540 | 1.8 |
| 625 | 10010 | 1090 | 9.2 | 3740 | 3710 | 3100 | 1.2 | 10000 | 6390 | 1.6 |
| 626 | 8450 | 1100 | 7.7 | 3770 | 3850 | 2580 | 1.5 | 9150 | 5400 | 1.7 |
| 627 | 10030 | 880 | 11.4 | 2380 | 2200 | 1330 | 1.7 | 10000 | 5860 | 1.7 |
| 628 | 10000 | 1220 | 8.2 | 2180 | 3220 | 3260 | 1 | 10000 | 5320 | 1.9 |
| 629 | 7450 | 430 | 17.3 | 2970 | 3410 | 3380 | 1 | 10000 | 4660 | 2.1 |
| 630 | 10000 | 680 | 14.7 | 2330 | 3730 | 2160 | 1.7 | 10000 | 3110 | 3.2 |
| 631 | | 6900 | 1.4 | | | | | | | |
| 632 | 5690 | 460 | 12.4 | 9410 | 570 | 1520 | 0.4 | 10000 | 3300 | 3 |
| 633 | 2060 | 190 | 10.8 | 2300 | 630 | 570 | 1.1 | 5740 | 1180 | 4.9 |
| 634 | 2210 | 220 | 10 | 1550 | 700 | 810 | 0.9 | 5920 | 600 | 9.9 |
| 635 | 2000 | 270 | 7.4 | 6960 | 7630 | 7840 | 1 | 10000 | 7630 | 1.3 |
| 636 | 10000 | 380 | 26.3 | 9970 | 4220 | 5440 | 0.8 | 10000 | 5240 | 1.9 |
| 637 | 10000 | 580 | 17.2 | 10000 | 10000 | 10000 | 1 | | 10000 | |
| 638 | 6790 | 210 | 32.3 | 4240 | 5490 | 6380 | 0.9 | 10000 | 6110 | 1.6 |
| 639 | 6160 | 870 | 7.1 | 2280 | 2580 | 2550 | 1 | 9310 | 3400 | 2.7 |
| 640 | 2490 | 400 | 6.2 | 1260 | 700 | 920 | 0.8 | 4900 | 1430 | 3.4 |
| 641 | 1670 | 250 | 6.7 | 770 | 290 | 530 | 0.5 | 4110 | 690 | 6 |
| 642 | 3090 | 630 | 4.9 | 4070 | 9610 | 2810 | 3.4 | 10000 | 8430 | 1.2 |
| 643 | 10000 | 470 | 21.3 | 4170 | 8380 | 7160 | 1.2 | 10000 | 9250 | 1.1 |
| 644 | 10000 | 900 | 11.1 | | 10000 | 10000 | 1 | | 10000 | |
| 645 | 8490 | 240 | 35.4 | 4690 | 9970 | 6080 | 1.6 | 10000 | 6800 | 1.5 |
| 646 | 4230 | 1030 | 4.1 | 4590 | 5270 | 3900 | 1.4 | 8000 | 2330 | 3.4 |
| 647 | 4170 | 860 | 4.8 | 3270 | 2990 | 2590 | 1.2 | 6550 | 1080 | 6.1 |
| 648 | 5050 | 990 | 5.1 | 2810 | 5140 | 4100 | 1.3 | 8410 | 2280 | 3.7 |
| 649 | 4830 | 1190 | 4.1 | 3130 | 2400 | 2740 | 0.9 | 6390 | 1810 | 3.5 |
| 650 | 9900 | 1880 | 5.3 | 7290 | 2270 | 2690 | 0.8 | 9100 | 3510 | 2.6 |
| 651 | 10000 | 3260 | 3.1 | 9040 | 6410 | 3590 | 1.8 | 9210 | 3860 | 2.4 |
| 652 | 8870 | 1190 | 7.5 | | | 10000 | | | | |
| 653 | 10000 | 1360 | 7.4 | 10000 | | 10000 | | | 10000 | |
| 654 | 8470 | 1440 | 5.9 | | | 10000 | | 10000 | 10000 | 1 |
| 655 | 6520 | 1160 | 5.6 | | | 10000 | | 10000 | 10000 | 1 |
| 656 | 5520 | 870 | 6.3 | | | 10000 | | 10000 | 10000 | 1 |
| 657 | 9320 | 2150 | 4.3 | | | | | 10000 | | |
| 658 | 10000 | 2640 | 3.8 | | 10000 | 10000 | 1 | 10000 | | |
| 659 | 9710 | 1930 | 5 | | | 10000 | | 10000 | 10000 | 1 |
| 660 | 3560 | 610 | 5.8 | 10000 | 10000 | 10000 | 1 | 7480 | 4930 | 1.5 |
| 661 | 8030 | 1530 | 5.2 | | | 10000 | | 10000 | 10000 | 1 |
| 662 | 5650 | 1250 | 4.5 | | | 10000 | | 10000 | 10000 | 1 |
| 663 | 9190 | 870 | 10.6 | 2010 | 2940 | 1530 | 1.9 | 10000 | 2980 | 3.4 |
| 664 | 8050 | 1060 | 7.6 | 2930 | 3080 | 2790 | 1.1 | 10000 | 3670 | 2.7 |
| 665 | 7260 | 800 | 9.1 | 2770 | 3030 | 2660 | 1.1 | 8940 | 4210 | 2.1 |
| 666 | 10000 | 650 | 15.4 | 1590 | 2160 | 2170 | 1 | 10000 | 2290 | 4.4 |
| 667 | 10000 | 1080 | 9.3 | 2500 | 2700 | 2160 | 1.3 | 7760 | 2560 | 3 |
| 668 | 7340 | 790 | 9.3 | 2950 | 3830 | 2920 | 1.3 | 10000 | 3730 | 2.7 |
| 669 | 7900 | 960 | 8.2 | 2740 | 2650 | 2550 | 1 | 10000 | 3330 | 3 |
| 670 | 10000 | 8660 | 1.2 | | | | | | | |
| 671 | 5470 | 280 | 19.5 | 9280 | 5730 | 6360 | 0.9 | 10000 | 7820 | 1.3 |
| 672 | 7420 | 310 | 23.9 | 5050 | 5930 | 4050 | 1.5 | 10000 | 3300 | 3 |
| 673 | 1880 | 200 | 9.4 | 6560 | 5370 | 4330 | 1.2 | 10000 | 4570 | 2.2 |
| 674 | 3010 | 290 | 10.4 | 9430 | 2740 | 2390 | 1.1 | 7730 | 1740 | 4.4 |
| 675 | 8630 | 490 | 17.6 | 10000 | 7760 | 6230 | 1.2 | 10000 | 5990 | 1.7 |
| 676 | 1850 | 160 | 11.6 | 3870 | 4270 | 3790 | 1.1 | 7960 | 3250 | 2.4 |
| 677 | 2860 | 220 | 13 | 2790 | 1780 | 1820 | 1 | 6350 | 2230 | 2.8 |
| 678 | 8950 | 790 | 11.3 | | | | | | | |
| 679 | 10000 | 480 | 20.8 | | 10000 | 10000 | 1 | | | |
| 680 | 10000 | 1310 | 7.6 | | 10000 | 10000 | 1 | | | |
| 681 | 9890 | 160 | 61.8 | 4820 | 5930 | | | 10000 | 2620 | 3.8 |
| 682 | 6640 | 530 | 12.5 | 10000 | 2310 | | | 10000 | 2160 | 4.6 |
| 683 | 2770 | 260 | 10.7 | 5530 | 5450 | | | 10000 | 6110 | 1.6 |
| 684 | 8030 | 650 | 12.4 | 4650 | 4670 | | | 10000 | 5350 | 1.9 |
| 685 | 1870 | 180 | 10.4 | 380 | 440 | | | 4710 | 1010 | 4.7 |
| 686 | 2470 | 260 | 9.5 | 1170 | 1200 | | | 4420 | 1100 | 4 |
| 687 | 4110 | 530 | 7.8 | 2610 | 1650 | | | 10000 | 2390 | 4.2 |
| 688 | 3400 | 220 | 15.5 | 250 | 450 | 170 | 2.6 | 4590 | 450 | 10.2 |
| 689 | | | | | | | | | | |
| 690 | 10000 | 450 | 22.2 | 10000 | 10000 | | | 10000 | 10000 | 1 |
| 691 | 4030 | 530 | 7.6 | 5830 | 7350 | | | 4940 | 2220 | 2.2 |
| 692 | 7750 | 780 | 9.9 | 9810 | 7920 | 2480 | 3.2 | 10000 | 10000 | 1 |
| 693 | 1770 | 340 | 5.2 | 10000 | 7950 | 2770 | 2.9 | 10000 | 6640 | 1.5 |
| 694 | 2110 | 300 | 7 | 3080 | 2470 | 1000 | 2.5 | 7510 | 2220 | 3.4 |
| 695 | 5120 | 780 | 6.6 | 9870 | 8580 | 5580 | 1.5 | 10000 | 10000 | 1 |
| 696 | 7070 | 940 | 7.5 | 10000 | 9920 | 6350 | 1.6 | 10000 | 8330 | 1.2 |
| 697 | 3780 | 710 | 5.3 | | 10000 | 8910 | 1.1 | 10000 | 10000 | 1 |
| 698 | 2700 | 470 | 5.7 | 3020 | 3020 | 2680 | 1.1 | 5600 | 2480 | 2.3 |
| 699 | 7860 | 2540 | 3.1 | | 10000 | 10000 | 1 | 10000 | | |
| 700 | 3550 | 1250 | 2.8 | | | 10000 | | | | |
| 701 | 10000 | 2210 | 4.5 | | | 10000 | | | 10000 | |
| 702 | 3950 | 890 | 4.4 | 10000 | 7500 | 3790 | 2 | 9860 | 7280 | 1.4 |
| 703 | 6490 | 830 | 7.8 | 1860 | 4140 | 1390 | 3 | 8180 | 4830 | 1.7 |
| 704 | 10000 | 1650 | 6.1 | 10000 | 10000 | 8750 | 1.1 | | 10000 | |
| 705 | 10000 | 2470 | 4 | 10000 | 10000 | 10000 | 1 | 10000 | 10000 | 1 |
| 706 | 3950 | 850 | 4.6 | 2870 | 4020 | 740 | 5.4 | 7980 | 3090 | 2.6 |
| 707 | 10000 | | | | | 10000 | | | | |
| 708 | 7670 | 3030 | 2.5 | | | 10000 | | 10000 | 10000 | 1 |
| 709 | 7500 | 2340 | 3.2 | | 10000 | 10000 | 1 | 10000 | | |
| 710 | 5010 | 1490 | 3.4 | 10000 | 10000 | 10000 | 1 | 10000 | 10000 | 1 |
| 711 | 10000 | 5160 | 1.9 | 10000 | | 10000 | | | | |
| 712 | 10000 | 6020 | 1.7 | | | 10000 | | 10000 | 10000 | 1 |
| 713 | | | | | | 10000 | | | | |
| 714 | 5770 | 2290 | 2.5 | | 10000 | 10000 | 1 | 10000 | | |
| 715 | 10000 | 1380 | 7.2 | | 10000 | 7510 | 1.3 | 10000 | 10000 | 1 |
| 716 | 6040 | 220 | 27.5 | 10000 | 10000 | 6840 | 1.5 | 7760 | 6540 | 1.2 |
| 717 | 8630 | 490 | 17.6 | | 10000 | 9980 | 1 | 10000 | 10000 | 1 |
| 718 | 4710 | 150 | 31.4 | 10000 | 5680 | 3670 | 1.5 | 10000 | 10000 | 1 |
| 719 | 1240 | 110 | 11.3 | 1780 | 730 | 640 | 1.1 | 2730 | 750 | 3.6 |
| 720 | 830 | 160 | 5.2 | 4500 | 3950 | 2050 | 1.9 | 7010 | 2450 | 2.9 |
| 721 | 4860 | 510 | 9.5 | 10000 | 8440 | 4320 | 2 | 10000 | 4570 | 2.2 |
| 722 | 1210 | 230 | 5.3 | 2760 | 3680 | 2660 | 1.4 | 8040 | 2560 | 3.1 |
| 723 | 3140 | 490 | 6.4 | 7860 | 6220 | 5100 | 1.2 | 10000 | 4890 | 2 |
| 724 | 980 | 90 | 10.9 | 1410 | 2240 | 830 | 2.7 | 5230 | 1210 | 4.3 |
| 725 | 1630 | 140 | 11.6 | 3600 | 2310 | 680 | 3.4 | 6700 | 2780 | 2.4 |
| 726 | 2770 | 900 | 3.1 | | 10000 | 10000 | 1 | 10000 | 10000 | 1 |
| 727 | 4100 | 950 | 4.3 | | | 10000 | | | 10000 | |
| 728 | 3640 | 1560 | 2.3 | | | 10000 | | 10000 | | |
| 729 | 6900 | 150 | 46 | 8240 | 8130 | 6780 | 1.2 | 10000 | 9300 | 1.1 |
| 730 | 9100 | 460 | 19.8 | | 10000 | 10000 | 1 | 10000 | 10000 | 1 |
| 731 | 5380 | 230 | 23.4 | 10000 | 6360 | 6670 | 1 | 10000 | 10000 | 1 |
| 732 | 920 | 60 | 15.3 | 3810 | 570 | 760 | 0.8 | 2890 | 620 | 4.7 |
| 733 | 500 | 50 | 10 | 6160 | 4610 | 1410 | 3.3 | 7270 | 3990 | 1.8 |
| 734 | 3390 | 130 | 26.1 | 10000 | 8700 | 2270 | 3.8 | 10000 | 5670 | 1.8 |
| 735 | 690 | 70 | 9.9 | 3510 | 4550 | 1460 | 3.1 | 6370 | 3710 | 1.7 |
| 736 | 2200 | 130 | 16.9 | 10000 | 6640 | 2730 | 2.4 | 10000 | 3320 | 3 |
| 737 | 860 | 40 | 21.5 | 2180 | 2060 | 520 | 4 | 4230 | 1280 | 3.3 |
| 738 | 1270 | 120 | 10.6 | 5750 | 2250 | 520 | 4.3 | 5360 | 2340 | 2.3 |
| 739 | 2890 | 1000 | 2.9 | | 10000 | 10000 | 1 | 8860 | 10000 | 0.9 |
| 740 | 3910 | 850 | 4.6 | 10000 | 10000 | 10000 | 1 | 10000 | 10000 | 1 |
| 741 | 3860 | 1310 | 2.9 | 10000 | 10000 | 10000 | 1 | 10000 | 10000 | 1 |
| 742 | 3360 | 1050 | 3.2 | | | 10000 | | | | |
| 743 | 3370 | 1460 | 2.3 | | | 10000 | | 10000 | 10000 | 1 |
| 744 | 4110 | 940 | 4.4 | 10000 | 10000 | | | 10000 | 10000 | 1 |
| 745 | 4220 | 1210 | 3.5 | 10000 | 10000 | 10000 | 1 | 8680 | 10000 | 0.9 |
| 746 | | 10000 | | | 10000 | 10000 | 1 | | | |
| 747 | 10000 | 5050 | 2 | 10000 | 9950 | 5570 | 1.8 | 10000 | 8800 | 1.1 |
| 748 | 10000 | 5240 | 1.9 | 10000 | 10000 | 8420 | 1.2 | 10000 | 10000 | 1 |
| 749 | | 10000 | | 10000 | 10000 | 10000 | 1 | | | |
| 750 | 2330 | 170 | 13.7 | 3370 | 10000 | 740 | 13.5 | 3420 | 990 | 3.5 |
| 751 | 3930 | 300 | 13.1 | 4530 | 10000 | 1480 | 6.8 | 4720 | 1350 | 3.5 |
| 752 | 3700 | 280 | 13.2 | 4910 | 5860 | 1880 | 3.1 | 8600 | 2810 | 3.1 |
| 753 | 3800 | 330 | 11.5 | 4450 | 10000 | 1630 | 6.1 | 7420 | 1510 | 4.9 |
| 754 | | 7220 | 1.4 | 10000 | | 10000 | | | | |
| 755 | 4480 | 750 | 6 | 5250 | 10000 | 2490 | 4 | 8170 | 3880 | 2.1 |
| 756 | 3900 | 390 | 10 | 1590 | 3520 | 1200 | 2.9 | 4190 | 1370 | 3.1 |
| 757 | 5790 | 550 | 10.5 | 2220 | 6060 | 2290 | 2.6 | 10000 | 5390 | 1.9 |
| 758 | 2540 | 390 | 6.5 | 2130 | 4060 | 1970 | 2.1 | 3560 | 1880 | 1.9 |
| 759 | 3670 | 480 | 7.6 | 2270 | 3550 | 2070 | 1.7 | 5100 | 2180 | 2.3 |
| 760 | 2870 | 190 | 15.1 | 2800 | 4620 | 730 | 6.3 | 10000 | 3550 | 2.8 |
| 761 | 1600 | 150 | 10.7 | 4190 | 10000 | 1710 | 5.8 | 8540 | 4610 | 1.9 |
| 762 | 6260 | 390 | 16.1 | 2630 | 10000 | 1840 | 5.4 | 10000 | 7230 | 1.4 |
| 763 | 2040 | 270 | 7.6 | 6440 | 7020 | 960 | 7.3 | 10000 | 2190 | 4.6 |
| 764 | 1020 | 110 | 9.3 | 780 | 2900 | 430 | 6.7 | 3240 | 830 | 3.9 |
| 765 | 3400 | 420 | 8.1 | 2010 | 4600 | 1500 | 3.1 | 6710 | 2250 | 3 |
| 766 | 1690 | 130 | 13 | 2650 | 4200 | 2380 | 1.8 | 3720 | 2070 | 1.8 |
| 767 | 5370 | 570 | 9.4 | 10000 | 10000 | 9570 | 1 | 10000 | 5140 | 1.9 |
| 768 | 4170 | 390 | 10.7 | 4570 | 6820 | 3560 | 1.9 | 10000 | 4420 | 2.3 |
| 769 | 4980 | 340 | 14.6 | 4720 | 5360 | 3780 | 1.4 | 8490 | 1470 | 5.8 |
| 770 | 10000 | 2290 | 4.4 | | 10000 | 10000 | 1 | 10000 | 10000 | 1 |
| 771 | 10000 | 2010 | 5 | 10000 | 5440 | 3040 | 1.8 | 10000 | 10000 | 1 |
| 772 | | 5670 | 1.8 | | 10000 | 10000 | 1 | 10000 | | |
| 773 | | 9880 | 1 | | 10000 | 10000 | 1 | | | |
| 774 | 9980 | 4320 | 2.3 | | 6630 | 10000 | 0.7 | 10000 | 6450 | 1.6 |
| 775 | 10000 | 5780 | 1.7 | 10000 | 8660 | 1620 | 5.3 | 10000 | 10000 | 1 |
| 776 | 10000 | 9370 | 1.1 | | | 10000 | | | | |
| 777 | 10000 | 10000 | 1 | | | 10000 | | | | |
| 778 | | 10000 | | | | 10000 | | | | |
| 779 | 10000 | 10000 | 1 | | | 10000 | | 10000 | 10000 | 1 |
| 780 | 10000 | 10000 | 1 | | 10000 | | | 10000 | 10000 | 1 |

### Example 51

### L5/L6 Spinal Nerve Ligation (SNL)-Chung Pain Model

The Spinal Nerve Ligation is an animal model representing peripheral nerve injury generating a neuropathic pain syndrome. In this model experimental animals develop the clinical symptoms of tactile allodynia and hyperalgesia. L5/L6 Spinal nerve ligation (SNL) injury was induced using the procedure of Kim and Chung (Kim et al., Pain 50:355-363 (1992)) in male Sprague-Dawley rats (Harlan; Indianapolis, Ind.) weighing 200 to 250 grams.

Anaesthesia was induced with 2% isofluorane in O₂ at 2 L/min and maintained with 0.5% isofluorane in O₂. Rats were then shaved and aseptically prepared for surgeries. A 2 cm paraspinal incision was made at the level of L4-S2. L4/L5 was exposed by removing the transverse process above the nerves with a small rongeur. The L5 spinal nerve is the larger of the two visible nerves below the transverse process and lies closest to the spine. The L6 spinal nerve is located beneath the corner of the slope bone. A home-made glass Chung rod was used to hook L5 or L6 and a pre-made slip knot of 4.0 silk suture was placed on the tip of the rod just above the nerve and pulled underneath to allow for the tight ligation. The L5 and L6 spinal nerves were tightly ligated distal to the dorsal root ganglion. The incision was closed, and the animals were allowed to recover for 5 days. Rats that exhibited motor deficiency (such as paw-dragging) or failure to exhibit subsequent tactile allodynia were excluded from further testing.

Sham control rats underwent the same operation and handling as the experimental animals, but without SNL.

Prior to initiating drug delivery, baseline behavioural testing data is obtained. At selected times after infusion of the Test or Control Article behavioural data can then be collected again.

### A. Assessment of Tactile Allodynia—Von Frey

The assessment of tactile allodynia consisted of measuring the withdrawal threshold of the paw ipsilateral to the site of nerve injury in response to probing with a series of calibrated von Frey filaments (innocuous stimuli). Animals were acclimated to the suspended wire-mesh cages for 30 min before testing. Each von Frey filament was applied perpendicularly to the plantar surface of the ligated paw of rats for 5 sec. A positive response was indicated by a sharp withdrawal of the paw. For rats, the first testing filament is 4.31. Measurements were taken before and after administration of test articles. The paw withdrawal threshold was determined by the non-parametric method of Dixon (Dixon, Ann. Rev. Pharmacol. Toxicol. 20:441-462 (1980)), in which the stimulus was incrementally increased until a positive response was obtained, and then decreased until a negative result was observed. The protocol was repeated until three changes in behaviour were determined (“up and down” method) (Chaplan et al., J. Neurosci. Methods 53:55-63 (1994)). The 50% paw withdrawal threshold was determined as (10^{[Xf+kδ]})/10,000, where X_{f}=the value of the last von Frey filament employed, k=Dixon value for the positive/negative pattern, and δ=the logarithmic difference between stimuli. The cut-off values for rats were no less than 0.2 g and no higher than 15 g (5.18 filament); for mice no less than 0.03 g and no higher than 2.34 g (4.56 filament). A significant drop of the paw withdrawal threshold compared to the pre-treatment baseline is considered tactile allodynia.

### B. Assessment of Thermal Hypersensitivity—Hargreaves

The method of Hargreaves and colleagues (Hargreaves et al., Pain 32:77-8 (1988)) can be employed to assess paw-withdrawal latency to a noxious thermal stimulus. Rats may be allowed to acclimate within a Plexiglas enclosure on a clear glass plate for 30 minutes. A radiant heat source (e.g., halogen bulb coupled to an infrared filter) can then be activated with a timer and focused onto the plantar surface of the affected paw of treated rats. Paw-withdrawal latency can be determined by a photocell that halts both lamp and timer when the paw is withdrawn. The latency to withdrawal of the paw from the radiant heat source can be determined prior to L5/L6 SNL, 7-14 days after L5/L6 SNL but before drug, as well as after drug administration. A maximal cut-off of 33 seconds is typically employed to prevent tissue damage. Paw withdrawal latency can be thus determined to the nearest 0.1 second. A significant drop of the paw withdrawal latency from the baseline indicates the status of thermal hyperalgesia. Antinociception is indicated by a reversal of thermal hyperalgesia to the pre-treatment baseline or a significant (p<0.05) increase in paw withdrawal latency above this baseline. Data is converted to % anti hyperalgesia or % anti nociception by the formula: (100×(test latency−baseline latency)/(cut-off−baseline latency) where cut-off is 21 seconds for determining anti hyperalgesia and 40 seconds for determining anti nociception.

### Example 52

### 6 Hz Psychomotor Seizure Model of Partial Epilepsy

Compounds can also be evaluated for the protection against seizures induced by a 6 Hz, 0.2 ms rectangular pulse width of 3 s duration, at a stimulus intensity of 32 mA (CC97) applied to the cornea of male CF1 mice (20-30 g) according to procedures described by Barton et al, “Pharmacological Characterization of the 6 Hz Psychomotor Seizure Model of Partial Epilepsy,” Epilepsy Res. 47(3):217-27 (2001). Seizures are characterised by the expression of one or more of the following behaviours: stun, forelimb clonus, twitching of the vibrissae and Straub-tail immediately following electrical stimulation. Animals can be considered “protected” if following pre-treatment with a compound the 6 Hz stimulus failed to evoke a behavioural response as describe above. Exemplary data are shown in Table 7.

TABLE 7

| | | Epilepsy 6 Hz (% protected) | | | | |
|---|---|---|---|---|---|---|
| | | 0.25 | 0.5 | | | |
| Compound | No. | hr | hr | 1 hr | 2 hr | 4 hr |
| 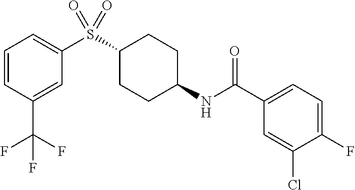 | 126 | 0 | 50 | 0 | 25 | 0 |
| 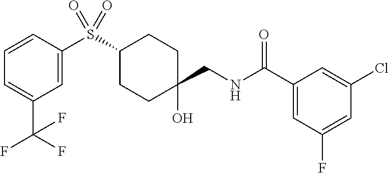 | 230 | 0 | 25 | 25 | 100 | 0 |
| 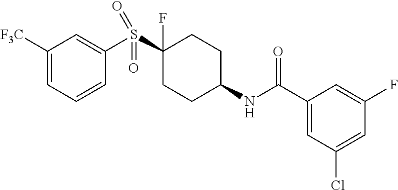 | 240 | 75 | 25 | 25 | 0 | 0 |
| 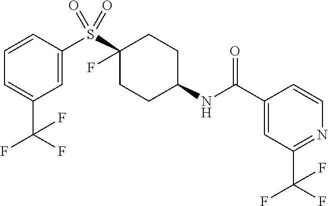 | 248 | 0 | 50 | 0 | 0 | 0 |
| 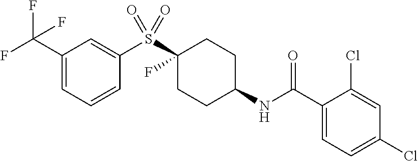 | 272 | 0 | 0 | 0 | 0 | 0 |
| 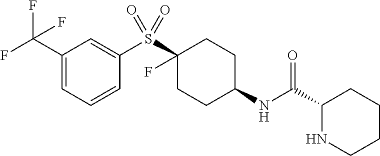 | Enan- tiomers of 285 | 0 | 100 | 100 | 100 | 0 |
| 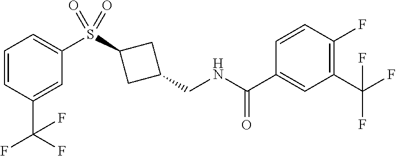 | 356 | 25 | 0 | 0 | 0 | 0 |
| 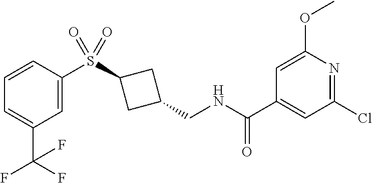 | 365 | 25 | 50 | 50 | 25 | 25 |
| 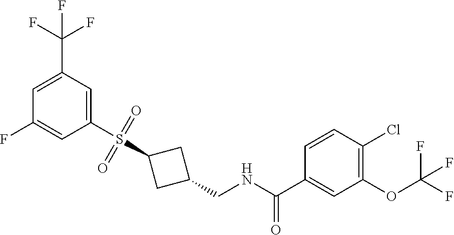 | 396 | 50 | 0 | 0 | 25 | 0 |
| 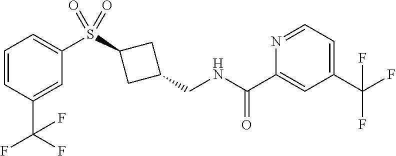 | 374 | 0 | 0 | 0 | 0 | 0 |
| 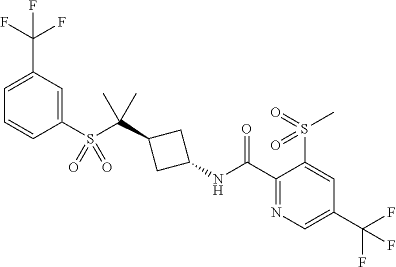 | 438 | 75 | 0 | 0 | 25 | 0 |
| 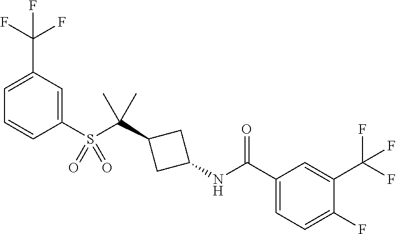 | 442 | 50 | 25 | 75 | 0 | 0 |
| 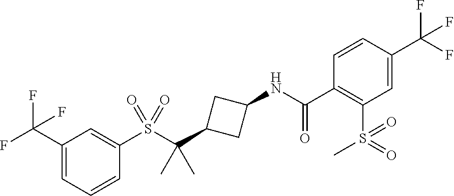 | 542 | 0 | 0 | 0 | 0 | 0 |
| 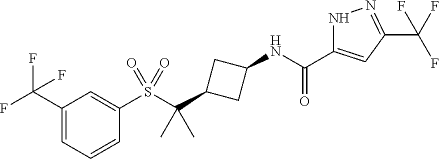 | 543 | 0 | 0 | 0 | 0 | 0 |
| 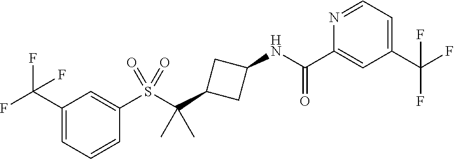 | 551 | 25 | 0 | 0 | 0 | 0 |

### Example 53

### Mouse Rotarod Assay

To assess a compound's undesirable side effects (toxicity), animals can be monitored for overt signs of impaired neurological or muscular function. In mice, the rotarod procedure (Dunham and Miya, J. Am. Pharmacol. Assoc. 46:208-209 (1957)) is used to disclose minimal muscular or neurological impairment (MMI). When a mouse is placed on a rod that rotates at a speed of 6 rpm, the animal can maintain its equilibrium for long periods of time. The animal is considered toxic if it falls off this rotating rod three times during a 1-min period. In addition to MMI, animals may exhibit a circular or zigzag gait, abnormal body posture and spread of the legs, tremors, hyperactivity, lack of exploratory behavior, somnolence, stupor, catalepsy, loss of placing response and changes in muscle tone. Exemplary data are shown in Table 8.

TABLE 8

| | |
|---|---|
| Compound | EPILEPSY ROTAROD (% IMPAIRED) |

| no. | 0.25 hr | 0.5 hr | 1 hr | 2 hr | 4 hr |
|---|---|---|---|---|---|
| 240 | 0 | 0 | 0 | 0 | 0 |
| 126 | 0 | 0 | 0 | 0 | 0 |
| 356 | 0 | 0 | 0 | 0 | 0 |
| 230 | 0 | 0 | 0 | 0 | 0 |
| 248 | 0 | 0 | 0 | 0 | 0 |
| 272 | 0 | 0 | 0 | 0 | 0 |
| 365 | 0 | 0 | 0 | 0 | 0 |
| 438 | 0 | 0 | 0 | 0 | 0 |
| 374 | 0 | 0 | 0 | 0 | 0 |
| 442 | 0 | 0 | 0 | 0 | 0 |
| 396 | 0 | 0 | 0 | 0 | 0 |
| Enantiomers | 100 | 100 | 100 | 100 | 0 |
| of 285 | | | | | |
| 542 | 0 | 0 | 0 | 0 | 0 |
| 543 | 0 | 0 | 0 | 0 | 0 |
| 551 | 0 | 0 | 0 | 0 | 0 |

### Example 54

### Lamina Assay and Data

### Recordings on Lamina I/II Spinal Cord Neurons.

Male Wistar rats (P6 to P9 for voltage-clamp and P15 to P18 for current-clamp recordings) were anaesthetized through intraperitoneal injection of Inactin (Sigma). The spinal cord was then rapidly dissected out and placed in an ice-cold solution protective sucrose solution containing (in mM): 50 sucrose, 92 NaCl, 15 D-Glucose, 26 NaHCO₃, 5 KCl, 1.25 NaH₂PO₄, 0.5 CaCl₂, 7 MgSO₄, 1 kynurenic acid, and bubbled with 5% CO₂/95% O₂. The meninges, dura, and dorsal and ventral roots were then removed from the lumbar region of the spinal cord under a dissecting microscope. The “cleaned” lumbar region of the spinal cord was glued to the vibratome stage and immediately immersed in ice cold, bubbled, sucrose solution. For current-clamp recordings, 300 to 350 μm parasagittal slices were cut to preserve the dendritic arbour of lamina I neurons, while 350 to 400 μm transverse slices were prepared for voltage-clamped Naᵥ channel recordings. Slices were allowed to recover for 1 hour at 35° C. in Ringer solution containing (in mM): 125 NaCl, 20 D-Glucose, 26 NaHCO₃, 3 KCl, 1.25 NaH₂PO₄, 2 CaCl₂, 1 MgCl₂, 1 kynurenic acid, 0.1 picrotoxin, bubbled with 5% CO₂/95% O₂. The slice recovery chamber was then returned to room temperature (20 to 22° C.) and all recordings were performed at this temperature.

Neurons were visualized using IR-DIC optics (Zeiss Axioskop 2 FS plus, Gottingen, Germany), and neurons from lamina I and the outer layer of lamina II were selected based on their location relative to the substantia gelatinosa layer. Neurons were patch-clamped using borosilicate glass patch pipettes with resistances of 3 to 6 MΩ. Current-clamp recordings of lamina I/II neurons in the intact slice, the external recording solution was the above Ringer solution, while the internal patch pipette solution contained (in mM): 140 KGluconate, 4 NaCl, 10 HEPES, 1 EGTA, 0.5 MgCl₂, 4 MgATP, 0.5 Na₂GTP, adjusted to pH 7.2 with 5 M KOH and to 290 mOsm with D-Mannitol (if necessary). Only tonic firing neurons were selected for current-clamp experiments, while phasic, delayed onset and single spike neurons were discarded (22). Recordings were digitized at 50 kHz and low-pass filtered at 2.4 kHz.

Exemplary data are shown in Table 9.

TABLE 9

| | LAMINA I and II | | |
|---|---|---|---|
| | % Spike Change | | |
| Structure | Mean | SEM | P <0.05? |
| 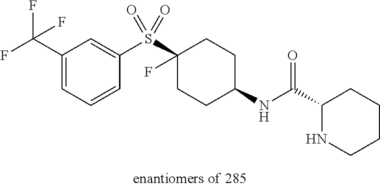 | −88.2 −57.1 | 5.9 11.4 | yes yes |
| 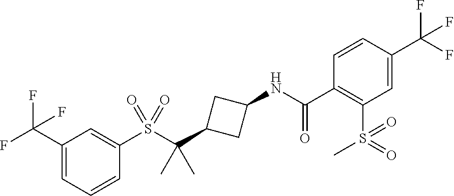 | −41 −40 −26 −10 | 6.3 12 13 15 | no yes |

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

All publications, patents and patent applications are herein incorporated by reference in their entirety to the same extent as if each individual publication, patent or patent application was specifically and individually indicated to be incorporated by reference in its entirety.

What is claimed is:

1. A compound having a structure according to the following formula, 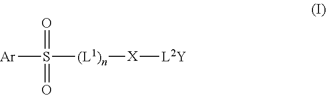
or a pharmaceutically acceptable salt, solvate, or prodrug thereof, or a stereoisomer thereof, or a conjugate thereof, wherein
Ar is an optionally substituted phenyl;
L¹ is methylenyl, ethylenyl, or propylenyl;
X is an optionally substituted cyclohexyl, an optionally substituted cyclobutyl, optionally substituted piperidinyl, or dimethylmethylenyl;
n is 0 or 1;
L² is (CH₂)₀₋₃CONR′(CH₂)₀₋₂, (CH₂)₀₋₃NR′CO, CH₂NR′CH₂CONR′, (CH₂)₀₋₃NR′CONR′, NR′COCH₂NR′, NR′CH₂CONR′, CH₂NHCH₂CONR′, NR′COO, NR′(CH₂)₁₋₃NR′CO, (CH₂)₀₋₃NR′SO₂, (CH₂)₀₋₃SO₂NR′(CH₂)₀₋₂, (CH₂)₁₋₂NR′(CH₂)₀₋₁, (CH₂)₁₋₂SO₂, or imidazolyl;
Y is H or an optionally substituted C1-C10 alkyl, C2-C10 alkenyl, C2-C10 alkynyl, C2-C10 heteroalkyl, C2-C10 heteroalkenyl, C2-C10 heteroalkynyl, C4-C10 heterocycloalkyl, C6-C10 aryl, heteroaryl (5-12 ring members), C3-C10 cycloalkyl, heterocyclyl (5-12 ring members), aryl(5-12 ring members)-C1-C10 alkyl; or R′ from L² and Y may together form an optionally substituted heterocyclic ring (4-8 ring members); and
each R′ is, independently, H, methyl, ethyl or propyl.

2. The compound of claim 1, wherein Ar comprises a substituent selected from halo, CN, CF₃, OCF₃, COOR″, CONR″2, OR″, SR″, SOR″, SO₂R″, C1-C6 alkyl, C2-C6 alkenyl, C2-C6 alkynyl, C2-C6 heteroalkyl, C2-C6 heteroalkenyl, C2-C6 heteroalkynyl; C6-C10 aryl, heteroaryl (5-12 ring members), O-(C6-C10)aryl, O-heteroaryl (5-12 ring members), C6-C10 aryl-C1-C6 alkyl, or heteroaryl (5-12 ring members)-alkyl (1-6C), and
wherein each R″ is independently H or an optionally substituted group selected from C1-C6 alkyl, C2-C6 alkenyl, C2-C6 alkynyl, C2-C6 heteroalkyl, C2-C6 heteroalkenyl, or C2-C6 heteroalkynyl
and/or
wherein Y comprises a substituent selected from halo, CN, CF₃, OCF₃, COOR″, CONR″2, OR″, SR″, SOR″, SO₂R″, C1-C6 alkyl, C2-C6 alkenyl, C2-C6 alkynyl, C2-C6 heteroalkyl, C2-C6 heteroalkenyl, C2-C6 heteroalkynyl; C6-C10 aryl, heteroaryl (5-12 ring members), O—(C6-C10)aryl, O-heteroaryl (5-12 ring members), C6-C10 aryl-C1-C6 alkyl, heteroaryl (5-12 ring members)-alkyl (1-6C), ═O, ═NOR″, NO₂, NR″2, NR″(CO)R″, or NR″SO₂R″, and
wherein each R″ is independently H or an optionally substituted group selected from C1-C6 alkyl, C2-C6 alkenyl, C2-C6 alkynyl, C2-C6 heteroalkyl, C2-C6 heteroalkenyl, C2-C6 heteroalkynyl;
and/or
wherein said optional substituents on X are selected, independently, from halo, methyl, ethyl, propyl, and OR′, and each R′ is, independently, H, methyl, ethyl or propyl
and/or
Ar is phenyl substituted by F, CF₃, or OCF₃;
and/or
Y is phenyl, heteroaryl, or C₁-C₆ alkyl comprising a substituent selected from CF₃, F, Cl, OCF₃, SO₂Me, and SO₂(ⁱPr);
and/or
wherein L² is —NHCO—, —NCH₃CO—, or —NHSO₂—.

3.-5. (canceled)

6. The compound of claim 1, wherein when X is an optionally substituted cyclohexyl or optionally substituted piperidinyl, one of ArSO₂(L¹)ₙX-and-L² is located at C1 and the other is located at C4 or N4, or when X is an optionally substituted cyclobutyl, one of ArSO₂(L¹)ₙX-and-L² is located at C1 and the other is located at C3.

7. The compound of claim 1, wherein when X is cyclohexyl, and said cyclohexyl is unsubstituted or substituted by a methyl group.

8.-9. (canceled)

10. The compound of claim 1, wherein said compound has a structure according to one of the following formulas,
(A) 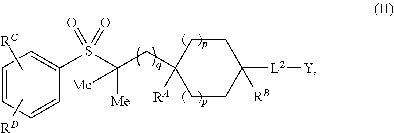
wherein
each of R^{A} and R^{B} is selected, independently, from H, OH, optionally substituted C1-C3 alkyl, and halogen;
R^{C} is CF₃ or OCF₃;
R^{D} is H, halogen, or CF₃;
both p are 0, or both p are 1;
q is 0 or 1;
L² is selected from —NR′CO—, —CONR′—, —NR′CH₂CONH—, —CH₂NR′CO—, —CH₂NR′CH₂CONR′—, —NR′COCH₂NR′—, —NR′CONR′—, —NR′COO—, —NR′SO₂—;
each R′ is selected, independently, from H or CH₃; and
Y is H, optionally substituted phenyl, optionally substituted heteroaryl, unsubstituted C1-C6 alkyl, C1-C6 haloalkyl, C3-C6 cycloalkyl, or heterocyclyl; or
(B) 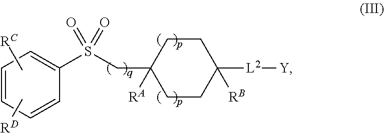
wherein
each of R^{A} and R^{B} is selected, independently, from H, OH, optionally substituted C1-C3 alkyl, and halogen;
R^{C} is CF₃ or OCF₃;
R^{D} is H, halogen, or CF₃;
both p are 0, or both p are 1;
q is 0 or 1;
L² is selected from —NR′CO—, —CONR′—, —NR′CH₂CONH—, —CH₂NR′CO—, —CH₂NR′CH₂CONR′—, —NR′COCH₂NR′—, —NR′CONR′—, —NR′COO—, —NR′SO₂—;
each R′ is selected from H or CH₃; and
Y is H, optionally substituted phenyl, optionally substituted heteroaryl, unsubstituted C1-C6 alkyl, C1-C6 haloalkyl, C3-C6 cycloalkyl, or heterocyclyl; or
(C) 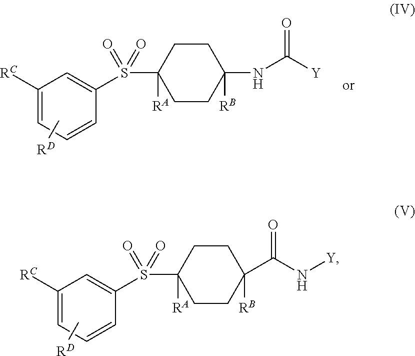
wherein
each of R^{A} and R^{B} is selected, independently, from H, OH, optionally substituted C1-C3 alkyl, and halogen;
R^{C} is CF₃ or OCF₃; and
R^{D} is H, halogen, or CF₃; or
(D) 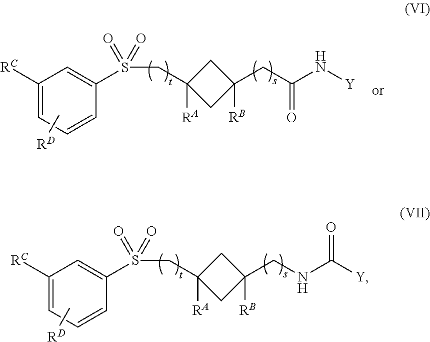
wherein
s is 0 or 1;
t is 0 or 1;
each of R^{A} and R^{B} is selected, independently, from H, OH, optionally substituted C1-C3 alkyl, and halogen;
R^{C} is CF₃ or OCF₃; and
R^{D} is H, halogen, or CF₃; or
(E) 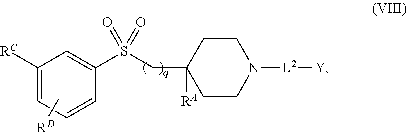
wherein
R^{A} is H, OH, optionally substituted C1-C3 alkyl, and halogen;
q is 0, 1, or 2;
R^{C} is CF₃ or OCF₃; and
R^{D} is H, halogen, or CF₃; or
(F) 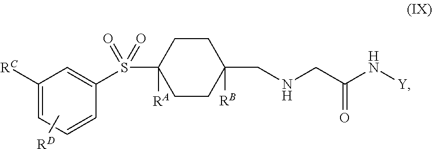
wherein
each of R^{A} and R^{B} is selected, independently, from H, OH, optionally substituted C1-C3 alkyl, and halogen;
R^{C} is CF₃ or OCF₃; and
R^{D} is H, halogen, or CF₃; or
(G) 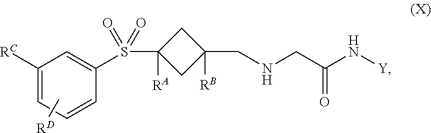
wherein
each of R^{A} and R^{B} is selected, independently, from H, OH, optionally substituted C1-C3 alkyl, and halogen;
R^{C} is CF₃ or OCF₃; and
R^{D} is H, halogen, or CF₃;
(H) 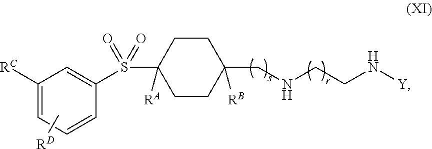
wherein
r is 1 or 2;
s is 0 or 1;
each of R^{A} and R^{B} is selected, independently, from H, OH, optionally substituted C1-C3 alkyl, and halogen;
R^{C} is CF₃ or OCF₃; and
R^{D} is H, halogen, or CF₃; or
(I) 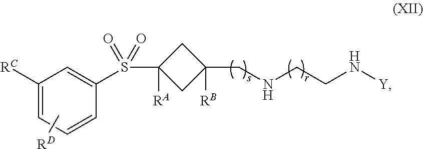
wherein
r is 1 or 2;
s is 0 or 1;
each of R^{A} and R^{B} is selected, independently, from H, OH, optionally substituted C1-C3 alkyl, and halogen;
R^{C} is CF₃ or OCF₃; and
R^{D} is H, halogen, or CF₃;
(I) 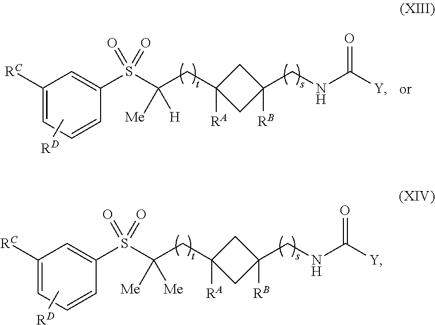
wherein
s is 0 or 1;
t is 0 or 1;
each of R^{A} and R^{B} is selected, independently, from H, OH, optionally substituted C1-C3 alkyl, and halogen;
R^{C} is CF₃ or OCF₃; and
R^{D} is H, halogen, or CF₃; or
(K) 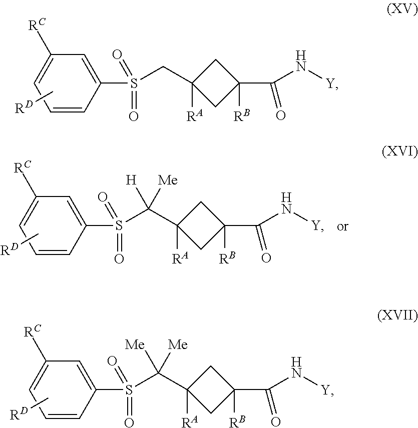
wherein
each of R^{A} and R^{B} is selected, independently, from H, OH, optionally substituted C1-C3 alkyl, and halogen;
R^{C} is CF₃ or OCF₃; and
R^{D} is H, halogen, or CF₃; or
(L) 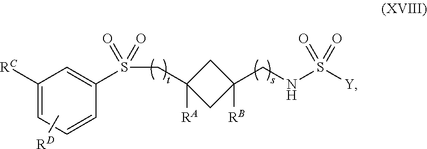
wherein
s is 0 or 1;
t is 0 or 1;
each of R^{A} and R^{B} is selected, independently, from H, OH, optionally substituted C1-C3 alkyl, and halogen;
R^{C} is CF₃ or OCF₃; and
R^{D} is H, halogen, or CF₃; or
(M) 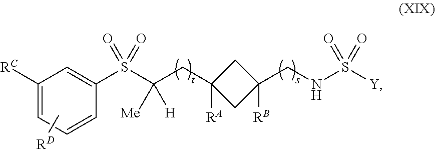
wherein
s is 0 or 1;
t is 0 or 1;
each of R^{A} and R^{B} is selected, independently, from H, OH, optionally substituted C1-C3 alkyl, and halogen;
R^{C} is CF₃ or OCF₃; and
R^{D} is H, halogen, or CF₃; or
(N) 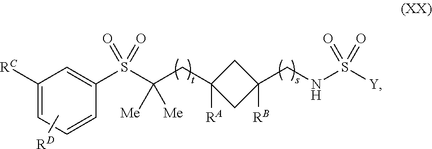
wherein
s is 0 or 1;
t is 0 or 1;
each of R^{A} and R^{B} is selected, independently, from H, OH, optionally substituted C1-C3 alkyl, and halogen;
R^{C} is CF₃ or OCF₃; and
R^{D} is H, halogen, or CF₃; or
(O) 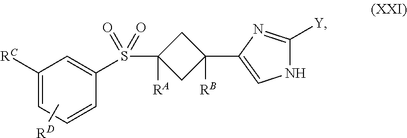
wherein
each of R^{A} and R^{B} is selected, independently, from H, OH, optionally substituted C1-C3 alkyl, and halogen;
R^{C} is CF₃ or OCF₃; and
R^{D} is H, halogen, or CF₃; or
(P) 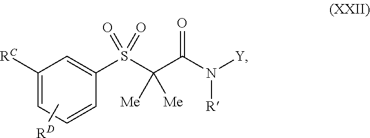
wherein
R′ is H or CH₃;
R^{C} is CF₃ or OCF₃; and
R^{D} is H, halogen, or CF₃; or
(Q) 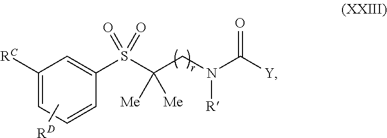
wherein
r is 1 or 2;
R′ is H or CH₃;
R^{C} is CF₃ or OCF₃; and
R^{D} is H, halogen, or CF₃; or
(R) 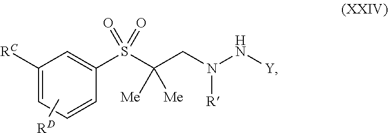
wherein
R′ is H or CH₃;
R^{C} is CF₃ or OCF₃; and
R^{D} is H, halogen, or CF₃; or
(S) 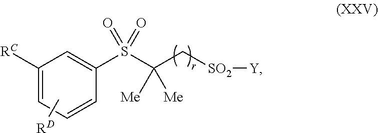
wherein
r is 1 or 2;
R′ is H or CH₃;
R^{C} is CF₃ or OCF₃; and
R^{D} is H, halogen, or CF₃; or
(T) 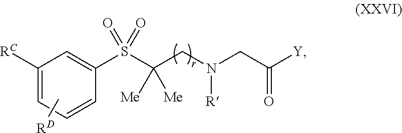
wherein
r is 1 or 2;
R′ is H or CH₃;
R^{C} is CF₃ or OCF₃; and
R^{D} is H, halogen, or CF₃; or
(U) 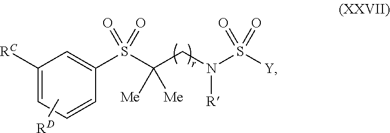
herein
r is 1 or 2;
R′ is H or CH₃;
R^{C} is CF₃ or OCF₃; and
R^{D} is H, halogen, or CF₃.

11.-46. (canceled)

47. The compound of claim 10, wherein
Y is optionally substituted C1-C10 alkyl, optionally substituted C2-C10 heteroalkyl, optionally substituted C6-C10 aryl, optionally substituted heteroaryl, optionally substituted C3-C10 cycloalkyl, or optionally substituted heterocyclyl (5-12 ring members).

48. The compound of claim 47, wherein Y is optionally substituted C1-C5 alkyl or optionally substituted C2-C6 heteroalkyl, or
wherein Y is optionally substituted tetrahydropyranyl, optionally substituted 1,4-morpholino, optionally substituted cyclobutyl, optionally substituted cyclopentyl, optionally substituted cyclohexyl, optionally substituted phenyl, optionally substituted pyrimidinyl, optionally substituted pyridyl, optionally substituted pyrazolyl, optionally substituted oxazolyl, optionally substituted isoxazolyl, optionally substituted benzimidazolyl, optionally substituted triazolyl, optionally substituted thiazolyl, optionally substituted isothiazolyl, optionally substituted furyl, optionally substituted thienyl, optionally substituted imidazolyl, optionally substituted imidazo[1,2-a]pyridine, optionally substituted 1,6-naphthyridine, optionally substituted 2,3-dihydroindolyl, optionally substituted phthalimido, or optionally substituted oxo-isoindolyl.

49. (canceled)

50. (canceled)

51. The compound of claim 47, wherein Y is optionally substituted phenyl, optionally substituted pyrimidinyl, or optionally substituted pyridyl.

52. The compound of claim 47, wherein
Y is substituted by F, Cl, CF₃, —SO₂Me, or —SO₂ⁱPr, and optionally substituted by halogen, C1-C3 alkoxy, C1-C3 alkyl, C1-C3 haloalkyl, C3-C6 cycloalkyl, halophenyl, or —SO₂(C1-C4 alkyl); or
wherein Y is unsubstituted or substituted by NH₂, halo, optionally substituted phenyl, optionally substituted benzyl, or optionally substituted pyridyl.

53. (canceled)

54. The compound of claim 10, wherein
R^{A} and R^{B} are cis to each other; or
the carbon substituted by R^{A} has the S configuration; or
the carbon substituted by R^{B} has the S configuration.

55. The compound of claim 10, wherein
R^{A} and R^{B} are trans to each other; or
the carbon substituted by R^{A} has the R configuration; or
the carbon substituted by R^{B} has the R configuration.

56.-59. (canceled)

60. The compound of claim 10, wherein R^{C} is CF₃ or OCF₃.

61. (canceled)

62. The compound of claim 1, wherein said compound has the structure of any of compounds 1-780 in Table 1, or a pharmaceutically acceptable salt, solvate, or prodrug thereof, or a stereoisomer thereof, or a conjugate thereof.

63. The compound of claim 62, wherein said compound is 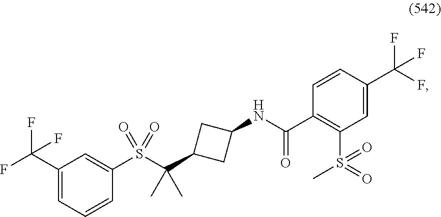
or a pharmaceutically acceptable salt, solvate, or prodrug thereof, or a stereoisomer thereof, or a conjugate thereof.

64. A pharmaceutical composition comprising the compound of claim 1, or a pharmaceutically acceptable salt, solvate, or prodrug thereof, or a stereoisomer thereof, or a conjugate thereof, and a pharmaceutically acceptable carrier or excipient.

65. The pharmaceutical composition of claim 64, wherein said pharmaceutical composition is formulated in unit dosage form.

66. The pharmaceutical composition of claim 65, wherein said unit dosage form is a tablet, caplet, capsule, lozenge, film, strip, gelcap, or syrup.

67. A method to treat a condition modulated by calcium channel activity, said method comprising administering to a subject in need of such treatment an effective amount of the compound of claim 1, or a pharmaceutically acceptable salt, solvate, or prodrug thereof, or a stereoisomer thereof, or a conjugate thereof, or the pharmaceutical composition thereof.

68. The method of claim 67, wherein said calcium channel is a T-type calcium channel or an N-type calcium channel.

69. The method of claim 68, wherein said calcium channel is the Cav 3.1, Cav 3.2, Cav 3.3 channel, or Cav 2.2 channel.

70.-71. (canceled)

72. The method of claim 67, wherein said condition is pain, epilepsy, Parkinson's disease, depression, psychosis, or tinnitus.

73. The method of claim 72, wherein
said psychosis is schizophrenia; or
said pain is inflammatory pain, neuropathic pain, or chronic pain.

74.-76. (canceled)

77. The method of claim 73, wherein said chronic pain is peripheral neuropathic pain; central neuropathic pain, musculoskeletal pain, headache, visceral pain, or mixed pain.

78. The method of claim 77, wherein
said peripheral neuropathic pain is post-herpetic neuralgia, diabetic neuropathic pain, neuropathic cancer pain, failed back-surgery syndrome, trigeminal neuralgia, or phantom limb pain;
said central neuropathic pain is multiple sclerosis related pain, Parkinson disease related pain, post-stroke pain, post-traumatic spinal cord injury pain, or pain in dementia;
said musculoskeletal pain is osteoarthritic pain and fibromyalgia syndrome; inflammatory pain such as rheumatoid arthritis, or endometriosis;
said headache is migraine, cluster headache, tension headache syndrome, facial pain, or headache caused by other diseases;
said visceral pain is interstitial cystitis, irritable bowel syndrome, or chronic pelvic pain syndrome; or
said mixed pain is lower back pain, neck and shoulder pain, burning mouth syndrome, or complex regional pain syndrome.

79. (canceled)

80. The method of claim 72, wherein said pain is acute pain.

81. The method of claim 80, wherein said acute pain is nociceptive pain or post-operative pain.

82.-83. (canceled)
